(19) United States
(12) Patent Application Publication
(10) Pub. No.: US 2009/0264401 A1
(43) Pub. Date: Oct. 22, 2009

(54) SUBSTITUTED PYRIMIDIN-5-CARBOXAMIDES 281

(75) Inventors: Adrian Liam GILL, Macclesfield (GB); Andrew Leach, Macclesfield (GB); Martin Packer, Macclesfield (GB); James Stewart Scott, Macclesfield (GB); Pernilla Sorme, Macclesfield (GB); John Gibbin Swales, Macclesfield (GB); Paul Robert Owen Whittamore, Macclesfield (GB)

Correspondence Address: MORGAN LEWIS & BOCKIUS LLP, 1111 PENNSYLVANIA AVENUE NW, WASHINGTON, DC, 20004, US

(73) Assignee: AstraZeneca AB, Sodertalje (SE)

(21) Appl. No.: 12/427,170

(22) Filed: Apr. 21, 2009

Related U.S. Application Data
(60) Provisional application No. 61/046,836, filed on Apr. 22, 2008.
(60) Provisional application No. 61/140,201, filed on Dec. 23, 2008.

(51) Int. Cl. A61K 31/5377; C07D 413/04; A61K 31/505; C07D 239/28; C07D 239/38; A61P 3/10; C07D 413/14; A61K 31/554; C07D 419/04

(52) U.S. Cl. 514/210.2; 544/122; 514/235.8; 514/256; 544/335; 544/316; 514/274; 544/82; 514/232.2; 514/211.15; 540/544

(57) ABSTRACT

A compound of formula (I):

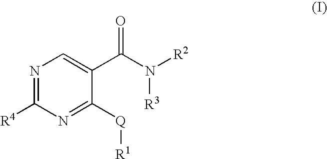

and pharmaceutically-acceptable salts thereof wherein the variable groups are defined within; their use in the inhibition of 11βHSD1, processes for making them and pharmaceutical compositions comprising them are also described.

This application claims the benefit under 35 U.S.C. § 119(e) of Application No. 61/046,836, filed on 22 Apr. 2008, and Application No. 61/140,201, filed on 23 Dec. 2008.

This invention relates to chemical compounds, or pharmaceutically-acceptable salts thereof. These compounds possess human 11-β-hydroxysteroid dehydrogenase type 1 enzyme (11βHSD1) inhibitory activity and accordingly have value in the treatment of disease states including metabolic syndrome and are useful in methods of treatment of a warm-blooded animal, such as man. The invention also relates to processes for the manufacture of said compounds, to pharmaceutical compositions containing them and to their use in the manufacture of medicaments to inhibit 11βHSD1 in a warm-blooded animal, such as man.

Glucocorticoids (cortisol in man, corticosterone in rodents) are counter regulatory hormones i.e. they oppose the actions of insulin (Dallman M F, Strack A M, Akana S F et al. 1993; Front Neuroendocrinol 14, 303-347). They regulate the expression of hepatic enzymes involved in gluconeogenesis and increase substrate supply by releasing glycerol from adipose tissue (increased lipolysis) and amino acids from muscle (decreased protein synthesis and increased protein degradation). Glucocorticoids are also important in the differentiation of pre-adipocytes into mature adipocytes which are able to store triglycerides (Bujalska I J et al. 1999; Endocrinology 140, 3188-3196). This may be critical in disease states where glucocorticoids induced by “stress” are associated with central obesity which itself is a strong risk factor for type 2 diabetes, hypertension and cardiovascular disease (Bjorntorp P & Rosmond R 2000; Int. J. Obesity 24, S80-S85).

It is now well established that glucocorticoid activity is controlled not simply by secretion of cortisol but also at the tissue level by intracellular interconversion of active cortisol and inactive cortisone by the 11-beta hydroxysteroid dehydrogenases, 11βHSD1 (which activates cortisone) and 11βHSD2 (which inactivates cortisol) (Sandeep T C & Walker B R 2001 Trends in Endocrinol & Metab. 12, 446-453). That this mechanism may be important in man was initially shown using carbenoxolone (an anti-ulcer drug which inhibits both 11βHSD1 and 2) treatment which (Walker B R et al. 1995; J. Clin. Endocrinol. Metab. 80, 3155-3159) leads to increased insulin sensitivity indicating that 11βHSD1 may well be regulating the effects of insulin by decreasing tissue levels of active glucocorticoids (Walker B R et al. 1995; J. Clin. Endocrinol. Metab. 80, 3155-3159).

Clinically, Cushing's syndrome is associated with cortisol excess which in turn is associated with glucose intolerance, central obesity (caused by stimulation of pre-adipocyte differentiation in this depot), dyslipidaemia and hypertension. Cushing's syndrome shows a number of clear parallels with metabolic syndrome. Even though the metabolic syndrome is not generally associated with excess circulating cortisol levels (Jessop D S et al. 2001; J. Clin. Endocrinol. Metab. 86, 4109-4114) abnormally high 11βHSD1 activity within tissues would be expected to have the same effect. In obese men it was shown that despite having similar or lower plasma cortisol levels than lean controls, 11βHSD1 activity in subcutaneous fat was greatly enhanced (Rask E et al. 2001; J. Clin. Endocrinol. Metab. 1418-1421). Furthermore, the central fat, associated with the metabolic syndrome expresses much higher levels of 11βHSD1 activity than subcutaneous fat (Bujalska I J et al. 1997; Lancet 349, 1210-1213). Thus there appears to be a link between glucocorticoids, 11βHSD1 and the metabolic syndrome.

11βHSD1 knock-out mice show attenuated glucocorticoid-induced activation of gluconeogenic enzymes in response to fasting and lower plasma glucose levels in response to stress or obesity (Kotelevtsev Y et al. 1997; Proc. Natl. Acad. Sci. USA 94, 14924-14929) indicating the utility of inhibition of 11βHSD1 in lowering of plasma glucose and hepatic glucose output in type 2 diabetes. Furthermore, these mice express an anti-atherogenic lipoprotein profile, having low triglycerides, increased HDL cholesterol and increased apo-lipoprotein AI levels. (Morton N M et al. 2001; J. Biol. Chem. 276, 41293-41300). This phenotype is due to an increased hepatic expression of enzymes of fat catabolism and PPARα. Again this indicates the utility of 11βHSD1 inhibition in treatment of the dyslipidaemia of the metabolic syndrome.

The most convincing demonstration of a link between the metabolic syndrome and 11βHSD1 comes from recent studies of transgenic mice over-expressing 11βHSD1 (Masuzaki H et al. 2001; Science 294, 2166-2170). When expressed under the control of an adipose specific promoter, 11βHSD1 transgenic mice have high adipose levels of corticosterone, central obesity, insulin resistant diabetes, hyperlipidaemia and hyperphagia. Most importantly, the increased levels of 11βHSD1 activity in the fat of these mice are similar to those seen in obese subjects. Hepatic 11βHSD1 activity and plasma corticosterone levels were normal, however, hepatic portal vein levels of corticosterone were increased 3 fold and it is thought that this is the cause of the metabolic effects in liver.

Overall it is now clear that the complete metabolic syndrome can be mimicked in mice simply by overexpressing 11βHSD1 in fat alone at levels similar to those in obese man.

11βHSD1 tissue distribution is widespread and overlapping with that of the glucocorticoid receptor. Thus, 11βHSD1 inhibition could potentially oppose the effects of glucocorticoids in a number of physiological/pathological roles. 11βHSD1 is present in human skeletal muscle and glucocorticoid opposition to the anabolic effects of insulin on protein turnover and glucose metabolism are well documented (Whorwood C B et al. 2001; J. Clin. Endocrinol. Metab. 86, 2296-2308). Skeletal muscle must therefore be an important target for 11βHSD1 based therapy.

Glucocorticoids also decrease insulin secretion and this could exacerbate the effects of glucocorticoid induced insulin resistance. Pancreatic islets express 11βHSD1 and carbenoxolone can inhibit the effects of 11-dehydocorticosterone on insulin release (Davani B et al. 2000; J. Biol. Chem. 275, 34841-34844). Thus in treatment of diabetes 11βHSD1 inhibitors may not only act at the tissue level on insulin resistance but also increase insulin secretion itself.

Skeletal development and bone function is also regulated by glucocorticoid action. 11βHSD1 is present in human bone osteoclasts and osteoblasts and treatment of healthy volunteers with carbenoxolone showed a decrease in bone resorption markers with no change in bone formation markers (Cooper M S et al 2000; Bone 27, 375-381). Inhibition of 11βHSD1 activity in bone could be used as a protective mechanism in treatment of osteoporosis.

Glucocorticoids may also be involved in diseases of the eye such as glaucoma. 11βHSD1 has been shown to affect intraocular pressure in man and inhibition of 11βHSD1 may be expected to alleviate the increased intraocular pressure associated with glaucoma (Rauz S et al. 2001; Investigative Ophthalmology & Visual Science 42, 2037-2042).

There appears to be a convincing link between 11βHSD1 and the metabolic syndrome both in rodents and in humans. Evidence suggests that a drug which specifically inhibits 11βHSD1 in type 2 obese diabetic patients will lower blood glucose by reducing hepatic gluconeogenesis, reduce central obesity, improve the atherogenic lipoprotein phenotype, lower blood pressure and reduce insulin resistance. Insulin effects in muscle will be enhanced and insulin secretion from the beta cells of the islet may also be increased.

Currently there are two main recognised definitions of metabolic syndrome.

1) The Adult Treatment Panel (ATP III 2001 JMA) definition of metabolic syndrome indicates that it is present if the patient has three or more of the following symptoms:
Waist measuring at least 40 inches (102 cm) for men, 35 inches (88 cm) for women;
Serum triglyceride levels of at least 150 mg/dl (1.69 mmol/l);
HDL cholesterol levels of less than 40 mg/dl (1.04 mmol/l) in men, less than 50 mg/dl (1.29 mmol/l) in women;
Blood pressure of at least 135/80 mm Hg; and/or Blood sugar (serum glucose) of at least 110 mg/dl (6.1 mmol/l).
2) The WHO consultation has recommended the following definition which does not imply causal relationships and is suggested as a working definition to be improved upon in due course:
The patient has at least one of the following conditions: glucose intolerance, impaired glucose tolerance (IGT) or diabetes mellitus and/or insulin resistance; together with two or more of the following:

### Raised Arterial Pressure;

Raised plasma triglycerides

### Central Obesity

### Microalbuminuria

We have found that the compounds defined in the present invention, or a pharmaceutically-acceptable salt thereof, are effective 11βHSD1 inhibitors, and accordingly have value in the treatment of disease states associated with metabolic syndrome.

Accordingly there is provided a compound of formula (1):

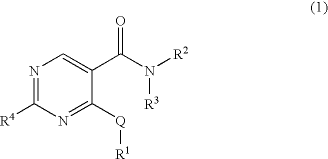

wherein:
Q is O, S, N(R⁸) or a single bond;
R⁸ is selected from hydrogen, C₁₋₄alkyl, C₃₋₅cycloalkyl and C₃₋₅cycloalkylmethyl (each of which is optionally substituted by 1, 2 or 3 fluoro atoms);
R¹ is selected from C₁₋₆alkyl, C₂₋₆alkenyl, C₂₋₆alkynyl, C₃₋₇cycloalkyl, heterocyclyl, heteroaryl, aryl, arylC₁₋₃alkyl, heteroarylC₁₋₃alkyl, C₃₋₇cycloalkylC₁₋₃alkyl, heterocyclylC₁₋₃alkyl, C₃₋₇cycloalkylC₂₋₃alkenyl and C₃₋₇cycloalkylC₂₋₃alkynyl, (each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₙ— (wherein n is 0, 1, 2 or 3), R⁵CON(R^{5′})-, (R^{5′})(R^{5″})N—, (R^{5′})(R^{5″})NC(O)—, R^{5′}OC(O)O—, R^{5′}OC(O)—, (R^{5′})(R^{5″})NC(O)N(R^{5′})-, R⁵SO₂N(R^{5″})-, (R^{5′})(R^{5″})NSO₂— and C₁₋₂alkyl optionally substituted by 1, 2 or 3 substituents independently selected from hydroxy, halo, carboxy and C₁₋₃alkoxy (wherein R⁵ is C₁₋₃alkyl optionally substituted by 1, 2 or 3 substituents independently selected from hydroxyl, halo and cyano; and
R^{5′}, R^{5″} and R^{5′″} are independently selected from hydrogen and C₁₋₃alkyl optionally substituted by 1, 2 or 3 substituents independently selected from hydroxyl, halo, C₁₋₃alkoxy, carboxy and cyano or R^{5′} and R^{5″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano]; or
R¹ and R⁸ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁹ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R² is selected from C₃₋₇cycloalkyl(CH₂)ₘ—, and C₆₋₁₂polycycloalkyl(CH₂)ₘ— (wherein m is 0, 1 or 2 and the rings optionally contain 1 or 2 ring atoms independently selected from nitrogen, oxygen and sulphur are optionally substituted, on available carbon atoms, by 1, 2 or 3 substituents independently selected from R⁶ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano);
R³ is selected from hydrogen, C₁₋₄alkyl, C₃₋₅cycloalkyl and C₃₋₅cycloalkylmethyl (each of which is optionally substituted by 1, 2 or 3 fluoro atoms);
R² and R³ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁷ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R⁴ is selected from hydrogen, R¹⁰, —OR¹⁰, —SR¹⁰ and —NR¹¹R¹²;
R¹⁰ is selected from C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, heterocyclylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₚ— (wherein p is 0, 1, 2 or 3), R¹³CON(R^{13′})-, (R^{13′})(R^{13″})N—, (R^{13′})(R^{13″})NC(O)—, R^{13′}OC(O)O—, R^{13′}OC(O)—, (R^{13′})(R^{13″})NC(O)N(R^{13′″})—, R¹³SO₂N(R^{13″})-, (R^{13′})(R^{13″})NSO₂— and C₁₋₂alkyl optionally substituted by 1, 2 or 3 substituents independently selected from hydroxy, halo, carboxy and C₁₋₃alkoxy (wherein R¹³ is C₁₋₃alkyl optionally substituted by 1, 2 or 3 substituents selected from hydroxyl, halo and cyano; and
R^{13′} R^{13″} and R^{13′″} are independently selected from hydrogen and C₁₋₃alkyl optionally substituted by 1, 2 or 3 substituents independently selected from hydroxyl, halo, C₁₋₃alkoxy, carboxy and cyano or R^{13′} and R^{13″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano];
R¹¹ is selected from hydrogen, C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, heterocyclylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})—, (R^{14′})(R^{14″})NC(O)—, R^{14′}OC(O)O—, R^{14′}OC(O)—, (R^{14′})(R^{14″})NC(O)N(R^{14′″})-, R¹⁴SO₂N(R^{14″})-, (R^{14′})(R^{14″})NSO₂— and C₁₋₂alkyl optionally substituted by 1, 2 or 3 substituents independently selected from hydroxy, halo, carboxy and C₁₋₃alkoxy (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo, C₁₋₃alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano]; and
R¹² is selected from hydrogen, C₁₋₄alkyl, C₃₋₅cycloalkyl and C₃₋₅cycloalkylmethyl (each of which is optionally substituted by 1, 2 or 3 fluoro atoms); or
R¹¹ and R¹² together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted, on available carbon atoms, by 1, 2, or 3 substituents independently selected from R¹⁵ and optionally substituted on an available nitrogen by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R⁶, R⁷, R⁹ and R¹⁵ are independently selected from hydroxyl, halo, oxo, carboxy, cyano, trifluoromethyl, R¹⁶, R¹⁶O—, R¹⁶CO—, R¹⁶C(O)O—, R¹⁶CON(R^{16′})-, (R^{16′})(R^{16″})NC(O)—, (R^{16′})(R^{16″})N—, R¹⁶S(O)ₐ— wherein a is 0 to 2, R^{16′}OC(O)—, (R^{16′})(R^{16″})NSO₂—, R¹⁶SO₂N(R^{16″})-, (R^{16′})(R^{16″})NC(O)N(R^{16′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted, on available carbon atoms, by 1, 2 or 3 substituents independently selected from C₁₋₄alkyl, hydroxyl, cyano, trifluoromethyl, trifluoromethoxy, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ— and C₁₋₄alkylS(O)ᵣC₁₋₄alkyl (wherein r is independently selected 0, 1 and 2) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano];
R¹⁶ is independently selected from, C₁₋₃alkyl optionally substituted by 1, 2 or 3 substituents independently selected from hydroxyl, halo, C₁₋₄alkoxy, carboxy and cyano;
R^{16′}, R^{16″} and R^{16′″} are independently selected from hydrogen and C₁₋₃alkyl optionally substituted by 1, 2, or 3 substituents independently selected from hydroxyl, halo, C₁₋₄alkoxy, carboxy and cyano);
or a pharmaceutically-acceptable salt thereof, for use as a medicament for the treatment of a disease mediated through 11βHSD1.

In another aspect the invention relates to a compound of the formula (1) as hereinabove defined or a pharmaceutically-acceptable salt thereof, for use as a medicament for the inhibition of 11βHSD1.

In another aspect the invention relates to a compound of formula (1): wherein:

Q is O, S, N(R⁸) or a single bond;
R⁸ is selected from hydrogen, C₁₋₄alkyl, C₃₋₅cycloalkyl and C₃₋₅cycloalkylmethyl (each of which is optionally substituted by 1, 2 or 3 fluoro atoms);
R¹ is selected from C₁₋₆alkyl, C₂₋₆alkenyl, C₂₋₆alkynyl, C₃₋₇cycloalkyl, heterocyclyl, heteroaryl, aryl, arylC₁₋₃alkyl, heteroarylC₁₋₃alkyl, C₃₋₇cycloalkylC₁₋₃alkyl, heterocyclylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₙ— (wherein n is 0, 1, 2 or 3), R⁵CON(R^{5′})-, (R^{5′})(R^{5″})N—, (R^{5′})(R^{5″})NC(O)—, R^{5′}OC(O)O—, R^{5′}OC(O)—, (R^{5′})(R^{5″})NC(O)N(R^{5′″})-, R⁵SO₂N(R^{5″})-, (R^{5′})(R^{5″})NSO₂— and C₁₋₂alkyl optionally substituted by 1, 2 or 3 substituents independently selected from hydroxy, halo, carboxy and C₁₋₃alkoxy (wherein R⁵ is C₁₋₃alkyl optionally substituted by 1, 2 or 3 substituents independently selected from hydroxyl, halo and cyano; and
R^{5′}, R^{5″} and R^{5′″} are independently selected from hydrogen and C₁₋₃alkyl optionally substituted by 1, 2 or 3 substituents independently selected from hydroxyl, halo, C₁₋₃alkoxy, carboxy and cyano or R^{5′} and R^{5″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano]; or
R¹ and R⁸ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁹ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R² is selected from adamantyl optionally substituted, on available carbon atoms, by 1 or substituents independently selected from R⁶;
R³ is hydrogen;
R⁴ is selected from hydrogen, R¹⁰, —OR¹⁰, —SR¹⁰ and —NR¹¹R¹²;
R¹⁰ is selected from C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, heterocyclylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₚ— (wherein p is 0, 1, 2 or 3), R¹³CON(R^{13′})-, (R^{13′})(R^{13″})N—, (R^{13′})(R^{13″})NC(O)—, R^{13′}C(O)O—, R^{13′}OC(O)—, (R^{13′})(R^{13″})NC(O)N(R^{13′″})-, R¹³SO₂N(R^{13″})-, (R^{13′})(R^{13″})NSO₂— and C₁₋₂alkyl optionally substituted by 1, 2 or 3 substituents independently selected from hydroxy, halo, carboxy and C₁₋₃alkoxy (wherein R¹³ is C₁₋₃alkyl optionally substituted by 1, 2 or 3 substituents selected from hydroxyl, halo and cyano; and
R^{13′}, R^{13′} and R^{13′″} are independently selected from hydrogen and C₁₋₃alkyl optionally substituted by 1, 2 or 3 substituents independently selected from hydroxyl, halo, C₁₋₃alkoxy, carboxy and cyano or R^{13′} and R^{13″} together with the nitrogen atom to which they are attached form a 4-7 membered saturated ring) and optionally substituted, on an available nitrogen, by a substituent independently selected from C₁₋₄alkyl, C₂₋₄alkanoyl and
C₁₋₄alkanesulphonyl each of which is optionally substituted by 1, 2 or 3 substituent independently selected from hydroxyl, halo, C₁₋₄alkoxy, carboxy and cyano];
R¹¹ is selected from hydrogen, C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, heterocyclylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})-, (R^{14′})(R^{14″})NC(O)—, R^{14″}C(O)O—, R^{14′}OC(O)—, (R^{14′})(R^{14″})NC(O)N(R^{14′″})-, R¹⁴SO₂N(R^{14″})-, (R^{4′})(R^{14″})NSO₂— and C₁₋₂alkyl optionally substituted by 1, 2 or 3 substituents independently selected from hydroxy, halo, carboxy and C₁₋₃alkoxy (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo, C₁₋₃alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano]; and
R¹² is selected from hydrogen, C₁₋₄alkyl, C₃₋₅cycloalkyl and C₃₋₅cycloalkylmethyl (each of which is optionally substituted by 1, 2 or 3 fluoro atoms); or
R¹¹ and R¹² together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted, on available carbon atoms, by 1, 2, or 3 substituents independently selected from R¹⁵ and optionally substituted on an available nitrogen by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R⁶, R⁷, R⁹ and R¹⁵ are independently selected from hydroxyl, halo, oxo, carboxy, cyano, trifluoromethyl, R¹⁶, R¹⁶O—, R¹⁶CO—, R¹⁶C(O)O—, R¹⁶CON(R^{16′})-, (R^{16′})(R^{16″})NC(O)—, (R^{16′})(R^{16″})N—, R¹⁶S(O)ₐ— wherein a is 0 to 2, R^{16′}OC(O)—, (R^{16′})(R^{16″})NSO₂—,
R¹⁶SO₂N(R^{16″})-, (R^{16′})(R^{16″})NC(O)N(R^{16′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted, on available carbon atoms, by 1, 2 or 3 substituents independently selected from C₁₋₄alkyl, hydroxyl, cyano, trifluoromethyl, trifluoromethoxy, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ— and C₁₋₄alkylS(O)ᵣC₁₋₄alkyl (wherein r is independently selected 0, 1 and 2) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano];
R¹⁶ is independently selected from, C₁₋₃alkyl optionally substituted by 1, 2 or 3 substituents independently selected from hydroxyl, halo, C₁₋₄alkoxy, carboxy and cyano;
R^{16′}, R^{16′} and R^{16″} are independently selected from hydrogen and C₁₋₃alkyl optionally substituted by 1, 2, or 3 substituents independently selected from hydroxyl, halo, C₁₋₄alkoxy, carboxy and cyano);
or a pharmaceutically-acceptable salt thereof.

In this specification the term “alkyl” includes both straight and branched chain alkyl groups but references to individual alkyl groups such as “propyl” are specific for the straight chain version only. For example, “C₁₋₄alkyl” includes propyl, isopropyl and t-butyl. However, references to individual alkyl groups such as ‘propyl’ are specific for the straight chain version only and references to individual branched chain alkyl groups such as ‘isopropyl’ are specific for the branched chain version only. A similar convention applies to other radicals therefore “arylC₁₋₄alkyl” would include 1-arylpropyl, 2-arylethyl and 3-arylbutyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A 4-7 membered saturated ring (for example formed between R^{5′} and R^{5″} and the nitrogen atom to which they are attached) is a monocyclic ring containing the nitrogen atom as the only ring atom.

“Heteroaryl”, unless otherwise specified, is a totally unsaturated, monocyclic ring containing 5 or 6 atoms of which at least 1, 2 or 3 ring atoms are independently chosen from nitrogen, sulphur or oxygen, which may, unless otherwise specified, be carbon-linked. A ring nitrogen atom may be optionally oxidised to form the corresponding N-oxide. Examples and suitable values of the term “heteroaryl” are thienyl, furyl, thiazolyl, pyrazolyl, isoxazolyl, imidazolyl, pyrrolyl, thiadiazolyl, isothiazolyl, triazolyl, pyrimidyl, pyrazinyl, pyridazinyl and pyridyl. Particularly “heteroaryl” refers to thienyl, furyl, thiazolyl, pyridyl, imidazolyl or pyrazolyl.

“Heterocylcyl” is a 4-7 saturated, monocyclic ring having 1-3 ring heteroatoms independently selected from nitrogen, oxygen and sulphur. The ring sulphur may be optionally oxidised to SO or SO₂.

“Aryl” is an aromatic carbocyclic ring i.e. phenyl or naphthyl.

A C₃₋₇cycloalkyl ring is a saturated carbon ring containing from 3 to 7 ring atoms.

A C₆₋₁₂polycycloalkyl ring is a ring system in which either at least 2 rings are fused together (fused or bridged) or in which 2 ring have one ring atom in common (spiro). An example of a polycycloalkyl ring is adamantly.

A “saturated mono, bicyclic or bridged ring system optionally containing 1 or 2 additional ring heteroatoms independently selected from nitrogen, oxygen and sulphur”, unless otherwise specified contains 4-14 ring atoms. Particularly a mono ring contains 4-7 ring atoms, a bicyclic ring 6-14 ring atoms and a bridged ring system 6-14 ring atoms. Examples of mono rings include piperidinyl, piperazinyl and morpholinyl. Examples of bicyclic rings include decalin and 2,3,3a,4,5,6,7,7a-octahydro-1H-indene.

Bridged ring systems are ring systems in which there are two or more bonds common to two or more constituent rings. Examples of bridged ring systems include 1,3,3-trimethyl-6-azabicyclo[3.2.1]octane, 2-aza-bicyclo[2.2.1]heptane and 7-azabicyclo(2,2,1)heptane, 1- and 2-adamantanyl.

A “saturated, partially saturated or unsaturated monocyclic ring” is, unless otherwise specified, a 4-7 membered carbon ring. Examples include, cyclopropyl, cyclobutyl, cyclopentyl, cyclopentenyl, cyclohexyl, cyclohexenyl and phenyl.

Examples of “C₁₋₄alkoxy” include methoxy, ethoxy and propoxy. Examples of “C₁₋₄alkoxyC₁₋₄alkyl” include methoxymethyl, ethoxymethyl, propoxymethyl, 2-methoxyethyl, 2-ethoxyethyl and 2-propoxyethyl. Examples of “C₁₋₄alkylS(O)_{n or p or q or r} wherein n or p or q or r is 0 to 2″ include methylthio, ethylthio, methylsulphinyl, ethylsulphinyl, mesyl and ethylsulphonyl. Examples of “C₁₋₄alkylS(O)ᵣC₁₋₄alkyl” wherein r is 0 to 2″ include methylthio, ethylthio, methylsulphinyl, ethylsulphinyl, mesyl, ethylsulphonyl, methylthiomethyl, ethylthiomethyl, methylsulphinylmethyl, ethylsulphinylmethyl, mesylmethyl and ethylsulphonylmethyl. Examples of “C₁₋₄alkanoyl” include propionyl and acetyl. Examples of “N—(C₁₋₄alkyl)amino” include methylamino and ethylamino. Examples of “N,N—(C₁₋₄alkyl)₂-amino” include N,N-dimethylamino, N,N-diethylamino and N-ethyl-N-methylamino. Examples of “C₂₋₄alkenyl” are vinyl, allyl and 1-propenyl. Examples of “C₂₋₄alkynyl” are ethynyl, 1-propynyl and 2-propynyl. Examples of “N—(C₁₋₄alkyl)carbamoyl” are methylaminocarbonyl and ethylaminocarbonyl. Examples of “N,N—(C₁₋₄alkyl)₂-carbamoyl” are dimethylaminocarbonyl and methylethylaminocarbonyl. Examples of “C₃₋₇cycloalkylC₁₋₃alkyl” include cyclopropymethyl, 2-cyclopropylethyl, cyclobutylmethyl, cyclopentylmethyl and cyclohexylmethyl. Examples of “C₃₋₇cycloalkylC₂₋₃alkenyl” include 2-cyclopropylethenyl, 2-cyclopentylethenyl and 2-cyclohexylethenyl. Examples of “C₃₋₇cycloalkylC₂₋₃alkynyl” include 2-cyclopropylethynyl, 2-cyclopentylethynyl and 2-cyclohexylethynyl.

Examples of “C₃₋₇cycloalkyl(CH₂)ₘ-” include cyclopropymethyl, 2-cyclopropylethyl, cyclobutylmethyl, cyclopentylmethyl and cyclohexylmethyl. Examples of C₆₋₁₂polycycloalkyl(CH₂)ₘ— include norbornyl bicyclo[2.2.2]octane(CH₂)ₘ—, bicyclo[3.2.1]octane(CH₂)ₘ— and 1- and 2-adamantanyl(CH₂)ₘ—,

A suitable pharmaceutically-acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically-acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (1) may have chiral centres and/or geometric isomeric centres (E- and Z-isomers), and it is to be understood that the invention encompasses all such optical, diastereoisomers and geometric isomers that possess 11βHSD1 inhibitory activity.

The invention relates to any and all tautomeric forms of the compounds of the formula (1) that possess 11βHSD1 inhibitory activity. It is also to be understood that certain compounds of the formula (1) can exist in solvated as well as unsolvated forms such as, for example, hydrated forms. It is to be understood that the invention encompasses all such solvated forms, which possess 11βHSD1 inhibitory activity.

In another, there is provided a compound of formula (1) wherein:

Q is O, S, N(R⁸) or a single bond;
R⁸ is selected from hydrogen, C₁₋₄alkyl, C₃₋₅cycloalkyl and C₃₋₅cycloalkylmethyl (each of which is optionally substituted by 1, 2 or 3 fluoro atoms);
R¹ is selected from C₁₋₆alkyl, C₂₋₆alkenyl, C₂₋₆alkynyl, C₃₋₇cycloalkyl, heterocyclyl, heteroaryl, aryl, arylC₁₋₃alkyl, heteroarylC₁₋₃alkyl, heterocyclylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, (each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₙ— (wherein n is 0, 1, 2 or 3), R⁵CON(R^{5′})-, (R^{5′})(R^{5″})N—, (R^{5′})(R^{5″})NC(O)—, R^{5″}C(O)O—, R^{5′}OC(O)—, (R^{5′})(R^{5″})NC(O)N(R^{5′″})-, R⁵SO₂N(R^{5″})-, (R^{5′})(R^{5″})NSO₂— and C₁₋₂alkyl optionally substituted by 1, 2 or 3 substituents independently selected from hydroxy, halo, carboxy and C₁₋₃alkoxy (wherein R⁵ is C₁₋₃alkyl optionally substituted by 1, 2 or 3 substituents independently selected from hydroxyl, halo and cyano; and
R^{5′}, R^{5″} and R^{5′″} are independently selected from hydrogen and C₁₋₃alkyl optionally substituted by 1, 2 or 3 substituents independently selected from hydroxyl, halo, C₁₋₃alkoxy, carboxy and cyano or R⁵ and R^{5″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl]; or
R¹ and R⁸ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁹ and optionally substituted, on an available nitrogen, by a substituent independently selected from C₁₋₄alkyl, C₂₋₄alkanoyl and C₁₋₄alkanesulphonyl;
R² is selected from C₃₋₇cycloalkyl(CH₂)ₘ—, and C₆₋₁₂polycycloalkyl(CH₂)ₘ— (wherein m is 0, 1 or 2 and the rings optionally contain 1 or 2 ring atoms independently selected from nitrogen, oxygen and sulphur are optionally substituted, on available carbon atoms, by 1, 2 or 3 substituents independently selected from R⁶ and optionally substituted, on an available nitrogen, by a substituent independently selected from C₁₋₄alkyl, C₂₋₄alkanoyl and C₁₋₄alkanesulphonyl);
R³ is selected from hydrogen, C₁₋₄alkyl, C₃₋₅cycloalkyl and C₃₋₅cycloalkylmethyl (each of which is optionally substituted by 1, 2 or 3 fluoro atoms);
R² and R³ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁷ and optionally substituted, on an available nitrogen, by a substituent independently selected from C₁₋₄alkyl, C₂₋₄alkanoyl and C₁₋₄alkanesulphonyl;
R⁴ is selected from hydrogen, R¹⁰, —OR¹⁰, —SR¹⁰ and —NR¹¹R¹²;
R¹⁰ is selected from C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₚ— (wherein p is 0, 1, 2 or 3), R¹³CON(R^{13′})-, (R^{13′})(R^{13″})N—, (R^{13′})(R^{13″})NC(O)—, R^{13″}C(O)O—, R^{13′}OC(O)—, (R^{13′})(R^{13″})NC(O)N(R¹³)—, R¹³SO₂N(R^{13″})-, (R^{13′})(R^{13″})NSO₂— and C₁₋₂alkyl optionally substituted by 1, 2 or 3 substituents independently selected from hydroxy, halo, carboxy and C₁₋₃alkoxy (wherein R¹³ is C₁₋₃alkyl optionally substituted by 1, 2 or 3 substituents selected from hydroxyl, halo and cyano; and
R^{13′}, R^{13″} and R^{13′″} are independently selected from hydrogen and C₁₋₃alkyl optionally substituted by 1, 2 or 3 substituents independently selected from hydroxyl, halo, C₁₋₃alkoxy, carboxy and cyano or R^{13′} and R^{13″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl];
R¹¹ is selected from hydrogen, C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})—, (R^{14′})(R^{14′})NC(O)—, R^{14′}C(O)O—, R^{14′}OC(O)—, (R^{14′})(R^{14′})NC(O)N(R^{14′″})—, R¹⁴SO₂N(R^{14″})—, (R^{14′})(R^{14′})NSO₂— and C₁₋₂alkyl optionally substituted by 1, 2 or 3 substituents independently selected from hydroxy, halo, carboxy and C₁₋₃alkoxy (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo,
C₁₋₃alkoxy, carboxy and cyano or R^{14′} and R^{14′} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl]; and
R¹² is selected from hydrogen, C₁₋₄alkyl, C₃₋₅cycloalkyl and C₃₋₅cycloalkylmethyl (each of which is optionally substituted by 1, 2 or 3 fluoro atoms); or
R¹¹ and R¹² together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted, on available carbon atoms, by 1, 2, or 3 substituents independently selected from R¹⁵ and optionally substituted on an available nitrogen by a substituent independently selected from C₁₋₄alkyl, C₂₋₄alkanoyl and C₁₋₄alkanesulphonyl;
R⁶, R⁷, R⁹ and R¹⁵ are independently selected from hydroxyl, halo, oxo, carboxy, cyano, trifluoromethyl, R¹⁶, R¹⁶O—, R¹⁶CO—, R¹⁶C(O)O—, R¹⁶CON(R^{16′})-, (R^{16′})(R^{16″})NC(O)—, (R^{6′})(R^{16″})N—, R¹⁶S(O)ₐ— wherein a is 0 to 2, R^{16′}OC(O)—, (R^{16′})(R^{16″})NSO₂—,
R¹⁶SO₂N(R^{16″})-, (R^{16′})(R^{16″})NC(O)N(R^{16′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted, on available carbon atoms, by 1, 2 or 3 substituents independently selected from C₁₋₄alkyl, hydroxyl, cyano, trifluoromethyl, trifluoromethoxy, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ— and C₁₋₄alkylS(O)ᵣC₁₋₄alkyl (wherein r is independently selected 0, 1 and 2) and optionally substituted, on an available nitrogen, by a substituent independently selected from C₁₋₄alkyl, C₂₋₄alkanoyl and C₁₋₄alkanesulphonyl];
R¹⁶ is independently selected from, C₁₋₃alkyl optionally substituted by 1, 2 or 3 substituents independently selected from hydroxyl, halo, C₁₋₄alkoxy, carboxy and cyano;
R^{16′}, R^{16′} and R^{16″} are independently selected from hydrogen and C₁₋₃alkyl optionally substituted by 1, 2, or 3 substituents independently selected from hydroxyl, halo, C₁₋₄alkoxy, carboxy and cyano);
or a pharmaceutically-acceptable salt thereof;
provided that:
i) when -QR¹ is N-(3-chloro-4-methoxybenzyl)amino then —NR²R³ is not N-(4-hydroxycyclohexyl)amino; and
ii) when -QR¹ is 2-fluorophenyl, 4-cyanophenyl or 3-methylphenyl then R⁴ is not morpholino, pyrrolidino, 4-methylpiperidino, cyclohexylmethylamino, 2-methoxyethylamino, 3-methoxypropylamino or cyclopropylmethylamino;
   and excluding: 2-[(2,4-dichlorophenyl)amino]-N-(tetrahydro-2H-pyran-4-yl)methyl]-4-(trifluoromethyl)-5-pyrimidinecarboxamide;
4-methyl-N-[2-(4-morpholinyl)ethyl]-2-(1-pyrrolidinyl)-5-pyrimidinecarboxamide;
N-cycloheptyl-2-(dimethylamino)-4-methyl-5-pyrimidinecarboxamide;
N-cyclopentyl-4-methyl-2-(4-morpholinyl)-5-pyrimidinecarboxamide;
N-cyclohexyl-2-(dimethylamino)-4-methyl-5-pyrimidinecarboxamide;
N-cycloheptyl-4-methyl-2-(1-piperidinyl)-5-pyrimidinecarboxamide;
N-cyclopropyl-4-methyl-2-(4-morpholinyl)-5-pyrimidinecarboxamide;
N-cyclopentyl-2-(dimethylamino)-4-methyl-5-pyrimidinecarboxamide;
4-methyl-2-(4-morpholinyl)-N-[2-(4-morpholinyl)ethyl]-5-pyrimidinecarboxamide;
N-cycloheptyl-4-methyl-2-(4-methyl-1-piperazinyl)-5-pyrimidinecarboxamide;
2-(dimethylamino)-4-methyl-N-[2-(4-morpholinyl)ethyl]-5-pyrimidinecarboxamide;
2-(4-ethyl-1-piperazinyl)-4-methyl-N-[2-(4-morpholinyl)ethyl]-5-pyrimidinecarboxamide;
N-cyclohexyl-2-(4-ethyl-1-piperazinyl)-4-methyl-5-pyrimidinecarboxamide;
N-cyclopropyl-2-(4-ethyl-1-piperazinyl)-4-methyl-5-pyrimidinecarboxamide;
N-cyclohexyl-4-methyl-2-(4-methyl-1-piperazinyl)-5-pyrimidinecarboxamide;
N-cyclohexyl-4-methyl-2-(4-morpholinyl)-5-pyrimidinecarboxamide;
N-cyclopentyl-2-[[(2-methoxyphenyl)methyl]amino]-4-methyl-5-pyrimidinecarboxamide;
N-cyclohexyl-2-[[(4-methoxyphenyl)methyl]amino]-4-methyl-5-pyrimidinecarboxamide;
N-cycloheptyl-2-[[(4-methoxyphenyl)methyl]amino]-4-methyl-5-pyrimidinecarboxamide;
N-cyclopentyl-2-[[(3-methoxyphenyl)methyl]amino]-4-methyl-5-pyrimidinecarboxamide;
2-ethyl-4-methyl-N—[(tetrahydro-2-furanyl)methyl]-5-pyrimidinecarboxamide;
N-cyclopentyl-2-[[(4-methoxyphenyl)methyl]amino]-4-methyl-5-pyrimidinecarboxamide;
N-cyclopropyl-2-ethyl-4-methyl-5-pyrimidinecarboxamide;
N-cyclohexyl-2-(methylthio)-4-propyl-5-pyrimidinecarboxamide;
N-cycloheptyl-4-ethyl-2-(methylthio)-5-pyrimidinecarboxamide;
N-cycloheptyl-2-[[(3-methoxyphenyl)methyl]amino]-4-methyl-5-pyrimidinecarboxamide;
N-cyclopentyl-2-(4-ethyl-1-piperazinyl)-4-methyl-5-pyrimidinecarboxamide;
N-cyclohexyl-2-[[(2-methoxyphenyl)methyl]amino]-4-methyl-5-pyrimidinecarboxamide;
N-cycloheptyl-4-methyl-2-(4-morpholinyl)-5-pyrimidinecarboxamide;
4-methyl-2-(4-morpholinyl)-N-[2-(1-pyrrolidinyl)ethyl]-5-pyrimidinecarboxamide;
N-cyclopentyl-4-methyl-2-[(phenylmethyl)amino]-5-pyrimidinecarboxamide;
N-cyclohexyl-2-[[(3-methoxyphenyl)methyl]amino]-4-methyl-5-pyrimidinecarboxamide;
N-cyclopropyl-2-(methylthio)-4-propyl-5-pyrimidinecarboxamide;
2-(2-benzoxazolylamino)-N-cyclohexyl-N,4-dimethyl-5-pyrimidinecarboxamide;
N-cyclohexyl-4-ethyl-2-(methylthio)-5-pyrimidinecarboxamide;
N-cyclohexyl-4-methyl-2-(methylthio)-5-pyrimidinecarboxamide;
N-cycloheptyl-4-methyl-2-(methylthio)-5-pyrimidinecarboxamide;
N-cyclohexyl-2-(cyclohexylamino)-4-methyl-5-pyrimidinecarboxamide;
(3α,3aβ,5α,6β,6aβ) 2-(dimethylamino)-4-methoxy-N-(octahydro-1-methyl-3,5-methanocyclopenta[b]pyrrol-6-yl)-5-pyrimidinecarboxamide;
N-[(1-ethyl-2-pyrrolidinyl)methyl]-4-methoxy-2-(methylthio)-5-pyrimidinecarboxamide;
N-[(1-ethyl-2-pyrrolidinyl)methyl]-4-methoxy-5-pyrimidinecarboxamide;
2-amino-4-methoxy-N-[(1-methyl-2-pyrrolidinyl)methyl]-5-pyrimidinecarboxamide;
4-ethoxy-N-[(1-ethyl-2-pyrrolidinyl)methyl]-2-methyl-5-pyrimidinecarboxamide;
(3α,3aβ,5α,6β,6aβ)-4-ethoxy-2-methyl-N-(octahydro-1-methyl-3,5-methanocyclopenta[b]pyrrol-6-yl)-5-pyrimidinecarboxamide;
(R)—N-[(1-ethyl-2-pyrrolidinyl)methyl]-4-methoxy-2-methyl-5-pyrimidinecarboxamide;
N-[(1-ethyl-2-pyrrolidinyl)methyl]-2-methyl-4-(1-methylethoxy)-5-pyrimidinecarboxamide;
2-amino-N-(1-ethyl-3-pyrrolidinyl)-4-methoxy-5-pyrimidinecarboxamide;
exo-2-amino-N-8-azabicyclo[3.2.1]oct-3-yl-4-methoxy-5-pyrimidinecarboxamide;
(S)-4-ethoxy-N-[(1-ethyl-2-pyrrolidinyl)methyl]-2-methyl-5-pyrimidinecarboxamide;
N-[(1-ethyl-2-pyrrolidinyl)methyl]-4-methoxy-2-propyl-5-pyrimidinecarboxamide;
2-ethyl-N-[(1-ethyl-2-pyrrolidinyl)methyl]-4-methoxy-5-pyrimidinecarboxamide;
4-methoxy-2-methyl-N-[(1-propyl-2-pyrrolidinyl)methyl]-5-pyrimidinecarboxamide;
N-[(1-butyl-2-pyrrolidinyl)methyl]-4-methoxy-2-methyl-5-pyrimidinecarboxamide;
4-ethoxy-2-ethyl-N-[(1-methyl-2-pyrrolidinyl)methyl]-5-pyrimidinecarboxamide;
4-ethoxy-2-methyl-N-[(1-methyl-2-pyrrolidinyl)methyl]-5-pyrimidinecarboxamide;
4-methoxy-2-methyl-N-[(1-methyl-2-pyrrolidinyl)methyl]-5-pyrimidinecarboxamide;
N-[(1-ethyl-2-pyrrolidinyl)methyl]-4-methoxy-2-(1-methylethyl)-5-pyrimidinecarboxamide;
4-ethoxy-2-ethyl-N-[(1-ethyl-2-pyrrolidinyl)methyl]-5-pyrimidinecarboxamide;
N—(N-ethylpyrrolidin-2-ylmethyl)-4-ethoxy-2-methyl-5-pyrimidinecarboxamide;
N—-ethylpyrrolidin-2-ylmethyl)-4-propoxy-2-methyl-5-pyrimidinecarboxamide;
N—-ethylpyrrolidin-2-ylmethyl)-4-isoproxy-2-methyl-5-pyrimidinecarboxamide;
N—(N-ethylpyrrolidin-2-ylmethyl)-4-ethoxy-2-amino-5-pyrimidinecarboxamide;
N—-ethylpyrrolidin-2-ylmethyl)-4-propoxy-2-amino-5-pyrimidinecarboxamide;
N—(N-ethylpyrrolidin-2-ylmethyl)-4-isopropoxy-2-amino-5-pyrimidinecarboxamide;
N—-ethylpyrrolidin-2-ylmethyl)-4-methoxy-2-methylamino-5-pyrimidinecarboxamide;
N-(cyclohexyl)-4-methyl-2-piperazin-1-yl-5-pyrimidinecarboxamide;
N-cyclooctyl-2,4-dimethyl-5-pyrimidinecarboxamide;
N-cycloheptyl-2,4-dimethyl-5-pyrimidinecarboxamide;
N-cyclopropyl-2,4-dimethyl-5-pyrimidinecarboxamide;
N-cyclopentyl-2,4-dimethyl-5-pyrimidinecarboxamide;
N-cyclohexyl-2,4-dimethyl-5-pyrimidinecarboxamide;
N-cyclopentyl-4-methyl-2-(1-pyrrolidinyl)-5-pyrimidinecarboxamide;
N-cycloheptyl-4-methyl-2-(4-methyl-1-pyrrolidinyl)-5-pyrimidinecarboxamide;
N-cycloheptyl-4-methyl-2-(1-pyrrolidinyl)-5-pyrimidinecarboxamide;
N-cyclohexyl-2-(4-ethyl-1-piperazinyl)-4-methyl-5-pyrimidinecarboxamide;
N-cyclohexyl-4-methyl-2-[(phenylmethyl)amino]-5-pyrimidinecarboxamide;
N-cyclopentyl-2-[[(2-fluorophenyl)methyl]amino]-4-methyl-5-pyrimidinecarboxamide;
N-cycloheptyl-2-(methylthio)-4-phenyl-5-pyrimidinecarboxamide;
N-cycloheptyl-2-(methylthio)-4-propyl-5-pyrimidinecarboxamide;
N-cyclohexyl-2-(methylthio)-4-phenyl-5-pyrimidinecarboxamide;
N-cycloheptyl-4-methyl-2-[(phenylmethyl)amino]-5-pyrimidinecarboxamide;
N-cycloheptyl-2-[[(2-fluorophenyl)methyl]amino]-4-methyl-5-pyrimidinecarboxamide;
and
N-cyclopropyl-2-(methylthio)-4-phenyl-5-pyrimidinecarboxamide.

In another aspect, there is provided a compound of formula (I):

wherein:
Q is O, S, N(R⁸) or a single bond;
R⁸ is selected from hydrogen, C₁₋₄alkyl, C₃₋₅cycloalkyl and C₃₋₅cycloalkylmethyl (each of which is optionally substituted by 1, 2 or 3 fluoro atoms);
R¹ is selected from C₁₋₆alkyl, C₂₋₆alkenyl, C₂₋₆alkynyl, C₃₋₇cycloalkyl, heterocyclyl, heteroaryl, aryl, arylC₁₋₃alkyl, heteroarylC₁₋₃alkyl, C₃₋₇cycloalkylC₁₋₃alkyl, heterocyclylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₙ— (wherein n is 0, 1, 2 or 3), R⁵CON(R^{5′})-, (R^{5′})(R^{5″})N—, (R^{5′})(R^{5″})NC(O)—, R^{5′}OC(O)O—, R^{5′}OC(O)—, (R^{5′})(R^{5″})NC(O)N(R^{5′})-, R⁵SO₂N(R^{5″})-, (R^{5′})(R^{5″})NSO₂— and C₁₋₂alkyl optionally substituted by 1, 2 or 3 substituents independently selected from hydroxy, halo, carboxy and C₁₋₃alkoxy (wherein R⁵ is C₁₋₃alkyl optionally substituted by 1, 2 or 3 substituents independently selected from hydroxyl, halo and cyano; and
R^{5′}, R^{5″} and R^{5′″} are independently selected from hydrogen and C₁₋₃alkyl optionally substituted by 1, 2 or 3 substituents independently selected from hydroxyl, halo, C₁₋₃alkoxy, carboxy and cyano or R^{5′} and R^{5″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano]provided that when Q is a single bond R¹ is not methyl; or
R¹ and R⁸ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁹ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R² is selected from C₃₋₇cycloalkyl(CH₂)ₘ—, and C₆₋₁₂polycycloalkyl(CH₂)ₘ— (wherein m is 0, 1 or 2 and the rings are optionally substituted, on available carbon atoms, by 1, 2 or 3 substituents independently selected from R⁶ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano);
R³ is selected from hydrogen, C₁₋₄alkyl, C₃₋₅cycloalkyl and C₃₋₅cycloalkylmethyl (each of which is optionally substituted by 1, 2 or 3 fluoro atoms);
R² and R³ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁷ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R⁴ is selected from hydrogen, R¹⁰, —OR¹⁰ and —NR¹¹R¹²;
R¹⁰ is selected from C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, heterocyclylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₚ— (wherein p is 0, 1, 2 or 3), R¹³CON(R^{13′})-, (R^{13′})(R^{13″})N—, (R^{13′})(R^{13″})NC(O)—, R^{13″}C(O)O—, R^{13′}OC(O)—, (R^{13′})(R^{13″})NC(O)N(R^{13′})-, R¹³SO₂N(R^{13″})-, (R^{13′})(R^{13″})NSO₂— and C₁₋₂alkyl optionally substituted by 1, 2 or 3 substituents independently selected from hydroxy, halo, carboxy and C₁₋₃alkoxy (wherein R¹³ is C₁₋₃alkyl optionally substituted by 1, 2 or 3 substituents selected from hydroxyl, halo and cyano; and
R^{13′} R^{13″} and R^{13′″} are independently selected from hydrogen and C₁₋₃alkyl optionally substituted by 1, 2 or 3 substituents independently selected from hydroxyl, halo,
C₁₋₃alkoxy, carboxy and cyano or R^{13′} and R^{13″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano];
R¹¹ is selected from hydrogen, C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, heterocyclylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})—, (R^{14′})(R^{14″})NC(O)—, R^{14″}C(O)O—, R^{14′}OC(O)—, (R^{14′})(R^{14″})NC(O)N(R^{14′″})-, R¹⁴SO₂N(R^{14″})-, (R^{14′})(R^{14″})NSO₂— and C₁₋₂alkyl optionally substituted by 1, 2 or 3 substituents independently selected from hydroxy, halo, carboxy and C₁₋₃alkoxy (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo, C₁₋₃alkoxy, carboxy and cyano or R^{14′} and R^{17″′} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano]; and
R¹² is selected from hydrogen, C₁₋₄alkyl, C₃₋₅cycloalkyl and C₃₋₅cycloalkylmethyl (each of which is optionally substituted by 1, 2 or 3 fluoro atoms); or
R¹¹ and R¹² together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted, on available carbon atoms, by 1, 2, or 3 substituents independently selected from R¹⁵ and optionally substituted on an available nitrogen by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R⁶, R⁷, R⁹ and R¹⁵ are independently selected from hydroxyl, halo, oxo, carboxy, cyano, trifluoromethyl, R¹⁶, R¹⁶O—, R¹⁶CO—, R¹⁶C(O)O—, R¹⁶CON(R^{16′})-, (R^{16′})(R^{16″})NC(O)—, (R^{16′})(R^{16″})N—, R¹⁶S(O)ₐ— wherein a is 0 to 2, R^{16′}OC(O)—, (R^{16′})(R^{16″})NSO₂—, R¹⁶SO₂N(R^{16″})-, (R^{16′})(R^{16″})NC(O)N(R^{16′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted, on available carbon atoms, by 1, 2 or 3 substituents independently selected from C₁₋₄alkyl, hydroxyl, cyano, trifluoromethyl, trifluoromethoxy, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ— and C₁₋₄alkylS(O)ᵣC₁₋₄alkyl (wherein r is independently selected 0, 1 and 2) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano];
R¹⁶ is independently selected from, C₁₋₃alkyl optionally substituted by 1, 2 or 3 substituents independently selected from hydroxyl, halo, C₁₋₄alkoxy, carboxy and cyano;
R^{16′}, R^{16″} and R^{16″′} are independently selected from hydrogen and C₁₋₃alkyl optionally substituted by 1, 2, or 3 substituents independently selected from hydroxyl, halo, C₁₋₄alkoxy, carboxy and cyano);
or a pharmaceutically-acceptable salt thereof;
provided that:
i) when -QR¹ is N-(3-chloro-4-methoxybenzyl)amino then —NR²R³ is not N-(4-hydroxycyclohexyl)amino; and
ii) when -QR¹ is 2-fluorophenyl, 4-cyanophenyl or 3-methylphenyl then R⁴ is not morpholino, pyrrolidino, 4-methylpiperidino, cyclohexylmethylamino, 2-methoxyethylamino, 3-methoxypropylamino or cyclopropylmethylamino.

In another aspect, there is provided a compound of formula (1):

wherein:
Q is O, S, N(R⁸) or a single bond;
R⁸ is selected from hydrogen, C₁₋₄alkyl, C₃₋₅cycloalkyl and C₃₋₅cycloalkylmethyl (each of which is optionally substituted by 1, 2 or 3 fluoro atoms);
R¹ is selected from C₁₋₆alkyl, C₂₋₆alkenyl, C₂₋₆alkynyl, C₃₋₇cycloalkyl, heterocyclyl, heteroaryl, heteroarylC₁₋₃alkyl, C₃₋₇cycloalkylC₁₋₃alkyl, heterocyclylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₙ— (wherein n is 0, 1, 2 or 3), R⁵CON(R^{5′})-, (R^{5′})(R^{5″})N—, (R^{5′})(R^{5″})NC(O)—, R^{5″}C(O)O—, R^{5′}OC(O)—, (R^{5′})(R^{5″})NC(O)N(R^{5′″})-, R⁵SO₂N(R^{5″})-, (R^{5′})(R^{5″})NSO₂— and C₁₋₂alkyl optionally substituted by 1, 2 or 3 substituents independently selected from hydroxy, halo, carboxy and C₁₋₃alkoxy (wherein R⁵ is C₁₋₃alkyl optionally substituted by 1, 2 or 3 substituents independently selected from hydroxyl, halo and cyano; and
R^{5′}, R^{5″} and R^{5′″} are independently selected from hydrogen and C₁₋₃alkyl optionally substituted by 1, 2 or 3 substituents independently selected from hydroxyl, halo,
C₁₋₃alkoxy, carboxy and cyano or R⁵ and R^{5″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano] provided that when Q is a single bond R¹ is not methyl; or
R¹ and R⁸ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁹ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R² is selected from C₃₋₇cycloalkyl(CH₂)ₘ—, and C₆₋₁₂polycycloalkyl(CH₂)ₘ— (wherein m is 0, 1 or 2 and the rings are optionally substituted, on available carbon atoms, by 1, 2 or 3 substituents independently selected from R⁶ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano);
R³ is selected from hydrogen, C₁₋₄alkyl, C₃₋₅cycloalkyl and C₃₋₅cycloalkylmethyl (each of which is optionally substituted by 1, 2 or 3 fluoro atoms);
R² and R³ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁷ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R⁴ is selected from hydrogen, R¹⁰, —OR¹⁰ and —NR¹¹R¹²;
R¹⁰ is selected from C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, heterocyclylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₚ— (wherein p is 0, 1, 2 or 3), R¹³CON(R^{13′})-, (R^{13′})(R^{13″})N—, (R^{13′})(R^{13″})NC(O)—, R^{13″}C(O)O—, R^{13′}OC(O)—, (R^{13′})(R^{13″})NC(O)N(R^{13′″})-, R¹³SO₂N(R^{13″})-, (R^{13′})(R^{13″})NSO₂— and C₁₋₂alkyl optionally substituted by 1, 2 or 3 substituents independently selected from hydroxy, halo, carboxy and C₁₋₃alkoxy (wherein R¹³ is C₁₋₃alkyl optionally substituted by 1, 2 or 3 substituents selected from hydroxyl, halo and cyano; and
R^{13′} R^{13″} and R^{13′″} are independently selected from hydrogen and C₁₋₃alkyl optionally substituted by 1, 2 or 3 substituents independently selected from hydroxyl, halo, C₁₋₃alkoxy, carboxy and cyano or R^{13′} and R^{13″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano];
R¹¹ is selected from hydrogen, C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, heterocyclylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})—, (R^{14′})(R^{14″})NC(O)—, R^{14′}C(O)O—, R^{14′}OC(O)—, (R^{14′})(R^{14″})NC(O)N(R^{14′″})—, R¹⁴SO₂N(R^{14″})—, (R^{14′})(R^{14″})NSO₂— and C₁₋₂alkyl optionally substituted by 1, 2 or 3 substituents independently selected from hydroxy, halo, carboxy and C₁₋₃alkoxy (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo,
C₁₋₃alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano]; and
R¹² is selected from hydrogen, C₁₋₄alkyl, C₃₋₅cycloalkyl and C₃₋₅cycloalkylmethyl (each of which is optionally substituted by 1, 2 or 3 fluoro atoms); or
R¹¹ and R¹² together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted, on available carbon atoms, by 1, 2, or 3 substituents independently selected from R¹⁵ and optionally substituted on an available nitrogen by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R⁶, R⁷, R⁹ and R¹⁵ are independently selected from hydroxyl, halo, oxo, carboxy, cyano, trifluoromethyl, R¹⁶, R¹⁶O—, R¹⁶CO—, R¹⁶C(O)O—, R¹⁶CON(R^{16′})-, (R^{16′})(R^{16″})NC(O)—, (R^{16′})(R^{16″})N—, R¹⁶S(O)ₐ— wherein a is 0 to 2, R^{16′}OC(O)—, (R^{16′})(R^{16″})NSO₂—, R¹⁶SO₂N(R^{16″})-, (R^{16′})(R^{16″})NC(O)N(R^{16′″})-, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted, on available carbon atoms, by 1, 2 or 3 substituents independently selected from C₁₋₄alkyl, hydroxyl, cyano, trifluoromethyl, trifluoromethoxy, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ— and C₁₋₄alkylS(O)ᵣC₁₋₄alkyl (wherein r is independently selected 0, 1 and 2) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano];
R¹⁶ is independently selected from, C₁₋₃alkyl optionally substituted by 1, 2 or 3 substituents independently selected from hydroxyl, halo, C₁₋₄alkoxy, carboxy and cyano;
R^{16′}, R^{16″} and R^{16″} are independently selected from hydrogen and C₁₋₃alkyl optionally substituted by 1, 2, or 3 substituents independently selected from hydroxyl, halo, C₁₋₄alkoxy, carboxy and cyano);
or a pharmaceutically-acceptable salt thereof.

The invention also relates to in vivo hydrolysable esters of a compound of the formula (I). In vivo hydrolysable esters are those esters that are broken down in the animal body to produce the parent carboxylic acid.

In one embodiment of the invention are provided compounds of formula (1). In an alternative embodiment are provided pharmaceutically-acceptable salts of compounds of formula (1).

### Definition of Q

a) In one aspect, the invention relates to a compound of the formula (I) as hereinabove defined wherein Q is O.
b) In another aspect Q is S.
c) In another aspect Q is a single bond.
d) In another aspect Q is N(R⁸).

### Definition of R¹

a) In one aspect R¹ is C₃₋₆cycloalkyl optionally substituted by 1, 2 or 3 substituents independently selected from C₁₋₃alkyl, hydroxy, halo, oxo, cyano, fluoro, trifluoromethyl and C₁₋₃alkoxy.
b) In another aspect R¹ is C₃₋₆cycloalkyl.
c) In another aspect R¹ is C₃₋₆cycloalkylC₁₋₂alkyl optionally substituted by 1, 2 or 3 substituents independently selected from C₁₋₃alkyl, hydroxy, halo, oxo, cyano, fluoro, trifluoromethyl and C₁₋₃alkoxy.
d) In another aspect R¹ is C₃₋₄cycloalkylC₁₋₂alkyl.
e) In another aspect R¹ is C₁₋₄alkyl optionally substituted by 1, 2 or 3 substituents independently selected from C₁₋₃alkyl, hydroxy, halo, oxo, cyano, trifluoromethyl and C₁₋₃alkoxy.
f) In another aspect R¹ is C₁₋₄alkyl.
g) In yet another aspect R¹ is selected from C₁₋₆alkyl, C₃₋₇cycloalkyl, heterocyclyl, arylC₁₋₃alkyl, heteroarylC₁₋₃alkyl and C₃₋₇cycloalkylC₁₋₃alkyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl and C₂₋₄alkanoyl.
h) In yet another aspect R¹ is selected from C₃₋₇cycloalkyl and heterocyclyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl and C₂₋₄alkanoyl.
i) In yet another aspect R¹ is selected from C₃₋₇cycloalkyl and heterocyclyl.

### Definition of R⁸

a) In one aspect R⁸ is selected from hydrogen, C₁₋₃alkyl, C₃₋₅cycloalkyl and C₃₋₅cycloalkylmethyl.
b) In another aspect R⁸ is selected from hydrogen, C₁₋₃alkyl, propyl and propylmethyl.
c) In another aspect R⁸ is selected from hydrogen and methyl.
d) In yet another aspect R⁸ is hydrogen.

### Definition of R¹ and R⁸ Together

a) In another aspect, R¹ and R⁸ together with the nitrogen atom to which they are attached form a saturated 5 or 6-membered mono, 6-12 membered bicyclic or 6-12 membered bridged ring system optionally containing 1 or 2 additional ring heteroatoms independently selected from nitrogen, oxygen and sulphur and which is optionally fused to a saturated, partially-saturated or aryl monocyclic ring wherein the resulting ring system is optionally substituted, on available carbon atoms, by 1, 2, or 3 substituents independently selected from R⁹ and optionally substituted, on an available nitrogen, by a substituent independently selected from C₁₋₄alkyl and C₂₋₄alkanoyl.
b) In another aspect, R¹ and R⁸ together with the nitrogen atom to which they are attached form a pyrrolidine ring optionally substituted, on available carbon atoms, by 1 or 2 substituents independently selected from R⁹ and optionally substituted, on an available nitrogen, by a substituent independently selected from C₁₋₄alkyl and C₂₋₄alkanoyl.

### Definition of R²

a) In one aspect, R² is selected from C₃₋₇cycloalkyl(CH₂)ₘ—, and C₆₋₁₂polycycloalkyl(CH₂)ₘ— (wherein m is 0, 1 or 2 and the rings are optionally substituted by 1, 2 or 3 substituents independently selected from R⁶) wherein m is 0, 1 or 2.
b) In another aspect, R² is selected from C₅₋₇cycloalkyl(CH₂)ₘ— and C₈₋₁₂polycycloalkyl(CH₂)ₘ— (wherein the rings are optionally substituted by 1, 2 or 3 substituents independently selected from R⁶) and wherein m is 0, 1 or 2.
c) In another aspect, R² is selected from C₅₋₇cycloalkyl(CH₂)ₘ—, C₇₋₁₀bicycloalkyl(CH₂)ₘ— and C₁₀tricycloalkyl(CH₂)ₘ— (wherein the cycloalkyl, bicycloalkyl and tricycloalkyl rings are optionally substituted by 1, 2 or 3 substituents independently selected from R⁶) and wherein m is 0, 1 or 2.
d) In yet another aspect, R² is selected from C₅₋₇cycloalkyl(CH₂)ₘ—, C₇₋₁₀bicycloalkyl(CH₂)ₘ— and adamantyl (wherein the cycloalkyl, bicycloalkyl and tricycloalkyl rings are optionally substituted by 1, 2 or 3 substituents independently selected from R⁶) and wherein m is 0, 1 or 2.
e) In yet another aspect, R² is selected from adamantyl optionally substituted by 1 or 2 substituents independently selected from R⁶.
f) In yet another aspect, R² is selected from adamantyl optionally substituted by 1 or 2 substituents independently selected from hydroxy and fluoro.
g) In yet another aspect, R² is selected from adamantyl optionally substituted by 1 hydroxy group.
h) In yet another aspect, R² is 5-hydroxy-2-adamantyl.
i) In yet another aspect, R² is (2r,5s)-5-hydroxyadamantyl-2-yl.
j) In yet another aspect R² is adamant-2-yl.
k) In another aspect R² is adamant-1-yl.
l) In yet another aspect, R² is cyclohexyl.

### Definition of m

a) In one aspect, m is 0 or 1.

### Definition of R³

a) In one aspect, R³ is C₁₋₄alkyl.
b) In another aspect, R³ is hydrogen, methyl or ethyl.
c) In another aspect, R³ is hydrogen.
d) In another aspect, R³ is methyl.
e) In another aspect, R³ is ethyl.
f) In another aspect, R³ is cyclopropyl.

### Definition of R² and R³ Together

a) In another aspect, R² and R³ together with the nitrogen atom to which they are attached form a saturated 5 or 6-membered mono, 6-12 membered bicyclic or 6-12 membered bridged ring system optionally containing 1 or 2 additional ring heteroatoms independently selected from nitrogen, oxygen and sulphur and which is optionally fused to a saturated, partially-saturated or aryl monocyclic ring wherein the resulting ring system is optionally substituted, on available carbon atoms, by 1, 2, or 3 substituents independently selected from R⁷ and optionally substituted, on an available nitrogen, by a substituent independently selected from C₁₋₄alkyl and C₂₋₄alkanoyl.
b) In another aspect, R² and R³ together with the nitrogen atom to which they are attached form a pyrrolidine ring optionally substituted, on available carbon atoms, by 1 or 2 substituents independently selected from R⁷ and optionally substituted, on an available nitrogen, by a substituent independently selected from C₁₋₄alkyl and C₂₋₄alkanoyl.

### Definition of R⁶

a) In one aspect, R⁶ is independently selected from hydroxyl, R¹⁶O—, R¹⁶CO— and R¹⁶C(O)O—.
b) In another aspect, R⁶ is independently selected from hydroxyl, R16O—, R16CO— and R16C(O)O—.
wherein R16 is C₁₋₃alkyl optionally substituted by C₁₋₄alkoxy or carboxy.
c) In another aspect, R⁶ is independently selected from R16CON(R16′)-, R16SO₂N(R16″)- and (R16′)(R16″)NC(O)N(R16′″)-.
d) In another aspect, R⁶ is independently selected from R16CON(R16′)-, R16SO₂N(R16″)- and (R16′)(R¹⁶)NC(O)N(R16′″)-;
R16 is C₁₋₃alkyl optionally substituted by C₁₋₄alkoxy or carboxy;
R16′, R16″ and R^{16″′} are independently selected from hydrogen and C₁₋₃alkyl optionally substituted by C₁₋₄alkoxy or carboxy).
e) In another aspect, R⁶ is independently selected from (R16′)(R16″)NC(O)— and (R16′)(R16′″)N—.
f) In another aspect, R⁶ is independently selected from (R16′)(R16″)NC(O)— and (R16′)(R16″)N—;
wherein R16′ and R16″ are independently selected from hydrogen and C₁₋₃alkyl optionally substituted by C₁₋₄alkoxy or carboxy.
g) In one aspect R⁶ is independently selected from methyl, trifluoromethyl, chloro, fluoro, bromo, methoxy, ethoxy, trifluoromethoxy, methanesulfonyl, ethanesulfonyl, methylthio, ethylthio, amino, N-methylamino, N-ethylamino, N-propylamino, N,N-dimethylamino, N,N-methylethylamino or N,N-diethylamino.
h) In another aspect, R⁶ is optionally substituted phenyl, pyridyl or pyrimidyl.
i) In another aspect, R⁶ is optionally substituted pyrid-2-yl, pyrid-3-yl or pyrid-4-yl.

### Definition of R⁷

a) In another aspect, R⁷ is independently selected from hydroxyl, halo, oxo, cyano, trifluoromethyl, R¹⁶ and R¹⁶O—.
b) In another aspect, R⁷ is independently selected from hydroxyl, halo, trifluoromethyl, R¹⁶ and R¹⁶O—.

### Definition of R⁹

a) In another aspect, R⁹ is independently selected from hydroxyl, halo, oxo, cyano, trifluoromethyl, R¹⁶ and R¹⁶O—.
b) In another aspect, R⁹ is independently selected from hydroxyl, halo, trifluoromethyl, R¹⁶ and R¹⁶O—.

### Definition of R¹⁵

a) In another aspect, R¹⁵ is independently selected from hydroxyl, halo, oxo, cyano, trifluoromethyl, R¹⁶ and R¹⁶O—.
b) In another aspect, R¹⁵ is independently selected from hydroxyl, halo, trifluoromethyl, R¹⁶ and R¹⁶O—.

### Definition of R¹⁶

a) In one aspect, R¹⁶ is independently selected from C₁₋₃alkyl.

### Definition of R^{16′} R^{16″} and R^{16′″}

a) In one aspect, R^{16′}, R^{16″} and R^{16′″} are independently selected from hydrogen and C₁₋₃alkyl.

### Definition of R⁴

a) In one aspect, the invention relates to a compound of the formula (I) as hereinabove defined wherein R⁴ is R¹⁰.
b) In another aspect R⁴ is OR¹⁰.
c) In another aspect R⁴ is SR¹⁰.
d) In another aspect R⁴ is —NR¹¹R¹².
e) In another aspect R⁴ is hydrogen.

### Definition of R¹⁰

a) In one aspect R¹⁰ is selected from C₁₋₆alkyl, C₃₋₇cycloalkyl, heterocyclyl, arylC₁₋₃alkyl, heteroarylC₁₋₃alkyl and C₃₋₇cycloalkylC₁₋₃alkyl [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₚ— (wherein p is 0, 1, 2 or 3), R¹³CON(R^{13′})-, (R^{13′})(R^{13″})N—, (R^{13′})(R^{13″})NC(O)—, R^{13″}C(O)O—, R^{13′}OC(O)—, (R^{13′})(R^{13″})NC(O)N(R^{13′″})—, R¹³SO₂N(R^{13″})-, and (R^{13′})(R^{13″})NSO₂— (wherein R¹³ is C₁₋₃alkyl and
R^{13′} R^{13″} and R^{13′″} are independently selected from hydrogen and C₁₋₃alkyl) and optionally substituted, on an available nitrogen, by a substituent independently selected from C₁₋₄alkyl and C₂₋₄alkanoyl].
b) In another aspect R¹⁰ is selected from C₁₋₆alkyl, C₃₋₇cycloalkyl, heterocyclyl and C₃₋₇cycloalkylC₁₋₃alkyl [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₚ— (wherein p is 0, 1, 2 or 3) and R¹³CON(R^{13′})— (wherein R¹³ is C₁₋₃alkyl and
R^{13′}, R^{13″} and R^{13′″} are independently selected from hydrogen and C₁₋₃alkyl) and optionally substituted, on an available nitrogen, by a substituent independently selected from C₁₋₄alkyl and C₂₋₄alkanoyl].

c) In another aspect R¹⁰ is selected from C₃₋₇cycloalkyl and heterocyclyl [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₚ— (wherein p is 0, 1, 2 or 3) and R¹³CON(R^{13′})— (wherein R¹³ is C₁₋₃alkyl and

R^{13′} R^{13″} and R^{13′″} are independently selected from hydrogen and C₁₋₃alkyl) and optionally substituted, on an available nitrogen, by a substituent independently selected from C₁₋₄alkyl and C₂₋₄alkanoyl].

### Definition of R¹¹

a) In one aspect R¹¹ is selected from hydrogen, C₁₋₆alkyl, C₃₋₇cycloalkyl, heterocyclyl, arylC₁₋₃alkyl, heteroarylC₁₋₃alkyl, C₃₋₇cycloalkylC₁₋₃alkyl and C₃₋₇cycloalkyl [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R⁴CON(R^{14′})—, (R^{14′})(R^{14″})NC(O)—, R^{14′}C(O)O—, R^{14′}C(O)—, (R^{14′})(R^{14″})NC(O)N(R^{14′″})—, R¹⁴SO₂N(R^{14″})- and (R^{14′})(R^{14″})NSO₂— (wherein R¹⁴ is C₁₋₃alkyl optionally substituted by 1, 2 or 3 substituents independently selected from hydroxyl, halo or cyano; and
R^{14′}, R^{14″} and R^{14′″} are independently selected from hydrogen and C₁₋₃alkyl optionally substituted by 1, 2 or 3 substituents independently selected from hydroxyl, halo, C₁₋alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl and C₂₋₄alkanoyl].
b) In another aspect R¹¹ is selected from C₁₋₆alkyl, C₃₋₇cycloalkyl, heterocyclyl, arylC₁₋₃alkyl, heteroarylC₁₋₃alkyl, C₃₋₇cycloalkylC₁₋₃alkyl and C₃₋₇cycloalkyl [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})— and (R^{14′})(R^{14″})NC(O)—, (wherein R¹⁴ is C₁₋₃alkyl and
R^{14′}, R^{14″} and R^{14′″} are independently selected from hydrogen and C₁₋₃alkyl optionally substituted by 1, 2 or 3 substituents independently selected from hydroxyl, halo, C₁₋₃alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl and C₂₋₄alkanoyl].
c) In another aspect R¹¹ is selected from C₃₋₇cycloalkyl and heterocyclyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})— and (R^{14′})(R^{14″})NC(O)—, (wherein R¹⁴ is C₁₋₃alkyl and
R^{14′}, R^{14″} and R^{14′″} are independently selected from hydrogen and C₁₋₃alkyl optionally substituted by 1, 2 or 3 substituents independently selected from hydroxyl, halo, C₁₋₃alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl and C₂₋₄alkanoyl].
d) In another aspect R¹¹ is selected from C₃₋₇cycloalkyl and heterocyclyl, [each of which is optionally substituted, on available carbon atoms, by 1 or 2 substituents independently selected from C₁₋₃alkyl, hydroxy, halo, oxo, cyano, trifluoromethyl and C₁₋₃alkoxy.
e) In another aspect R¹¹ and R¹² together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted, on available carbon atoms, by 1, 2, or 3 substituents independently selected from R¹⁵ and optionally substituted, on an available nitrogen, by a substituent independently selected from C₁₋₄alkyl and C₂₋₄alkanoyl.
f) In another aspect R¹¹ and R¹² together with the nitrogen atom to which they are attached form a saturated mono, bicyclic or bridged ring system optionally containing 1 or 2 additional ring heteroatoms independently selected from nitrogen, oxygen and sulphur.
g) In another aspect R¹¹ and R¹² together with the nitrogen atom to which they are attached form a heterocyclyl group which is optionally substituted by 1 or 2 substituents independently selected from R¹⁵
h) In another aspect R¹¹ and R¹² together with the nitrogen atom to which they are attached form a heterocyclyl group which is optionally substituted by 1 or 2 substituents independently selected from hydroxyl, halo, oxo, carboxy, cyano, trifluoromethyl, R¹⁶, R¹⁶O—, R¹⁶CO—, R¹⁶C(O)O—, R¹⁶CON(R^{16′})-, (R^{16′})(R^{16″})NC(O)—, (R^{16′})(R^{16″})N—, R¹⁶S(O)ₐ— wherein a is 0 to 2, R^{16′}OC(O)—, (R^{16′})(R^{16″})NSO₂—, R¹⁶SO₂N(R^{16″})-, (R^{16′})(R^{16″})NC(O)N(R^{16′″})— wherein R¹⁶ is selected from hydrogen and C₁₋₃alkyl.

### Definition of R¹²

a) In one aspect R¹² is selected from hydrogen, C₁₋₃alkyl, C₃₋₅cycloalkyl and C₃₋₅cycloalkylmethyl.
b) In another aspect R¹² is selected from hydrogen, C₁₋₃alkyl, propyl and propylmethyl.
c) In another aspect R¹² is selected from hydrogen and methyl.
d) In yet another aspect R¹² is hydrogen.
In one aspect R¹ is optionally substituted by 0 substituents.

In one aspect R¹ is optionally substituted by 1 substituent.

In one aspect R¹ is optionally substituted by 2 substituents.

In one aspect R¹ is optionally substituted by 3 substituents.

In one aspect R² is optionally substituted by 0 substituents.

In one aspect R² is optionally substituted by 1 substituent.

In one aspect R² is optionally substituted by 2 substituents.

In one aspect R² is optionally substituted by 3 substituents.

In one aspect R³ is optionally substituted by 0 substituents.

In one aspect R³ is optionally substituted by 1 substituent.

In one aspect R³ is optionally substituted by 2 substituents.

In one aspect R³ is optionally substituted by 3 substituents.

In one aspect the group formed by R² and R³ together is optionally substituted by 0 substituents.

In one aspect the group formed by R² and R³ together is optionally substituted by 1 substituent.

In one aspect the group formed by R² and R³ together is optionally substituted by 2 substituents.

In one aspect the group formed by R² and R³ together is optionally substituted by 3 substituents.

In one aspect the phenyl and heteroaryl groups in R⁶ and R⁷ are independently optionally substituted by 0 substituents.

In one aspect the phenyl and heteroaryl groups in R⁶ and R⁷ are independently optionally substituted by 1 substituent.

In one aspect the phenyl and heteroaryl groups in R⁶ and R⁷ are independently are optionally substituted by 2 substituents.

In one aspect the phenyl and heteroaryl groups in R⁶ and R⁷ are independently are optionally substituted by 3 substituents.

Particular values of variable groups are as follows. Such values may be used where appropriate with any of the definitions, claims or embodiments defined hereinbefore or hereinafter, for compounds of formula (I).

Particular classes of compounds of the present invention are disclosed in Table A using combinations of the definitions described hereinabove. For example, ‘a’ in the column headed R² in the table refers to definition (a) given for R² hereinabove and ‘1’ refers to the first definition given for the variables in the compound of formula (1) at the beginning of the description. The variables R⁵, R^{5′}, R^{5″}, R^{5′″}, R¹³, R^{13′}, R^{13″},R^{13′″}, R¹⁴, R^{14′}, R^{14″},R^{14′″}, R¹⁶, R^{16′}, R^{16″} and R^{16′″} are as hereinabove defined.

TABLE A

| Class | Q | R¹ and R⁸ | R¹ | R⁹ | R² | R⁶ | R³ | R⁷ | R⁴ | R¹⁰ | R¹¹ | R¹² | R¹⁵ |
|---|---|---|---|---|---|---|---|---|---|---|---|---|---|
| 1 | a | — | a | — | d | b | b | — | a | a | — | — | — |
| 2 | b | — | a | — | d | b | b | — | a | a | — | — | — |
| 3 | c | — | a | — | d | b | b | — | a | a | — | — | — |
| 4 | d | a | — | a | d | b | b | — | a | a | — | — | — |
| 5 | a | — | a | — | d | b | b | — | b | a | — | — | — |
| 6 | b | — | a | — | d | b | b | — | b | a | — | — | — |
| 7 | c | — | a | — | d | b | b | — | b | a | — | — | — |
| 8 | d | a | — | a | d | b | b | — | b | a | — | — | — |
| 9 | a | — | a | — | d | b | b | — | c | a | — | — | — |
| 10 | b | — | a | — | d | b | b | — | c | a | — | — | — |
| 11 | c | — | a | — | d | b | b | — | c | a | — | — | — |
| 12 | d | a | — | a | d | b | b | — | c | a | — | — | — |
| 13 | a | — | a | — | d | b | b | — | d | — | c | — | 1 |
| 14 | b | — | a | — | d | b | b | — | d | — | c | — | 1 |
| 15 | c | — | a | — | d | b | b | — | d | — | c | — | 1 |
| 16 | d | a | — | a | d | b | b | — | d | — | c | — | 1 |
| 17 | a | — | a | — | d | b | b | — | e | — | — | — | — |
| 18 | b | — | a | — | d | b | b | — | e | — | — | — | — |
| 19 | c | — | a | — | d | b | b | — | e | — | — | — | — |
| 20 | d | a | — | a | d | b | b | — | e | — | — | — | — |

A particular class of compound is that of formula (1) wherein:

Q is O, S, N(R⁸) or a single bond;
R⁸ is selected from hydrogen, C₁₋₄alkyl, C₃₋₅cycloalkyl and C₃₋₅cycloalkylmethyl (each of which is optionally substituted by 1, 2 or 3 fluoro atoms);
R¹ is selected from C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₙ— (wherein n is 0, 1, 2 or 3) and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl, C₂₋₄alkanoyl and C₁₋₄alkanesulphonyl; or
R¹ and R⁸ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁹ and optionally substituted, on an available nitrogen, by a substituent independently selected from C₁₋₄alkyl, C₂₋₄alkanoyl and C₁₋₄alkanesulphonyl;
R² is selected from C₃₋₇cycloalkyl(CH₂)ₘ—, and C₆₋₁₂polycycloalkyl(CH₂)ₘ— (wherein m is 0, 1 or 2 and the rings optionally contain 1 or 2 ring atoms independently selected from nitrogen, oxygen and sulphur are optionally substituted, on available carbon atoms, by 1, 2 or 3 substituents independently selected from R⁶ and optionally substituted, on an available nitrogen, by a substituent independently selected from C₁₋₄alkyl, C₂₋₄alkanoyl and C₁₋₄alkanesulphonyl);
R³ is selected from hydrogen, C₁₋₄alkyl, C₃₋₅cycloalkyl and C₃₋₅cycloalkylmethyl (each of which is optionally substituted by 1, 2 or 3 fluoro atoms);
R² and R³ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁷ and optionally substituted, on an available nitrogen, by a substituent independently selected from C₁₋₄alkyl, C₂₋₄alkanoyl and C₁₋₄alkanesulphonyl;
R⁴ is selected from hydrogen, R¹⁰, —OR¹⁰, —SR¹⁰ and —NR¹¹R¹²;
R¹⁰ is selected from C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₚ— (wherein p is 0, 1, 2 or 3)) and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl, C₂₋₄alkanoyl and C₁₋₄alkanesulphonyl;
R¹¹ is selected from hydrogen, C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, C₃₋₇cycloalkylC₁₋₃alkyl,
C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})—, (R^{14′})(R^{14″})NC(O)—, R^{14′}C(O)O—, R^{14′}C(O)—, (R^{14′})(R^{14″})NC(O)N(R^{14′″})-, R¹⁴SO₂N(R^{14″})-, and (R^{14′})(R^{14″})NSO₂— (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo, C₁₋₃alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl]; and
R¹² is selected from hydrogen, C₁₋₄alkyl, C₃₋₅cycloalkyl and C₃₋₅cycloalkylmethyl (each of which is optionally substituted by 1, 2 or 3 fluoro atoms); or
R¹¹ and R¹² together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted, on available carbon atoms, by 1, 2, or 3 substituents independently selected from R¹⁵ and optionally substituted, on an available nitrogen, by a substituent independently selected from C₁₋₄alkyl, C₂₋₄alkanoyl and C₁₋₄alkanesulphonyl;
R⁶, R⁷, R⁹ and R¹⁵ are independently selected from hydroxyl, halo, oxo, carboxy, cyano, trifluoromethyl, R¹⁶, R¹⁶O—, R¹⁶CO—, R¹⁶C(O)O—, R¹⁶CON(R^{16′})-, (R^{16′})(R^{16″})NC(O)—, (R^{16′})(R^{16″})N—, R¹⁶S(O)ₐ— wherein a is 0 to 2, R^{16′}OC(O)—, (R^{16′})(R^{16″})NSO₂—,
R¹⁶SO₂N(R^{16″})-, (R^{16′})(R^{16″})NC(O)N(R^{16′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted, on available carbon atoms, by 1, 2 or 3 substituents independently selected from C₁₋₄alkyl, hydroxyl, cyano, trifluoromethyl, trifluoromethoxy, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ— and C₁₋₄alkylS(O)ᵣC₁₋₄alkyl (wherein r is independently selected 0, 1 and 2) and optionally substituted, on an available nitrogen, by a substituent independently selected from C₁₋₄alkyl, C₂₋₄alkanoyl and C₁₋₄alkanesulphonyl];
R¹⁶ is independently selected from, C₁₋₃alkyl optionally substituted by 1, 2 or 3 substituents independently selected from hydroxyl, halo, C₁₋₄alkoxy, carboxy and cyano;
R^{16′}, R^{16′} and R^{16′″} are independently selected from hydrogen and C₁₋₃alkyl optionally substituted by 1, 2, or 3 substituents independently selected from hydroxyl, halo, C₁₋₄alkoxy, carboxy and cyano);
or a pharmaceutically-acceptable salt thereof;
provided that:
when -QR¹ is N-(3-chloro-4-methoxybenzyl)amino then —NR²R³ is not N-(4-hydroxycyclohexyl)amino.

Another class of compound is that of formula (I) wherein:

Q is O, S, N(R⁸) or a single bond;
R⁸ is selected from hydrogen, C₁₋₄alkyl, C₃₋₅cycloalkyl and C₃₋₅cycloalkylmethyl (each of which is optionally substituted by 1, 2 or 3 fluoro atoms);
R¹ is selected from C₁₋₆alkyl, C₃₋₇cycloalkyl, heterocyclyl, arylC₁₋₃alkyl, heteroarylC₁₋₃alkyl and C₃₋₇cycloalkylC₁₋₃alkyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl and C₂₋₄alkanoyl; or
R¹ and R⁸ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁹ and optionally substituted, on an available nitrogen, by a substituent independently selected from C₁₋₄alkyl and C₂₋₄alkanoyl;
R² is selected from C₃₋₇cycloalkyl(CH₂)ₘ—, and C₆₋₁₂polycycloalkyl(CH₂)ₘ— (wherein m is 0, 1 or 2 and the rings optionally contain 1 or 2 ring atoms independently selected from nitrogen, oxygen and sulphur are optionally substituted by 1, 2 or 3 substituents independently selected from R⁶);
R³ is selected from hydrogen, C₁₋₄alkyl, C₃₋₅cycloalkyl and C₃₋₅cycloalkylmethyl (each of which is optionally substituted by 1, 2 or 3 fluoro atoms);
R² and R³ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁷ and optionally substituted, on an available nitrogen, by a substituent independently selected from C₁₋₄alkyl and C₂₋₄alkanoyl;
R⁴ is selected from hydrogen, R¹⁰, —OR¹⁰ and —NR¹¹R¹²;
R¹⁰ is selected from C₁₋₆alkyl, C₃₋₇cycloalkyl, heterocyclyl, arylC₁₋₃alkyl, heteroarylC₁₋₃alkyl and C₃₋₇cycloalkylC₁₋₃alkyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl and C₂₋₄alkanoyl;
R¹¹ is selected from hydrogen, C₁₋₆alkyl, C₃₋₇cycloalkyl, heterocyclyl and C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})—, (R^{14′})(R^{14′″})NC(O)—, (R^{14′})(R^{14″})NC(O)N(R^{14′″})—, R¹⁴SO₂N(R^{14″})— and (R^{14′})(R^{14″})NSO₂— (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo, C₁₋₃alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl and C₂₋₄alkanoyl]; and
R¹² is selected from hydrogen, C₁₋₄alkyl, C₃₋₅cycloalkyl and C₃₋₅cycloalkylmethyl (each of which is optionally substituted by 1, 2 or 3 fluoro atoms); or
R¹¹ and R¹² together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted on available carbon atoms, by 1, 2, or 3 substituents independently selected from R¹⁵ and optionally substituted, on an available nitrogen, by a substituent independently selected from C₁₋₄alkyl and C₂₋₄alkanoyl;
R⁶, R⁷, R⁹ and R¹⁵ are independently selected from hydroxyl, halo, oxo, carboxy, cyano, trifluoromethyl, R¹⁶, R¹⁶O— and R¹⁶CO—,
R¹⁶ is independently selected from, C₁₋₃alkyl optionally substituted by 1, 2 or 3 substituents independently selected from hydroxyl, halo, C₁₋₄alkoxy, carboxy and cyano;
or a pharmaceutically-acceptable salt thereof;
provided that:
when -QR¹ is N-(3-chloro-4-methoxybenzyl)amino then —NR²R³ is not N-(4-hydroxycyclohexyl)amino.

Another class of compound is that of formula (1) wherein:

Q is O, S, N(R⁸) or a single bond;
R⁸ is selected from hydrogen, C₁₋₄alkyl;
R¹ is selected from C₁₋₆alkyl, C₃₋₇cycloalkyl, heterocyclyl, arylC₁₋₃alkyl, heteroarylC₁₋₃alkyl and C₃₋₇cycloalkylC₁₋₃alkyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl and C₂₋₄alkanoyl; provided that when Q is a single bond R¹ is not methyl; or
R¹ and R⁸ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁹ and optionally substituted, on an available nitrogen, by a substituent independently selected from C₁₋₄alkyl and C₂₋₄alkanoyl;
R² is selected from C₃₋₇cycloalkyl(CH₂)ₘ—, and C₆₋₁₂polycycloalkyl(CH₂)ₘ— (wherein the rings are optionally substituted by 1, 2 or 3 substituents independently selected from R⁶);
R³ is selected from hydrogen;
R⁴ is selected from hydrogen, R¹⁰, —OR¹⁰ and —NR¹¹R¹²;
R¹⁰ is selected from C₁₋₆alkyl, C₃₋₇cycloalkyl, heterocyclyl, arylC₁₋₃alkyl, heteroarylC₁₋₃alkyl and C₃₋₇cycloalkylC₁₋₃alkyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl and C₂₋₄alkanoyl;
R¹¹ is selected from hydrogen, C₁₋₆alkyl, C₃₋₇cycloalkyl, heterocyclyl and C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})—, (R^{14′})(R^{14″})NC(O)—, (R^{14′})(R^{14″})NC(O)N(R^{14′″})—, R¹⁴SO₂N(R^{14″})— and (R^{14′})(R^{14″})NSO₂— (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo, C₁₋₃alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl and C₂₋₄alkanoyl]; and
R¹² is selected from hydrogen, C₁₋₄alkyl, C₃₋₅cycloalkyl and C₃₋₅cycloalkylmethyl (each of which is optionally substituted by 1, 2 or 3 fluoro atoms); or
R¹¹ and R¹² together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted on available carbon atoms, by 1, 2, or 3 substituents independently selected from R¹⁵ and optionally substituted, on an available nitrogen, by a substituent independently selected from C₁₋₄alkyl and C₂₋₄alkanoyl;
R⁶, R⁷, R⁹ and R¹⁵ are independently selected from hydroxyl, halo, oxo, carboxy, cyano, trifluoromethyl, R¹⁶, R¹⁶O— and R¹⁶CO—,
R¹⁶ is independently selected from, C₁₋₃alkyl optionally substituted by 1, 2 or 3 substituents independently selected from hydroxyl, halo, C₁₋₄alkoxy, carboxy and cyano;
or a pharmaceutically-acceptable salt thereof;
provided that:
when -QR¹ is N-(3-chloro-4-methoxybenzyl)amino then —NR²R³ is not N-(4-hydroxycyclohexyl)amino.

Yet another class of compound is that of formula (1) wherein:

Q is a single bond;
R¹ is selected from C₁₋₆alkyl, C₃₋₇cycloalkyl, heterocyclyl, arylC₁₋₃alkyl, heteroarylC₁₋₃alkyl and C₃₋₇cycloalkylC₁₋₃alkyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl and C₂₋₄alkanoyl;
R² is adamantyl optionally substituted by 1, 2 or 3 substituents independently selected from R⁶;
R³ is hydrogen;
R⁴ is selected from hydrogen, R¹⁰, —SR¹⁰, —OR¹⁰ and —NR¹¹R¹²;
R¹⁰ is selected from C₁₋₆alkyl, C₃₋₇cycloalkyl, heterocyclyl, arylC₁₋₃alkyl, heteroarylC₁₋₃alkyl and C₃₋₇cycloalkylC₁₋₃alkyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl and C₂₋₄alkanoyl;
R¹¹ is selected from hydrogen, C₁₋₆alkyl, C₃₋₇cycloalkyl, heterocyclyl and C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})-, (R^{14′})(R^{14″})NC(O)—, (R^{14′})(R^{14″})NC(O)N(R^{14′″})-, R¹⁴SO₂N(R^{14″})- and (R^{4′})(R^{14″})NSO₂— (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo, C₁₋₃alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl and C₂₋₄alkanoyl]; and
R¹² is selected from hydrogen, C₁₋₄alkyl, C₃₋₅cycloalkyl and C₃₋₅cycloalkylmethyl (each of which is optionally substituted by 1, 2 or 3 fluoro atoms); or
R¹¹ and R¹² together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted on available carbon atoms, by 1, 2, or 3 substituents independently selected from R¹⁵ and optionally substituted, on an available nitrogen, by a substituent independently selected from C₁₋₄alkyl and C₂₋₄alkanoyl;
R⁶ and R¹⁵ are independently selected from hydroxyl, halo, oxo, carboxy, cyano, trifluoromethyl, R¹⁶, R¹⁶O— and R¹⁶CO—,
R¹⁶ is independently selected from, C₁₋₃alkyl optionally substituted by 1, 2 or 3 substituents independently selected from hydroxyl, halo, C₁₋₄alkoxy, carboxy and cyano;
or a pharmaceutically-acceptable salt thereof.

Yet another class of compound is that of formula (I) wherein:

Q is a single bond;
R¹ is selected from C₃₋₇cycloalkyl and heterocyclyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl and C₂₋₄alkanoyl;
R² is adamantyl optionally substituted by 1, 2 or 3 substituents independently selected from R⁶;
R³ is hydrogen;
R⁴ is selected from R¹⁰ and —NR¹¹R¹²;
R¹⁰ is selected from C₁₋₆alkyl, C₃₋₇cycloalkyl and heterocyclyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl and C₂₋₄alkanoyl;
R¹¹ is selected from hydrogen, C₁₋₆alkyl, C₃₋₇cycloalkyl, heterocyclyl [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})-, (R^{14′})(R^{14″})NC(O)—, (R¹⁴)(R^{14″})NC(O)N(R^{14′″})—, R¹⁴SO₂N(R^{14″})— and (R^{14′})(R^{14′″})NSO₂— (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo, C₁₋₃alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl and C₂₋₄alkanoyl];
R¹² is selected from hydrogen and C₁₋₃alkyl; or
R¹¹ and R¹² together with the nitrogen atom to which they are attached form a saturated monocyclic ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted on available carbon atoms, by 1, 2, or 3 substituents independently selected from R¹⁵ and optionally substituted, on an available nitrogen, by a substituent independently selected from C₁₋₄alkyl and C₂₋₄alkanoyl;
R⁶ and R¹⁵ are independently selected from hydroxyl, halo, oxo, carboxy, cyano, trifluoromethyl, R¹⁶, R¹⁶O— and R¹⁶CO—,
R¹⁶ is independently selected from, C₁₋₃alkyl optionally substituted by 1, 2 or 3 substituents independently selected from hydroxyl, halo, C₁₋₄alkoxy, carboxy and cyano;
or a pharmaceutically-acceptable salt thereof.

Yet another class of compound is that of formula (I) wherein:

Q is a single bond;
R¹ is selected from C₃₋₇cycloalkyl and heterocyclyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl and C₂₋₄alkanoyl;
R² is adamantyl optionally substituted by 1, 2 or 3 substituents independently selected from R⁶;
R³ is hydrogen;
R⁴ is selected from R¹⁰ and —NR¹¹R¹²;
R¹⁰ is selected from C₁₋₆alkyl, C₃₋₇cycloalkyl and heterocyclyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl and C₂₋₄alkanoyl;
R¹¹ is selected from hydrogen, C₁₋₆alkyl, C₃₋₇cycloalkyl, heterocyclyl [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})—, (R^{14′})(R^{14′″})NC(O)—, (R^{14′})(R^{14″})NC(O)N(R^{14′″})—, R¹⁴SO₂N(R^{14″})— and (R^{14′})(R^{14″})NSO₂— (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo, C₁₋₃alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl and C₂₋₄alkanoyl];
R¹² is selected from hydrogen and C₁₋₃alkyl; or
R¹¹ and R¹² together with the nitrogen atom to which they are attached form a saturated monocyclic ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted on available carbon atoms, by 1, 2, or 3 substituents independently selected from R¹⁵ and optionally substituted, on an available nitrogen, by a substituent independently selected from C₁₋₄alkyl and C₂₋₄alkanoyl;
R⁶ and R¹⁵ are independently selected from hydroxyl, halo, oxo, carboxy, cyano, trifluoromethyl, R¹⁶, R¹⁶O— and R¹⁶CO—,
R¹⁶ is independently selected from, C₁₋₃alkyl optionally substituted by 1, 2 or 3 substituents independently selected from hydroxyl, halo, C₁₋₄alkoxy, carboxy and cyano;
or a pharmaceutically-acceptable salt thereof.

Yet another class of compound is that of formula (I) wherein:

Q is a single bond;
R¹ is selected from C₃₋₇cycloalkyl and heterocyclyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₄alkyl and C₂₋₄alkanoyl;
R² is adamantyl optionally substituted by 1 hydroxy group;
R³ is hydrogen;

### R⁴ is —NR¹¹R¹²;

R¹¹ is selected from hydrogen, C₁₋₆alkyl, C₃₋₇cycloalkyl, heterocyclyl [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})-, (R^{14′})(R^{14″})NC(O)—, (R^{14′})(R^{14″})NC(O)N(R^{14′″})—, R¹⁴SO₂N(R^{14″})— and (R^{14′})(R^{14″})NSO₂— (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo, C₁₋₃alkoxy, carboxy and cyano) and optionally substituted, on an available nitrogen, by a substituent independently selected from C₁₋₄alkyl and C₂₋₄alkanoyl];
R¹² is hydrogen; or
R¹¹ and R¹² together with the nitrogen atom to which they are attached form a saturated monocyclic ring system optionally containing 1 or 2 additional ring heteroatoms independently selected from nitrogen, oxygen and sulphur and wherein the ring system is optionally substituted on available carbon atoms, by 1, 2, or 3 substituents independently selected from R¹⁵ and optionally substituted, on an available nitrogen, by a substituent independently selected from C₁₋₄alkyl and C₂₋₄alkanoyl;
R⁶ and R¹⁵ are independently selected from hydroxyl, halo, oxo, carboxy, cyano, trifluoromethyl, R¹⁶, R¹⁶O— and R¹⁶CO—,
R¹⁶ is independently selected from, C₁₋₃alkyl optionally substituted by 1, 2 or 3 substituents independently selected from hydroxyl, halo, C₁₋₄alkoxy, carboxy and cyano;

or a pharmaceutically-acceptable salt thereof.

In another aspect the invention relates to a compound of the formula (IA):

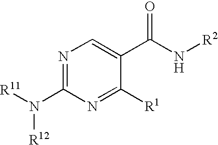

wherein R² is adamantyl optionally substituted by hydroxy and R¹, R¹¹ and R¹² are as hereinabove defined.

In another aspect, the invention relates to a compound of the formula 1 as hereinabove defined or a pharmaceutically-acceptable salt thereof excluding any one of the Examples and pharmaceutically-acceptable salts thereof.

In another aspect of the invention, suitable compounds of the invention are any one or more of the Examples or a pharmaceutically-acceptable salt thereof.

In another aspect of the invention, suitable compounds of the invention are any one or more of the following or a pharmaceutically-acceptable salt thereof:
4-cyclopropyl-N-[(2s,5r)-5-hydroxyadamantan-2-yl]-2-morpholin-4-ylpyrimidine-5-carboxamide;
4-cyclopropyl-N-[(2r,5s)-5-hydroxyadamantan-2-yl]-2-methylpyrimidine-5-carboxamide;
4-cyclopropyl-N-[(2r,5s)-5-hydroxyadamantan-2-yl]pyrimidine-5-carboxamide;
4-tert-butyl-N-[(2r,5s)-5-hydroxyadamantan-2-yl]-2-morpholin-4-ylpyrimidine-5-carboxamide;
N-[(2r,5 s)-5-hydroxyadamantan-2-yl]-4-methyl-2-morpholin-4-ylpyrimidine-5-carboxamide;
4-tert-butyl-N-[(2r,5s)-5-hydroxyadamantan-2-yl]-2-methylpyrimidine-5-carboxamide;
4-tert-butyl-N-[(2r,5s)-5-hydroxyadamantan-2-yl]pyrimidine-5-carboxamide;
N-[(2r,5s)-5-hydroxyadamantan-2-yl]-2-morpholin-4-yl-4-propylsulfanyl-pyrimidine-5-carboxamide;
N-[(2r,5s)-5-hydroxyadamantan-2-yl]-2-methyl-4-propylsulfanylpyrimidine-5-carboxamide;
N-[(2r,5s)-5-hydroxyadamantan-2-yl]-4-propylsulfanylpyrimidine-5-carboxamide;
4-cyclopropyl-N-[(2r,5s)-5-hydroxyadamantan-2-yl]-2-methylsulfanyl-pyrimidine-5-carboxamide;
N-(2-adamantyl)-4-cyclopropyl-2-methyl-pyrimidine-5-carboxamide;
N-(2-adamantyl)-4-cyclopropyl-2-morpholino-pyrimidine-5-carboxamide;
N-(2-adamantyl)-4-tert-butyl-2-morpholin-4-ylpyrimidine-5-carboxamide;
N-(2-adamantyl)-4-methyl-2-morpholin-4-ylpyrimidine-5-carboxamide;
N-[(2r,5s)-5-hydroxyadamantan-2-yl]-2,4-bis(propylsulfanyl)pyrimidine-5-carboxamide;
2-dimethylamino-N-[(2r,5s)-5-hydroxyadamantan-2-yl]-4-propylsulfanylpyrimidine-5-carboxamide;
4-dimethylamino-N-[(2r,5s)-5-hydroxyadamantan-2-yl]-2-propylsulfanylpyrimidine-5-carboxamide;
{(3S)-1-[5-(cyclohexylcarbamoyl)-4-(propylthio)pyrimidin-2-yl]piperidin-3-yl}acetic acid;
N-[(2r,5s)-5-hydroxyadamantan-2-yl]-2-methylamino-4-propylsulfanylpyrimidine-5-carboxamide;
N-[(2r,5s)-5-hydroxyadamantan-2-yl]-4-methylamino-2-propylsulfanylpyrimidine-5-carboxamide;
2-[(2S,6R)-2,6-dimethylmorpholin-4-yl]-N-[(2r,5s)-5-hydroxyadamantan-2-yl]-4-propylsulfanylpyrimidine-5-carboxamide;
4-[(2S,6R)-2,6-dimethylmorpholin-4-yl]-N-[(2r,5s)-5-hydroxyadamantan-2-yl]-2-propylsulfanylpyrimidine-5-carboxamide;
4-(4-acetylpiperazin-1-yl)-N-[(2r,5s)-5-hydroxyadamantan-2-yl]-2-propylsulfanylpyrimidine-5-carboxamide;
2-(4-acetylpiperazin-1-yl)-N-[(2r,5s)-5-hydroxyadamantan-2-yl]-4-propylsulfanylpyrimidine-5-carboxamide;
2-(4-acetylpiperazin-1-yl)-N-(2-adamantyl)-4-propylsulfanyl-pyrimidine-5-carboxamide;
N-(2-adamantyl)-2-(4-methylsulfonylpiperazin-1-yl)-4-propylsulfanyl-pyrimidine-5-carboxamide;
N-(2-adamantyl)-2-[4-(dimethylcarbamoyl)piperazin-1-yl]-4-propylsulfanyl-pyrimidine-5-carboxamide;
4-cyclopentyl-N-[(2s,5r)-5-hydroxyadamantan-2-yl]-2-morpholin-4-ylpyrimidine-5-carboxamide;
N-[(2s,5r)-5-hydroxyadamantan-2-yl]-2-morpholin-4-yl-4-propoxypyrimidine-5-carboxamide;
4-cyclopropyl-N-[(2r,5s)-5-hydroxyadamantan-2-yl]-2-[(3R)-oxolan-3-ylamino]pyrimidine-5-carboxamide;
N-[(2r,5s)-5-hydroxyadamantan-2-yl] 4-cyclopropyl-2-[(3S)-oxolan-3-yl]amino]pyrimidine-5-carboxamide;
N-[(2s,5r)-5-hydroxyadamantan-2-yl]-2,4-dimorpholin-4-ylpyrimidine-5-carboxamide;
4-cyclopropyl-N-[(2r,5s)-5-hydroxyadamantan-2-yl]-2-methoxypyrimidine-5-carboxamide;
4-cyclopropyl-N-[(2r,5s)-5-hydroxyadamantan-2-yl]-2-methylaminopyrimidine-5-carboxamide;
4-cyclopropyl-N-[(2r,5s)-5-hydroxyadamantan-2-yl]-2-thiomorpholin-4-ylpyrimidine-5-carboxamide
4-cyclopropyl-N-[(2r,5 s)-5-hydroxyadamantan-2-yl]-2-(1-oxo-1,4-thiazinan-4-yl)pyrimidine-5-carboxamide;
4-cyclopropyl-2-(1,1-dioxo-1,4-thiazinan-4-yl)-N-[(2r,5 s)-5-hydroxyadamantan-2-yl]pyrimidine-5-carboxamide;
4-cyclohexyl-N-[(2r,5 s)-5-hydroxyadamantan-2-yl]-2-morpholin-4-ylpyrimidine-5-carboxamide;
4-cyclopentyl-N-[(2r,5s)-5-hydroxyadamantan-2-yl]-2-methylpyrimidine-5-carboxamide;
4-cyclobutyl-N-[(2r,5s)-5-hydroxyadamantan-2-yl]-2-morpholin-4-ylpyrimidine-5-carboxamide;
4-cyclobutyl-N-[(2r,5 s)-5-hydroxyadamantan-2-yl]-2-thiomorpholin-4-ylpyrimidine-5-carboxamide
4-cyclopropyl-2-(2,6-dimethylmorpholin-4-yl)-N-[(2r,5s)-5-hydroxyadamantan-2-yl]pyrimidine-5-carboxamide;
4-cyclopropyl-2-(3,3-difluoroazetidin-1-yl)-N-[(2r,5s)-5-hydroxyadamantan-2-yl]pyrimidine-5-carboxamide;
2-(azetidin-1-yl)-4-cyclopropyl-N-[(2r,5s)-5-hydroxyadamantan-2-yl]pyrimidine-5-carboxamide;
2-(cyclobutylamino)-4-cyclopropyl-N-[(2r,5s)-5-hydroxyadamantan-2-yl]pyrimidine-5-carboxamide;
4-cyclopropyl-N-[(2r,5s)-5-hydroxyadamantan-2-yl]-2-[4-(2-methoxyethyl)piperazin-1-yl]pyrimidine-5-carboxamide;
4-cyclopropyl-2-(cyclopropylamino)-N-[(2r,5s)-5-hydroxyadamantan-2-yl]pyrimidine-5-carboxamide;
2-(cyclopentylamino)-4-cyclopropyl-N-[(2r,5s)-5-hydroxyadamantan-2-yl]pyrimidine-5-carboxamide;
4-cyclopropyl-N-[(2r,5s)-5-hydroxyadamantan-2-yl]-2-[(1S,4S)-2-oxa-5-azabicyclo[2.2.1]hept-5-yl]pyrimidine-5-carboxamide;
4-cyclopropyl-N-[(2r,5s)-5-hydroxyadamantan-2-yl]-2-[2-(hydroxymethyl)morpholin-4-yl]pyrimidine-5-carboxamide;
4-cyclopropyl-N-[(2r,5s)-5-hydroxyadamantan-2-yl]-2-[3-(hydroxymethyl)morpholin-4-yl]pyrimidine-5-carboxamide;
4-cyclopropyl-2-(dimethylamino)-N-[(2r,5s)-5-hydroxyadamantan-2-yl]pyrimidine-5-carboxamide;
4-cyclopropyl-2-[(3R,5S)-3,5-dimethylpiperazin-1-yl]-N-[(2r,5 s)-5-hydroxyadamantan-2-yl]pyrimidine-5-carboxamide;
4-cyclopropyl-2-[(2R,6R)-2,6-dimethylmorpholin-4-yl]-N-[(2r,5 s)-5-hydroxyadamantan-2-yl]pyrimidine-5-carboxamide;
4-cyclopropyl-N-[(2r,5s)-5-hydroxyadamantan-2-yl]-2-(isopropylamino)pyrimidine-5-carboxamide;
4-cyclopropyl-N-[(2r,5s)-5-hydroxyadamantan-2-yl]-2-[(2-hydroxy-1,1-dimethylethyl)amino]pyrimidine-5-carboxamide;
4-cyclopropyl-N-[(2r,5s)-5-hydroxyadamantan-2-yl]-2-(tetrahydro-2H-pyran-4-ylamino)pyrimidine-5-carboxamide;
4-cyclopropyl-N-[(2r,5s)-5-hydroxyadamantan-2-yl]-2-[(2-hydroxy-2-methylpropyl)amino]pyrimidine-5-carboxamide;
4-cyclopropyl-2-[(1,1-dioxidotetrahydro-2H-thiopyran-4-yl)amino]-N-[(2r,5s)-5-hydroxyadamantan-2-yl]pyrimidine-5-carboxamide;
4-cyclopropyl-N-[(2r,5s)-5-hydroxyadamantan-2-yl]-2-[(2-hydroxyethyl)amino]pyrimidine-5-carboxamide;
4-cyclopropyl-N-[(2r,5s)-5-hydroxyadamantan-2-yl]-2-(4-methylsulfonylpiperazin-1-yl)pyrimidine-5-carboxamide;
4-cyclopropyl-N-[(2r,5s)-5-hydroxyadamantan-2-yl]-2-(oxetan-3-ylamino)pyrimidine-5-carboxamide;
4-cyclopropyl-N-[(2r,5s)-5-hydroxyadamantan-2-yl]-2-[(2-morpholin-4-ylethyl)amino]pyrimidine-5-carboxamide;
4-cyclopropyl-2-({2-[(2R,6S)-2,6-dimethylmorpholin-4-yl]ethyl}amino)-N-[(2r,5 s)-5-hydroxyadamantan-2-yl]pyrimidine-5-carboxamide;
4-cyclopropyl-N-[(2r,5s)-5-hydroxyadamantan-2-yl]-2-{[2-(4-methylpiperazin-1-yl)ethyl]amino}pyrimidine-5-carboxamide;
2-(cyclobutyloxy)-4-cyclopropyl-N-[(2r,5s)-5-hydroxyadamantan-2-yl]pyrimidine-5-carboxamide;
4-cyclopropyl-N-[(2r,5s)-5-hydroxyadamantan-2-yl]-2-isopropoxypyrimidine-5-carboxamide;
2-(cyclopentyloxy)-4-cyclopropyl-N-[(2r,5s)-5-hydroxyadamantan-2-yl]pyrimidine-5-carboxamide;
4-cyclopropyl-N-[(2r,5s)-5-hydroxyadamantan-2-yl]-2-(oxetan-3-yloxyl)pyrimidine-5-carboxamide;
(4-cyclopropyl-2-morpholinopyrimidin-5-yl)(3-(pyridin-3-yl)pyrrolidin-1-yl)methanone
1-(4-(4-cyclopropyl-5-(3-(pyridin-3-yl)pyrrolidine-1-carbonyl)pyrimidin-2-yl)piperazin-1-yl)ethanone;
(4-cyclopropyl-2-((2S,6R)-2,6-dimethylmorpholino)pyrimidin-5-yl)(3-(pyridin-3-yl)pyrrolidin-1-yl)methanone;
4-cyclobutyl-N-[(2r,5 s)-5-hydroxyadamantan-2-yl]-2-(1-oxo-1,4-thiazinan-4-yl)pyrimidine-5-carboxamide;
4-cyclobutyl-2-(1,1-dioxo-1,4-thiazinan-4-yl)-N-[(2r,5 s)-5-hydroxyadamantan-2-yl]pyrimidine-5-carboxamide;
2-amino-4-cyclobutyl-N-[(2r,5s)-5-hydroxyadamantan-2-yl]pyrimidine-5-carboxamide;
2-azetidin-1-yl-4-cyclobutyl-N-[(2r,5 s)-5-hydroxyadamantan-2-yl]pyrimidine-5-carboxamide;
4-cyclobutyl-2-(dimethylamino)-N-[(2r,5 s)-5-hydroxyadamantan-2-yl]pyrimidine-5-carboxamide;
4-cyclobutyl-N-[(2r,5 s)-5-hydroxyadamantan-2-yl]-2-[4-(2-methoxyethyl)piperazin-1-yl]pyrimidine-5-carboxamide;
4-cyclobutyl-2-(cyclopropylamino)-N-[(2r,5s)-5-hydroxyadamantan-2-yl]pyrimidine-5-carboxamide;
4-cyclobutyl-2-(3,3-difluoroazetidin-1-yl)-N-[(2r,5s)-5-hydroxyadamantan-2-yl]pyrimidine-5-carboxamide;
4-cyclobutyl-N-[(2r,5 s)-5-hydroxyadamantan-2-yl]-2-[3-(hydroxymethyl)morpholin-4-yl]pyrimidine-5-carboxamide;
4-cyclobutyl-N-[(2r,5s)-5-hydroxyadamantan-2-yl]-2-(methylamino)pyrimidine-5-carboxamide;
4-cyclobutyl-2-[(2R,6S)-2,6-dimethylmorpholin-4-yl]-N-[(2r,5s)-5-hydroxyadamantan-2-yl]pyrimidine-5-carboxamide;
4-cyclobutyl-N-[(2r,5 s)-5-hydroxyadamantan-2-yl]-2-[2-(hydroxymethyl)morpholin-4-yl]pyrimidine-5-carboxamide;
4-cyclobutyl-N-[(2r,5s)-5-hydroxyadamantan-2-yl]-2-(isopropylamino)pyrimidine-5-carboxamide;
4-cyclobutyl-N-[(2r,5s)-5-hydroxyadamantan-2-yl]-2-[(2-hydroxy-1,1-dimethylethyl)amino]pyrimidine-5-carboxamide;
4-cyclobutyl-N-[(2r,5 s)-5-hydroxyadamantan-2-yl]-2-(tetrahydro-2H-pyran-4-ylamino)pyrimidine-5-carboxamide;
4-cyclobutyl-N-[(2r,5s)-5-hydroxyadamantan-2-yl]-2-[(2-hydroxyethyl)amino]pyrimidine-5-carboxamide;
N-[(2r,5s)-5-hydroxyadamantan-2-yl]4-cyclobutyl-2-(cyclobutylamino) pyrimidine-5-carboxamide;
4-cyclobutyl-2-[(3R,5S)-3,5-dimethylpiperazin-1-yl]-N-[(2r,5 s)-5-hydroxyadamantan-2-yl]pyrimidine-5-carboxamide;
4-cyclobutyl-N-[(2r,5s)-5-hydroxyadamantan-2-yl]-2-[(2-hydroxy-2-methylpropyl)amino]pyrimidine-5-carboxamide;
4-cyclobutyl-2-[(2R,6R)-2,6-dimethylmorpholin-4-yl]-N-[(2r,5 s)-5-hydroxyadamantan-2-yl]pyrimidine-5-carboxamide;
4-cyclobutyl-2-(cyclopentylamino)-N-[(2r,5s)-5-hydroxyadamantan-2-yl]pyrimidine-5-carboxamide;
4-cyclobutyl-N-[(2r,5s)-5-hydroxyadamantan-2-yl]-2-[(1S,4S)-2-oxa-5-azabicyclo[2.2.1]hept-5-yl]pyrimidine-5-carboxamide;
4-cyclobutyl-N-[(2r,5s)-5-hydroxyadamantan-2-yl]-2-(oxetan-3-ylamino)pyrimidine-5-carboxamide;
4-cyclobutyl-2-[(1,1-dioxidotetrahydro-2H-thiopyran-4-yl)amino]-N-[(2r,5 s)-5-hydroxyadamantan-2-yl]pyrimidine-5-carboxamide;
4-cyclobutyl-2-(cyclopentyloxy)-N-[(2r,5s)-5-hydroxyadamantan-2-yl]pyrimidine-5-carboxamide;
4-cyclobutyl-N-[(2r,5s)-5-hydroxyadamantan-2-yl]-2-isopropoxypyrimidine-5-carboxamide;
4-cyclobutyl-2-(cyclobutyloxy)-N-[(2r,5s)-5-hydroxyadamantan-2-yl]pyrimidine-5-carboxamide;
4-cyclobutyl-N-[(2r,5 s)-5-hydroxyadamantan-2-yl]-2-(oxetan-3-yloxyl)pyrimidine-5-carboxamide;
4-cyclopentyl-N-[(2r,5s)-5-hydroxyadamantan-2-yl]-2-propan-2-yloxypyrimidine-5-carboxamide;
2-cyclobutyloxy-4-cyclopentyl-N-[(2r,5s)-5-hydroxyadamantan-2-yl]pyrimidine-5-carboxamide;
4-cyclopentyl-2-cyclopentyloxy-N-[(2r,5s)-5-hydroxyadamantan-2-yl]pyrimidine-5-carboxamide;
4-cyclopentyl-N-[(2r,5 s)-5-hydroxyadamantan-2-yl]-2-(oxetan-3-yloxyl)pyrimidine-5-carboxamide;
4-cyclopentyl-N-[(2s,5r)-5-hydroxyadamantan-2-yl]-2-thiomorpholin-4-ylpyrimidine-5-carboxamide;
4-cyclopentyl-N-[(2r,5 s)-5-hydroxyadamantan-2-yl]-2-(1-oxo-1,4-thiazinan-4-yl)pyrimidine-5-carboxamide;
4-cyclopentyl-2-(1,1-dioxo-1,4-thiazinan-4-yl)-N-[(2r,5 s)-5-hydroxyadamantan-2-yl]pyrimidine-5-carboxamide;
4-cyclopentyl-N-[(2r,5 s)-5-hydroxyadamantan-2-yl]-2-methoxypyrimidine-5-carboxamide;
4-cyclopentyl-N-[(2r,5 s)-5-hydroxyadamantan-2-yl]-2-methylaminopyrimidine-5-carboxamide;
4-cyclopentyl-2-[(2S,6R)-2,6-dimethylmorpholin-4-yl]-N-[(2r,5s)-5-hydroxyadamantan-2-yl]pyrimidine-5-carboxamide;
4-cyclopentyl-N-[(2r,5s)-5-hydroxyadamantan-2-yl]-2-[(1S,4S)-2-oxa-5-azabicyclo[2.2.1]heptan-5-yl]pyrimidine-5-carboxamide;
4-cyclopentyl-N-[(2r,5s)-5-hydroxyadamantan-2-yl]-2-(propan-2-ylamino)pyrimidine-5-carboxamide;
4-cyclopentyl-2-(cyclopropylamino)-N-[(2r,5s)-5-hydroxyadamantan-2-yl]pyrimidine-5-carboxamide;
4-cyclopentyl-N-[(2r,5s)-5-hydroxyadamantan-2-yl]-2-[(3S)-3-methylmorpholin-4-yl]pyrimidine-5-carboxamide;
4-cyclopentyl-2-[(2S,6S)-2,6-dimethylmorpholin-4-yl]-N-[(2r,5 s)-5-hydroxyadamantan-2-yl]pyrimidine-5-carboxamide;
4-cyclopentyl-N-[(2r,5 s)-5-hydroxyadamantan-2-yl]-2-[4-(2-methoxyethyl)piperazin-1-yl]pyrimidine-5-carboxamide;
2-(4-acetylpiperazin-1-yl)-4-cyclopentyl-N-[(2r,5s)-5-hydroxyadamantan-2-yl]pyrimidine-5-carboxamide;
4-cyclopentyl-N-[(2r,5 s)-5-hydroxyadamantan-2-yl]-2-(3-oxo-4-propan-2-ylpiperazin-1-yl)pyrimidine-5-carboxamide;
4-cyclopentyl-N-[(2r,5 s)-5-hydroxyadamantan-2-yl]-2-(4-methyl-3-oxopiperazin-1-yl)pyrimidine-5-carboxamide;
4-cyclopentyl-2-(cyclobutylamino)-N-[(2r,5s)-5-hydroxyadamantan-2-yl]pyrimidine-5-carboxamide;
4-cyclopentyl-2-(cyclopentylamino)-N-[(2r,5s)-5-hydroxyadamantan-2-yl]pyrimidine-5-carboxamide;
2-(azetidin-1-yl)-4-cyclopentyl-N-[(2r,5s)-5-hydroxyadamantan-2-yl]pyrimidine-5-carboxamide;
4-cyclopentyl-N-[(2r,5 s)-5-hydroxyadamantan-2-yl]-2-(oxetan-3-ylamino)pyrimidine-5-carboxamide;
4-cyclopentyl-2-dimethylamino-N-[(2r,5s)-5-hydroxyadamantan-2-yl]pyrimidine-5-carboxamide;
4-cyclopentyl-2-[(3S,5R)-3,5-dimethylpiperazin-1-yl]-N-[(2r,5 s)-5-hydroxyadamantan-2-yl]pyrimidine-5-carboxamide;
2-amino-4-cyclopentyl-N-[(2r,5s)-5-hydroxyadamantan-2-yl]pyrimidine-5-carboxamide;
4-cyclopentyl-2-[(1,1-dioxothian-4-yl)amino]-N-[(2r,5 s)-5-hydroxyadamantan-2-yl]pyrimidine-5-carboxamide;
4-cyclopentyl-N-[(2r,5 s)-5-hydroxyadamantan-2-yl]-2-[(2-hydroxy-2-methylpropyl)amino]pyrimidine-5-carboxamide;
4-cyclopentyl-N-[(2r,5s)-5-hydroxyadamantan-2-yl]-2-(2-hydroxyethylamino)pyrimidine-5-carboxamide;
4-cyclopentyl-N-[(2r,5 s)-5-hydroxyadamantan-2-yl]-2-[(1-hydroxy-2-methylpropan-2-yl)amino]pyrimidine-5-carboxamide;
4-cyclopentyl-N-[(2r,5s)-5-hydroxyadamantan-2-yl]-2-(oxan-4-ylamino)pyrimidine-5-carboxamide;
4-cyclopentyl-N-[(2r,5 s)-5-hydroxyadamantan-2-yl]-2-[3-(hydroxymethyl)morpholin-4-yl]pyrimidine-5-carboxamide;
4-cyclopentyl-N-[(2r,5 s)-5-hydroxyadamantan-2-yl]-2-[[(3R)-oxolan-3-yl]amino]pyrimidine-5-carboxamide;
4-cyclopentyl-N-[(2r,5s)-5-hydroxyadamantan-2-yl]-2-(4-methylsulfonylpiperazin-1-yl)pyrimidine-5-carboxamide;
4-cyclopentyl-N-[(2r,5 s)-5-hydroxyadamantan-2-yl]-2-[[(3S)-oxolan-3-yl]amino]pyrimidine-5-carboxamide;
4-cyclopentyl-N-[(2r,5 s)-5-hydroxyadamantan-2-yl]-2-[2-(hydroxymethyl)morpholin-4-yl]pyrimidine-5-carboxamide;
4-cyclopentyl-2-(3,3-difluoroazetidin-1-yl)-N-[(2r,5s)-5-hydroxyadamantan-2-yl]pyrimidine-5-carboxamide;
4-cyclopentyl-N-[(2r,5 s)-5-hydroxytricyclo[3.3.1.13.7]dec-2-yl]-2-[(2-morpholin-4-ylethyl)amino]pyrimidine-5-carboxamide;
4-cyclopentyl-2-({2-[(2R,6S)-2,6-dimethylmorpholin-4-yl]ethyl}amino)-N-[(2r,5 s)-5-hydroxytricyclo[3.3.1.13.7]dec-2-yl]pyrimidine-5-carboxamide;
4-cyclopentyl-2-cyclopropyl-N-[(2r,5s)-5-hydroxyadamantan-2-yl]pyrimidine-5-carboxamide;
4-cyclopentyl-N-[(2r,5s)-5-hydroxyadamantan-2-yl]-2-propan-2-ylpyrimidine-5-carboxamide2-(1-aminocyclopropyl)-4-cyclopentyl-N-[(2r,5s)-5-hydroxyadamantan-2-yl]pyrimidine-5-carboxamide;
2-(aminomethyl)-4-cyclopentyl-N-[(2r,5 s)-5-hydroxyadamantan-2-yl]pyrimidine-5-carboxamide;
4-(3,3-difluorocyclobutyl)-N-[(2s,5r)-5-hydroxyadamantan-2-yl]-2-methylpyrimidine-5-carboxamide;
4-(3,3-difluorocyclobutyl)-N-[(2r,5s)-5-hydroxyadamantan-2-yl]-2-methylaminopyrimidine-5-carboxamide;
2-(cyclopropylamino)-4-(3,3-difluorocyclobutyl)-N-[(2r,5 s)-5-hydroxyadamantan-2-yl]pyrimidine-5-carboxamide;
4-(3,3-difluorocyclobutyl)-2-[(2S,6R)-2,6-dimethylmorpholin-4-yl]-N-[(2r,5s)-5-hydroxyadamantan-2-yl]pyrimidine-5-carboxamide;
2-cyclobutyloxy-4-(3,3-difluorocyclobutyl)-N-[(2r,5s)-5-hydroxyadamantan-2-yl]pyrimidine-5-carboxamide;
N-[(2r,5s)-5-hydroxyadamantan-2-yl]-2-methyl-4-(oxolan-2-yl)pyrimidine-5-carboxamide;
N-[(2r,5s)-5-hydroxyadamantan-2-yl]-4-(oxolan-2-yl)-2-(propan-2-ylamino)pyrimidine-5-carboxamide;
2-(cyclopropylamino)-N-[(2r,5s)-5-hydroxyadamantan-2-yl]-4-(oxolan-2-yl)pyrimidine-5-carboxamide;
N-[(2r,5 s)-5-hydroxyadamantan-2-yl]-2-methylamino-4-(oxolan-2-yl)pyrimidine-5-carboxamide;
2-(cyclobutylamino)-N-[(2r,5s)-5-hydroxyadamantan-2-yl]-4-(oxolan-2-yl)pyrimidine-5-carboxamide;
2-[(2S,6R)-2,6-dimethylmorpholin-4-yl]-N-[(2r,5 s)-5-hydroxyadamantan-2-yl]-4-(oxolan-2-yl)pyrimidine-5-carboxamide;
N-[(2r,5s)-5-hydroxyadamantan-2-yl]-2-(oxetan-3-ylamino)-4-(oxolan-2-yl)pyrimidine-5-carboxamide;
N-[(2r,5s)-5-hydroxyadamantan-2-yl]-4-(oxolan-2-yl)-2-propan-2-yloxypyrimidine-5-carboxamide;
N-[(2r,5s)-5-hydroxyadamantan-2-yl]-2-methylsulfanyl-4-[(2R)-oxolan-2-yl]pyrimidine-5-carboxamide;
N-[(2r,5 s)-5-hydroxyadamantan-2-yl]-2-methylamino-4-[(2R)-oxolan-2-yl]pyrimidine-5-carboxamide;
2-(cyclopropylamino)-N-[(2r,5s)-5-hydroxyadamantan-2-yl]-4-[(2R)-oxolan-2-yl]pyrimidine-5-carboxamide;
N-[(2r,5 s)-5-hydroxyadamantan-2-yl]-4-[(2R)-oxolan-2-yl]-2-(propan-2-ylamino)pyrimidine-5-carboxamide;
2-[(3S,5R)-3,5-dimethylpiperazin-1-yl]-N-[(2r,5s)-5-hydroxyadamantan-2-yl]-4-[(2R)-oxolan-2-yl]pyrimidine-5-carboxamide;
N-[(2r,5s)-5-hydroxyadamantan-2-yl]-2-(oxetan-3-ylamino)-4-[(2R)-oxolan-2-yl]pyrimidine-5-carboxamide;
N-[(2r,5s)-5-hydroxyadamantan-2-yl]-2-(oxan-4-ylamino)-4-[(2R)-oxolan-2-yl]pyrimidine-5-carboxamide;
2-(cyclobutylamino)-N-[(2r,5 s)-5-hydroxyadamantan-2-yl]-4-[(2R)-oxolan-2-yl]pyrimidine-5-carboxamide;
N-[(2r,5 s)-5-hydroxyadamantan-2-yl]-4-[(2R)-oxolan-2-yl]-2-propan-2-yloxypyrimidine-5-carboxamide;
N-[(2r,5s)-5-hydroxyadamantan-2-yl]-2-methylsulfanyl-4-[(2S)-oxolan-2-yl]pyrimidine-5-carboxamide;
N-[(2r,5 s)-5-hydroxyadamantan-2-yl]-2-methylamino-4-[(2S)-oxolan-2-yl]pyrimidine-5-carboxamide;
N-[(2r,5s)-5-hydroxyadamantan-2-yl]-4-[(2S)-oxolan-2-yl]-2-(propan-2-ylamino)pyrimidine-5-carboxamide;
2-(cyclopropylamino)-N-[(2r,5 s)-5-hydroxyadamantan-2-yl]-4-[(2S)-oxolan-2-yl]pyrimidine-5-carboxamide;
N-[(2r,5s)-5-hydroxyadamantan-2-yl]-4-[(2S)-oxolan-2-yl]-2-propan-2-yloxypyrimidine-5-carboxamide;
2-[(2S,6R)-2,6-Dimethylmorpholin-4-yl]-N-[(2r,5s)-5-hydroxyadamantan-2-yl]-4-[(2R)-oxolan-2-yl]pyrimidine-5-carboxamide;
2-[(2S,6R)-2,6-Dimethylmorpholin-4-yl]-N-[(2r,5s)-5-hydroxyadamantan-2-yl]-4-[(2S)-oxolan-2-yl]pyrimidine-5-carboxamide;
4-(3,3-Difluorocyclopentyl)-N-[(2r,5s)-5-hydroxyadamantan-2-yl]-2-methylpyrimidine-5-carboxamide;
N-[(2r,5 s)-5-hydroxyadamantan-2-yl]-4-(1-methylcyclopropyl)-2-morpholin-4-ylpyrimidine-5-carboxamide;
N-[(2r,5 s)-5-hydroxyadamantan-2-yl]-4-(1-methylcyclopropyl)-2-morpholin-4-ylpyrimidine-5-carboxamide;
(Z)-3-dimethylamino-2-(1-methylcyclopropanecarbonyl)-N-(5-phenylmethoxy-2-adamantyl)prop-2-enamide;
N-[(2r,5s)-5-hydroxyadamantan-2-yl]-2-methyl-4-phenylpyrimidine-5-carboxamide;
4-(2-Chlorophenyl)-N-[(2r,5s)-5-hydroxyadamantan-2-yl]-2-methylpyrimidine-5-carboxamide;
4-(cyclopentylmethyl)-N-[(2r,5 s)-5-hydroxyadamantan-2-yl]-2-methylpyrimidine-5-carboxamide;
4-butyl-N-[(2r,5s)-5-hydroxyadamantan-2-yl]-2-methylpyrimidine-5-carboxamide;
N-[(2s,5r)-5-hydroxyadamantan-2-yl]-4-isobutyl-2-methylpyrimidine-5-carboxamide;
4-(2,2-dimethylpropyl)-N-[(2r,5s)-5-hydroxyadamantan-2-yl]-2-methylpyrimidine-5-carboxamide;
4-(cyclopropylmethyl)-N-[(2r,5s)-5-hydroxyadamantan-2-yl]-2-methylpyrimidine-5-carboxamide
4-cyclohexyl-N-[(2r,5 s)-5-hydroxyadamantan-2-yl]-2-(methylthio)pyrimidine-5-carboxamide;
4-cyclohexyl-N-[(2r,5 s)-5-hydroxyadamantan-2-yl]-2-thiomorpholin-4-ylpyrimidine-5-carboxamide;
4-cyclohexyl-N-[(2r,5 s)-5-hydroxyadamantan-2-yl]-2-(1-oxidothiomorpholin-4-yl)pyrimidine-5-carboxamide;
4-cyclohexyl-2-(1,1-dioxidothiomorpholin-4-yl)-N-[(2r,5 s)-5-hydroxyadamantan-2-yl]pyrimidine-5-carboxamide;
2,4-bis(dimethylamino)-N-[(2r,5 s)-5-hydroxyadamantan-2-yl]pyrimidine-5-carboxamide;
2,4-bis(3,3-difluoroazetidin-1-yl)-N-[(2r,5s)-5-hydroxyadamantan-2-yl]pyrimidine-5-carboxamide;
2,4-bis(azetidin-1-yl)-N-[(2r,5s)-5-hydroxyadamantan-2-yl]pyrimidine-5-carboxamide;
N-[(2r,5s)-5-hydroxyadamantan-2-yl]-2-methyl-4-propan-2-yloxypyrimidine-5-carboxamide;
4-cyclobutyloxy-N-[(2r,5s)-5-hydroxyadamantan-2-yl]-2-methylpyrimidine-5-carboxamide;
4-cyclopentyloxy-N-[(2r,5s)-5-hydroxyadamantan-2-yl]-2-methylpyrimidine-5-carboxamide;
2-[(2R,6S)-2,6-Dimethylmorpholin-4-yl]-N-[(2r,5 s)-5-hydroxytricyclo[3.3.1.13.7]dec-2-yl]-4-methoxypyrimidine-5-carboxamide;
2-(cyclopropylamino)-N-[(2r,5 s)-5-hydroxytricyclo[3.3.1.13.7]dec-2-yl]-4-methoxypyrimidine-5-carboxamide;
2-(cyclobutylamino)-N-[(2r,5s)-5-hydroxytricyclo[3.3.1.13.7]dec-2-yl]-4-methoxypyrimidine-5-carboxamide;
2-(cyclobutyloxy)-N-[(2r,5s)-5-hydroxytricyclo[3.3.1.13.7]dec-2-yl]-4-methoxypyrimidine-5-carboxamide;
2-[(2S,6R)-2,6-dimethylmorpholin-4-yl]-4-ethoxy-N-[(2r,5s)-5-hydroxyadamantan-2-yl]pyrimidine-5-carboxamide;
2-(cyclopropylamino)-4-ethoxy-N-[(2r,5s)-5-hydroxytricyclo[3.3.1.13.7]dec-2-yl]pyrimidine-5-carboxamide;
4-ethoxy-N-[(2r,5 s)-5-hydroxytricyclo[3.3.1.13.7]dec-2-yl]-2-(oxetan-3-ylamino)pyrimidine-5-carboxamide;
2-(cyclobutylamino)-4-ethoxy-N-[(2r,5s)-5-hydroxytricyclo[3.3.1.13.7]dec-2-yl]pyrimidine-5-carboxamide;
2-(cyclobutyloxy)-4-ethoxy-N-[(2r,5s)-5-hydroxytricyclo[3.3.1.13.7]dec-2-yl]pyrimidine-5-carboxamide;
2-[(2R,6S)-2,6-dimethylmorpholin-4-yl]-N-[(2r,5s)-5-hydroxytricyclo[3.3.1.13.7]dec-2-yl]-4-(1-methylethoxyl)pyrimidine-5-carboxamide;
2-(cyclopropylamino)-N-[(2r,5s)-5-hydroxytricyclo[3.3.1.13.7]dec-2-yl]-4-(1-methylethoxyl)pyrimidine-5-carboxamide;
N-[(2r,5s)-5-hydroxytricyclo[3.3.1.13.7]dec-2-yl]-4-(1-methylethoxy)-2-(oxetan-3-ylamino)pyrimidine-5-carboxamide;
2-(cyclobutylamino)-N-[(2r,5 s)-5-hydroxytricyclo[3.3.1.13.7]dec-2-yl]-4-(1-methylethoxyl)pyrimidine-5-carboxamide;
2-(cyclobutyloxy)-N-[(2r,5s)-5-hydroxytricyclo[3.3.1.13.7]dec-2-yl]-4-(1-methylethoxyl)pyrimidine-5-carboxamide;
N-[(2r,5s)-5-hydroxyadamantan-2-yl]2-[(2S,6R)-2,6-dimethylmorpholin-4-yl]-4-[(2R)-oxolan-2-yl]pyrimidine-5-carboxamide;
4-cyclopropyl-2-[(2S,6R)-2,6-dimethylmorpholin-4-yl]-N-[(2r,5s)-5-hydroxyadamantan-2-yl]pyrimidine-5-carboxamide; and
2-[(2S,6R)-2,6-dimethylmorpholin-4-yl]-N-[(2r,5s)-5-hydroxyadamantan-2-yl]-4-(methoxymethyl)pyrimidine-5-carboxamide.

Another aspect of the present invention provides a process for preparing a compound of formula 1 or a pharmaceutically acceptable salt thereof which process [wherein variable groups are, unless otherwise specified, as defined in formula 1] comprises any one of the following processes;

a) suitable for when Q is a single bond linked to a carbon atom:

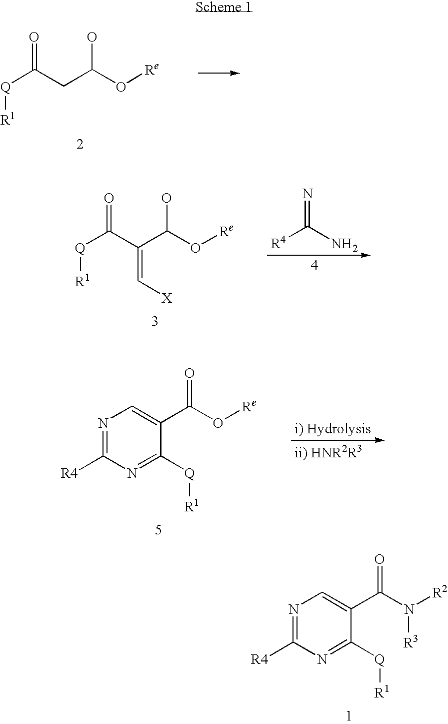

According to this method, a β-ketoester of formula 2 is converted to a compound of formula 3 where X represents dialkylamino (e.g. dimethylamino) or lower alkoxy (e.g. ethoxy). The compound of formula 3 is then treated with an appropriately substituted amidine or guanidine of formula 4. The ester protecting group in the compound of formula 5 is then cleaved and the resulting carboxylic acid is coupled with an amine of formula NHR²R³ to give the desired compound of formula 1.

Methods for conversion of compounds of formula 2 to enamines of formula 3 (X is dialklyamino) are well known to the art and examples are described in the following references; Tetrahedron Lett., 1984, 25, 3743; Synthesis, 1983, 566; Synthesis, 1990, 70. When X=dimethylamino, the reaction typically involves treating a compound of formula 2 with N,N-dimethylformamide dimethyl acetal in an inert solvent, typically 1,4-dioxane or toluene, at temperature between 50-100° C.

Methods for conversion of compounds of formula 2 to enol ethers of formula 3 (X is alkoxy) and well known to the art and examples are described in the following references; Liebigs Ann. Chem., 1897, 297, 1; J. Chem. Soc., Perkin Trans. 1, 1979, 464; J. Med. Chem., 2000, 43, 3995; Tetrahedron, 2002, 58, 8581; When X is ethoxy, the reaction typically involves treating a compound of formula 2 with triethylorthoformate in the presence of acetic anhydride at reflux.

Methods for conversion of compounds of formula 3 to pyrimidines of formula 5 are well known in the art and examples are described in the following references; Bioorg. Med. Chem. Lett., 2005, 15, 4898; Bioorg. Med. Chem. Lett., 2003, 13, 567; US 2005096353. The compound of formula 3 is treated with an appropriate amidine or guanidine of formula 4 in an inert solvent (e.g. methanol, ethanol) with an appropriate base (e.g. sodium ethoxide) at temperatures ranging from 50-100° C., preferably at reflux.

Methods for conversion of compounds of formula 5 to pyrimidines of formula 1 are well known to the art. Cleavage of a compound of formula 5 to the corresponding carboxylic acid will be dependent on the nature of the ester group used and many procedures are outlined in the following reference; T. W. Green, Protective Groups in Organic Synthesis, John Wiley and Sons, 1991. For example, in the case where R^{e} represents lower alkoxy (e.g. methyl or ethyl), the reaction can be carried out by hydrolysis with a suitable base such as an alkaline metal hydroxide (e.g. sodium hydroxide, potassium hydroxide or lithium hydroxide) in a suitable solvent (e.g. methanol, THF, water) at temperatures ranging from 0-50° C. but preferably at ambient temperature. In the case where R^{e} is an acid labile ester (e.g. t-butyl), the reaction may be carried out by treatment with an inorganic acid (e.g. hydrochloric acid) or an organic acid (e.g. trifluoroacetic acid) in a suitable solvent (e.g. dichloromethane) at temperatures ranging from 0-ambient but preferably at ambient temperature. In the case where R^{e} is an ester labile to hydrogenation (e.g. benzyl), the reaction may be carried out with a suitable catalyst (e.g. palladium-on-carbon) in the presence of an inert solvent (e.g. ethanol, methanol, toluene) typically at room temperature and a suitable pressure (typically atmospheric pressure) Formation of an amide from a carboxylic acid is a process well known to the art. Typical processes include, but are not limited to, formation of an acyl halide by treatment with a suitable reagent (e.g. oxalyl chloride, POCl₃) in a suitable solvent such as dichloromethane or N,N-dimethylformamide for example at temperatures ranging from 0-50° C. but preferably at ambient temperature. Alternatively, in situ conversion of the acid to an active ester derivative may be utilised with the addition of a suitable coupling agent (or combination of agents) to form an active ester such as HATU, 1-hydroxybenzotriazole (HOBT), and 1-ethyl-3-(3-dimethylaminopropyl)carbodimide hydrochloride (EDAC) for example, optionally in the presence of a suitable base such as triethylamine or N,N-di-iso-propylamine for example. Typically the reaction is carried out at temperatures ranging from 0-50° C. but preferably at ambient temperature.

Direct conversions of esters to amides are known in the art with examples described in the following references; J. Med. Chem., 2007, 50, 1675; Heterocycles, 2006, 67, 519 and typically involve heating of the two components, optionally in the presence of a suitable additive (e.g. AlMe₃). Typically reactions are carried out in inert solvents (e.g. toluene, benzene) at elevated temperatures (e.g. 50-150° C.) achieved through conventional or microwave heating.

b) suitable for when Q is a single bond linked to a carbon atom:

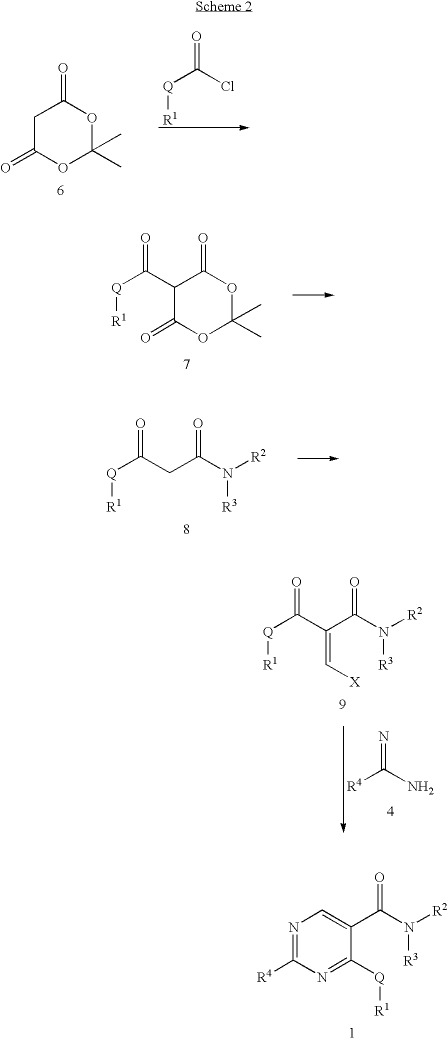

According to this method, Meldrum's acid of formula 6 is converted to a compound of formula 7. The compound of formula 7 is then treated with an amine of formula NHR²R³ to form a β-ketoamide of formula 8. This compound of formula 8 is then converted to a compound of formula 9 where X represents dialkylamino (e.g. dimethylamino) or lower alkoxy (e.g. ethoxy). The compound of formula 9 is then treated with an appropriately substituted amidine or guanidine of formula 4 to give the desired compound of formula 1.

Methods for conversion of compounds of formula 6 to compounds of formula 7 are well known in the art and examples are described in the following references; J. Org. Chem., 2001, 26, 6756; J. Med. Chem., 1998, 41, 3186. The Meldrum's acid is treated with an acyl chloride of formula R¹QCOCl in an anhydrous inert solvent (e.g. dichloromethane) in the presence of an organic base (e.g. pyridine, triethylamine, or N,N-diisopropylamine) at temperatures between 0-50° C., but preferably at 0° C. to ambient temperature. Methods for conversion of compounds of formula 7 to compounds of formula 8 are well known in the art and examples are described in the following reference; Synthesis., 1992, 1213. The compound of formula 7 is treated with a stoichiometric amount of amine of formula HNR²R³ in an inert solvent (e.g. toluene) at elevated temperature, preferably at reflux.

Methods for conversion of compounds of formula 8 to compounds of formula 9 are analogous to those previously outlined for the conversion of compounds of formula 2 to compounds of formula 3 described above. Methods for conversion of compounds of formula 9 to compounds of formula 1 are analogous to those previously outlined for the conversion of compounds of formula 3 to compounds of formula 5.

c) suitable for when Q is a single bond linked to a carbon atom:

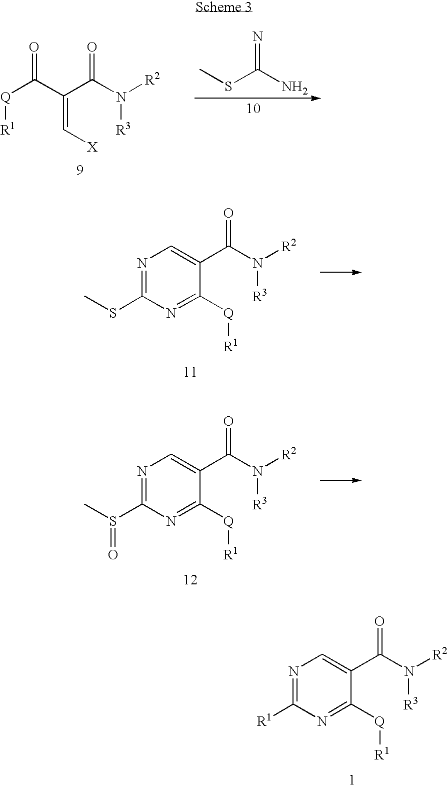

According to this method, a compound of formula 9 is converted to a compound of formula 11 by treatment with methylsulfonylformadine 10. The compound of formula 11 is then oxidised to give a sulphoxide of formula 12 which is reacted with an appropriate nucleophile to give the desired compound of formula 1.

Methods for conversion of compounds of formula 9 to pyrimidines of formula 11 are well known in the art and examples are described in the following patent reference; WO2006050476. The compound of formula 9 is treated with isothiourea sulphate 10 in an inert solvent (e.g. DMF) with an appropriate base (e.g. sodium acetate) and heated at temperatures of between 50-100° C., ideally at 80-90° C. to give pyrimidines of formula 11. Methods for conversion of thioethers of formula 11 to sulphoxides of formula 12 are well known in the art and examples are described in the following patent reference; WO2006050476. The compound of formula 11 is treated with an appropriate oxidising agent (e.g. m-chloroperbenzoic acid) in an inert solvent (e.g. dichloromethane) at temperatures ranging from −78° C. to ambient temperature, preferably at −10° C. to ambient temperature. It will be appreciated by those skilled in the art that the potential to further oxidise the sulphur to the corresponding sulphoxide also exists and that these compounds would also be suitable for the activation of this group towards nucleophilic displacement in the subsequent step.

Methods for conversion of compounds of formula 12 to compounds of formula 1 are well known in the art and examples are described in the following references; WO2006050476, Synth. Commun., 2007, 37, 2231; Bioorg. Med. Chem., 2005, 13, 5717. The compound of formula 12 is treated with an appropriate nucleophilic reagent in an inert solvent (e.g. THF, DMF, 1,4-dioxane) at temperatures ranging from ambient temperature to 100° C. dependant of the nucleophilicity of the reagent.

d) suitable for when Q is O, S, N(R⁸) or a single bond linked to a heteroatom;

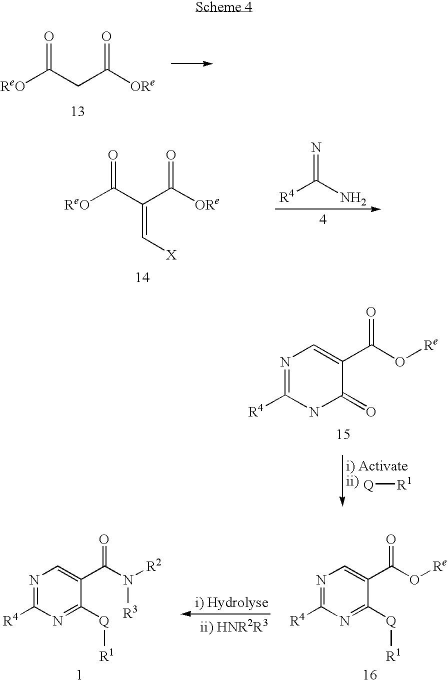

According to this method, a malonate of formula 13 is converted to a compound of formula 14. The compound of formula 14 is then treated with an appropriately substituted amidine or guanidine of formula 4 to give a pyrimidone of formula 15. The pyrimidone is then converted to a suitably reactive species and treated with a nucleophile to give pyrimidines of formula 16. The ester protecting group (R^{e}) in the compound of formula 16 is then cleaved and the resulting carboxylic acid is coupled with an amine of formula NHR²R³ to give the desired compound of formula 1.

Methods for conversion of malonates of formula 13 to compounds of formula 14 where X represents dialkylamino (e.g. dimethylamino) or lower alkoxy (e.g. ethoxy) are well known in the art and examples are described in the following references; J. Org. Chem., 1995, 60, 1900; Organic Synthesis; J. Wiley & Sons: New York, 1996: Collect. Vol 3, p395; EP 413918; EP 411417; WO 2002034710. When X is ethoxy, the reaction typically involves treating a compound of formula 13 with triethylorthoformate in the presence of acetic anhydride at reflux.

Methods for conversion of compounds of formula 14 to compounds of formula 15 are analogous to those previously outlined for the conversion of compounds of formula 3 to compounds of formula 5 described above.

Methods for conversion of compounds of formula 15 to pyrimidines of formula 16 are well known in the art and examples are described in the following references; J. Med. Chem., 2007, 50, 591. The compound of formula 15 is treated with a suitable halogenating system (e.g. POCl₃/PCl₅ or Cl₂P(═O)OPh) in an inert solvent (e.g. DMF) or neat with an and heated at temperatures of between 50-190° C., ideally at reflux to give halo pyrimidines which are then displaced with appropriate nucleophiles in an inert solvent (e.g. DMF, butyronitrile, DMF) in the presence of an appropriate base (e.g. potassium carbonate, sodium carbonate) at temperatures ranging from ambient temperature to 100° C. dependant of the nucleophilicity of the reagent to give compounds of formula 16. Optionally, the anion of the nucleophile may be prepared by treatment with a suitable base (e.g. sodium hydride, lithium hexamethyldisilazide).

Methods for conversion of compounds of formula 16 to compounds of formula 1 are analogous to those previously outlined for the conversion of compounds of formula 5 to compounds of formula 1 described above.

e) suitable for when Q is O, S, N(R⁸) or a single bond linked to a heteroatom;

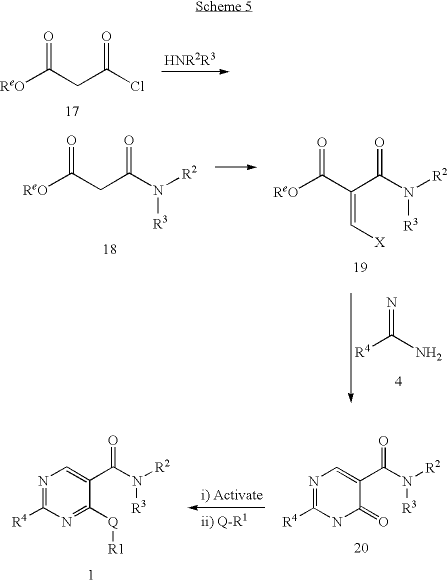

According to this method, an acid chloride of formula 17 is coupled with an amine of formula NHR²R³ and converted to an amide of formula 18. The amide of formula 19 is then converted to a compound of formula 19 where X represents dialkylamino (e.g. dimethylamino) or lower alkoxy (e.g. ethoxy). The amide of formula 19 is then treated with an appropriately substituted amidine or guanidine of formula 4 to give a pyrimidone of formula 20. The pyrimidone is then converted to a suitably reactive species and treated with a nucleophile to give the desired compound of formula 1.

Methods for conversion of compounds of formula 17 to amides of formula 18 are well known in the art and examples are described in the following references; J. Org. Chem., 2007, 72, 7058; Bioorg. Med. Chem. Lett., 2007, 17, 1951. The compound of formula 17 is treated with an amine of formula NHR²R³ in the presence of a suitable base (e.g. triethylamine, pyridine) in a suitable solvent (e.g. dichloromethane) at temperatures of 0-50° C., typically at 0° C. to ambient temperature.

Methods for conversion of compounds of formula 18 to compounds of formula 19 are analogous to those previously outlined for the conversion of compounds of formula 2 to compounds of formula 3 described above.

Methods for conversion of compounds of formula 19 to compounds of formula 20 are analogous to those previously outlined for the conversion of compounds of formula 3 to compounds of formula 5 described above.

Methods for conversion of compounds of formula 20 to compounds of formula 1 are analogous to those previously outlined for the conversion of compounds of formula 15 to compounds of formula 16 described above.

f) suitable for when Q is O, S, N(R⁸) or a single bond linked to a heteroatom;

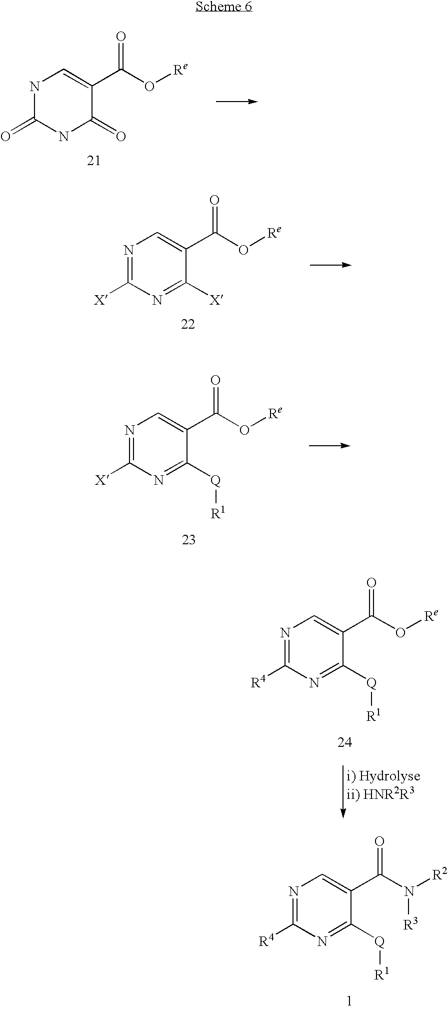

According to this method, a pyrimidinedione ester of formula 21 is halogenated to give a di-halo (or equivalent) compound of formula 22 wherein X′ is halo. The compound is treated with a stoichiometric quantity of an appropriate nucleophile (Q-R¹) to give compounds of formula 23 and then reacted with another nucleophile (R⁴) to give a pyrimidine of formula 24. The ester protecting group (R^{e}) in the compound of formula 24 is then cleaved and the resulting carboxylic acid is coupled with an amine of formula NHR²R³ to give the desired compound of formula 1.

Methods for conversion of compounds of formula 21 to compounds of formula 22 are well known in the art and examples are described in the following references; J. Med. Chem., 2007, 50, 591. The compound of formula 21 is treated with a suitable halogenating system (e.g. POCl₃/PCl₅ or Cl₂P(═O)OPh) in an inert solvent (e.g. DMF) or neat and heated at temperatures of between 50-190° C., ideally at reflux to give halo pyrimidines. Methods for conversion of compounds of formula 22 to compounds of formula 23 are well known in the art and examples are described in the following references; J. Med. Chem., 2007, 50, 591. Compounds of formula 22 are treated with appropriate nucleophiles in an inert solvent (e.g. DMF, butyronitrile, dichloromethane) in the presence of an appropriate base (e.g. potassium carbonate, sodium carbonate, N,N-diethylamine) at temperatures ranging from ambient temperature to 100° C. dependant of the nucleophilicity of the reagent to give compounds of formula 23. Optionally, the anion of the nucleophile may be prepared by treatment with a suitable base (e.g. sodium hydride, lithium hexamethyldisilazide). It will be appreciated by those skilled in the art that regioisomeric mixtures may result in this reaction and that separation techniques may be required to obtain the desired regioisomer.

Methods for conversion of compounds of formula 23 to compounds of formula 24 are analogous to those previously outlined for the conversion of compounds of formula 22 to compounds of formula 23 described above.

Methods for conversion of compounds of formula 24 to compounds of formula 1 are analogous to those previously outlined for the conversion of compounds of formula 5 to compounds of formula 1 described above.

g) suitable for when Q is O, S, N(R⁸) or a single bond linked to a heteroatom;

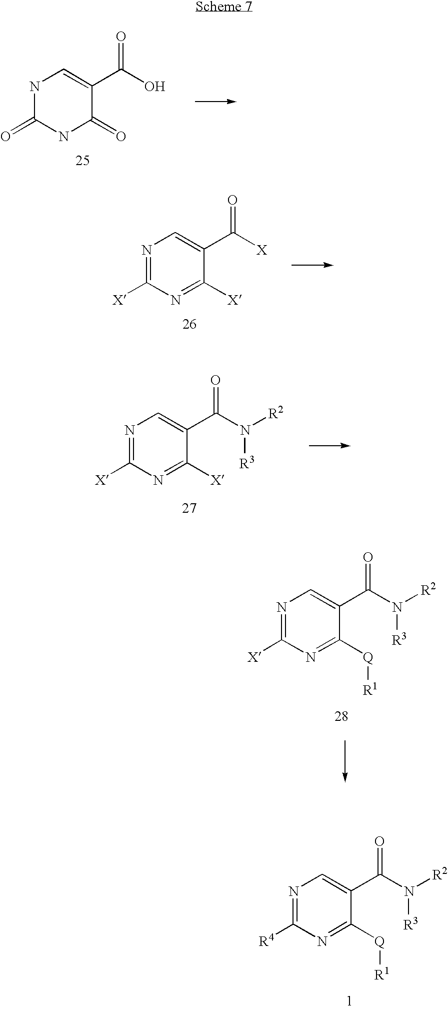

According to this method, a pyrimidinedione acid of formula 25 is halogenated to give a di-halo acyl halide (or equivalent) compound of formula 26 wherein X′ is halo. The compound is treated with an amine of formula NHR²R³ to give compounds of formula 27. The di-halo amide is then treated with a stoichiometric quantity of an appropriate nucleophile (Q-R¹) to give a compound of formula 28 and then reacted with another nucleophile (R⁴) to give the desired compound of formula 1.

Methods for conversion of compounds of formula 25 to compounds of formula 26 are analogous to those previously outlined for the conversion of compounds of formula 21 to compounds of formula 22 described above.

Methods for conversion of compounds of formula 26 to compounds of formula 27 are analogous to those previously outlined for the conversion of compounds of formula 17 to compounds of formula 18 described above.

Methods for conversion of compounds of formula 27 to compounds of formula 28 are compounds of formula 28 to compounds of formula 1 are analogous to those previously outlined for the conversion of compounds of formula 22 to compounds of formula 23 described above.

A significant number of β-ketoamides and β-ketoesters are commercially available as listed in the Available Chemicals Directory and a further number have been described in the chemical literature. A listing of many of the methods suitable for preparation of β-ketoesters is contained within ‘Comprehensive Organic Transformations; A Guide to Functional Group Preparations’, VCH Publishers, Inc, NY, 1989, p685, 694 & 768]. Additional methods may be found in ‘Advanced Organic Chemistry’, 3^{rd} Ed, J. Wiley & Sons, Inc, NY, 1985 p437 & 823]. A sample method for the conversion of β-ketoesters to β-ketoamides has been described above in the preparation of compounds of formula 8.

A number of substituted amidines and guanidines are commercially available as listed in the Available Chemicals Directory and a further number have been described in the chemical literature. A listing of many of the methods suitable for preparation of amidines and guanidines is contained within ‘Comprehensive Organic Functional Group Transformations; Elsevier Publishers, Inc, Oxford, 1995, vol 5, p741 and vol 6, p639]. Additional methods may be found in ‘Advanced Organic Chemistry’, 4^{rd} Ed, J. Wiley & Sons, Inc, NY, 1991 p769 & 903]. A sample method for the conversion of amines to guanidines is given in patent WO1997045108.

It will be appreciated that certain of the various substituents in the compounds of the present invention may be introduced by standard aromatic substitution reactions or generated by conventional functional group modifications either prior to or immediately following the processes mentioned above, and as such are included in the process aspect of the invention. Such reactions and modifications include, introduction of a substituent by means of an aromatic substitution reaction, reduction of substituents, oxidation of substituents and alkylation of substituents, for example, alkylation reactions such as conversion of a secondary amide to a primary amide typically carried out using strong base (e.g. sodium hydride or lithium or potassium hexamethyldisilylazides) and a suitable alkylating agent (e.g. methyl iodide). The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (e.g. aluminium trichloride) under Friedel Crafts conditions; the introduction of an alkyl group using an alkyl halide and Lewis acid (e.g. aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl; removal of alkylthio groups by reductive de-sulphurisation by for example treatment with a nickel catalyst.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein. A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example hydroxylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Accordingly, another aspect of the present invention provides a process for preparing a compound of formula (I) or a pharmaceutically acceptable salt thereof which process (wherein variable groups are, unless otherwise specified, as defined in formula (I)) comprises:

i) reacting a compound of formula:

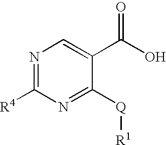

or a reactive derivative thereof with an amine of formula HNR²R³;
ii) reacting together compounds of the formulae:

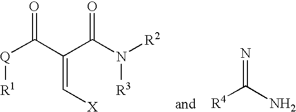

wherein X is dialkylamino or lower alkoxy;
iii) when R⁴ is —SR¹⁰, reacting a compound of the formula:

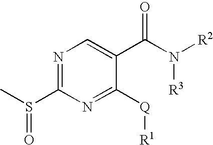

with the appropriate nucleophile to convert —SOMe to —R⁴;
iv) reacting an activated derivative of a compound of the formula:

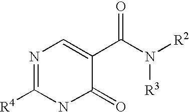

with a nucleophile of the formula Q-R¹;
v) reacting a compound of the formula:

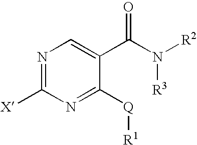

wherein X′ is halo with a nucleophile R⁴;
and thereafter if necessary or desirable:
i) converting a compound of the formula (I) into another compound of the formula (I);
ii) removing any protecting groups;
iii) resolving enantiomers;
iv) forming a pharmaceutically-acceptable salt thereof.

As stated hereinbefore the compounds defined in the present invention possess 11βHSD1 inhibitory activity. These properties may be assessed using the following assay.

### Assays

The conversion of cortisone to the active steroid cortisol by 11βHSD1 oxo-reductase activity, can be measured using a competitive homogeneous time resolved fluorescence assay (HTRF) (CisBio International, R&D, Administration and Europe Office, In Vitro Technologies—HTRF®/Bioassays BP 84175, 30204 Bagnols/Cèze Cedex, France. Cortisol bulk HTRF kit: Cat No. 62CO2PEC).

The evaluation of compounds described herein was carried out using a baculovirus expressed N terminal 6-His tagged full length human 11βHSD1 enzyme(*1). The enzyme was purified from a detergent solubilised cell lysate, using a copper chelate column. Inhibitors of 11βHSD1 reduce the conversion of cortisone to cortisol, which is identified by an increase in signal, in the above assay.

Compounds to be tested were dissolved in dimethyl sulphoxide (DMSO) to 10 mM and diluted further in assay buffer containing 1% DMSO to 10 fold the final assay concentration. Diluted compounds were then plated into black 384 well plates (Matrix, Hudson N.H., USA).

The assay was carried out in a total volume of 20 μl consisting of cortisone (Sigma, Poole, Dorset, UK, 160 nM), glucose-6-phosphate (Roche Diagnostics, 1 mM), NADPH (Sigma, Poole, Dorset, 100 μM), glucose-6-phosphate dehydrogenase (Roche Diagnostics, 12.5 μg/ml), EDTA (Sigma, Poole, Dorset, UK, 1 mM), assay buffer (K₂HPO₄/KH₂PO₄, 100 mM) pH 7.5, recombinant 11βHSD1 [using an appropriate dilution to give a viable assay window—an example of a suitable dilution may be 1 in 1000 dilution of stock enzyme] plus test compound. The assay plates were incubated for 25 minutes at 37° C. after which time the reaction was stopped by the addition of 10111 of 0.5 mM glycerrhetinic acid plus conjugated cortisol(D2). 101 of anti-cortisol Cryptate was then added and the plates sealed and incubated for 6 hours at room temperature. Fluorescence at 665 nm and 620 nm was measured and the 665 nm:620 nm ratio calculated using an Envision plate reader.

These data were then used to calculate IC₅₀ values for each compound (Origin 7.5, Microcal software, Northampton Mass., USA) and/or the % inhibition at 30 μM of compound. *I The Journal of Biological Chemistry, Vol. 26, No 25, pp16653-16658

Compounds of the present invention typically show an IC₅₀ of less than 30 μM, and preferably less than 5 μM.

For example, the following results were obtained:

| Ex. No. | IC50 (uM) |
|---|---|
| 3 | 0.370 |
| 9 | 0.017 |
| 10 | 0.005 |
| 11 | 0.070 |
| 12 | 0.045 |
| 13 | 0.014 |
| 16 | 0.002 |
| 17 | 0.330 |
| 18 | 0.003 |
| 19 | 0.014 |
| 20 | 0.002 |
| 29 | 0.013 |
| 30 | 0.019 |
| 32 | 0.220 |
| 33 | 0.033 |
| 38 | 0.041 |

The following table displays % inhibition of human 11-βHSD at a test concentration of 30 μM of compound

| Example | % inhibition |
|---|---|
| Number | at 30 μM |
| 1 | 101.8 |
| 2 | 103.3 |
| 3 | 105.8 |
| 4 | 89.2 |
| 5 | 96.0 |
| 6 | 83.3 |
| 7 | 88.3 |
| 8 | 93.3 |
| 9 | 98.7 |
| 10 | 88.5 |
| 11 | 97.5 |
| 12 | 97.3 |
| 13 | 111.5 |
| 14 | 108.0 |
| 15 | 95.4 |
| 16 | 95.8 |
| 17 | 94.0 |
| 18 | 98.5 |
| 19 | 100.0 |
| 20 | 97.6 |
| 21 | 93.0 |
| 22 | 89.5 |
| 23 | 97.4 |
| 24 | 68.0 |
| 25 | 101.0 |
| 26 | 89.0 |
| 27 | 95.5 |
| 28 | 103.0 |
| 29 | 100.0 |
| 30 | 93.5 |
| 31 | 93.5 |
| 32 | 97.0 |
| 33 | 97.5 |
| 34 | 98.0 |
| 35 | 105.5 |
| 36 | 99.5 |
| 37 | 93.5 |
| 38 | 104.0 |
| 39 | 99.5 |
| 40 | 91.5 |
| 41 | 94.0 |
| 42 | 99.4 |
| 43 | 99.2 |
| 44 | 98.0 |
| 45 | 98.5 |
| 46 | 104.5 |
| 47 | 97.3 |
| 48 | 100.2 |
| 49 | 99.3 |
| 50 | 99.0 |
| 51 | 101.9 |
| 52 | 92.5 |
| 53 | 101.6 |
| 54 | 101.5 |
| 55 | 96.9 |
| 56 | 90.2 |
| 57 | 101.6 |
| 58 | 99.6 |
| 59 | 95.6 |
| 60 | 95.1 |
| 61 | 105.1 |
| 62 | 99.9 |
| 63 | 92.7 |
| 64 | 95.9 |
| 65 | 100.2 |
| 66 | 99.5 |
| 67 | 105.5 |
| 68 | 98.3 |
| 69 | 101.7 |
| 70 | 98.0 |
| 71 | 100.4 |
| 72 | 91.6 |
| 73 | 95.2 |
| 74 | 88.5 |
| 75 | 98.6 |
| 76 | 96.1 |
| 77 | 90.9 |
| 78 | 93.1 |
| 79 | 89.7 |
| 80 | 99.2 |
| 81 | 100.3 |
| 82 | 95.2 |
| 83 | 103.2 |
| 84 | 97.5 |
| 85 | 88.8 |
| 86 | 97.0 |
| 87 | 95.7 |
| 88 | 94.9 |
| 89 | 95.5 |
| 90 | 100.0 |
| 91 | 95.7 |
| 92 | 97.1 |
| 93 | 93.1 |
| 94 | 103.4 |
| 95 | 97.3 |
| 96 | 97.4 |
| 97 | 98.7 |
| 98 | 97.8 |
| 99 | 93.3 |
| 100 | 99.4 |
| 101 | 101.6 |
| 102 | 93.8 |
| 103 | 95.7 |
| 104 | 99.3 |
| 105 | 97.5 |
| 106 | 101.3 |
| 107 | 97.3 |
| 108 | 96.0 |
| 109 | 90.3 |
| 110 | 100.2 |
| 111 | 108.4 |
| 112 | 104.3 |
| 113 | 98.4 |
| 114 | 106.0 |
| 115 | 93.4 |
| 116 | 98.1 |
| 117 | 97.4 |
| 118 | 93.7 |
| 119 | 103.9 |
| 120 | 100.8 |
| 121 | 103.8 |
| 122 | 97.5 |
| 123 | 99.1 |
| 124 | 98.5 |
| 125 | 95.0 |
| 126 | 96.6 |
| 127 | 102.6 |
| 128 | 99.1 |
| 129 | 95.5 |
| 130 | 96.1 |
| 131 | 101.2 |
| 132 | 99.9 |
| 133 | 94.0 |
| 134 | 94.1 |
| 135 | 100.5 |
| 136 | 97.7 |
| 137 | 96.9 |
| 138 | 100.6 |
| 139 | 99.5 |
| 140 | 101.2 |
| 141 | 104.9 |
| 142 | 99.7 |
| 143 | 95.0 |
| 144 | 98.8 |
| 145 | 92.9 |
| 146 | 93.4 |
| 147 | 103.1 |
| 148 | 102.3 |
| 149 | 92.0 |
| 150 | 96.0 |
| 151 | 100.9 |
| 152 | 97.8 |
| 153 | 91.7 |
| 154 | 88.0 |
| 155 | 92.6 |
| 156 | 65.5 |
| 157 | 87.1 |
| 158 | 96.5 |
| 159 | 98.2 |
| 160 | 103.8 |
| 161 | 105.4 |
| 162 | 97.7 |
| 163 | 100.0 |
| 164 | 98.9 |
| 165 | 104.9 |
| 166 | 102.2 |
| 167 | 100.8 |
| 170 | 94.4 |
| 171 | 103.3 |
| 172 | 97.2 |
| 173 | 101.4 |
| 174 | 95.0 |
| 175 | 103.4 |
| 176 | 97.9 |
| 177 | 95.8 |
| 178 | 95.3 |
| 179 | 95.1 |
| 180 | 98.8 |
| 181 | 95.0 |
| 182 | 102.2 |
| 183 | 100.9 |
| 184 | 108.5 |
| 185 | 93.5 |
| 186 | 95.5 |
| 187 | 99.0 |
| 188 | 78.9 |
| 189 | 95.5 |
| 190 | 99.2 |
| 191 | 93.2 |
| 192 | 105.4 |
| 193 | 102.2 |
| 194 | 99.0 |
| 195 | 102.8 |
| 196 | 99.8 |
| 197 | 97.8 |
| 198 | 94.0 |
| 199 | 96.8 |
| 200 | 52.8 |
| 201 | 95.0 |
| 202 | 102.1 |
| 203 | 98.6 |
| 204 | 106.4 |
| 205 | 102.4 |
| 206 | 105.1 |
| 207 | 93.0 |
| 208 | 101.6 |
| 209 | 99.4 |
| 210 | 97.2 |
| 211 | 98.4 |
| 212 | 105.4 |
| 213 | 107.0 |
| 214 | 103.1 |
| 215 | 94.9 |
| 216 | 97.1 |
| 217 | 94.2 |
| 218 | 96.0 |
| 219 | 98.6 |

According to a further aspect of the invention there is provided a pharmaceutical composition, which comprises a compound of the Examples, or a pharmaceutically-acceptable salt thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing). In general, compositions in a form suitable for oral use are preferred.

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

We have found that the compounds defined in the present invention, or a pharmaceutically-acceptable salt thereof, are effective 11βHSD1 inhibitors, and accordingly have value in the treatment of disease states associated with metabolic syndrome.

It is to be understood that where the term “metabolic syndrome” is used herein, this relates to metabolic syndrome as defined in 1) and/or 2) or any other recognised definition of this syndrome. Synonyms for “metabolic syndrome” used in the art include Reaven's Syndrome, Insulin Resistance Syndrome and Syndrome X. It is to be understood that where the term “metabolic syndrome” is used herein it also refers to Reaven's Syndrome, Insulin Resistance Syndrome and Syndrome X.

According to a further aspect of the present invention there is provided a compound of formula (1), or a pharmaceutically-acceptable salt thereof, as defined hereinbefore for use in a method of prophylactic or therapeutic treatment of a warm-blooded animal, such as man.

Thus according to this aspect of the invention there is provided a compound of formula (1), or a pharmaceutically-acceptable salt thereof, as defined hereinbefore for use as a medicament.

According to another feature of the invention there is provided the use of a compound of formula (1), or a pharmaceutically-acceptable salt thereof, as defined hereinbefore in the manufacture of a medicament for use in the production of an 11βHSD1 inhibitory effect in a warm-blooded animal, such as man.

Where production of or producing an 11βHSD1 inhibitory effect is referred to suitably this refers to the treatment of metabolic syndrome. Alternatively, where production of an 11βHSD1 inhibitory effect is referred to this refers to the treatment of diabetes, obesity, hyperlipidaemia, hyperglycaemia, hyperinsulinemia or hypertension. In particularly where production of an 11βHSD1 inhibitory effect is referred to this refers to the treatment of diabetes and obesity. In one aspect, type 2 diabetes. In another aspect, obesity. Alternatively, where production of an 11βHSD1 inhibitory effect is referred to this refers to the treatment of glaucoma, osteoporosis, tuberculosis, dementia, cognitive disorders or depression.

Alternatively, where production of an 11βHSD1 inhibitory effect is referred to this refers to the treatment of cognitive disorders, such as improving the cognitive ability of an individual, for example by improvement of verbal fluency, verbal memory or logical memory, or for treatment of mild cognitive disorders. See for example WO03/086410 and references contained therein, and Proceedings of National Academy of Sciences (PNAS), 2001, 98(8), 4717-4721.

Alternatively, where production of an 11βHSD1 inhibitory effect is referred to this refers to the treatment of, delaying the onset of and/or reducing the risk of atherosclerosis—see for example J. Experimental Medicine, 2005, 202(4), 517-527.

Alternatively, where production of an 11βHSD1 inhibitory effect is referred to this refers to the treatment of Alzheimers and/or neurodegenerative disorders.

According to a further feature of this aspect of the invention there is provided a method for producing an 11βHSD1 inhibitory effect in a warm-blooded animal, such as man, in need of such treatment which comprises administering to said animal an effective amount of a compound of formula (1), or a pharmaceutically-acceptable salt thereof.

In addition to their use in therapeutic medicine, the compounds of formula (1), or a pharmaceutically-salt thereof, are also useful as pharmacological tools in the development and standardisation of in vitro and in vivo test systems for the evaluation of the effects of inhibitors of 11βHSD1 in laboratory animals such as cats, dogs, rabbits, monkeys, rats and mice, as part of the search for new therapeutic agents.

The inhibition of 11βHSD1 described herein may be applied as a sole therapy or may involve, in addition to the subject of the present invention, one or more other substances and/or treatments. Such conjoint treatment may be achieved by way of the simultaneous, sequential or separate administration of the individual components of the treatment. Simultaneous treatment may be in a single tablet or in separate tablets. For example agents than might be co-administered with 11βHSD1 inhibitors, particularly those of the present invention, may include the following main categories of treatment:

1) Insulin and insulin analogues;
2) Insulin secretagogues including sulphonylureas (for example glibenclamide, glipizide), prandial glucose regulators (for example repaglinide, nateglinide), glucagon-like peptide 1 agonist (GLP1 agonist) (for example exenatide, liraglutide) and dipeptidyl peptidase IV inhibitors (DPP-IV inhibitors);
3) Insulin sensitising agents including PPARγ agonists (for example pioglitazone and rosiglitazone);
4) Agents that suppress hepatic glucose output (for example metformin);
5) Agents designed to reduce the absorption of glucose from the intestine (for example acarbose);
6) Agents designed to treat the complications of prolonged hyperglycaemia; e.g. aldose reductase inhibitors
7) Other anti-diabetic agents including phosotyrosine phosphatase inhibitors, glucose 6-phosphatase inhibitors, glucagon receptor antagonists, glucokinase activators, glycogen phosphorylase inhibitors, fructose 1,6 bisphosphastase inhibitors, glutamine:fructose-6-phosphate amidotransferase inhibitors
8) Anti-obesity agents (for example sibutramine and orlistat);
9) Anti-dyslipidaemia agents such as, HMG-CoA reductase inhibitors (statins, e.g. pravastatin); PPARα agonists (fibrates, e.g. gemfibrozil); bile acid sequestrants (cholestyramine); cholesterol absorption inhibitors (plant stanols, synthetic inhibitors); ileal bile acid absorption inhibitors (IBATi), cholesterol ester transfer protein inhibitors and nicotinic acid and analogues (niacin and slow release formulations);
10) Antihypertensive agents such as, β blockers (e.g. atenolol, inderal); ACE inhibitors (e.g. lisinopril); calcium antagonists (e.g. nifedipine); angiotensin receptor antagonists (e.g. candesartan), α antagonists and diuretic agents (e.g. furosemide, benzthiazide);
11) Haemostasis modulators such as, antithrombotics, activators of fibrinolysis and antiplatelet agents; thrombin antagonists; factor Xa inhibitors; factor VIIa inhibitors; antiplatelet agents (e.g. aspirin, clopidogrel); anticoagulants (heparin and Low molecular weight analogues, hirudin) and warfarin;
12) Anti-inflammatory agents, such as non-steroidal anti-inflammatory drugs (e.g. aspirin) and steroidal anti-inflammatory agents (e.g. cortisone); and
13) Agents that prevent the reabsorption of glucose by the kidney (SGLT inhibitors).

In the above other pharmaceutical composition, process, method, use and medicament manufacture features, the alternative and preferred embodiments of the compounds of the invention described herein also apply.

### EXAMPLES

The invention will now be illustrated by the following Examples in which, unless stated otherwise:

(i) temperatures are given in degrees Celsius (° C.); operations were carried out at room or ambient temperature, that is, at a temperature in the range of 18-25° C. and under an atmosphere of an inert gas such as argon;
(ii) evaporation of solvent was carried out using a rotary evaporator under reduced pressure (600-4000 Pa; 4.5-30 mmHg) with a bath temperature of up to 60° C.;
(iii) chromatography means flash chromatography on silica gel;
(iv) in general, the course of reactions was followed by TLC and reaction times are given for illustration only;
(v) yields are given for illustration only and are not necessarily those which can be obtained by diligent process development; preparations were repeated if more material was required;
(vi) where given, NMR data (¹H) is in the form of delta values for major diagnostic protons, given in parts per million (ppm) relative to tetramethylsilane (TMS), determined at 300 or 400 MHz (unless otherwise stated) using perdeuterio dimethyl sulfoxide (DMSO-d₆) as solvent, unless otherwise stated; peak multiplicities are shown thus: s, singlet; d, doublet; dd, doublet of doublets; dt, doublet of triplets; dm, doublet of multiplets; t, triplet, m, multiplet; br, broad; protons attached to oxygen or nitrogen may give rise to very broad peaks which are not reported;
(vii) chemical symbols have their usual meanings; SI units and symbols are used;
(viii) solvent ratios are given in volume: volume (v/v) terms;
(ix) mass spectra (MS) were run with an electron energy of 70 electron volts in the chemical ionisation (CI) mode using a direct exposure probe; where indicated ionisation was effected by electron impact (EI), fast atom bombardment (FAB) or electrospray (ESP); values for m/z are given; generally, only ions which indicate the parent mass are reported;
(x) where examples are indicated by chemical name and/or structure to be an enantiomer, in some cases the product may contain a small amount of the other enantiomer;
(xi) The following abbreviations may be used below or in the process section hereinbefore:
Et₂O diethyl ether
DMF dimethylformamide
DCM dichloromethane
DME 1,2-dimethoxyethane
MeOH methanol
EtOH ethanol
H₂O water
TFA trifluoroacetic acid
THF tetrahydrofuran
DMSO dimethylsulfoxide
HOBt 1-hydroxybenzotriazole
EDCI (EDAC) 1-ethyl-3-(3-dimethylaminopropyl)carbodi-imide hydrochloride
DIPEA diisopropylethylamine
DEAD diethyl azodicarboxylate
EtOAc ethyl acetate
NaHCO₃ sodium bicarbonate
K₃PO₄ potassium phosphate
MgSO₄ magnesium sulfate
PS polymer supported
BINAP 2,2′-bis(diphenylphosphino)-1,1′binaphthyl
Dppf 1,1′-bis(diphenylphosphino)ferrocene
dba dibenzylidineacetone
PS-CDI polymer supported carbonyldiimidazole

### Example 1

### 4-Cyclopropyl-N-[(2s,5r)-5-hydroxyadamantan-2-yl]-2-morpholin-4-ylpyrimidine-5-carboxamide

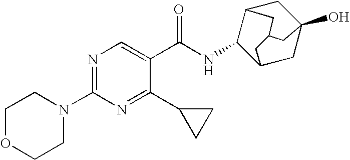

(1s,4r)-4-Aminoadamant-1-ol (335 mg, 2.01 mmol) was added in one portion to a mixture of 4-cyclopropyl-2-morpholinopyrimidine-5-carboxylic acid (Intermediate 3, 500 mg, 2.01 mmol), 1-hydroxybenzotriazole (298 mg, 2.21 mmol), 1-(3-Dimethylaminopropyl)-3-ethylcarbodiimide hydrochloride (461 mg, 2.41 mmol) and N,N-Diisopropylethylamine (1.22 mL, 7.02 mmol) in DMF (10 mL) under nitrogen. The resulting suspension was stirred at room temperature for 16 hours. The reaction mixture was diluted with water/ice (50 mL) and the resulting precipitate was extracted with EtOAc (2×25 mL). The combined extracts were washed with brine (25 mL), dried over MgSO₄, filtered and evaporated to give crude product. The crude product was purified by flash silica (40 g) chromatography, elution gradient 0 to 100% 10% MeOH/EtOAc in EtOAc. Pure fractions were evaporated to dryness to afford 4-cyclopropyl-N-[(2s,5r)-5-hydroxyadamantan-2-yl]-2-morpholin-4-ylpyrimidine-5-carboxamide (115 mg, 14%) as a white solid.

1H NMR (400.13 MHz, DMSO-d₆) δ 0.91-0.98 (2H, m), 0.99-1.04 (2H, m), 1.32 (2H, d), 1.62 (4H, s), 1.71 (2H, s), 1.93 (2H, d), 1.99 (1H, s), 2.04 (2H, s), 2.41-2.46 (1H, m), 3.61 (4H, d), 3.67 (4H, t), 3.92 (1H, t), 4.37 (1H, s), 8.05 (1H, d), 8.23 (1H, t)

m/z (ESI+) (M+H)+=399; HPLC t_{R}=1.64 min.

### Intermediate 1

### Ethyl 2-(cyclopropanecarbonyl)-3-(dimethylamino)acrylate

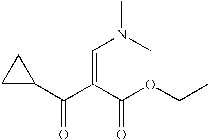

N,N-Dimethylformamide dimethyl acetal (4.26 mL, 32.01 mmol) was added in one portion to ethyl 3-cyclopropyl-3-oxopropanoate (5.00 g, 32.01 mmol) in dioxane (50 mL) and warmed to 100° C. over a period of 5 minutes under nitrogen. The resulting solution was stirred at this temperature for 4 hours. The resulting mixture was evaporated to dryness and the residue was azeotroped with toluene to afford crude ethyl 2-(cyclopropanecarbonyl)-3-(dimethylamino)acrylate (6.70 g, 99%), which was used without further purification.

1H NMR (400.13 MHz, CDCl₃) δ 0.72-0.76 (2H, m), 0.92-0.98 (2H, m), 1.18-1.24 (3H, m), 2.31 (1H, s), 2.72-2.91 (6H, m), 4.16 (2H, q), 7.52 (1H, s)

m/z (ESI+) (M+H)+=212; HPLC t_{R}=1.38 min.

### Intermediate 2

### Ethyl 4-cyclopropyl-2-morpholinopyrimidine-5-carboxylate

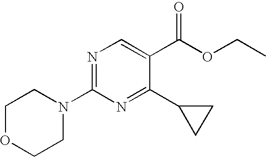

A solution of ethyl 2-(cyclopropanecarbonyl)-3-(dimethylamino)acrylate (Intermediate 1, 6.76 g, 32 mmol) in ethanol (25 mL) was added dropwise to a stirred suspension of morpholine-4-carboximidamide hydrochloride (5.30 g, 32.00 mmol) and sodium ethoxide (2.18 g, 32.00 mmol) in ethanol (75 mL) over a period of 5 minutes under nitrogen. The resulting suspension was stirred at room temperature for 16 hours. The reaction mixture was evaporated to dryness and redissolved in water/ice (150 mL), The precipitate was collected by filtration, washed with water (25 mL) and dried under vacuum to afford ethyl 4-cyclopropyl-2-morpholinopyrimidine-5-carboxylate (3.12 g, 35%) as a orange solid, which was used without further purification.

1H NMR (400.13 MHz, DMSO-d₆) δ 1.01-1.06 (2H, m), 1.07-1.12 (2H, m), 1.30 (3H, t), 3.11-3.17 (1H, m), 3.64 (4H, d), 3.74 (4H, d), 4.26 (2H, q), 8.71 (1H, s)

m/z (ESI+) (M+H)+=278; HPLC t_{R}=2.43 min.

### Intermediate 3

### 4-cyclopropyl-2-morpholinopyrimidine-5-carboxylic acid

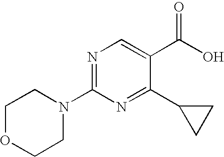

A solution of sodium hydroxide (9.01 mL, 18.03 mmol) was added in one portion to a stirred solution of ethyl 4-cyclopropyl-2-morpholinopyrimidine-5-carboxylate (Intermediate 2, 2.00 g, 7.21 mmol) in methanol (50 mL) and warmed to 100° C. over a period of 5 minutes under air. The resulting solution was stirred at this temperature for 4 hours. The reaction mixture was evaporated to dryness and redissolved in water (20 mL) and acidified with 2M HCl. The precipitate was collected by filtration, washed with water (20 mL) and dried under vacuum to afford 4-cyclopropyl-2-morpholinopyrimidine-5-carboxylic acid (1.70 g, 95%) as a cream solid, which was used without further purification.

1H NMR (400.13 MHz, DMSO-d₆) δ 0.99-1.05 (2H, m), 1.07-1.10 (2H, m), 3.22-3.28 (1H, m), 3.62-3.67 (4H, m), 3.73 (4H, t), 8.71 (1H, s), 12.76 (1H, s)

m/z (ESI+) (M+H)+=250; HPLC t_{R}=1.64 min.

The following Examples were prepared in a similar manner to Example 1, using Intermediate 1 and an appropriate amidine or guanidine starting material:

| Structure | Ex | Name | ¹H NMR δ | MS m/e MH⁺ |
|---|---|---|---|---|
| 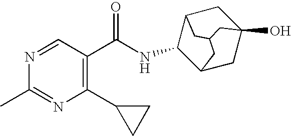 | 2 | 4-cyclopropyl-N- [(2r,5s)-5- hydroxyadamantan- 2-yl]-2- methylpyrimidine-5- carboxamide | 1H NMR (400.132 MHz, CDCl3) δ 1.05-1.12 (2H, m), 1.26-1.32 (2H, m), 1.59 (3H, d), 1.67-1.75 (2H, m), 1.78-1.86 (4H, m), 1.95 (2H, d), 2.19 (1H, s), 2.27 (2H, s), 2.35-2.42 (1H, m), 2.62 (3H, s), 4.25 (1H, d), 6.10 (1H, d), 8.53 (1H, s) | 328; HPLC t_{R} = 1.33 min. |
| 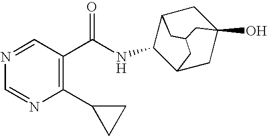 | 3 | 4-cyclopropyl-N- [(2r,5s)-5- hydroxyadamantan- 2-yl]pyrimidine-5- carboxamide | 1H NMR (400.132 MHz, CDCl3) δ 1.11-1.17 (2H, m), 1.29-1.34 (2H, m), 1.60 (3H, d), 1.69-1.76 (2H, m), 1.78-1.86 (4H, m), 1.96 (2H, d), 2.19 (1H, s), 2.28 (2H, s), 2.40 (1H, septet), 4.27 (1H, d), 6.11 (1H, d), 8.61 (1H, s), 9.00 (1H, s) | 314; HPLC t_{R} = 1.2 min. |

The following intermediates were used and were prepared as described below.

### Intermediate 4

### Methyl 2-methyl-4-cyclopropylpyrimidine-5-carboxylate

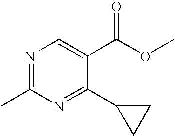

Prepared by the same process used for Intermediate 2 from methyl 2-(cyclopropanecarbonyl)-3-(dimethylamino)acrylate.

1H NMR (400.13 MHz, DMSO-d₆) δ 1.09-1.17 (4H, m), 2.55 (3H, s), 2.96-3.02 (1H, m), 3.88 (3H, s), 8.88 (1H, s)

m/z (ESI+) (M+H)+=193; HPLC t_{R}=1.79 min.

### Intermediate 5

### 2-Methyl-4-cyclopropylpyrimidine-5-carboxylic acid

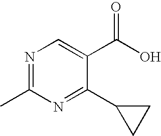

Prepared from methyl 2-methyl-4-cyclopropylpyrimidine-5-carboxylate (Intermediate 4) by the same process used for Intermediate 3.

1H NMR (400.13 MHz, DMSO-d₆) δ 1.06-1.15 (4H, m), 2.54 (3H, s), 3.08-3.14 (1H, m), 8.87 (1H, s), 13.49 (1H, s)

m/z (ESI+) (M+H)+=179; HPLC t_{R}=1.07 min.

### Intermediate 6

### Methyl 4-cyclopropylpyrimidine-5-carboxylate

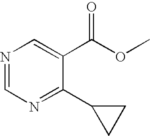

Prepared by the same process used for Intermediate 2 from methyl 2-(cyclopropanecarbonyl)-3-(dimethylamino)acrylate.

1H NMR (400.132 MHz, CDCl3) δ 1.15-1.20 (2H, m), 1.29-1.34 (2H, m), 3.12 (1H, septet), 3.97 (3H, s), 9.02 (2H, s)

m/z (ESI+) (M+H)+=179; HPLC t_{R}=1.46 min.

### Intermediate 7

### 4-cyclopropylpyrimidine-5-carboxylic acid

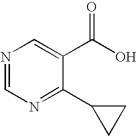

Prepared from methyl 4-cyclopropylpyrimidine-5-carboxylate (Intermediate 6) by the same process used for Intermediate 3.

1H NMR (400.132 MHz, DMSO) δ 2.49 (4H, quintet), 3.09 (1H, quintet), 8.96 (1H, s), 9.05 (1H, s), 13.10-14.12 (1H, m)

m/z (ESI+) (M+H)+=165; HPLC t_{R}=0.93 min.

### Example 4

### 4-tert-Butyl-N-[(2r,5s)-5-hydroxyadamantan-2-yl]-2-morpholin-4-ylpyrimidine-5-carboxamide

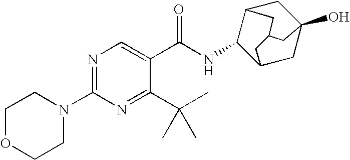

O-(7-Azabenzotriazol-1-yl)-N,N,N′,N′-tetramethyluronium hexafluorophosphate (456 mg, 1.20 mmol) was added in one portion to 4-tert-butyl-2-morpholinopyrimidine-5-carboxylic acid (Intermediate 10, 265 mg, 1.00 mmol), (1s,4r)-4-aminoadamantan-1-ol hydrochloride (203 mg, 1.00 mmol) and N-ethyldiisopropylamine (0.518 mL, 3.00 mmol) in DMF (10 mL) at 20° C. under nitrogen. The resulting suspension was stirred at 20° C. for 2 hours. The reaction mixture was diluted with EtOAc (100 mL), and washed sequentially with water (4×25 mL) and saturated brine (25 mL). The organic layer was dried over Na2SO4, filtered and evaporated to afford crude product. The crude product was purified by flash silica chromatography, elution gradient 0 to 5% MeOH in EtOAc. Pure fractions were evaporated to dryness to afford 4-tert-butyl-N-[(2r,5s)-5-hydroxyadamantan-2-yl]-2-morpholin-4-ylpyrimidine-5-carboxamide (296 mg, 72%) as a white solid.

1H NMR (300.13 MHz, DMSO-d₆) δ 1.28-1.35 (11H, m), 1.61-1.74 (6H, m), 1.89-2.02 (5H, m), 3.64-3.74 (8H, m), 3.88-3.95 (1H, m), 4.39 (1H, s), 8.09 (1H, s), 8.18 (1H, d)

m/z (ESI+) (M+H)+=415; HPLC t_{R}=1.94 min.

### Intermediate 8

### Ethyl 2-((dimethylamino)methylene)-4,4-dimethyl-3-oxopentanoate

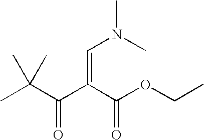

N,N-Dimethylformamide dimethyl acetal (3.86 mL, 29.03 mmol) was added to ethyl pivaloylacetate (5.21 mL, 29.03 mmol) in dioxane (40 mL) under nitrogen. The resulting solution was stirred at 100° C. for 9 hours. The reaction mixture was evaporated to afford the crude product as a yellow oil that was used in the following step without further purification.

1H NMR (400.13 MHz, CDCl₃) δ 1.24 (9H, s), 1.26-1.30 (3H, m), 2.89 (6H, s), 4.18 (2H, q), 7.36 (1H, s)

m/z (ESI+) (M+H)+=228; HPLC t_{R}=1.95 min.

### Intermediate 9

### Ethyl 4-tert-butyl-2-morpholinopyrimidine-5-carboxylate

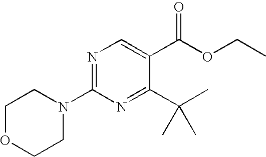

Morpholinoformamidine hydrobromide (2.098 g, 9.99 mmol) was added to sodium methoxide (19.97 ml, 9.99 mmol). Ethyl 2-((dimethylamino)methylene)-4,4-dimethyl-3-oxopentanoate (Intermediate 8, 2.27 g, 9.99 mmol) was then added and the resulting mixture was stirred at 70° C. for 5 hours under nitrogen. The reaction mixture was diluted with EtOAc (100 mL), and washed sequentially with water (2×50 mL) and saturated brine (50 mL). The organic layer was dried over MgSO4, filtered and evaporated to afford crude product. The crude product was purified by flash silica chromatography, elution gradient 0 to 20% EtOAc in isohexane. Pure fractions were evaporated to dryness to afford ethyl 4-tert-butyl-2-morpholinopyrimidine-5-carboxylate (1.310 g, 45%) as a colourless oil.

1H NMR (400.13 MHz, DMSO-d₆) δ 1.28 (3H, t), 1.32 (9H, s), 3.64-3.67 (4H, m), 3.75-3.79 (4H, m), 4.25 (2H, q), 8.48 (1H, s)

m/z (ESI+) (M+H)+=294; HPLC t_{R}=2.77 min.

### Intermediate 10

### 4-tert-butyl-2-morpholinopyrimidine-5-carboxylic acid

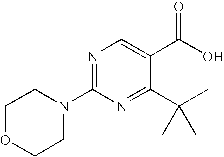

A solution of sodium hydroxide (11.16 mL, 22.33 mmol) was added to a stirred solution of ethyl 4-tert-butyl-2-morpholinopyrimidine-5-carboxylate (Intermediate 9, 1.31 g, 4.47 mmol) in methanol (20 mL) at 20° C. The resulting mixture was stirred at 100° C. for 24 hours. The reaction mixture was concentrated and diluted with water (100 mL) and washed with ether (50 mL).The reaction mixture was acidified with 2M HCl and extracted with EtOAc (2×50 ml). The organic layers were combined and washed sequentially with water (50 mL) and saturated brine (50 mL). The organic layer was dried over MgSO4, filtered and evaporated to afford desired product that was used without further purification.

1H NMR (400.13 MHz, DMSO-d₆) δ 1.35 (9H, s), 3.64-3.66 (4H, m), 3.74-3.79 (4H, m), 8.51 (1H, s), 12.86 (1H, s)

m/z (ESI+) (M+H)+=266; HPLC t_{R}=1.91 min.

### Example 5

### N-[(2r,5 s)-5-hydroxyadamantan-2-yl]-4-methyl-2-morpholin-4-ylpyrimidine-5-carboxamide

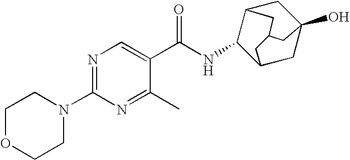

O-(7-Azabenzotriazol-1-yl)-N,N,N′,N′-tetramethyluronium hexafluorophosphate (456 mg, 1.20 mmol) was added in one portion to (1s,4r)-4-aminoadamantan-1-ol hydrochloride (204 mg, 1.00 mmol), 4-methyl-2-morpholinopyrimidine-5-carboxylic acid (Intermediate 13, 223 mg, 1.00 mmol) and N-ethyldiisopropylamine (0.519 mL, 3.00 mmol) in DMF (10 mL) at 20° C. under nitrogen. The resulting suspension was stirred at 20° C. for 2 hours. The reaction mixture was diluted with EtOAc (100 mL), and washed sequentially with water (4×25 mL) and saturated brine (25 mL). The organic layer was dried over MgSO4, filtered and evaporated to afford crude product. The crude product was purified by preparative HPLC (Phenomenex Gemini C18 110A (axia) column, 5μ silica, 30 mm diameter, 100 mm length), using decreasingly polar mixtures of water (containing 0.1% NH3) and MeCN as eluents. Fractions containing the desired compound were evaporated to dryness to afford N-[(2r,5 s)-5-hydroxyadamantan-2-yl]-4-methyl-2-morpholin-4-ylpyrimidine-5-carboxamide (114 mg, 31%) as a white solid.

1H NMR (400.13 MHz, DMSO-d₆) δ 1.30-1.34 (2H, m), 1.60-1.63 (4H, m), 1.69-1.72 (2H, m), 1.92 (2H, d), 1.99 (1H, s), 2.03 (2H, s), 2.38 (3H, s), 3.62-3.64 (4H, m), 3.72-3.75 (4H, m), 3.90 (1H, t), 4.38 (1H, s), 7.96-7.98 (1H, m), 8.31 (1H, s)

m/z (ESI+) (M+H)+=373; HPLC t_{R}=1.43 min.

### Intermediate 11

### Methyl 2-((dimethylamino)methylene)-3-oxobutanoate

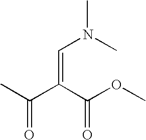

Prepared from methyl 3-oxobutanoate by the same process used for Intermediate 8.

1H NMR (400.13 MHz, DMSO-d₆) δ 2.13 (3H, s), 2.51-3.08 (6H, m), 3.63 (3H, s), 7.61 (1H, s)

### Intermediate 12

### Methyl 4-methyl-2-morpholinopyrimidine-5-carboxylate

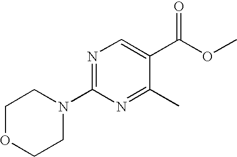

Prepared from methyl 2-((dimethylamino)methylene)-3-oxobutanoate (Intermediate 11) by the same process used for Intermediate 9.

1H NMR (400.13 MHz, DMSO-d₆) δ 2.56 (3H, s), 3.62-3.66 (4H, m), 3.77 (3H, s), 3.80-3.82 (4H, m), 8.74 (1H, s)

m/z (ESI+) (M+H)+=238; HPLC t_{R}=1.74 min.

### Intermediate 13

### 4-methyl-2-morpholinopyrimidine-5-carboxylic acid

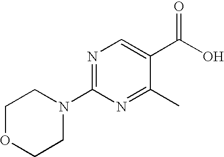

Prepared from methyl 4-methyl-2-morpholinopyrimidine-5-carboxylate (Intermediate 12) by the same process used for Intermediate 10.

1H NMR (400.13 MHz, DMSO-d₆) δ 2.56 (3H, s), 3.62-3.66 (4H, m), 3.79-3.81 (4H, m), 8.73 (1H, s), 12.63 (1H, s)

m/z (ESI+) (M+H)+=224; HPLC t_{R}=1.3 min.

### Example 6

### 4-tert-Butyl-N-[(2r,5s)-5-hydroxyadamantan-2-yl]-2-methylpyrimidine-5-carboxamide

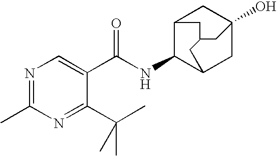

Oxalyl chloride (0.337 mL, 3.86 mmol) was added to 4-tert-butyl-2-methylpyrimidine-5-carboxylic acid (Intermediate 15, 250 mg, 1.29 mmol) in CH2Cl2 (25 mL). One drop of DMF was added and the resulting suspension was stirred at 20° C. for 3 hours. The resulting mixture was evaporated to dryness and the residue was azeotroped with toluene to afford crude 4-tert-butyl-2-methylpyrimidine-5-carbonyl chloride, which was dissolved in DCM (2 mL) and added dropwise to a stirred suspension of (1s,4r)-4-aminoadamantan-1-ol hydrochloride (0.263 g, 1.29 mmol) and N-ethyldiisopropylamine (0.491 mL, 2.84 mmol) in THF (4 mL). The resulting solution was stirred at 20° C. for 3 hours. The reaction mixture was evaporated to dryness and redissolved in EtOAc (25 mL), and washed sequentially with water (5 mL), 1N citric acid (5 mL), saturated NaHCO3 (5 mL) and saturated brine (5 mL). The organic layer was dried over MgSO4, filtered and evaporated to afford crude product. The crude residue was triturated with Et2O to give a solid which was collected by filtration and dried under vacuum to give 4-tert-butyl-N-[(2r,5s)-5-hydroxyadamantan-2-yl]-2-methylpyrimidine-5-carboxamide (0.240 g, 54%)

1H NMR (400.132 MHz, CDCl3) δ 1.42 (10H, s), 1.58 (2H, d), 1.64-1.72 (2H, m), 1.77-1.86 (4H, m), 1.95 (2H, d), 2.16 (1H, s), 2.26 (2H, s), 2.70 (3H, s), 4.22 (1H, d), 5.91 (1H, d), 8.41 (1H, s)

m/z (ESI+) (M+H)+=344; HPLC t_{R}=1.63 min.

### Intermediate 14

### Ethyl 4-tert-butyl-2-methylpyrimidine-5-carboxylate

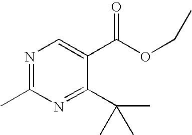

Prepared from ethyl 2-((dimethylamino)methylene)-4,4-dimethyl-3-oxopentanoate (Intermediate 8) by the same process used for Intermediate 9.

1H NMR (400.132 MHz, CDCl3) δ 1.39 (3H, t), 1.40 (9H, s), 2.71 (3H, s), 4.39 (2H, q), 8.54 (1H, s)

m/z (ESI+) (M+H)+=223; HPLC t_{R}=2.36 min.

### Intermediate 15

### 4-tert-butyl-2-methylpyrimidine-5-carboxylic acid

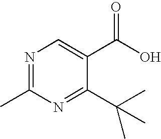

Prepared from ethyl 4-tert-butyl-2-methylpyrimidine-5-carboxylate (Intermediate 14) by the same process used for Intermediate 10.

1H NMR (400.132 MHz, CDCl3) δ 1.46 (9H, s), 2.77 (3H, s), 8.79 (1H, s)

m/z (ESI+) (M+H)+=195; HPLC t_{R}=1.67 min.

The following Example was prepared in a similar manner to Example 6, using an appropriate carboxylic acid starting material (intermediate 17):

| Structure | Ex | Name | ¹H NMR δ | MS m/e MH⁺ |
|---|---|---|---|---|
| 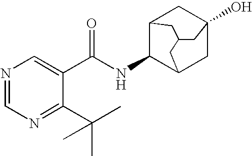 | 7 | 4-tert-butyl-N- [(2r,5s)-5- hydroxyadamantan- 2-yl]pyrimidine-5- carboxamide | 1H NMR (400.132 MHz, CDCl3) δ 1.41 (1H, s), 1.44 (9H, s), 1.59 (2H, d), 1.65-1.74 (2H, m), 1.78-1.86 (4H, m), 1.96 (2H, d), 2.18 (1H, s), 2.27 (2H, s), 4.24 (1H, d), 5.94 (1H, d), 8.52 (1H, s), 9.15 (1H, s) | 330; HPLC t_{R} = 1.45 min. |

The following intermediates were used and were prepared as described below.

### Intermediate 16

### Ethyl 4-tert-butylpyrimidine-5-carboxylate

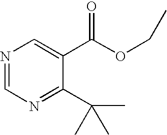

Prepared from Methyl 2-((dimethylamino)methylene)-3-oxobutanoate (Intermediate 11) by the same process used for Intermediate 12.

1H NMR (400.132 MHz, CDCl3) δ 1.41 (3H, t), 1.43 (9H, s), 4.41 (2H, q), 8.64 (1H, s), 9.15 (1H, s)

m/z (ESI+) (M+HOAc)+=269; HPLC t_{R}=2.11 min.

### Intermediate 17

### 4-tert-butylpyrimidine-5-carboxylic acid

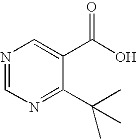

Prepared from ethyl 4-tert-butylpyrimidine-5-carboxylate (intermediate 16) by the same process used for Intermediate 10.

1H NMR (400.132 MHz, CDCl3) δ 1.48 (9H, s), 8.85 (1H, s), 9.23 (1H, s)

m/z (ESI+) (M+H)+=181; HPLC t_{R}=1.07 min.

### Example 8

### N-[(2r,5s)-5-hydroxyadamantan-2-yl]-2-morpholin-4-yl-4-propylsulfanyl-pyrimidine-5-carboxamide

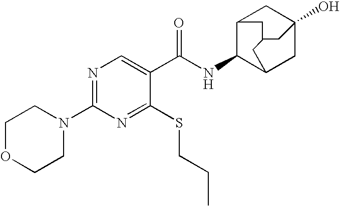

Oxalyl chloride (0.187 mL, 2.14 mmol) was added dropwise to a stirred solution of 2-morpholino-4-(propylthio)pyrimidine-5-carboxylic acid (Intermediate 21, 303 mg, 1.07 mmol) in dichloromethane (20 mL) cooled to 5° C., over a period of 5 minutes under air. The resulting solution was stirred at 20° C. for 1 hour until the gas evolution had stopped. The solution was evaporated under reduced pressure and redissolved in DCM. It was then added to a suspension of (1s,4r)-4-aminoadamantan-1-ol (179 mg, 1.07 mmol) and N-ethyldiisopropylamine (0.559 mL, 3.21 mmol) in THF (10 mL) at room temperature over a period of 20 minutes under air. The resulting solution was stirred at room temperature for 2 days.

The reaction mixture was concentrated and diluted with EtOAc (75 mL), and washed sequentially with water (2×20 mL) and saturated brine (10 mL). The organic layer was dried over MgSO4, filtered and evaporated to afford crude product.

The crude product was purified by flash silica chromatography eluting with 10% MeOH in DCM. Pure fractions were evaporated to dryness to afford N-[(2r,5s)-5-hydroxyadamantan-2-yl]-2-morpholin-4-yl-4-propylsulfanyl-pyrimidine-5-carboxamide (127 mg, 28%) as a white solid.

1H NMR (400.13 MHz, DMSO-d₆) δ 0.96 (3H, t), 1.32 (2H, d), 1.58-1.66 (6H, m), 1.71 (2H, d), 1.94-2.03 (5H, m), 3.01 (2H, t), 3.65-3.67 (4H, m), 3.75-3.79 (4H, m), 3.84-3.88 (1H, m), 4.44 (1H, s), 7.87 (1H, d), 8.32 (1H, s)

m/z (ESI+) (M+H)+=433; HPLC t_{R}=1.94 min.

### Intermediate 18

### Ethyl 2-morpholino-6-oxo-1,6-dihydropyrimidine-5-carboxylate

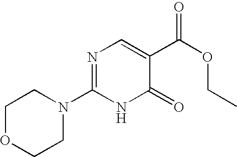

Morpholinoformamidine hydrobromide (4.20 g, 20.0 mmol) was added portionwise to diethyl ethoxymethylenemalonate (4.04 mL, 20.0 mmol) and potassium carbonate (3.04 g, 22.0 mmol) in ethanol (80 mL) at room temperature and under air. The resulting suspension was stirred at 80° C. for 2 hours causing formation of a white slurry. The reaction mixture was evaporated to dryness and acidified to pH 4-5. A white solid precipitated and was extracted with EtOAc (100 mL) and washed with saturated brine (20 mL). The organic layer was dried over MgSO4, filtered and evaporated to afford ethyl 2-morpholino-6-oxo-1,6-dihydropyrimidine-5-carboxylate (2.13 g, 42%).

1H NMR (400.13 MHz, DMSO-d₆) δ 1.24 (3H, t), 3.61-3.67 (4H, m), 3.69-3.74 (4H, m), 4.17 (2H, q),845 (1H, s), 11.53 (1H, s)

m/z (ESI+) (M+H)+=254; HPLC t_{R}=1.12 min.

### Intermediate 19

### Ethyl 4-chloro-2-morpholinopyrimidine-5-carboxylate

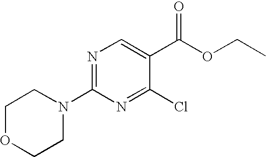

Phosphorus oxychloride (20 mL, 214 mmol) was added to ethyl 2-morpholino-6-oxo-1,6-dihydropyrimidine-5-carboxylate (Intermediate 18, 2.130 g, 8.41 mmol), and warmed to 100° C. over a period of 5 minutes under nitrogen. The resulting suspension was stirred at 100° C. for 40 minutes and then allowed to cool down to room temperature. The reaction mixture was evaporated to dryness, redissolved in EtOAc (75 mL) and washed sequentially with water (15 mL) and saturated brine (10 mL). The organic layer was dried over MgSO4, filtered and evaporated to afford crude product.

It was then purified by flash silica chromatography, elution gradient 0 to 50% EtOAc in isohexane. Pure fractions were evaporated to dryness to afford ethyl 4-chloro-2-morpholinopyrimidine-5-carboxylate (2.04 g, 89%) as a white solid.

1H NMR (400.13 MHz, DMSO-d₆) δ 1.30 (3H, t), 3.67-3.68 (4H, m), 3.80 (4H, s), 4.27 (2H, q), 8.82 (1H, s)

m/z (ESI+) (M+H)+=272; HPLC t_{R}=2.14 min.

### Intermediate 20

### ethyl 2-morpholino-4-(propylthio)pyrimidine-5-carboxylate

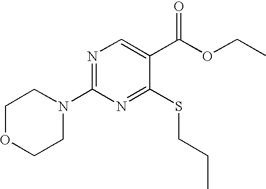

A solution of sodium bis(trimethylsilyl)amide (2.21 mL, 2.21 mmol) in THF (1M) was added to a stirred solution of 1-propanethiol (0.183 mL, 2.02 mmol) in DMF (10 mL) at room temperature, over a period of 3 minutes under air. The resulting suspension was stirred for 15 minutes and was added portionwise to a solution of ethyl 4-chloro-2-morpholinopyrimidine-5-carboxylate (Intermediate 19, 500 mg, 1.84 mmol) in DMF (5 mL). The resulting suspension was stirred at room temperature for 2 hours.

The reaction mixture was diluted with water (50 mL), extracted with DCM (100 mL) and washed with saturated brine (20 mL). The organic layer was dried over MgSO4, filtered and evaporated to afford ethyl 2-morpholino-4-(propylthio)pyrimidine-5-carboxylate (529 mg, 92%).

1H NMR (400.13 MHz, DMSO-d₆) δ 0.97 (3H, t), 1.28 (3H, t), 1.60-1.70 (2H, m), 3.03 (2H, t),3.65-3.72 (4H, m),3.80-3.87 (4H, m),4.23 (2H, q), 8.64 (1H, s)

m/z (ESI+) (M+H)+=312; HPLC t_{R}=2.60 min.

### Intermediate 21

### 2-morpholino-4-(propylthio)pyrimidine-5-carboxylic acid

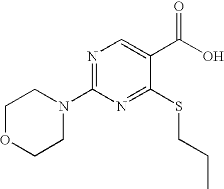

A solution of aqueous 2N sodium hydroxide (8.49 mL, 16.99 mmol) was added to a stirred suspension of ethyl 2-morpholino-4-(propylthio)pyrimidine-5-carboxylate (Intermediate 20, 529 mg, 1.70 mmol) in ethanol (15 mL). The resulting suspension was stirred at room temperature for 18 hours.

The reaction mixture was evaporated to dryness and redissolved in water (10 mL). It was then acidified with 2M HCl to pH 4-5, extracted with DCM (75 mL), and washed with saturated brine (10 mL). The organic layer was dried over MgSO4, filtered and evaporated to afford 2-morpholino-4-(propylthio)pyrimidine-5-carboxylic acid (313 mg, 65%).

1H NMR (400.13 MHz, DMSO-d₆) δ 0.97 (3H, t), 1.60-1.69 (2H, m), 3.01 (2H, t), 3.68 (4H, t), 3.83 (4H, t), 8.62 (1H, s), 12.76 (1H, s)

m/z (ESI+) (M+H)+=284; HPLC t_{R}=1.91 min.

### Example 9

### N-[(2r,5s)-5-hydroxyadamantan-2-yl]-2-methyl-4-propylsulfanylpyrimidine-5-carboxamide

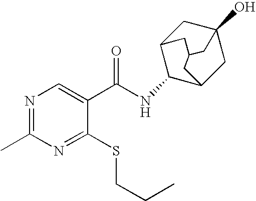

Oxalyl chloride (0.20 mL, 2.36 mmol) was added dropwise to a suspension of 2-methyl-4-(propylthio)pyrimidine-5-carboxylic acid (Intermediate 24, 456 mg, 2.15 mmol) in DCM (20 mL) containing 3 drops of DMF at 20° C. under nitrogen. The resulting mixture was stirred at 20° C. for 2 hours. The reaction mixture was evaporated, redissolved in DCM (10 mL) and added dropwise to a suspension of 4-aminoadamantan-1-ol (359 mg, 2.15 mmol) and N,N-di-isopropylamine (1.10 mL, 6.44 mmol) in tetrahydrofuran (30 mL). The resulting mixture was stirred at 20° C. for 2 hours. The reaction mixture was diluted with EtOAc (100 mL), and then washed sequentially with water (2×100 mL). The organic layer was dried over MgSO4, filtered and evaporated to afford the crude product. The crude product was purified by flash silica chromatography, elution gradient 0 to 10% MeOH in DCM. Pure fractions were evaporated to dryness to afford N-[(2r,5s)-5-hydroxyadamantan-2-yl]-2-methyl-4-propylsulfanylpyrimidine-5-carboxamide (578 mg, 74%) as a yellow oil.

1H NMR (400.13 MHz, CDCl₃) δ 0.99 (3H, t), 1.51 (2H, d), 1.65-1.74 (5H, m), 1.75 (2H, s), 1.74-1.79 (1H, m), 1.87 (2H, d), 2.13 (1H, s), 2.20 (2H, s), 2.62 (3H, s), 3.19 (2H, t), 4.14-4.19 (1H, m), 6.64 (1H, d), 8.61 (1H, s)

m/z (ESI+) (M+H)+=362; HPLC t_{R}=1.79 min.

### Intermediate 22

### Methyl 2-methyl-4-oxo-3H-pyrimidine-5-carboxylate

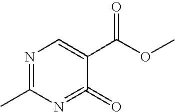

Diethyl 2-(ethoxymethylene)malonate (2.11 mL, 10.53 mmol) and acetimidamide (611 mg, 10.53 mmol) was added in one portion to sodium methoxide in methanol (0.5 M, 70 mL, 35 mmol) at room temperature. The resulting mixture was refluxed for 4 hours. The precipitate was collected by filtration, washed with MeOH (125 mL) and dried under vacuum to afford methyl 2-methyl-4-oxo-3H-pyrimidine-5-carboxylate (1.14 g, 64%), which was used without further purification.

1H NMR (400.13 MHz, DMSO-d₆) δ 2.12 (3H, s), 3.16 (1H, s), 3.63 (3H, s), 8.27 (1H, s)

m/z (ESI+) (M+H)+=169; HPLC t_{R}=0.60 min

### Intermediate 23

### Methyl 2-methyl-4-propylsulfanylpyrimidine-5-carboxylate

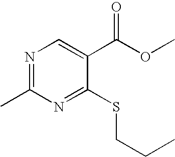

Phosphorous oxychloride (15 mL, 6.78 mmol) was added to methyl 2-methyl-6-oxo-1,6-dihydropyrimidine-5-carboxylate (Intermediate 22, 1.14 g, 6.78 mmol). The insoluble mixture was refluxed for 3 hours. The excess POCl3 was removed under vacuum. The mixture was evaporated to dryness and redissolved in EtOAc (100 mL) and washed sequentially with water (2×75 mL). The organic layer was dried over MgSO4, filtered and evaporated to afford methyl 4-chloro-2-methylpyrimidine-5-carboxylate, which was used without further purification or characterisation.

Sodium carbonate (819 mg, 7.73 mmol) was added to methyl 4-chloro-2-methylpyrimidine-5-carboxylate (490 mg, 2.63 mmol) and 1-propane thiol (0.24 mL, 2.63 mmol) in DMF (10 mL). The resulting solution was stirred at 60° C. for 30 minutes. The reaction mixture was diluted with EtOAc (150 mL) and washed sequentially with water (2×100 mL). The organic layer was dried over MgSO4, filtered and evaporated to afford crude product. The crude product was purified by flash silica chromatography, elution gradient 10 to 50% EtOAc in isohexane. Pure fractions were evaporated to dryness to afford methyl 2-methyl-4-(propylthio)pyrimidine-5-carboxylate (425 mg, 72%).

1H NMR (400.13 MHz, CDCl₃) δ 0.98 (3H, t), 1.62-1.71 (2H, m), 2.62 (3H, s), 3.10 (2H, t), 3.85 (3H, s), 8.81 (1H, s)

m/z (ESI+) (M+H)+=227; HPLC t_{R}=2.28 min

### Intermediate 24

### 2-methyl-4-propylsulfanylpyrimidine-5-carboxylic acid

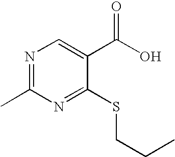

Prepared from methyl 2-methyl-4-propylsulfanylpyrimidine-5-carboxylate (intermediate 23) by the same process used for Intermediate 21.

1H NMR (400.13 MHz, CDCl₃) δ 1.00 (3H, t), 1.65-1.74 (2H, m), 2.72 (3H, s), 3.14 (2H, t), 7.19 (1H, s), 8.99 (1H, s)

m/z (ESI+) (M+H)+=213; HPLC t_{R}=1.66 min.

The following Examples were prepared in a similar manner to Example 9, using (1s,4r)-4-aminoadamantan-1-ol and an appropriate carboxylic acid starting material (intermediate 27):

| Structure | Ex | Name | ¹H NMR δ | MS m/e MH⁺ |
|---|---|---|---|---|
| 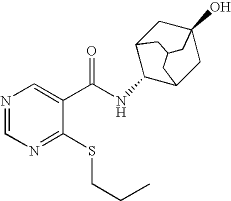 | 10 | N-[(2r,5s)-5- hydroxyadamantan-2-yl]-4- propylsulfanylpyrimidine-5- carboxamide | 1H NMR (400.13 MHz, CDCl3) δ 0.99 (3H, t), 1.51 (2H, d), 1.65-1.73 (5H, m), 1.76 (2H, s), 1.79 (1H, s), 1.86-1.89 (2H, m), 2.13 (1H, s), 2.20 (2H, s), 3.19 (2H, t), 4.14-4.20 (1H, m), 6.65 (1H, d), 8.65 (1H, s), 8.86 (1H, s) | 348; HPLC t_{R} = 1.64 min |

The following intermediates were used and were prepared as described below.

### Intermediate 25

### Ethyl 4-oxo-3H-pyrimidine-5-carboxylate

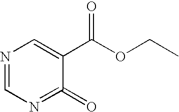

Prepared by the same process used for Intermediate 22.

1H NMR (400.13 MHz, DMSO-d₆) δ 1.25 (3H, t), 3.52 (1H, s), 4.16 (2H, q), 8.27 (1H, s), 8.42 (1H, s)

m/z (ESI+) (M+H)+=169; HPLC t_{R}=0.50 min.

### Intermediate 26

### Ethyl 4-propylsulfanylpyrimidine-5-carboxylate

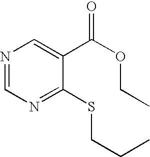

Prepared from ethyl 4-oxo-3H-pyrimidine-5-carboxylate (intermediate 25) by the same process used for Intermediate 23.

1H NMR (400.13 MHz, CDCl₃) δ 0.77 (3H, t), 1.01 (3H, t), 1.67 (2H, q), 3.11 (2H, t), 4.35 (2H, q), 8.90 (1H, d), 8.92 (1H, d)

m/z (ESI+) (M+H)+=227; HPLC t_{R}=2.30 min.

### Intermediate 27

### 4-Propylsulfanylpyrimidine-5-carboxylate

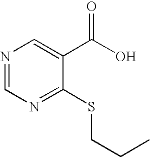

Prepared from ethyl 4-propylsulfanylpyrimidine-5-carboxylate (intermediate 26) by the same process used for Intermediate 21.

m/z (ESI+) (M+H)+=199; HPLC t_{R}=1.56 min.

### Example 11

### 4-Cyclopropyl-N-[(2r,5s)-5-hydroxyadamantan-2-yl]-2-methylsulfanyl-pyrimidine-5-carboxamide

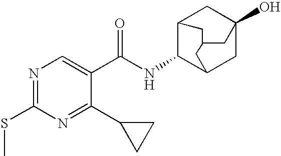

O-(7-azabenzotriazol-1-yl)-N,N,N′,N′-tetramethyluronium hexafluorophosphate (458 mg, 1.21 mmol) was added to 4-cyclopropyl-2-(methylthio)pyrimidine-5-carboxylic acid (Intermediate 29, 230 mg, 1.09 mmol) and N,N-di-isopropylamine (0.375 mL, 2.19 mmol) in DMF (7 mL). The resulting solution was stirred at room temperature for 15 minutes then (1r,4s)-4-aminoadamantan-1-ol hydrochloride (268 mg, 1.32 mmol) was added and the reaction was allowed to stir at room temperature for 2 hours. The reaction mixture was evaporated to dryness and redissolved in EtOAc (150 mL) and then washed sequentially with water (2×150 mL). The organic layer was dried over MgSO4, filtered and evaporated to afford the crude product. The crude product was purified by flash silica chromatography, elution gradient 0 to 10% MeOH in DCM. Pure fractions were evaporated to dryness to afford 4-cyclopropyl-N-[(2r,5s)-5-hydroxyadamantan-2-yl]-2-methylsulfanyl-pyrimidine-5-carboxamide (311 mg, 79%) as a yellow oil.

1H NMR (400.13 MHz, CDCl₃) δ 1.01-1.05 (2H, m), 1.20-1.22 (2H, m), 1.49 (2H, d), 1.68-1.75 (5H, m), 1.84-1.87 (2H, m), 2.08 (2H, s), 2.17 (2H, s), 2.31-2.37 (1H, m), 2.41 (3H, s), 4.13-4.18 (1H, m), 6.41 (1H, d), 8.29 (1H, s)

m/z (ESI+) (M+H)+=360; HPLC t_{R}=1.89 min.

### Intermediate 28

### Ethyl 4-cyclopropyl-2-(methylthio)pyrimidine-5-carboxylate

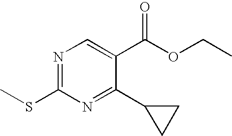

Ethyl 2-(cyclopropanecarbonyl)-3-(dimethylamino)acrylate (Intermediate 1, 557 mg, 2.64 mmol) was dissolved in DMF (10 mL). To the solution was added 2-methyl-2-thiopseudourea sulfate (850 mg, 3.05 mmol) and sodium acetate (919 mg, 11.21 mmol). The reaction was heated at 80° C. for 2 hours. Water was added to the cooled solution and the aqueous layer was washed sequentially with EtOAc (3×200 mL). The combined organic layers were washed with saturated aqueous NaHCO3 (100 mL). The organic layer was dried over MgSO4, filtered and evaporated to afford crude product. The crude product was purified by flash silica chromatography, elution gradient 0 to 10% EtOAc in isohexane. Pure fractions were evaporated to dryness to afford ethyl 4-cyclopropyl-2-(methylthio)pyrimidine-5-carboxylate (596 mg, 95%) as a colourless oil.

1H NMR (400.13 MHz, CDCl₃) δ 0.98-1.02 (2H, m), 1.15-1.19 (2H, m), 1.28 (3H, t), 2.39 (3H, s), 3.05-3.12 (1H, m), 4.27 (2H, q), 8.71 (1H, s)

m/z (ESI+) (M+H)+=239; HPLC t_{R}=2.77 min.

### Intermediate 29

### 4-cyclopropyl-2-(methylthio)pyrimidine-5-carboxylic acid

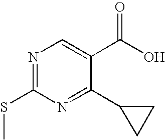

Ethyl 4-cyclopropyl-2-(methylthio)pyrimidine-5-carboxylate (Intermediate 28, 298 mg, 1.25 mmol) was dissolved in methanol (10 mL) and 2M aqueous sodium hydroxide (2 mL) was added. The resulting solution was stirred at room temperature for 3 hours. The reaction mixture was evaporated to dryness and redissolved in water (100 mL) then was acidified to pH 4 with 2N HCl. The aqueous layer was washed sequentially with DCM (2×75 mL). The organic layer was dried over MgSO4, filtered and evaporated to afford crude 4-cyclopropyl-2-(methylthio)pyrimidine-5-carboxylic acid (230 mg, 87%) as a white solid, which was used without further purification and characterisation.

m/z (ESI+) (M+H)+=211; HPLC t_{R}=1.96 min.

The following Examples were prepared in a similar manner to Example 1, using 2-adamantylamine and an appropriate carboxylic acid starting material (Intermediate 5 & Intermediate 3 respectively) as described previously:

| Structure | Ex | Name | ¹H NMR δ | MS m/e MH⁺ |
|---|---|---|---|---|
| 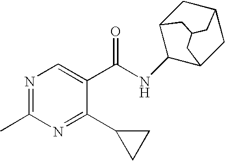 | 12 | N-(2- adamantyl)-4- cyclopropyl-2- methyl- pyrimidine-5- carboxamide | 1H NMR (400.13 MHz, DMSO-d6) δ 1.01-1.08 (4H, m), 1.51 (2H, d), 1.70 (2H, s), 1.76-1.83 (5H, m), 1.84 (1H, s), 1.94 (2H, s), 2.04 (2H, d), 2.25-2.31 (1H, m), 2.52 (3H, s), 4.04-4.07 (1H, m), 8.41 (1H, s), 8.46 (1H, d) | 312; HPLC t_{R} = 2.32 min. |
| 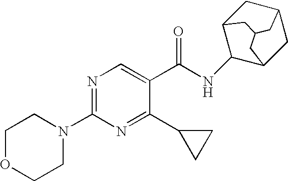 | 13 | N-(2- adamantyl)-4- cyclopropyl-2- morpholino- pyrimidine-5- carboxamide | 1H NMR (400.13 MHz, DMSO-d6) δ 0.92-0.97 (2H, m), 0.99-1.04 (2H, m), 1.50 (2H, d), 1.70 (2H, s), 1.76-1.81 (5H, m), 1.83 (1H, s), 1.92 (2H, s), 2.03-2.06 (2H, m), 2.43-2.47 (1H, m), 3.59-3.63 (4H, m), 3.64-3.68 (4H, m), 3.98-4.02 (1H, m), 8.10 (1H, d), 8.23 (1H, s) | 383; HPLC t_{R} = 2.65 min. |

### Example 14

### N-(2-Adamantyl)-4-tert-butyl-2-morpholin-4-ylpyrimidine-5-carboxamide

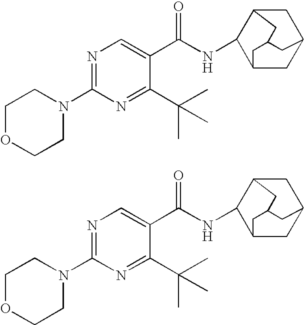

Morpholine-4-carboximidamide hydrobromide (213 mg, 1.01 mmol) and N-(2-adamantyl)-2-(dimethylaminomethylidene)-4,4-dimethyl-3-oxopentanamide (Intermediate 30, 340 mg, 1.02 mmol) was added at room temperature to a solution of sodium methoxide (2.23 mL, 1.11 mmol) in methanol (10 mL). The mixture was refluxed for 3.5 hours. The reaction mixture was evaporated to dryness and redissolved in DCM (125 mL), and washed with saturated brine (2×75 mL). The organic layer was dried over MgSO4, filtered and evaporated to afford the crude product. The crude product was purified by flash silica chromatography, elution gradient 10 to 40% EtOAc in isohexane. Pure fractions were evaporated to dryness to afford N-(2-adamantyl)-4-tert-butyl-2-morpholin-4-ylpyrimidine-5-carboxamide (96 mg, 24%) as a white solid.

1H NMR (400.13 MHz, DMSO-d6) δ 1.32 (9H, s), 1.49 (2H, d), 1.70 (2H, s), 1.76 (1H, s), 1.80 (3H, s), 1.84 (1H, s), 1.90 (3H, s), 2.04 (2H, s), 3.66 (4H, d), 3.71-3.73 (4H, m), 8.10 (1H, s), 8.27 (1H, d)

m/z (ESI+) M+=399; HPLC t_{R}=3.00 min

### Intermediate 30

### N-(2-adamantyl)-2-(dimethylaminomethylidene)-4,4-dimethyl-3-oxo-pentanamide

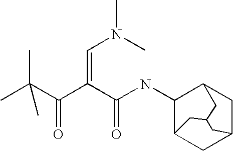

A 1M solution of solution of lithium bis(trimethylsilyl)amide in THF (22.84 ml, 22.84 mmol) was added to THF (25 mL) and cooled under nitrogen to −78° C. A solution of 3,3-dimethyl-2-butanone (2.287 g, 22.84 mmol) in THF (25 mL) was added drop wise over a period of 5 minutes. The resulting solution was stirred at −78° C. under nitrogen for 15 minutes. A solution of 2-isocyanatoadamantane (prepared from 2-adamantylamine hydrochloride by the method of R. Reck & C. Jochims, Chem. Ber., 1982, 115, 864) (3.68 g, 20.76 mmol) in THF (20 mL) was added over a period of 5 minutes. The resulting solution was stirred at −78° C. for 1 hour and then allowed to warm to 20° C. over 1 h. The reaction mixture was poured into saturated NH₄Cl (150 mL) and extracted with EtOAc (2×100 mL), the organic layer was washed with water (50 mL) and brine (50 mL), dried over MgSO4, filtered and evaporated to afford a yellow oil. The crude product was purified by flash silica chromatography, elution gradient 0 to 50% EtOAc in isohexane. Pure fractions were evaporated to dryness to afford N-(2-adamantyl)-4,4-dimethyl-3-oxo-pentanamide (4.64 g, 81%) as a white solid.

1H NMR (400.13 MHz, DMSO-d₆) δ 1.08-1.09 (9H, m), 1.50 (2H, d), 1.66-1.89 (10H, m), 1.95-2.00 (2H, m), 3.53 (1.4H, s), 3.80-3.94 (1H, m), 5.30 (0.3H, s), 7.77-7.87 (1H, m), 14.43 (0.3H, s) (2:1 mixture of keto and enol forms)

m/z (ESI+) (M+H)+=278

N,N-Dimethylformamide dimethyl acetal (3.02 mL, 22.71 mmol) was added to a stirred suspension of N-(2-adamantyl)-4,4-dimethyl-3-oxo-pentanamide (5.25 g, 18.93 mmol) in 1,4-dioxane (50 mL) under nitrogen. The resulting mixture was stirred at 100° C. for 2 hours. The reaction mixture was evaporated to dryness and the resulting pale cream solid was dried under vacuum to afford N-(2-adamantyl)-2-(dimethylaminomethylidene)-4,4-dimethyl-3-oxo-pentanamide (5.83 g, 93%).

1H NMR (400.13 MHz, DMSO-d₆) δ 1.13 (9H, s), 1.47 (2H, d), 1.69-1.83 (10H, m), 2.03 (2H, d), 2.92 (6H, s), 3.90 (1H, d), 7.24 (1H, s), 7.94 (1H, d)

m/z (ESI+) (M+H)+=333

The following Examples were prepared in a similar manner to Example 1, using 2-adamantylamine and an appropriate carboxylic acid starting material:

| Structure | Ex | Name | ¹H NMR δ | MS m/e MH⁺ |
|---|---|---|---|---|
| 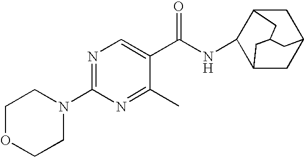 | 15 | N-(2- adamantyl)-4- methyl-2- morpholin-4- ylpyrimidine-5- carboxamide | 1H NMR (400.13 MHz, DMSO-d6) δ 1.51 (2H, d), 1.71 (2H, s), 1.79-1.84 (6H, m), 1.92 (2H, s), 2.05 (2H, d), 2.40 (3H, s), 3.63-3.66 (4H, m), 3.74-3.76 (4H, m), 3.96-4.01 (1H, m), 8.07 (1H, d), 8.32 (1H, s) | 357; HPLC t_{R} = 2.43 min |

### Intermediate 31

### N-(2-adamantyl)-2-(dimethylaminomethylidene)-3-oxo-butanamide

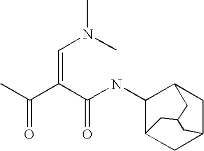

2-Adamantanamine hydrochloride (23.70 g, 126.23 mmol) was added to 5-acetyl-2,2-dimethyl-1,3-dioxane-4,6-dione (23.5 g, 126.23 mmol) and N-Ethyldiisopropylamine (21.84 mL, 126.23 mmol) in toluene (300 mL). The resulting suspension was stirred at 110° C. for 2 hours. The reaction mixture was diluted with EtOAc (50 mL), and washed sequentially with 2M HCl (25 mL) and water (2×50 mL). The organic layer was dried over MgSO4, filtered and evaporated to afford crude product. The crude product was purified by flash silica chromatography, elution gradient 50 to 80% EtOAc in isohexane. Pure fractions were evaporated to dryness to afford N-(2-adamantyl)-3-oxo-butanamide (15.80 g, 53%) as an orange oil which crystallised on standing.

1H NMR (400.13 MHz, DMSO-d6) δ 1.48-1.54 (2H, m), 1.69-1.85 (10H, m), 1.92-2.00 (2H, s), 2.13 (3H, s), 3.38 (2H, s), 3.84 (1H, d), 7.95 (1H, d) N-(2-adamantyl)-2-(dimethylaminomethylidene)-3-oxo-butanamide was then prepared from ethyl N-(2-adamantyl)-3-oxo-butanamide by the same process used for Intermediate 30 described above.

1H NMR (400.13 MHz, DMSO-d₆) δ 1.46-1.52 (2H, m), 1.65-1.70 (2H, m), 1.72-1.85 (8H, m), 1.92-2.00 (2H, m), 2.04 (3H, s), 2.99 (6H, s), 3.91-3.96 (1H, m), 7.44 (1H, s), 8.35 (1H, d)

### Example 16

### N-[(2r,5s)-5-hydroxyadamantan-2-yl]-2,4-bis(propylsulfanyl)pyrimidine-5-carboxamide

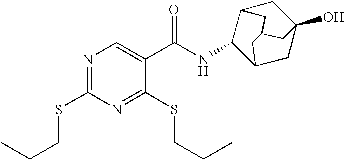

O-(7-Azabenzotriazol-1-yl)-N,N,N′,N′-tetramethyluronium hexafluorophosphate (335 mg, 0.88 mmol) was added in one portion to 2,4-bis(propylthio)pyrimidine-5-carboxylic acid (Intermediate 36, 200 mg, 0.73 mmol), (1s,4r)-4-aminoadamantan-1-ol hydrochloride (150 mg, 0.73 mmol) and N-ethyldiisopropylamine (0.384 mL, 2.20 mmol) in DMF (10 mL) at 25° C. under nitrogen. The resulting solution was stirred at 25° C. for 3 hours. The reaction mixture was diluted with EtOAc (50 mL), and washed sequentially with saturated NaHCO3 (25 mL), water (2×25 mL) and saturated brine (25 mL). The organic layer was dried over MgSO4, filtered and evaporated to afford crude product. The crude product was purified by flash silica chromatography, elution gradient 0 to 25% EtOAc in isohexane. Pure fractions were evaporated to dryness dried under high vacuum to afford N-[(2r,5s)-5-hydroxyadamantan-2-yl]-2,4-bis(propylsulfanyl)pyrimidine-5-carboxamide (229 mg, 74%) as a white solid foam.

1H NMR (400.13 MHz, DMSO-d₆) δ 0.98 (6H, q), 1.32 (2H, d), 1.60-1.74 (10H, m), 1.92-2.04 (5H, m), 3.07-3.14 (4H, m), 3.88 (1H, t), 4.39 (1H, s), 8.20 (1H, d), 8.34 (1H, s)

m/z (ESI+) (M+H)+=422; HPLC t_{R}=2.47 min.

### Intermediate 32

### Ethyl 2,4-dichloropyrimidine-5-carboxylate

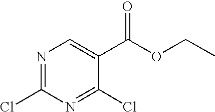

Ethyl 2,4-dioxo-1,2,3,4-tetrahydropyrimidine-5-carboxylate (5.0 g, 27.15 mmol) was added to phenyl phosphorodichloridate (32.4 ml, 217.21 mmol) under nitrogen. The resulting suspension was stirred at 180° C. for 1 hour. The reaction mixture was poured into ice/water (500 mL) and adjusted to pH 7 with saturated NaHCO3. The mixture was extracted with EtOAc (3×100 mL). The organic layers were combined and washed with water (2×100 mL), dried over Na2SO4, filtered and evaporated to afford crude product. The crude product was purified by flash silica chromatography, elution gradient 0 to 10% EtOAc in isohexane. Pure fractions were evaporated to dryness to afford ethyl 2,4-dichloropyrimidine-5-carboxylate (4.22 g, 70%) as a colourless oil which crystallised on standing.

1H NMR (400.13 MHz, DMSO-d₆) δ 1.33 (3H, t), 4.37 (2H, q), 9.15 (1H, s)

Intermediate 33: ethyl 2,4-bis(propylthio)pyrimidine-5-carboxylate,

Intermediate 34: ethyl 2-(dimethylamino)-4-(propylthio)pyrimidine-5-carboxylate and

Intermediate 35: ethyl 4-(dimethylamino)-2-(propylthio)pyrimidine-5-carboxylate

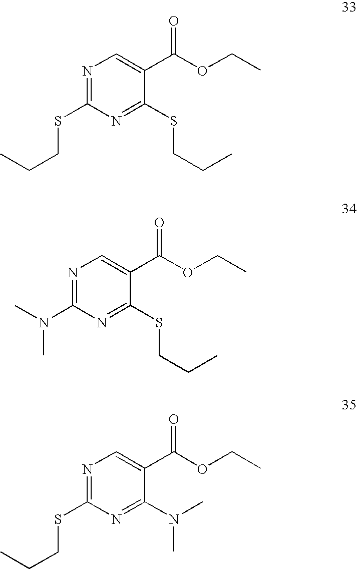

1-Propanethiol (0.408 mL, 4.51 mmol) was added in one portion to ethyl 2,4-dichloropyrimidine-5-carboxylate (Intermediate 32, 997 mg, 4.51 mmol) and sodium carbonate (1.434 g, 13.53 mmol) in DMF (10 mL) under nitrogen. The resulting suspension was stirred at 20° C. for 18 hours. The reaction mixture was diluted with EtOAc (100 mL), and washed sequentially with water (2×25 mL) and saturated brine (20 mL). The organic layer was dried over MgSO4, filtered and evaporated to afford crude product. The crude product was purified by flash silica chromatography, elution gradient 0 to 20% EtOAc in isohexane. Pure fractions were evaporated to dryness to afford a clear colourless oil which was added to a 2M solution of dimethylamine (23.01 ml, 46.02 mmol) in THF. The resulting mixture was stirred at 22° C. for 2 hours. The reaction mixture was evaporated to dryness and redissolved in EtOAc (100 mL) and washed sequentially with water (2×50 mL) and saturated brine (50 mL). The organic layer was dried over MgSO4, filtered and evaporated to afford a crude product containing three components. The crude product was purified by flash silica chromatography, elution gradient 0 to 25% EtOAc in isohexane. Pure fractions were evaporated to dryness to afford the following products as colourless oils.

Ethyl 2,4-bis(propylthio)pyrimidine-5-carboxylate (Intermediate 33, 410 mg, 30%)

1H NMR (400.13 MHz, CDCl₃) δ 0.98 (6H, t), 1.30 (3H, t), 1.64-1.73 (4H, m), 3.05-3.15 (4H, m), 4.25 (2H, q), 8.72 (1H, s)

m/z (ESI+) (M+H)+=301; HPLC t_{R}=3.35 min.

Ethyl 2-(dimethylamino)-4-(propylthio)pyrimidine-5-carboxylate (Intermediate 34, 200 mg, 17%)

1H NMR (400.13 MHz, CDCl₃) δ 0.98 (3H, t), 1.30 (3H, t), 1.64-1.73 (2H, m), 3.00-3.04 (2H, m), 3.22 (6H, s), 4.25 (2H, q), 8.64 (1H, s)

m/z (ESI+) (M+H)+=270; HPLC t_{R}=2.88 min.

Ethyl 4-(dimethylamino)-2-(propylthio)pyrimidine-5-carboxylate (Intermediate 35, 306 mg, 25%)

1H NMR (400.13 MHz, CDCl₃) δ 1.04-1.10 (3H, m), 1.36-1.42 (3H, m), 1.74-1.83 (2H, m), 3.11-3.16 (8H, m), 4.31-4.40 (2H, m), 8.51 (1H, s)

m/z (ESI+) (M+H)+=270; HPLC t_{R}=2.54 min.

### Intermediate 36

### 2,4-Bis(propylthio)pyrimidine-5-carboxylic acid

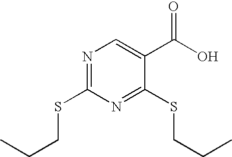

A solution of sodium hydroxide (3.41 mL, 6.82 mmol) was added to a stirred solution of ethyl 2,4-bis(propylthio)pyrimidine-5-carboxylate (Intermediate 33, 410 mg, 1.36 mmol) in MeOH (10 mL). The resulting mixture was stirred at 20° C. for 18 hours. The reaction mixture was concentrated and diluted with water (10 mL) and adjusted to pH4 with 2M HCl. The mixture was diluted with EtOAc (50 mL) and washed sequentially with water (2×25 mL) and saturated brine (25 mL). The organic layer was dried over MgSO4, filtered and evaporated to afford 2,4-bis(propylthio)pyrimidine-5-carboxylic acid (312 mg, 84%).

1H NMR (400.13 MHz, DMSO-d₆) δ 0.97-1.01 (6H, m), 1.63-1.75 (4H, m), 3.09 (2H, t), 3.14 (2H, t), 8.71 (1H, s)

m/z (ESI+) (M+H)+=273; HPLC t_{R}=1.54 min.

### Example 17

### 2-Dimethylamino-N-[(2r,5s)-5-hydroxyadamantan-2-yl]-4-propylsulfanylpyrimidine-5-carboxamide

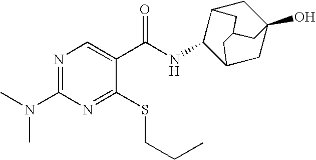

O-(7-Azabenzotriazol-1-yl)-N,N,N′,N′-tetramethyluronium hexafluorophosphate (248 mg, 0.65 mmol) was added in one portion to 2-(dimethylamino)-4-(propylthio)pyrimidine-5-carboxylic acid (Intermediate 37, 131 mg, 0.54 mmol), (1s,4r)-4-aminoadamantan-1-ol hydrochloride (111 mg, 0.54 mmol) and N-ethyldiisopropylamine (0.284 mL, 1.63 mmol) in DMF (5 mL) at 25° C. under nitrogen. The resulting solution was stirred at 25° C. for 3 hours.

The reaction mixture was diluted with EtOAc (50 mL), and washed sequentially with saturated NaHCO3 (25 mL), water (2×25 mL) and saturated brine (25 mL). The organic layer was dried over MgSO4, filtered and evaporated to afford crude product. The crude product was purified by flash silica chromatography, elution gradient 0 to 100% EtOAc in isohexane. Pure fractions were evaporated to dryness to afford 2-dimethylamino-N-[(2r,5s)-5-hydroxyadamantan-2-yl]-4-propylsulfanylpyrimidine-5-carboxamide (145 mg, 68%) as a white solid foam.

1H NMR (400.13 MHz, DMSO-d₆) δ 0.96 (3H, t), 1.31 (2H, d), 1.57-1.71 (8H, m), 1.93-2.02 (5H, m), 2.98-3.04 (2H, m), 3.16 (6H, s), 3.85 (1H, t), 4.37 (1H, s), 7.75 (1H, d), 8.29 (1H, s)

m/z (ESI+) (M+H)+=391; HPLC t_{R}=2.13 min.

### Intermediate 37

### 2-(Dimethylamino)-4-(propylthio)pyrimidine-5-carboxylic acid

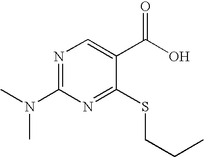

Prepared from ethyl 2-(dimethylamino)-4-(propylthio)pyrimidine-5-carboxylate (Intermediate 34) by the same process used for Intermediate 36.

1H NMR (400.13 MHz, DMSO-d₆) δ 0.97 (3H, t), 1.61-1.70 (2H, m), 3.00-3.04 (2H, m), 3.19 (6H, s), 8.58 (1H, s), 12.57 (1H, s)

m/z (ESI+) (M+H)+=242; HPLC t_{R}=0.86 min.

### Example 18

### 4-Dimethylamino-N-[(2r,5s)-5-hydroxyadamantan-2-yl]-2-propylsulfanylpyrimidine-5-carboxamide

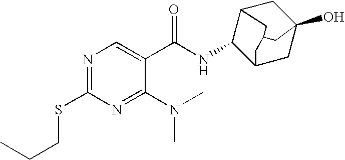

O-(7-Azabenzotriazol-1-yl)-N,N,N′,N′-tetramethyluronium hexafluorophosphate (454 mg, 1.19 mmol) was added in one portion to 4-(dimethylamino)-2-(propylthio)pyrimidine-5-carboxylic acid (Intermediate 38, 240 mg, 0.99 mmol), (1s,4r)-4-aminoadamantan-1-ol hydrochloride (203 mg, 0.99 mmol) and N-ethyldiisopropylamine (0.520 mL, 2.98 mmol) in DMF (10 mL) at 25° C. under nitrogen. The resulting solution was stirred at 25° C. for 3 hours.

The reaction mixture was diluted with EtOAc (50 mL), and washed sequentially with saturated NaHCO3 (25 mL), water (2×25 mL) and saturated brine (25 mL). The organic layer was dried over MgSO4, filtered and evaporated to afford crude product. The crude product was purified by flash silica chromatography, elution gradient 0 to 100% EtOAc in isohexane. Pure fractions were evaporated to dryness to afford 4-dimethylamino-N-[(2r,5s)-5-hydroxyadamantan-2-yl]-2-propylsulfanylpyrimidine-5-carboxamide (163 mg, 42%) as a white solid foam.

1H NMR (400.13 MHz, DMSO-d₆) δ 0.96 (3H, t), 1.32 (2H, d), 1.60-1.71 (8H, m), 1.89-2.01 (5H, m), 3.00-3.04 (8H, m), 3.88 (1H, t), 4.38 (1H, s), 7.91 (1H, s), 8.27 (1H, d)

m/z (ESI+) (M+H)+=391; HPLC t_{R}=1.87 min.

### Intermediate 38

### 4-(Dimethylamino)-2-(propylthio)pyrimidine-5-carboxylic acid

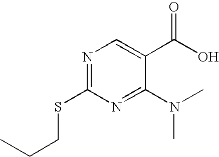

Prepared from ethyl 4-(dimethylamino)-2-(propylthio)pyrimidine-5-carboxylate (Intermediate 35) by the same process used for Intermediate 36.

1H NMR (400.13 MHz, DMSO-d₆) δ 0.97 (3H, t), 1.63-1.70 (2H, m), 3.01-3.06 (8H, m), 8.34 (1H, s)

m/z (ESI+) (M+H)+=242; HPLC t_{R}=0.71 min.

### Example 19

### (S)-Methyl 2-(1-(5-(cyclohexylcarbamoyl)-4-(propylthio)pyrimidin-2-yl)piperidin-3-yl)acetate

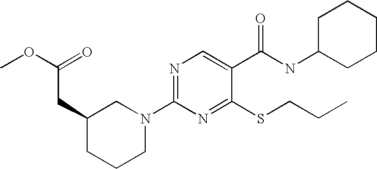

Potassium carbonate (0.363 g, 2.63 mmol) was added in one portion to 2-chloro-N-cyclohexyl-4-(propylthio)pyrimidine-5-carboxamide (Intermediate 40, 0.275 g, 0.88 mmol) and (S)-methyl 2-(piperidin-3-yl)acetate hydrochloride (0.170 g, 0.88 mmol) in butyronitrile (5 mL) at 20° C. under nitrogen. The resulting suspension was stirred at 120° C. for 24 hours. The reaction mixture was diluted with EtOAc (75 mL), and washed with saturated brine (20 mL). The organic layer was dried over MgSO4, filtered and evaporated to afford crude product. The crude product was purified by flash silica chromatography, elution gradient 0 to 50% EtOAc in DCM. Pure fractions were evaporated to dryness to afford (S)-methyl 2-(1-(5-(cyclohexylcarbamoyl)-4-(propylthio)pyrimidin-2-yl)piperidin-3-yl)acetate (0.351 g, 92%) as a white solid.

1H NMR (400.13 MHz, DMSO-d6) δ 0.95 (3H, t), 1.09-1.42 (7H, m), 1.55-1.90 (10H, m), 2.28 (2H, d), 2.84-3.09 (4H, m), 3.60-3.65 (4H, m), 4.41-4.49 (1H, m), 4.51-4.54 (1H, m), 7.89 (1H, d), 8.29 (1H, s)

m/z (ESI+) (M+H)+=435.36; HPLC t_{R}=2.95 min.

### Intermediate 39

### 2,4-dichloro-N-cyclohexylpyrimidine-5-carboxamide

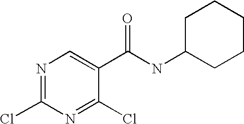

A solution of cyclohexylamine (0.951 mL, 8.32 mmol) and N-Ethyldiisopropylamine (1.44 mL, 8.32 mmol) in dichloromethane (5 mL) was added dropwise to a solution of 2,4-dichloropyrimidine-5-carbonyl chloride (CAS No. 2972-52-3; 1.76 g, 8.32 mmol) in DCM (20 mL) cooled to 0° C. over a period of 5 minutes under nitrogen. The resulting suspension was stirred at 0° C. for 2 hours then the temperature was increased to 20° C. and the reaction mixture was stirred for a further 2 hours. The reaction mixture was diluted with DCM (200 mL) and washed with water (50 mL). The organic layer was dried over MgSO4, filtered and evaporated to afford crude product. The crude product was purified by flash silica chromatography, elution gradient 0 to 10% EtOAc in DCM. Pure fractions were evaporated to dryness to afford 2,4-dichloro-N-cyclohexylpyrimidine-5-carboxamide (1.07 g, 47%) as a white solid.

1H NMR (400.13 MHz, DMSO-d6) δ 1.12-1.37 (5H, m), 1.53-1.58 (1H, m), 1.68-1.72 (2H, m), 1.83-1.85 (2H, m), 3.69-3.77 (1H, m), 8.57 (1H, d), 8.84 (1H, s)

m/z (ESI−) (M−H)-=272.13; HPLC t_{R}=2.03 min.

### Intermediate 40

### 2-chloro-N-cyclohexyl-4-(propylthio)pyrimidine-5-carboxamide

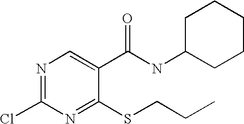

Sodium carbonate (0.199 g, 1.88 mmol) was added in one portion to 2,4-dichloro-N-cyclohexylpyrimidine-5-carboxamide (Intermediate 39, 0.515 g, 1.88 mmol) and 1-propanethiol (0.170 ml, 1.88 mmol) in DMF at 18° C. under nitrogen. The resulting suspension was stirred at 18° C. for 18 hours. The reaction mixture was diluted with EtOAc (75 mL), and washed sequentially with water (20 mL) and saturated brine (20 mL). The organic layer was dried over MgSO4, filtered and evaporated to afford crude product. The crude product was purified by flash silica chromatography, elution gradient 0 to 10% EtOAc in DCM. Pure fractions were evaporated to dryness to afford 2-chloro-N-cyclohexyl-4-(propylthio)pyrimidine-5-carboxamide (0.551 g, 93%) as a white solid.

1H NMR (400.13 MHz, DMSO-d6) δ 0.97 (3H, t), 1.09-1.35 (5H, m), 1.56-1.73 (5H, m), 1.80-1.83 (2H, m), 3.08 (2H, t), 3.65-3.73 (1H, m), 8.47 (1H, d), 8.50 (1H, s)

m/z (ESI+) (M+H)+=314.17; HPLC t_{R}=2.60 min.

### Example 20

### {(3S)-1-[5-(Cyclohexylcarbamoyl)-4-(propylthio)pyrimidin-2-yl]piperidin-3-yl}acetic acid

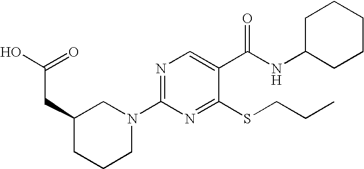

A solution of 2M sodium hydroxide (1.90 mL, 3.81 mmol) was added dropwise to a stirred solution of (S)-methyl 2-(1-(5-(cyclohexylcarbamoyl)-4-(propylthio)pyrimidin-2-yl)piperidin-3-yl)acetate (Example 19, 0.331 g, 0.76 mmol) in MeOH (5 mL) at ambient temperature and stirred for 18 hours. The reaction mixture was diluted with water (10 mL) then the pH was adjusted to ˜4.5 with 1M HCl aq. The suspension was diluted with EtOAc (50 mL), and washed with saturated brine (20 mL). The organic layer was dried over MgSO4, filtered and evaporated to afford crude product. The crude product was purified by preparative HPLC (Waters Xbridge column, 5μ silica, 50 mm diameter, 150 mm length), using decreasingly polar mixtures of water (containing 0.5% NH3) and MeCN as eluents. Fractions containing the desired compound were evaporated combined and the bulk of the CH3CN removed under reduced pressure. The clear colourless solution was acidified to ˜pH 4.5 with 1M HCl aq. and the white suspension extracted with EtOAc (50 mL). The organic layer was separated and dried over MgSO4, filtered and evaporated to afford (S)-2-(1-(5-(cyclohexylcarbamoyl)-4-(propylthio)pyrimidin-2-yl)piperidin-3-yl)acetic acid (0.200 g, 62%) as a white solid.

1H NMR (400.13 MHz, DMSO-d6) δ 0.95 (3H, t), 1.07-1.45 (7H, m), 1.55-1.90 (10H, m), 2.14-2.21 (2H, m), 2.82-3.09 (4H, m), 3.60-3.67 (1H, m), 4.41-4.50 (1H, m), 4.52-4.60 (1H, m), 7.89 (1H, d), 8.28 (1H, s), 12.07 (1H, s)

m/z (ESI+) (M+H)+=421.24; HPLC tt_{R}=2.52 min.

### Example 21

### N-[(2r,5s)-5-hydroxyadamantan-2-yl]-2-methylamino-4-propylsulfanylpyrimidine-5-carboxamide

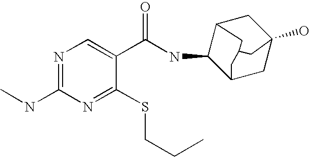

2-Chloro-N-[(2r,5s)-5-hydroxyadamantan-2-yl]-4-propylsulfanylpyrimidine-5-carboxamide (Intermediate 43, 383 mg, 1.00 mmol) was added to a solution of methylamine in ethanol (10.0 ml, 80.33 mmol). The resulting solution was stirred at 22° C. for 1 hour. The reaction mixture was evaporated to dryness to afford crude product that was purified by preparative HPLC (Waters XBridge Prep C18 OBD column, 5μ silica, 50 mm diameter, 150 mm length), using decreasingly polar mixtures of water (containing 0.5% NH3) and MeCN as eluents. Fractions containing the desired compound were evaporated to dryness to afford N-[(2r,5s)-5-hydroxyadamantan-2-yl]-2-methylamino-4-propylsulfanylpyrimidine-5-carboxamide (260 mg, 70%) as a white solid.

1H NMR (400.13 MHz, DMSO-d6) δ 0.96 (3H, t), 1.30-1.33 (2H, m), 1.60-1.71 (8H, m), 1.93-2.02 (5H, m), 2.84 (3H, d), 2.95-3.08 (2H, m), 3.85 (1H, t), 4.37 (1H, s), 7.31-7.52 (1H, m), 7.73 (1H, d), 8.23 (1H, s)

m/z (ESI+) (M+H)+=377; HPLC t_{R}=1.89 min.

### Intermediate 42

### 2,4-dichloro-N-[(2s,5r)-5-hydroxyadamantan-2-yl]pyrimidine-5-carboxamide

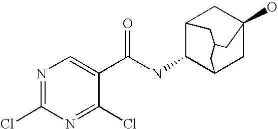

A suspension of (1r,4s)-4-aminoadamantan-1-ol.hydrochloride (2.89 g, 14.19 mmol) in THF (20 mL) was added drop wise to a stirred solution of 2,4-dichloropyrimidine-5-carbonyl chloride (3.0 g, 14.19 mmol) and N-ethyldiisopropylamine (4.91 mL, 28.38 mmol) in dichloromethane (20 mL) at −10° C., over a period of 5 minutes under nitrogen. The resulting suspension was stirred at 0° C. for 4 hours. The reaction mixture was diluted with DCM (150 mL), and washed sequentially with 0.1M HCl (50 mL), water (50 mL) and saturated brine (75 mL). The organic layer was dried over MgSO4, filtered and evaporated to afford the desired product. The crude solid was triturated with ice-cold DCM to give a solid which was collected by filtration and dried under vacuum to give 2,4-dichloro-N-[(2s,5r)-5-hydroxyadamantan-2-yl]pyrimidine-5-carboxamide (3.20 g, 66%) as a tan solid.

1H NMR (400.13 MHz, DMSO-d6) δ 1.36 (2H, d), 1.63 (4H, d), 1.71-1.77 (3H, m), 1.86 (2H, d), 1.98-2.00 (1H, m), 2.06 (2H, s), 3.95 (1H, t), 8.51 (1H, d), 8.83-8.85 (1H, m) HPLC t_{R}=1.44 min (no mass ion observed).

### Intermediate 43

### 2-chloro-N-[(2s,5r)-5-hydroxyadamantan-2-yl]-4-(propylthio)pyrimidine-5-carboxamide

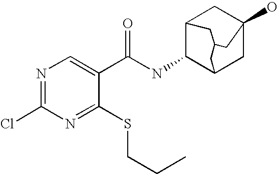

1-Propanethiol (0.151 mL, 1.67 mmol) was added in one portion to 2,4-dichloro-N-[(2s,5r)-5-hydroxyadamantan-2-yl]pyrimidine-5-carboxamide (Intermediate 42, 570 mg, 1.67 mmol), and Sodium carbonate (0.070 mL, 1.67 mmol) in DMF (10 mL) under nitrogen. The resulting suspension was stirred at room temperature for 4 hours. The reaction mixture was diluted with EtOAc (100 mL), and washed sequentially with water (25 mL) and saturated brine (25 mL). The organic layer was dried over MgSO4, filtered and evaporated to afford crude product. The crude product was purified by flash silica (40 g) chromatography, elution gradient 50 to 100% EtOAc in isohexane. Pure fractions were evaporated to dryness to afford 2-chloro-N-[(2s,5r)-5-hydroxyadamantan-2-yl]-4-(propylthio)pyrimidine-5-carboxamide (310 mg, 49%) as a white solid.

1H NMR (400.13 MHz, DMSO-d6) δ 0.97 (3H, t), 1.33 (2H, d), 1.62 (4H, d), 1.66 (2H, t), 1.70-1.73 (2H, m), 1.92 (2H, d), 1.99 (1H, s), 2.05 (2H, s), 3.11 (2H, t), 3.91 (1H, t), 4.40 (1H, s), 8.37 (1H, d), 8.47 (1H, s)

m/z (ESI+) (M+H)+=382; HPLC t_{R}=2.1 min.

### Example 22

### N-[(2r,5s)-5-hydroxyadamantan-2-yl]-4-methylamino-2-propylsulfanylpyrimidine-5-carboxamide

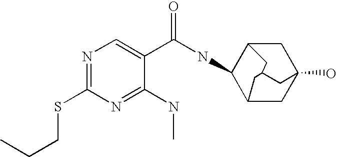

Prepared from 4-(methylamino)-2-(propylthio)pyrimidine-5-carboxylic acid (Intermediate 45) by the same process used for Example 16.

1H NMR (400.13 MHz, DMSO-d6) δ 0.97 (3H, t), 1.30-1.33 (2H, m), 1.60-1.73 (8H, m), 1.94-1.98 (3H, m), 2.05 (2H, s), 2.89 (3H, d), 3.04 (2H, t), 3.89 (1H, t), 4.38 (1H, s), 7.91-7.92 (1H, m), 8.38-8.42 (2H, m)

m/z (ESI+) (M+H)+=377; HPLC t_{R}=2.18 min.

### Intermediate 44

### Ethyl 4-(methylamino)-2-(propylthio)pyrimidine-5-carboxylate

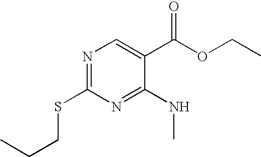

Prepared from ethyl 2,4-dichloropyrimidine-5-carboxylate (Intermediate 32) and methylamine by the same process used for Intermediate 35.

1H NMR (400.13 MHz, DMSO-d6) δ 0.97 (3H, t), 1.29 (3H, t), 1.64-1.73 (2H, m), 2.96-2.97 (3H, m), 3.04-3.08 (2H, m), 4.27 (2H, q), 8.21-8.22 (1H, m), 8.51 (1H, s)

m/z (ESI+) (M+H)+=256; HPLC t_{R}=2.84 min.

### Intermediate 45

### 4-methylamino-2-propylsulfanylpyrimidine-5-carboxylic acid

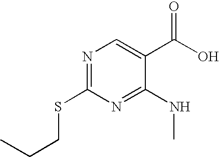

Prepared from ethyl 4-(methylamino)-2-(propylthio)pyrimidine-5-carboxylate (Intermediate 44) by the same process used for Intermediate 36.

1H NMR (400.13 MHz, DMSO-d6) δ 0.97 (3H, t), 1.64-1.73 (2H, m), 2.96 (3H, d), 3.06 (2H, t), 8.32-8.33 (1H, m), 8.47 (1H, s), 13.09 (1H, s)

m/z (ESI+) (M+H)+=228; HPLC t_{R}=1.33 min.

### Example 23

### 2-[(2s,6r)-2,6-Dimethylmorpholin-4-yl]-N-[(2r,5s)-5-hydroxyadamantan-2-yl]-4-propylsulfanylpyrimidine-5-carboxamide

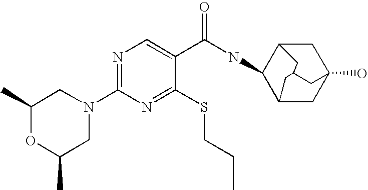

Prepared from 2-((2S,6R)-2,6-dimethylmorpholino)-4-(propylthio)pyrimidine-5-carboxylic acid (Intermediate 47) by the same process used for Example 16.

1H NMR (400.13 MHz, DMSO-d6) δ 0.96 (3H, t), 1.14 (6H, d), 1.31 (2H, d), 1.60-1.71 (8H, m), 1.92-2.02 (5H, m), 2.58-2.67 (2H, m), 2.99 (2H, t), 3.50-3.57 (2H, m), 3.85 (1H, t), 4.37 (1H, s), 4.52-4.55 (2H, m), 7.81 (1H, d), 8.28 (1H, s)

m/z (ESI+) (M+H)+=461; HPLC t_{R}=2.37 min.

### Intermediate 46

### Ethyl 2-((2S,6R)-2,6-dimethylmorpholino)-4-(propylthio)pyrimidine-5-carboxylate

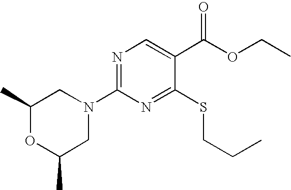

Prepared from ethyl 2,4-dichloropyrimidine-5-carboxylate (Intermediate 32) by the same process used for Intermediate 34.

1H NMR (400.13 MHz, DMSO-d6) δ 0.97 (3H, t), 1.15 (6H, s), 1.27 (3H, t), 1.60-1.69 (2H, m), 2.63-2.69 (2H, m), 3.00-3.01 (2H, m), 3.54-3.58 (2H, m), 4.22 (2H, q),456-4.60 (2H, m), 8.62 (1H, s)

m/z (ESI+) (M+H)+=340; HPLC t_{R}=3.24 min.

### Intermediate 47

### 2-((2S,6R)-2,6-dimethylmorpholino)-4-(propylthio)pyrimidine-5-carboxylic acid

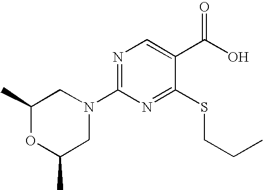

Prepared from ethyl 2-((2S,6R)-2,6-dimethylmorpholino)-4-(propylthio)pyrimidine-5-carboxylate (Intermediate 46) by the same process used for Intermediate 36.

1H NMR (400.13 MHz, DMSO-d6) δ 0.97 (3H, t), 1.14 (6H, d), 1.60-1.69 (2H, m), 2.62-2.67 (2H, m), 2.98 (2H, s), 3.54-3.58 (2H, m), 4.56-4.60 (2H, m), 8.59 (1H, s), 12.68 (1H, s)

m/z (ESI+) (M+H)+=312; HPLC t_{R}=1.14 min.

### Example 24

### 4-[(2S,6R)-2,6-Dimethylmorpholin-4-yl]-N-[(2r,5s)-5-hydroxyadamantan-2-yl]-2-propylsulfanylpyrimidine-5-carboxamide

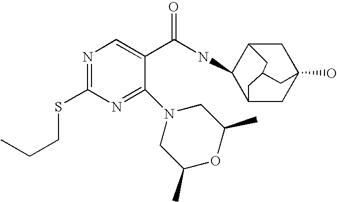

Prepared from 4-((2S,6R)-2,6-dimethylmorpholino)-2-(propylthio)pyrimidine-5-carboxylic acid (Intermediate 49) by the same process used for Example 16

1H NMR (400.13 MHz, DMSO-d6) δ 0.96 (3H, t), 1.07 (6H, d), 1.32 (2H, d), 1.60-1.72 (8H, m), 1.89 (2H, d), 2.00 (3H, d), 2.58-2.67 (2H, m), 3.00 (2H, t), 3.50-3.57 (2H, m), 3.85-3.87 (1H, m), 3.99 (2H, d), 4.40 (1H, s), 7.97 (1H, s), 8.35 (1H, d)

m/z (ESI+) (M+H)+=461; HPLC t_{R}=2.13 min.

### Intermediate 48

### Ethyl 4-((2S,6R)-2,6-dimethylmorpholino)-2-(propylthio)pyrimidine-5-carboxylate

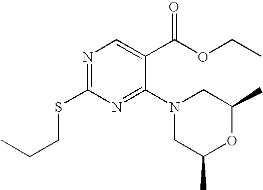

Prepared from ethyl 2,4-dichloropyrimidine-5-carboxylate (Intermediate 32) by the same process used for Intermediate 35

1H NMR (400.13 MHz, DMSO-d6) δ 0.97 (3H, t), 1.10 (6H, d), 1.28 (3H, t), 1.63-1.72 (2H, m), 2.66-2.75 (2H, m), 3.00-3.04 (2H, m), 3.54-3.62 (2H, m), 3.85-3.88 (2H, m), 4.25 (2H, q), 8.43-8.44 (1H, m)

m/z (ESI+) (M+H)+=340; HPLC t_{R}=2.82 min.

### Intermediate 49

### 4-((2S,6R)-2,6-dimethylmorpholino)-2-(propylthio)pyrimidine-5-carboxylic acid

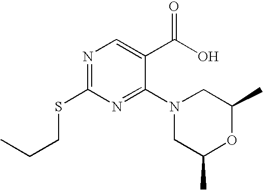

prepared from ethyl 4-((2S,6R)-2,6-dimethylmorpholino)-2-(propylthio)pyrimidine-5-carboxylate (Intermediate 48) by the same process used for Intermediate 36.

1H NMR (400.13 MHz, DMSO-d6) δ 0.97 (3H, t), 1.09-1.11 (6H, m), 1.63-1.72 (2H, m), 2.65-2.74 (2H, m), 3.00-3.03 (2H, m), 3.54-3.62 (2H, m), 3.92-3.95 (2H, m), 8.42 (1H, s), 13.02 (1H, s)

m/z (ESI+) (M+H)+=312; HPLC t_{R}=1.03 min.

### Example 25

### 4-(4-Acetylpiperazin-1-yl)-N-[(2r,5 s)-5-hydroxyadamantan-2-yl]-2-propylsulfanylpyrimidine-5-carboxamide

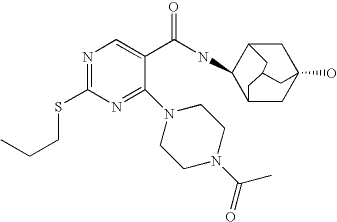

see below Example 26

### Example 26

### 2-(4-Acetylpiperazin-1-yl)-N-[(2r,5 s)-5-hydroxyadamantan-2-yl]-4-propylsulfanylpyrimidine-5-carboxamide

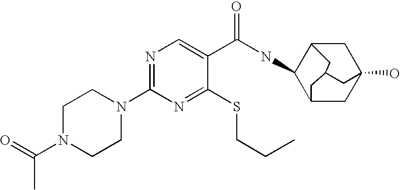

O-(7-Azabenzotriazol-1-yl)-N,N,N′,N′-tetramethyluronium hexafluorophosphate (549 mg, 1.44 mmol) was added in one portion to a mixture of 2-(4-acetylpiperazin-1-yl)-4-(propylthio)pyrimidine-5-carboxylic acid and 4-(4-acetylpiperazin-1-yl)-2-(propylthio)pyrimidine-5-carboxylic acid (Intermediate 50) (390 mg, 0.60 mmol), (1s,4r)-4-aminoadamantan-1-ol hydrochloride (245 mg, 1.20 mmol) and N-ethyldiisopropylamine (0.63 mL, 3.61 mmol) in DMF (10 mL) at 25° C. under nitrogen. The resulting solution was stirred at 25° C. for 3 hours.

The reaction mixture was diluted with EtOAc (150 mL), and washed sequentially with saturated NaHCO3 (50 mL), water (2×50 mL) and saturated brine (50 mL). The organic layer was dried over MgSO4, filtered and evaporated to afford crude product containing two components. The crude product was purified and the products separated by preparative HPLC (Waters XBridge Prep C18 OBD column, 5μ silica, 50 mm diameter, 150 mm length), using decreasingly polar mixtures of water (containing 0.1% NH3) and MeCN as eluents. Fractions containing the desired compounds were evaporated to dryness to afford 2-(4-acetylpiperazin-1-yl)-N-[(2r,5s)-5-hydroxyadamantan-2-yl]-4-propylsulfanylpyrimidine-5-carboxamide (76 mg, 27%) as a white solid and 4-(4-acetylpiperazin-1-yl)-N-[(2r,5s)-5-hydroxyadamantan-2-yl]-2-propylsulfanylpyrimidine-5-carboxamide (45 mg, 16%) as a white solid.

4-(4-acetylpiperazin-1-yl)-N-[(2r,5s)-5-hydroxyadamantan-2-yl]-2-propylsulfanylpyrimidine-5-carboxamide (Example 25):

1H NMR (400.13 MHz, DMSO-d6) δ 0.97 (3H, t), 1.33 (2H, d), 1.61-1.71 (8H, m), 1.85-2.04 (8H, m), 3.02 (2H, t), 3.50-3.58 (8H, m), 3.90 (1H, t), 4.39 (1H, s), 7.99 (1H, s), 8.32 (1H, d)

m/z (ESI+) (M+H)+=474; HPLC t_{R}=1.75 min.

2-(4-acetylpiperazin-1-yl)-N-[(2r,5s)-5-hydroxyadamantan-2-yl]-4-propylsulfanylpyrimidine-5-carboxamide (Example 26):

1H NMR (400.13 MHz, DMSO-d6) δ 0.97 (3H, t), 1.31 (2H, d), 1.60-1.71 (8H, m), 1.92-2.04 (8H, m), 3.01 (2H, t), 3.51-3.53 (4H, m), 3.76-3.78 (2H, m), 3.71-3.78 (3H, m), 4.37 (1H, s), 7.82 (1H, d), 8.31 (1H, s)

m/z (ESI+) (M+H)+=474; HPLC t_{R}=1.79 min

### Intermediate 50

### (a) 2-(4-acetylpiperazin-1-yl)-4-(propylthio)pyrimidine-5-carboxylic acid and (b) 4-(4-acetylpiperazin-1-yl)-2-(propylthio)pyrimidine-5-carboxylic acid

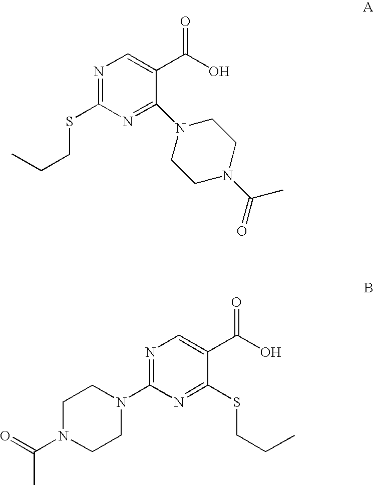

1-Propanethiol (1.73 mL, 19.09 mmol) was added in one portion to ethyl 2,4-dichloropyrimidine-5-carboxylate (4.22 g, 19.09 mmol) and sodium carbonate (6.07 g, 57.27 mmol) in DMF (40 mL) under nitrogen. The resulting suspension was stirred at 20° C. for 18 hours. The reaction mixture was diluted with EtOAc (300 mL), and washed sequentially with water (3×100 mL) and saturated brine (50 mL). The organic layer was dried over MgSO4, filtered and evaporated to afford crude product containing both possible regioisomers together with a quantity of the bis substituted product. The crude product was purified by flash silica chromatography, elution gradient 0 to 20% EtOAc in isohexane. Fractions were evaporated to dryness to afford a clear colourless oil (3.60 g).

1-Acetylpiperazine (418 mg, 3.26 mmol) and potassium carbonate (451 mg, 3.26 mmol) added to 850 mg of the above prepared mixture of chloro pyrimidines in butyronitrile (10 mL). The resulting mixture was stirred at 20° C. for 18 hours. The reaction mixture was diluted with EtOAc (50 mL), and washed sequentially with water (2×25 mL) and saturated brine (25 mL). The organic layer was dried over MgSO4, filtered and evaporated to afford crude product that was purified by flash silica chromatography elution gradient 0 to 10% MeOH in EtOAc. Fractions were evaporated to dryness to afford a mixture of ethyl 2-(4-acetylpiperazin-1-yl)-4-(propylthio)pyrimidine-5-carboxylate and ethyl 4-(4-acetylpiperazin-1-yl)-2-(propylthio)pyrimidine-5-carboxylate (818 mg).

Sodium hydroxide (5.21 mL, 10.43 mmol) was added to the mixture of ethyl 2-(4-acetylpiperazin-1-yl)-4-(propylthio)pyrimidine-5-carboxylate compound with ethyl 4-(4-acetylpiperazin-1-yl)-2-(propylthio)pyrimidine-5-carboxylate (735 mg, 1.04 mmol) in methanol (20 mL). The resulting solution was stirred at 22° C. for 18 hours. The reaction mixture was concentrated and diluted with water (20 mL). The pH was adjusted to pH4 with 2M HCl and the mixture was extracted with EtOAc (2×25 mL). The combined extracts were washed sequentially with water (25 mL) and saturated brine (20 mL). The organic layer was dried over MgSO4, filtered and evaporated to afford an inseperable mixture of 2-(4-acetylpiperazin-1-yl)-4-(propylthio)pyrimidine-5-carboxylic acid and 4-(4-acetylpiperazin-1-yl)-2-(propylthio)pyrimidine-5-carboxylic acid (399 mg).

1H NMR (400.13 MHz, DMSO-d₆) δ 0.96-1.00 (3H, m), 1.61-1.72 (2H, m), 2.02 (1H, s), 2.04 (2H, s), 2.99-3.06 (2H, m), 3.48-3.61 (5H, m), 3.75-3.89 (3H, m), 8.46 (0.33H, s), 8.61 (0.66H, s), 12.56 (1H, s) (inseperable mixture)

m/z (ESI+) (M+H)+=325; t_{R}=0.89 min. (inseperable mixture)

### Example 27

### 2-(4-Acetylpiperazin-1-yl)-N-(2-adamantyl)-4-propylsulfanyl-pyrimidine-5-carboxamide

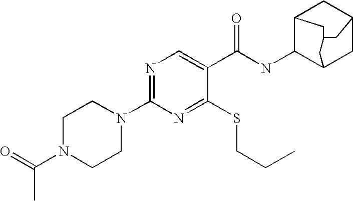

1-Acetylpiperazine (219 mg, 1.71 mmol), and N-adamantan-2-yl-2-chloro-4-(propylthio)pyrimidine-5-carboxamide (Intermediate 52, 250 mg, 0.68 mmol) were suspended in THF (3 mL) and sealed into a microwave tube. The reaction was heated using microwaves to 150° C. for 2 hours and then cooled to room temperature. The reaction mixture was diluted with EtOAc (25 mL) and washed sequentially with water (10 mL) and saturated brine (10 mL). The organic layer was dried over MgSO4, filtered and evaporated to afford crude product. The crude solid was triturated with DMSO/CH₃CN/water (7:2:1) (5 mL) to give a solid which was collected by filtration, washed with CH₃CN/water (2:1) and dried under vacuum to give 2-(4-acetylpiperazin-1-yl)-N-(2-adamantyl)-4-propylsulfanyl-pyrimidine-5-carboxamide (267 mg, 85%) as a white solid.

1H NMR (400.13 MHz, DMSO-d6) δ 0.97 (3H, t), 1.48-1.51 (2H, m), 1.59-1.66 (2H, m), 1.69 (2H, d), 1.75-1.83 (6H, m), 1.90 (2H, s), 2.04 (4H, s), 2.07 (1H, s), 3.02 (2H, t), 3.52 (4H, t), 3.76-3.78 (2H, m), 3.84 (2H, t), 3.93-3.95 (1H, m), 7.86 (1H, d), 8.31 (1H, s)

m/z (ESI+) (M+H)+=458; HPLC t_{R}=2.71 min.

### Intermediate 51

### N-adamantan-2-yl-2,4-dichloropyrimidine-5-carboxamide

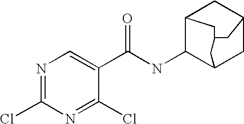

A suspension of 2-adamantanamine hydrochloride (1.776 g, 9.46 mmol) and N-ethyldiisopropylamine (3.27 mL, 18.92 mmol) in THF (10.00 mL) was added dropwise to a stirred solution of 2,4-dichloropyrimidine-5-carbonyl chloride (2.00 g, 9.46 mmol) in dichloromethane (20 mL) at −10° C. under an atmosphere of nitrogen. The resulting solution was stirred at 0° C. for 1 hour. The reaction mixture was diluted with DCM (100 mL) and washed sequentially with 0.1M HCl (25 mL), saturated NaHCO3 (25 mL) and saturated brine (25 mL). The organic layer was dried over MgSO4, filtered and evaporated to afford crude product. The crude solid was triturated with isohexane to give a solid which was collected by filtration and dried under vacuum to give N-adamantan-2-yl-2,4-dichloropyrimidine-5-carboxamide (2.50 g, 81%) as a yellow powder.

1H NMR (400.13 MHz, DMSO-d6) δ 1.53 (2H, d), 1.71 (2H, s), 1.81 (5H, d), 1.85 (1H, s), 1.94-1.96 (3H, m), 2.00 (1H, s), 4.02-4.04 (1H, m), 8.56 (1H, d), 8.84-8.86 (1H, m)

m/z (ESI+) (M+H)+=326; HPLC t_{R}=2.65 min.

### Intermediate 52

### N-adamantan-2-yl-2-chloro-4-(propylthio)pyrimidine-5-carboxamide

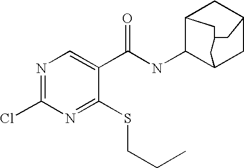

Sodium carbonate (0.812 g, 7.66 mmol) was added in one portion to a mixture of N-adamantan-2-yl-2,4-dichloropyrimidine-5-carboxamide (Intermediate 51, 2.5 g, 7.66 mmol) and 1-Propanethiol (0.694 mL, 7.66 mmol) in DMF (15 mL) at room temperature under nitrogen. The resulting suspension was stirred at room temperature for 16 hours. The reaction mixture was diluted with EtOAc (150 mL) and washed sequentially with water (50 mL) and saturated brine (50 mL). The organic layer was dried over MgSO4, filtered and evaporated to afford crude product. The crude product was purified by flash silica chromatography, elution gradient 10 to 40% EtOAc in isohexane. Pure fractions were evaporated to dryness to afford N-adamantan-2-yl-2-chloro-4-(propylthio)pyrimidine-5-carboxamide (2.60 g, 93%) as a white solid.

1H NMR (400.13 MHz, DMSO-d6) δ 0.97 (3H, t), 1.51 (2H, d), 1.64 (2H, q), 1.69 (1H, s), 1.80-1.84 (7H, m), 1.93 (2H, s), 2.04 (2H, d), 3.11 (2H, t), 4.00 (1H, t), 8.42 (1H, d), 8.47 (1H, s)

m/z (ESI+) (M+H)+=366; HPLC t_{R}=3.19 min.

The following Examples were prepared in a similar manner to Example 27, using Intermediate 51 and an appropriate amine starting material:

| Structure | Ex | Name | ¹H NMR δ | MS m/e MH⁺ |
|---|---|---|---|---|
| 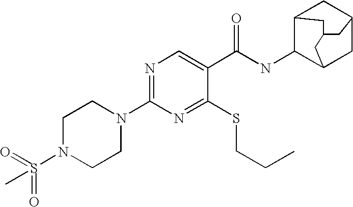 | 28 | N-(2-adamantyl)-2-(4- methylsulfonylpiperazin-1-yl)-4- propylsulfanyl-pyrimidine-5- carboxamide | 1H NMR (400.13 MHz, DMSO-d6) δ 0.97 (3H, t), 1.50 (2H, s), 1.59-1.66 (2H, m), 1.70 (2H, s), 1.75-1.83 (6H, m), 1.90 (2H, s), 2.04-2.07 (2H, m), 2.89 (3H, s), 3.02 (2H, t), 3.19 (4H, t), 3.91-3.95 (5H, m), 7.88 (1H, d), 8.32 (1H, s) | 494; HPLC t_{R} = 3.04 min. |
| 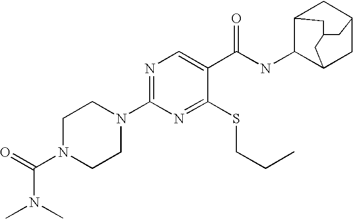 | 29 | N-(2-adamantyl)-2-[4- (dimethylcarbamoyl)piperazin-1- yl]-4-propylsulfanyl-pyrimidine- 5-carboxamide | 1H NMR (400.13 MHz, DMSO-d6) δ 0.96 (3H, t), 1.49 (2H, s), 1.63 (2H, q), 1.69 (2H, d), 1.78 (4H, d), 1.75-1.83 (2H, m), 1.90 (2H, s), 2.04-2.07 (2H, m), 2.78 (6H, s), 3.01 (2H, t), 3.18 (4H, t), 3.80-3.82 (4H, m), 3.94 (1H, t), 7.85 (1H, d), 8.30 (1H, s) | 487; HPLC t_{R} = 2.94 min. |

### Example 30

### 4-Cyclopentyl-N-[(2s,5r)-5-hydroxyadamantan-2-yl]-2-morpholin-4-ylpyrimidine-5-carboxamide

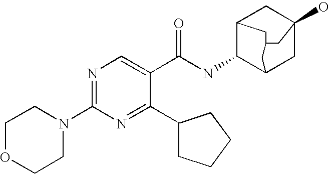

O-(7-Azabenzotriazol-1-yl)-N,N,N′,N′-tetramethyluronium hexafluorophosphate (0.456 g, 1.2 mmol) was added in one portion to 4-cyclopentyl-2-morpholinopyrimidine-5-carboxylic acid (Intermediate 55, 0.277 g, 1.0 mmol) and N-ethyldiisopropylamine (0.523 ml, 3.00 mmol) in DMF (5.00 ml) at 25° C. under nitrogen. After stirring for 10 minutes (1r,4s)-4-aminoadamantan-1-ol (0.224 g, 1.10 mmol) was added and the solution was stirred at 25° C. for 3 hours. The reaction mixture was concentrated and diluted with DCM (100 mL) and washed sequentially with saturated NaHCO3 (100 mL), saturated brine (100 mL) and water (100 mL). The organic layer was dried over MgSO4, filtered and evaporated to afford crude product. The crude product was purified by preparative HPLC (Waters XBridge Prep C18 OBD column, 5μ silica, 50 mm diameter, 150 mm length), using decreasingly polar mixtures of water (containing 0.5% NH3) and MeCN as eluents. Fractions containing the desired compound were evaporated to dryness to afford 4-cyclopentyl-N-[(2s,5r)-5-hydroxyadamantan-2-yl]-2-morpholin-4-ylpyrimidine-5-carboxamide (0.224 g, 52%) as a white solid.

1H NMR (400.13 MHz, DMSO-d6) δ 1.30-1.33 (2H, m), 1.52-1.63 (6H, m), 1.69-1.78 (6H, m), 1.80-1.83 (1H, m), 1.83-1.87 (1H, m), 1.90 (1H, s), 1.93 (1H, s), 1.98 (1H, s), 2.02 (2H, s), 3.41-3.49 (1H, m), 3.65 (4H, q), 3.70-3.74 (4H, m), 3.90 (1H, t), 4.38 (1H, s), 8.04-8.06 (1H, m), 8.22 (1H, t)

m/z (ESI+) (M+H)+=427; HPLC t_{R}=2.01 min.

### Intermediate 53

### Methyl 2-(cyclopentanecarbonyl)-3-(dimethylamino)acrylate

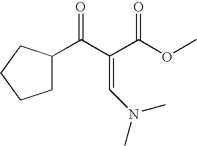

N,N-Dimethylformamide dimethyl acetal (3.28 mL, 24.68 mmol) was added in one portion to methyl 3-cyclopentyl-3-oxopropanoate (3.50 g, 20.56 mmol) in dioxane (40 mL) at room temperature under nitrogen. The resulting solution was stirred at 100° C. for 4 hours. The reaction mixture was evaporated to afford crude product. The crude product was purified by flash silica (120 g) chromatography, elution gradient 50 to 80% EtOAc in isohexane. Pure fractions were evaporated to dryness to afford methyl 2-(cyclopentanecarbonyl)-3-(dimethylamino)acrylate (4.50 g, 97%) as a yellow oil.

1H NMR (400.13 MHz, DMSO-d6) δ 1.45-1.73 (8H, m), 2.81-2.86 (1H, m), 2.95 (6H, s), 3.62 (3H, s), 7.57 (1H, s)

m/z (ESI+) (M+H)+=226; HPLC t_{R}=1.66 min.

### Intermediate 54

### Methyl 4-cyclopentyl-2-morpholinopyrimidine-5-carboxylate

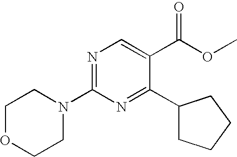

A solution of methyl 2-(cyclopentanecarbonyl)-3-(dimethylamino)acrylate (Intermediate 53, 1.50 g, 6.66 mmol) in methanol (5 mL) was added dropwise to a stirred suspension of morpholinoformamidine hydrobromide (1.399 g, 6.66 mmol) and sodium methoxide (13.32 mL, 6.66 mmol) in methanol (25 mL) under nitrogen. The resulting solution was stirred at 80° C. for 6 hours then at room temperature for 16 hours. The reaction mixture was evaporated to dryness and then dissolved in DCM (50 mL) and washed sequentially with water (2×20 mL), and saturated brine (25 mL). The organic layer was dried over MgSO4, filtered and evaporated to afford crude product. The crude product was purified by flash silica (40 g) chromatography, elution gradient 20 to 50% EtOAc in isohexane. Pure fractions were evaporated to dryness to afford methyl 4-cyclopentyl-2-morpholinopyrimidine-5-carboxylate (1.210 g, 62%) as a colourless oil which solidified on standing.

1H NMR (400.13 MHz, DMSO-d6) δ 1.58-1.66 (2H, m), 1.70-1.81 (4H, m), 1.86-1.93 (2H, m), 3.64-3.67 (4H, m), 3.77 (3H, s), 3.79-3.81 (4H, m), 3.96 (1H, q), 8.72 (1H, s)

m/z (ESI+) (M+H)+=292; HPLC t_{R}=2.78 min.

### Intermediate 55

### 4-cyclopentyl-2-morpholinopyrimidine-5-carboxylic acid

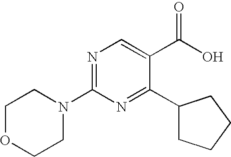

Sodium hydroxide (10.38 mL, 20.77 mmol) was added in one portion to methyl 4-cyclopentyl-2-morpholinopyrimidine-5-carboxylate (Intermediate 54, 1.21 g, 4.15 mmol) in methanol (50 mL) under air. The resulting solution was stirred at 60° C. for 4 hours then at room temperature for 16 hours. The reaction mixture was concentrated and diluted with water (15 mL) and acidified with 2M HCl. The precipitate was collected by filtration, washed with water (25 mL) and dried under vacuum to afford 4-cyclopentyl-2-morpholinopyrimidine-5-carboxylic acid (1.10 g, 96%) as a white solid that was used without further purification.

1H NMR (400.13 MHz, DMSO-d6) δ 1.55-1.65 (2H, m), 1.70-1.80 (4H, m), 1.84-1.93 (2H, m), 3.64-3.66 (4H, m), 3.78-3.80 (4H, m), 4.03-4.11 (1H, m), 8.72 (1H, s), 12.62 (1H, s)

m/z (ESI+) (M+H)+=278; HPLC t_{R}=2.31 min.

### Example 31

### N-[(2s,5r)-5-Hydroxyadamantan-2-yl]-2-morpholin-4-yl-4-propoxypyrimidine-5-carboxamide

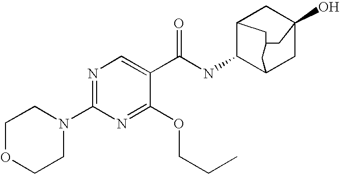

Morpholine (1.047 mL, 12.00 mmol) and 2-chloro-N-[(2s,5r)-5-hydroxyadamantan-2-yl]-4-propoxypyrimidine-5-carboxamide (Intermediate 57, 366 mg, 1.00 mmol) were suspended in THF (5 mL) and sealed into a microwave tube. The reaction was heated using microwave heating to 100° C. for 30 minutes and then cooled to room temperature. The reaction mixture was diluted with DCM (50 mL) and washed sequentially with water (25 mL) and saturated brine (25 mL). The organic layer was dried over MgSO4, filtered and evaporated to afford crude product. The crude product was purified by preparative HPLC (Waters XBridge Prep C18 OBD column, 5μ silica, 50 mm diameter, 150 mm length), using decreasingly polar mixtures of water (containing 0.5% NH3) and MeCN as eluents. Fractions containing the desired compound were evaporated to dryness to afford N-[(2s,5r)-5-hydroxyadamantan-2-yl]-2-morpholin-4-yl-4-propoxypyrimidine-5-carboxamide (140 mg, 34%)

1H NMR (400.13 MHz, DMSO-d6) δ 0.98 (3H, t), 1.43-1.46 (2H, m), 1.63-1.65 (4H, m), 1.70-1.75 (4H, m), 1.77-1.82 (2H, m), 2.02 (3H, s), 3.63-3.66 (4H, m), 3.75-3.78 (4H, m), 3.97 (1H, t), 4.40 (2H, t), 4.42 (1H, s), 7.63 (1H, d), 8.65 (1H, s)

m/z (ESI+) (M+H)+=417; HPLC t_{R}=1.97 min.

### Intermediate 56

### 2,4-dichloro-N-[(2s,5r)-5-hydroxyadamantan-2-yl]pyrimidine-5-carboxamide

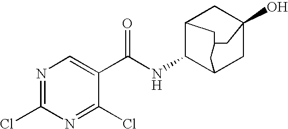

A suspension of (1r,4s)-4-aminoadamantan-1-ol.hydrochloride (2.89 g, 14.19 mmol) in THF (20.00 mL) was added dropwise to a stirred solution of 2,4-dichloropyrimidine-5-carbonyl chloride (3.00 g, 14.19 mmol) and N-ethyldiisopropylamine (4.91 mL, 28.38 mmol) in dichloromethane (20 mL) at −10° C., over a period of 5 minutes under nitrogen. The resulting suspension was stirred at 0° C. for 4 hours. The reaction mixture was diluted with DCM (150 mL) and washed sequentially with 0.1M HCl (50 mL), water (50 mL) and saturated brine (75 mL). The organic layer was dried over MgSO4, filtered and evaporated to afford desired product. The crude solid was triturated with ice-cold DCM to give a solid which was collected by filtration and dried under vacuum to give 2,4-dichloro-N-[(2s,5r)-5-hydroxyadamantan-2-yl]pyrimidine-5-carboxamide (3.20 g, 66%) as a tan solid.

1H NMR (400.13 MHz, DMSO-d6) δ 1.36 (2H, d), 1.63 (4H, d), 1.71-1.77 (3H, m), 1.86 (2H, d), 1.98-2.00 (1H, m), 2.06 (2H, s), 3.95 (1H, t), 8.51 (1H, d), 8.83-8.85 (1H, m)

m/z (ESI+) (M+H)+=342; HPLC t_{R}=1.44 min.

### Intermediate 57

### 2-chloro-N-[(2s,5r)-5-hydroxyadamantan-2-yl]-4-propoxypyrimidine-5-carboxamide

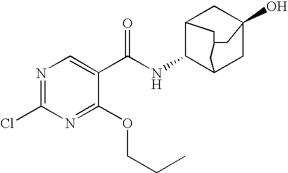

Sodium bis(trimethylsilyl)amide (1M solution in THF, 1.00 mL, 1.00 mmol) was added in one portion to 1-propanol (0.075 mL, 1.00 mmol), in THF (1 mL) at room temperature under nitrogen. The resulting suspension was stirred at room temperature for 5 minutes. This suspension was added dropwise to 2,4-dichloro-N-[(2s,5r)-5-hydroxyadamantan-2-yl]pyrimidine-5-carboxamide (Intermediate 56, 0.342 g, 1 mmol) in THF (10 mL) at room temperature under nitrogen. The resulting suspension was stirred for a further 4 hours. The reaction mixture was diluted with EtOAc (75 mL) and washed sequentially with 0.1M HCl (25 mL), water (25 mL) and saturated brine (25 mL). The organic layer was dried over MgSO4, filtered and evaporated to afford crude product as yellow foam. Used directly in next stage.

m/z (EI+) (M+H)+=366; HPLC t_{R}=2.03 min.

The following Examples were prepared in a similar manner to Example 31, using Intermediate 57 and an appropriate amine starting material:

| Structure | Ex | Name | ¹H NMR δ | MS m/e MH⁺ |
|---|---|---|---|---|
| 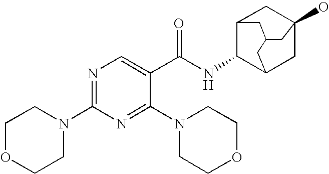 | 32 | N-[(2s,5r)-5- hydroxyadamantan- 2-yl]-2,4- dimorpholin-4- ylpyrimidine-5- carboxamide | 1H NMR (400.13 MHz, DMSO-d6) δ 1.30-1.33 (2H, m), 1.59-1.62 (4H, m), 1.67-1.70 (2H, m), 1.90 (2H, d), 1.99 (3H, s), 3.43 (4H, t), 3.58-3.62 8H, m), 3.64-3.66 (4H, m), 3.85-3.87 (1H, m), 4.37 (1H, s), 7.95 (1H, s), 8.04 (1H, d) | 444; HPLC t_{R} = 1.40 min. |

### Example 33

### 4-Cyclopropyl-N-[(2r,5s)-5-hydroxyadamantan-2-yl]-2-methoxypyrimidine-5-carboxamide

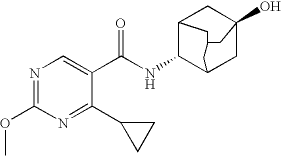

[Dimethylamino-(triazolo[5,4-b]pyridin-3-yloxy)methylidene]-dimethylazanium hexafluorophosphate (479 mg, 1.26 mmol) was added to 4-cyclopropyl-2-methoxypyrimidine-5-carboxylic acid (Intermediate 59, 155 mg, 0.80 mmol) and N-ethyl-N-propan-2-ylpropan-2-amine (0.274 mL, 1.60 mmol) in DMF (5 mL). The resulting solution was stirred at room temperature for 15 minutes. (1s,4r)-4-Aminoadamantan-1-ol hydrochloride (179 mg, 0.88 mmol) was added and the reaction mixture was stirred at room temperature for 2 hours. The reaction mixture was evaporated to dryness and redissolved in EtOAc (150 mL) and washed sequentially with water (2×150 mL). The organic layer was dried over MgSO4, filtered and evaporated to afford the crude product.

The crude product was purified by flash silica chromatography, elution gradient 0 to 10% MeOH in DCM. Pure fractions were evaporated to dryness to afford 4-cyclopropyl-N-[(2r,5s)-5-hydroxyadamantan-2-yl]-2-methoxypyrimidine-5-carboxamide (67 mg, 24%) as a white solid.

1H NMR (400.13 MHz, CDCl₃) δ 1.02-1.06 (2H, m), 1.22-1.25 (2H, m), 1.50 (2H, d), 1.67 (1H, s), 1.72 (3H, d), 1.75 (1H, s), 1.87 (2H, d), 1.97 (1H, s), 2.11 (1H, s), 2.19 (2H, s), 2.37-2.43 (1H, m), 3.89 (3H, s), 4.13-4.17 (1H, m), 6.26 (1H, d), 8.37 (1H, s)

m/z (ESI+) (M+H)+=344; HPLC t_{R}=1.58 min.

### Intermediate 58

### Ethyl 4-cyclopropyl-2-methoxypyrimidine-5-carboxylate

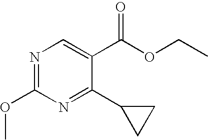

Ethyl 2-(cyclopropanecarbonyl)-3-(dimethylamino)acrylate (499 mg, 2.36 mmol) was dissolved in DMF (10 mL). To this solution was added methyl carbamimidate hydrochloride (279 mg, 2.52 mmol) and sodium acetate (915 mg, 11.16 mmol). The reaction was heated at 85° C. for 8 hours and then allowed to cool to room temperature and water (50 mL) was added. The reaction mixture was diluted with EtOAc (100 mL) and washed sequentially with water (2×100 mL), saturated aqueous NaHCO3 (100 mL) and water (100 mL). The organic layer was dried over MgSO4, filtered and evaporated to afford the crude product. The crude product was purified by flash silica chromatography, elution gradient 0 to 10% EtOAc in isohexane. Pure fractions were evaporated to dryness to afford ethyl 4-cyclopropyl-2-methoxypyrimidine-5-carboxylate (178 mg, 34%) as a colourless oil.

1H NMR (400.13 MHz, CDCl₃) δ 1.00-1.07 (2H, m), 1.17-1.24 (2H, m), 1.32 (3H, t), 3.12-3.19 (1H, m), 3.90 (3H, s), 4.30 (2H, q), 8.83 (1H, s)

m/z (ESI+) (M+H)+=223; HPLC t_{R}=2.32 min.

### Intermediate 59

### 4-cyclopropyl-2-methoxypyrimidine-5-carboxylic acid

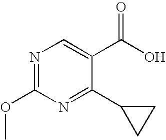

Ethyl 4-cyclopropyl-2-methoxypyrimidine-5-carboxylate (Intermediate 58, 178 mg, 0.80 mmol) was dissolved in methanol (5 mL) and 2M aqueous sodium hydroxide (2.0 mL, 4.0 mmol) was added. The resulting solution was stirred at room temperature for 3 hours. The reaction mixture was evaporated to dryness and then dissolved in water (50 mL) and then acidified to pH=4 with 2N HCl. The aqueous layer was washed sequentially with EtOAc (2×100 mL). The organic layer was dried over MgSO4, filtered and evaporated to afford crude 4-cyclopropyl-2-methoxypyrimidine-5-carboxylic acid (107 mg, 69%) as a white solid, which was used without further purification.

m/z (ESI+) (M+H)+=195; HPLC t_{R}=1.56 min.

### Example 34

### 4-Cyclopropyl-N-[(2r,5s)-5-hydroxyadamantan-2-yl]-2-methylaminopyrimidine-5-carboxamide

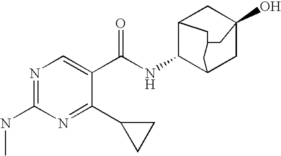

4-Cyclopropyl-N-[(2r,5s)-5-hydroxyadamantan-2-yl]-2-methylsulfinylpyrimidine-5-carboxamide (Intermediate 60, 347 mg, 0.92 mmol) and 2M methylamine in THF (2.31 mL, 4.62 mmol) were dissolved in THF (2 mL) and sealed into a microwave tube. The reaction was heated to 110° C. for 30 min in the microwave reactor and then cooled to room temperature. The reaction mixture was evaporated to dryness and redissolved in EtOAc (75 mL) and washed sequentially with saturated brine (2×50 mL). The organic layer was dried over MgSO4, filtered and evaporated to afford crude product. The crude product was purified by flash silica chromatography, elution gradient 0 to 10% MeOH in DCM. Pure fractions were evaporated to dryness to afford 4-cyclopropyl-N-[(2r,5s)-5-hydroxyadamantan-2-yl]-2-methylaminopyrimidine-5-carboxamide (137 mg, 43%) as a white solid.

1H NMR (400.13 MHz, CDCl₃) δ 0.91-0.96 (2H, m), 1.14-1.20 (2H, m), 1.49 (2H, d), 1.66 (2H, d), 1.71 (3H, s), 1.74 (1H, s), 1.86 (2H, d), 2.10 (1H, s), 2.17 (2H, s), 2.38-2.44 (1H, m), 2.89 (3H, d), 4.10-4.15 (1H, m), 5.31 (1H, s), 6.08 (1H, d), 8.24 (1H, s)

m/z (ESI+) (M+H)+=343; HPLC t_{R}=1.60 min.

### Intermediate 60

### 4-Cyclopropyl-N-[(2r,5s)-5-hydroxyadamantan-2-yl]-2-methylsulfinylpyrimidine-5-carboxamide

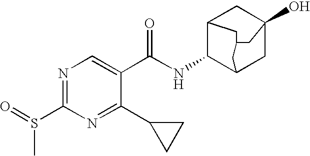

3-Chloroperoxybenzoic acid (88 mg, 0.36 mmol) was added as a solid to a cold (0° C.) solution of 4-cyclopropyl-N-[(2r,5 s)-5-hydroxyadamantan-2-yl]-2-methylsulfanylpyrimidine-5-carboxamide (Example 11, 107 mg, 0.30 mmol) in DCM (10 mL) under an atmosphere of nitrogen. After 30 minutes the reaction had gone to completion and saturated aqueous NaHCO3 (50 mL) was added to quench the reaction. The organic layer was separated and the aqueous layer was washed sequentially with EtOAc (5×150 mL). The combined organic layers were dried over MgSO4, filtered and evaporated to afford crude product as a colourless oil. The product was used in the next reaction step without further purification and characterisation.

m/z (ESI+) (M+H)+=376; HPLC t_{R}=1.25 min.

### Intermediate 80

### 4-cyclopropyl-N-[(2r,5s)-5-hydroxyadamantan-2-yl]-2-methylsulfonylpyrimidine-5-carboxamide

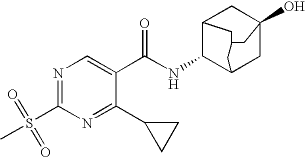

3-Chloroperoxybenzoic acid (70%) (19.20 g, 77.89 mmol) was added in one portion to 4-cyclopropyl-N-[(2r,5 s)-5-hydroxyadamantan-2-yl]-2-methylsulfanylpyrimidine-5-carboxamide (Example 11, 14 g, 38.94 mmol) in DCM (450 mL) at 0° C. The resulting solution was stirred at 20° C. for 24 hours. The reaction mixture was diluted with DCM (300 mL), and washed sequentially with saturated NaHCO3 (4×200 mL) and saturated brine (200 mL). The organic layer was dried over MgSO4, filtered and evaporated to afford 4-cyclopropyl-N-[(2r,5s)-5-hydroxyadamantan-2-yl]-2-methylsulfonylpyrimidine-5-carboxamide (12.10 g, 79%) as a white solid.

1H NMR (400.132 MHz, CDCl3) δ 1.28-1.31 (2H, m), 1.39-1.42 (2H, m), 1.56-1.62 (2H, m), 1.73-1.85 (7H, m), 1.94-1.97 (2H, m), 2.19-2.23 (1H, m), 2.28-2.32 (2H, m), 2.45-2.52 (1H, m), 3.27 (3H, s), 4.25-4.31 (1H, m), 6.37 (1H, d), 8.70 (1H, s)

m/z (ESI+) (M+H)+=392; HPLC t_{R}=1.41 min.

### Example 35

### 4-Cyclopropyl-N-[(2r,5s)-5-hydroxyadamantan-2-yl]-2-thiomorpholin-4-ylpyrimidine-5-carboxamide

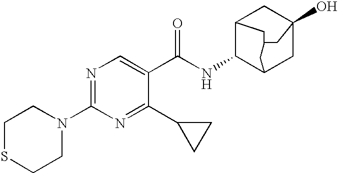

4-cyclopropyl-N-[(2r,5s)-5-hydroxyadamantan-2-yl]-2-methylsulfonylpyrimidine-5-carboxamide (Intermediate 60, 693 mg, 1.77 mmol) and thiomorpholine (2.00 mL, 21.09 mmol) were dissolved in THF (4 mL) and sealed into a microwave tube. The reaction was heated to 150° C. for 10 hours in the microwave reactor and then cooled to room temperature. The reaction mixture was evaporated to dryness and redissolved in EtOAc (150 mL) and washed sequentially with saturated brine (2×75 mL). The organic layer was dried over MgSO4, filtered and evaporated to afford crude product. The crude product was purified by flash silica chromatography, elution gradient 0 to 7% MeOH in DCM. Pure fractions were evaporated to dryness to afford 4-cyclopropyl-N-[(2r,5s)-5-hydroxyadamantan-2-yl]-2-thiomorpholin-4-ylpyrimidine-5-carboxamide (444 mg, 60%) as a colourless oil which solidified on standing.

1H NMR (400.13 MHz, DMSO-d₆) δ 0.93-0.96 (2H, m), 0.98-1.03 (2H, m), 1.32 (2H, d), 1.60-1.63 (4H, m), 1.69-1.72 (2H, m), 1.94 (2H, d), 1.99-1.99 (1H, m), 2.04 (2H, s), 2.20 (1H, s), 2.55-2.57 (4H, m), 3.16 (1H, d), 3.90-3.94 (1H, m), 4.01-4.04 (4H, m), 8.07 (1H, d), 8.23 (1H, s)

m/z (ESI+) (M+H)+=415; HPLC t_{R}=2.18 min.

### Example 36

### 4-Cyclopropyl-N-[(2r,5s)-5-hydroxyadamantan-2-yl]-2-(1-oxo-1,4-thiazinan-4-yl)pyrimidine-5-carboxamide

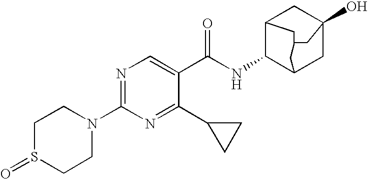

3-Chloroperoxybenzoic acid (153.1 mg, 0.62 mmol) was added as a solid to a cold (0° C.) solution of 4-cyclopropyl-N-[(2r,5 s)-5-hydroxyadamantan-2-yl]-2-thiomorpholin-4-ylpyrimidine-5-carboxamide (Example 35, 223.2 mg, 0.54 mmol) in dichloromethane (10 mL) and stirred for 15 minutes. Saturated aqueous NaHCO3 (50 mL) was added to quench the reaction and the organic layer was separated. The aqueous layer was washed with EtOAc (3×100 mL) and the combined organic layers were dried over MgSO4, filtered and evaporated to afford crude product. The crude product was purified by flash silica chromatography, elution gradient 0 to 20% MeOH in DCM. Pure fractions were evaporated to dryness to afford 4-cyclopropyl-N-[(2r,5s)-5-hydroxyadamantan-2-yl]-2-(1-oxo-1,4-thiazinan-4-yl)pyrimidine-5-carboxamide (74 mg, 32%) as a white solid.

1H NMR (400.13 MHz, CDCl3) δ 0.97-1.02 (2H, m), 1.11-1.15 (2H, m), 1.50 (2H, d), 1.67 (2H, d), 1.72 (3H, s), 1.75 (1H, s), 1.86-1.88 (2H, m), 2.10 (1H, s), 2.17 (2H, s), 2.41-2.47 (1H, m), 2.61-2.68 (2H, m), 2.72-2.77 (2H, m), 4.04-4.11 (2H, m), 4.11-4.16 (1H, m), 4.43-4.49 (2H, m), 6.13 (1H, d), 8.30 (1H, s)

m/z (ESI+) (M+H)+=431; HPLC t_{R}=1.39 min.

### Example 37

### 4-Cyclopropyl-2-(1,1-dioxo-1,4-thiazinan-4-yl)-N-[(2r,5s)-5-hydroxyadamantan-2-yl]pyrimidine-5-carboxamide

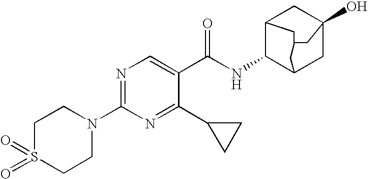

3-Chloroperoxybenzoic acid (606 mg, 2.46 mmol) was added as a solid to a cold (0° C.) solution of 4-cyclopropyl-N-[(2r,5s)-5-hydroxyadamantan-2-yl]-2-thiomorpholin-4-ylpyrimidine-5-carboxamide (Example 35, 679 mg, 1.64 mmol) in dichloromethane (20 mL) and stirred for 20 minutes. Saturated aqueous NaHCO3 (150 mL) was then added to quench the reaction. The organic layer was separated. The aqueous layer was washed with EtOAc (3×100 mL) and the combined organic layers were dried over MgSO4, filtered and evaporated to afford crude product. The crude product was purified by preparative HPLC (Phenomenex Gemini C18 110A (axia) column, 5μ silica, 30 mm diameter, 100 mm length), using decreasingly polar mixtures of water (containing 0.1% AcOH) and MeCN as eluents. Fractions containing the desired compound were evaporated to dryness to afford 4-cyclopropyl-2-(1,1-dioxo-1,4-thiazinan-4-yl)-N-[(2r,5 s)-5-hydroxyadamantan-2-yl]pyrimidine-5-carboxamide (120 mg, 16%) as a white solid.

1H NMR (400.13 MHz, CDCl₃) δ 1.00-1.04 (2H, m), 1.09-1.12 (2H, m), 1.51 (2H, d), 1.66 (2H, d), 1.72 (3H, s), 1.75 (1H, s), 1.86-1.89 (2H, m), 2.11 (1H, s), 2.18 (2H, s), 2.42-2.46 (1H, m), 2.94 (4H, t), 4.13-4.17 (1H, m), 4.27 (4H, t), 6.05 (1H, d), 8.31 (1H, s)

m/z (ESI+) (M+H)+=447; HPLC t_{R}=1.70 min.

### Example 38

### 4-Cyclohexyl-N-[(2r,5s)-5-hydroxyadamantan-2-yl]-2-morpholin-4-ylpyrimidine-5-carboxamide

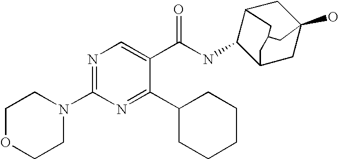

N-Ethyldiisopropylamine (0.285 mL, 1.65 mmol) was added in one portion to 4-aminoadamantan-1-ol hydrochloride (0.308 g, 1.51 mmol), 4-cyclohexyl-2-morpholinopyrimidine-5-carboxylic acid (Intermediate 63, 0.4 g, 1.37 mmol) and O-(7-Azabenzotriazol-1-yl)-N,N,N′,N′-tetramethyluronium hexafluorophosphate (0.626 g, 1.65 mmol) in DMF (8 mL) at 18° C. under nitrogen. The resulting suspension was stirred at 18° C. for 70 hours. The reaction was incomplete and further (1s,4r)-4-aminoadamantan-1-ol hydrochloride (0.308 g, 1.51 mmol) and N-ethyldiisopropylamine (0.57 mL, 3.30 mmol) was added in one portion and the suspension was stirred at 18° C. for a further 4 hours. The reaction mixture was diluted with EtOAc (75 mL), and washed sequentially with water (25 mL) and saturated brine (25 mL). The organic layer was dried over MgSO4, filtered and evaporated to afford crude product. The crude product was purified by flash silica chromatography (40 g column), elution gradient 0 to 100% EtOAc:MeOH (9:1) in DCM. Pure fractions were evaporated to dryness to afford 4-cyclohexyl-N-[(2r,5s)-5-hydroxyadamantan-2-yl]-2-morpholin-4-ylpyrimidine-5-carboxamide (0.402 g, 66%) as a white solid.

1H NMR (400.13 MHz, DMSO-d6) δ 1.15-1.34 (5H, m), 1.45-1.57 (2H, m), 1.60-1.75 (11H, m), 1.90-2.03 (5H, m), 2.97-3.03 (1H, m), 3.61-3.67 (4H, m), 3.69-3.76 (4H, m), 3.88-3.93 (1H, m), 4.38 (1H, s), 8.06 (1H, d), 8.22 (1H, s)

m/z (ESI+) (M+H)+=441; HPLC t_{R}=2.12 min.

### Intermediate 61

### Methyl 2-(cyclohexanecarbonyl)-3-(dimethylamino)acrylate

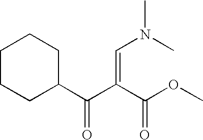

N,N-Dimethylformamide dimethyl acetal (3.47 mL, 26.05 mmol) was added in one portion to methyl 3-cyclohexyl-3-oxopropanoate (4.0 g, 21.71 mmol) in dioxane (40 mL) under nitrogen. The resulting solution was stirred at 105° C. for 6 hours. The reaction mixture was evaporated to give the product as a greenish-yellow oil. The crude product was purified by flash silica (120 g) chromatography, elution gradient 60 to 100% EtOAc in isohexane. Pure fractions were evaporated to dryness to afford methyl 2-(cyclohexanecarbonyl)-3-(dimethylamino)acrylate (4.99 g, 96%) as a yellow oil.

1H NMR (400.13 MHz, DMSO-d6) δ 1.07-1.27 (5H, m), 1.59-1.68 (5H, m), 2.78-2.98 (7H, m), 3.62 (3H, s), 7.57 (1H, s)

m/z (ESI+) (M+H)+=240; HPLC t_{R}=1.83 min.

### Intermediate 62

### Methyl 4-cyclohexyl-2-morpholinopyrimidine-5-carboxylate

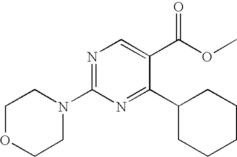

A solution of methyl 2-(cyclohexanecarbonyl)-3-(dimethylamino)acrylate (Intermediate 61, 1.61 g, 6.73 mmol) in MeOH (5 mL) was added dropwise to a stirred solution of morpholinoformamidine hydrobromide (1.413 g, 6.73 mmol) and 0.5M Sodium Methoxide (13.46 mL, 6.73 mmol) at 18° C., over a period of 3 minutes under nitrogen. The resulting solution was stirred at 80° C. for 6 hours then room temperature for 12 hours. The reaction mixture was quenched with saturated NH4Cl aq. (10 mL) then diluted with DCM (50 mL) and washed with water (20 mL). The organic layer was dried over MgSO4, filtered and evaporated to afford crude product. The crude product was purified by flash silica chromatography, elution gradient 0 to 10% EtOAc in DCM. Pure fractions were evaporated to dryness to afford methyl 4-cyclohexyl-2-morpholinopyrimidine-5-carboxylate (1.610 g, 78%) as a white solid.

1H NMR (400.13 MHz, DMSO-d6) δ 1.18-1.38 (3H, m), 1.45-1.54 (2H, m), 1.67-1.78 (5H, m), 3.49-3.57 (1H, m), 3.63-3.67 (4H, m), 3.77-3.82 (7H, m), 8.73 (1H, s)

m/z (ESI+) (M+H)+=306; HPLC t_{R}=2.98 min.

### Intermediate 63

### 4-Cyclohexyl-2-morpholinopyrimidine-5-carboxylic acid

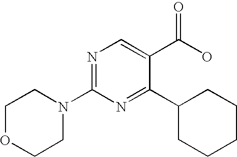

A 2M solution of sodium hydroxide in water (12.93 mL, 25.87 mmol) was added dropwise to a stirred suspension of methyl 4-cyclohexyl-2-morpholinopyrimidine-5-carboxylate (Intermediate 62, 1.58 g, 5.17 mmol) in MeOH (60 mL) at 18° C., over a period of 5 minutes. The resulting suspension was stirred at 18° C. for 18 hours. The reaction was incomplete so the temperature was increased to 60° C. and the reaction mixture was stirred for a further 4 hours to give a clear colourless solution. The reaction mixture was acidified with 2M HCl to pH 4.5 and the white precipitate was filtered off and washed with water (3×20 mL). The combined aqueous washings and the mother liquors were extracted with DCM (3×20 mL) and the organic solution was combined with the original solid (DCM was used for this although the solid was only sparingly soluble in DCM). Evaporation gave a white solid that was azeotroped with toluene (30 mL) to afford 4-cyclohexyl-2-morpholinopyrimidine-5-carboxylic acid (1.430 g, 95%) as a white solid.

1H NMR (400.13 MHz, DMSO-d6) δ 1.15-1.37 (3H, m), 1.45-1.54 (2H, m), 1.67-1.78 (5H, m), 3.59-3.67 (5H, m), 3.78-3.81 (4H, m), 8.72 (1H, s), 12.60 (1H, s)

m/z (ESI+) (M+H)+=292; HPLC t_{R}=2.30 min.

### Example 39

### 4-Cyclopentyl-N-[(2r,5s)-5-hydroxyadamantan-2-yl]-2-methylpyrimidine-5-carboxamide

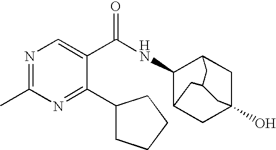

Prepared from Intermediate 65 by the Same Process Used for Example 31

1H NMR (400.132 MHz, CDCl3) δ 1.56-1.72 (9H, m), 1.78-2.01 (10H, m), 2.18 (1H, s), 2.26 (2H, s), 2.70 (3H, s), 3.41-3.46 (1H, m), 4.20-4.25 (1H, m), 5.94 (1H, d), 8.52 (1H, S)

m/z (ESI+) (M+H)+=356; HPLC t_{R}=1.70 min.

### Intermediate 64

### Methyl 4-cyclopentyl-2-methylpyrimidine-5-carboxylate

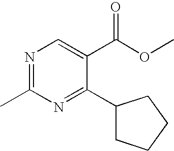

Prepared from Intermediate 53 by the Same Process Used for Intermediate 54

1H NMR (400.132 MHz, CDCl3) δ 1.64-1.73 (2H, m), 1.83-1.92 (4H, m), 1.97-2.04 (2H, m), 2.72 (3H, s), 3.93 (3H, s), 3.91-3.97 (1H, m), 8.94 (1H, s)

m/z (ESI+) (M+H)+=221; HPLC t_{R}=2.31 min.

### Intermediate 65

### 4-Cyclopentyl-2-methylpyrimidine-5-carboxylic acid

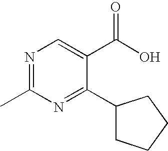

Prepared from Intermediate 64 by the same process used for Intermediate 2.

1H NMR (400.132 MHz, CDCl3) δ 1.67-1.76 (2H, m), 1.84-1.96 (4H, m), 2.01-2.08 (2H, m), 2.79 (3H, s), 4.05-4.16 (1H, m), 8.35 (1H, bs), 9.16 (1H, s)

m/z (ESI+) (M+H)+=207; HPLC t_{R}=1.63 min.

### Example 40

### 4-Cyclobutyl-N-[(2r,5s)-5-hydroxyadamantan-2-yl]-2-morpholin-4-ylpyrimidine-5-carboxamide

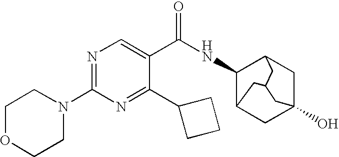

Prepared from Intermediate 68 by the Same Process Used for Example 38

1H NMR (400.132 MHz, CDCl3) δ 1.43 (1H, s), 1.54-1.56 (2H, m), 1.69 (2H, d), 1.76-1.82 (4H, m), 1.86-2.07 (4H, m), 2.13-2.18 (1H, m), 2.21-2.28 (4H, m), 2.35-2.46 (2H, m), 3.77 (4H, t), 3.91 (4H, t), 3.94-4.03 (1H, m), 4.14-4.19 (1H, m), 5.81 (1H, d), 8.33 (1H, s)

m/z (ESI+) (M+H)+=413; HPLC t_{R}=1.83 min.

### Intermediate 66

### Methyl 2-(cyclobutanecarbonyl)-3-(dimethylamino)acrylate

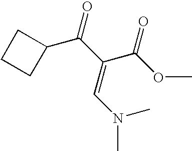

N,N-Dimethylformamide dimethyl acetal (5.62 mL, 42.26 mmol) was added in one portion to methyl 3-cyclobutyl-3-oxopropanoate (5.5 g, 35.22 mmol) in dioxane (50 mL) at room temperature under nitrogen. The resulting solution was stirred at 100° C. for 4 hours. The reaction mixture was evaporated, to afford crude product. The crude product was purified by flash silica (120 g) chromatography, elution gradient 50 to 80% EtOAc in isohexane. Pure fractions were evaporated to dryness to afford (Z)-methyl 2-(cyclobutanecarbonyl)-3-(dimethylamino)acrylate (4.60 g, 61.8%) as a yellow oil.

1H NMR (400.132 MHz, CDCl3) δ 1.72-1.82 (1H, m), 1.85-1.97 (1H, m), 2.06-2.13 (2H, m), 2.18-2.29 (2H, m), 3.02 (6H, s), 3.68-3.75 (1H, m), 3.73 (3H, s), 7.62 (1H, s)

m/z (ESI+) (M+Na)+=234; HPLC t_{R}=1.42 min.

### Intermediate 67

### Methyl 4-cyclobutyl-2-morpholinopyrimidine-5-carboxylate

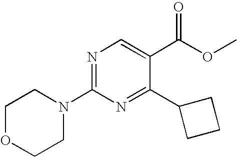

Prepared from Intermediate 66 by the Same Process Used for Intermediate 2

1H NMR (400.132 MHz, CDCl3) δ 1.79-1.90 (1H, m), 1.97-2.08 (1H, m), 2.23-2.32 (2H, m), 2.34-2.42 (2H, m), 3.76-3.79 (4H, m), 3.83 (3H, s), 3.94-3.99 (4H, m), 4.31 (1H, quintet), 8.78 (1H, s)

m/z (ESI+) (M+H)+=278; HPLC t_{R}=2.57 min.

### Intermediate 68

### 4-Cyclobutyl-2-morpholinopyrimidine-5-carboxylic acid

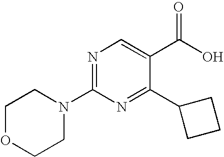

Prepared from Intermediate 67 by the same process used for Intermediate 3.

1H NMR (400.132 MHz, DMSO) δ 1.73-1.81 (1H, m), 1.91-2.01 (1H, m), 2.14-2.22 (2H, m), 2.25-2.36 (2H, m), 3.67 (4H, t), 3.82-3.88 (4H, m), 4.30 (1H, quintet), 8.70 (1H, s), 12.38 (1H, s)

m/z (ESI+) (M+H)+=264; HPLC t_{R}=0.91 min.

### Example 41

### 4-Cyclobutyl-N-[(2r,5s)-5-hydroxyadamantan-2-yl]-2-thiomorpholin-4-ylpyrimidine-5-carboxamide

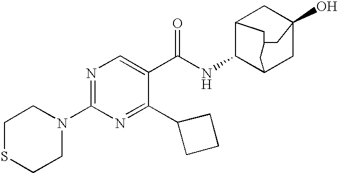

Prepared from Intermediate 72 by the same process used for Example 33. ¹H NMR (400.132 MHz, CDCl3) δ 1.37 (1H, s), 1.54-1.59 (2H, m), 1.67-1.73 (2H, m), 1.77-1.82 (4H, m), 1.87-2.09 (4H, m), 2.16-2.19 (1H, m), 2.22-2.28 (4H, m), 2.34-2.44 (2H, m), 2.64-2.70 (4H, m), 3.98 (1H, quintet), 4.14-4.19 (1H, m), 4.23-4.26 (4H, m), 5.81 (1H, d), 8.33 (1H, s)

m/z (ESI+) (M+H)+=429; HPLC t_{R}=2.27 min.

### Example 42

### 4-Cyclopropyl-2-(2,6-dimethylmorpholin-4-yl)-N-[(2r,5s)-5-hydroxyadamantan-2-yl]pyrimidine-5-carboxamide (as approximately 90% 2S,6R diastereoisomer)

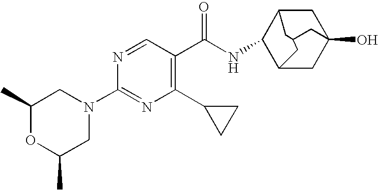

Prepared from Intermediate 74 by the same process used for Example 1.

1H NMR (400.132 MHz, CDCl3) δ 0.92-0.97 (2H, m), 1.11-1.16 (2H, m), 1.18 (6H, s), 1.32 (1H, s), 1.50 (2H, d), 1.59-1.77 (6H, m), 1.87 (2H, d), 2.11 (1H, s), 2.17 (2H, s), 2.40-2.46 (1H, m), 2.49 (2H, d), 3.47-3.56 (2H, m), 4.14 (1H, d), 4.47 (2H, d), 5.96 (1H, d), 8.29 (1H, s)

m/z (ESI+) (M+H)+=427; HPLC t_{R}=1.97 min.

### Intermediate 73

### Methyl 4-cyclopropyl-2-(2,6-dimethylmorpholino)pyrimidine-5-carboxylate (as approximately 90% 2S,6R diastereoisomer)

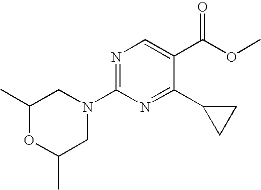

Prepared from methyl 2-(cyclopropanecarbonyl)-3-(dimethylamino)acrylate by the same process used for Intermediate 4.

1H NMR (400.132 MHz, CDCl3) δ 1.00-1.05 (2H, m), 1.14-1.19 (2H, m), 1.24 (6H, d), 2.58 (2H, dd), 3.22 (1H, septet), 3.54-3.63 (2H, m), 3.87 (3H, s), 4.61 (2H, s), 8.75 (1H, s)

m/z (ESI+) (M+H)+=292; HPLC t_{R}=2.72 min

### Intermediate 74

### 4-cyclopropyl-2-(2,6-dimethylmorpholino)pyrimidine-5-carboxylic acid (as approximately 90% 2S,6R diastereoisomer)

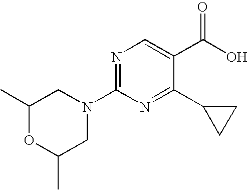

Prepared from Intermediate 73 by the Same Process Used for Intermediate 3

1H NMR (400.132 MHz, CDCl3) δ 1.02-1.08 (2H, m), 1.17-1.22 (2H, m), 1.25 (6H, d), 2.61 (2H, dd), 3.23-3.31 (1H, m), 3.55-3.65 (2H, m), 4.62 (2H, d), 8.87 (1H, s)

m/z (ESI+) (M+H)+=278; HPLC t_{R}=2.13 min.

### Example 43

### 4-Cyclopropyl-N-[(2r,5s)-5-hydroxyadamantan-2-yl]-2-(1,4-thiazepan-4-yl)pyrimidine-5-carboxamide

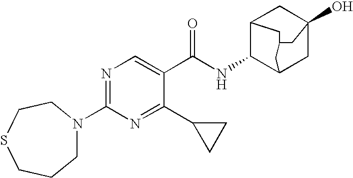

Prepared from Intermediate 80 by the Same Process Used for Example 36

1H NMR (400.132 MHz, CDCl3) δ 0.97-1.03 (2H, m), 1.15-1.22 (2H, m), 1.41 (1H, s), 1.57 (2H, d), 1.67-1.84 (6H, m), 1.94 (2H, d), 2.03-2.15 (2H, m), 2.17 (1H, s), 2.24 (2H, s), 2.49-2.58 (3H, m), 2.72-2.80 (2H, m), 3.84-3.92 (2H, m), 3.97-4.07 (2H, m), 4.21 (1H, d), 6.03 (1H, d), 8.36 (1H, s)

m/z (ESI+) (M+H)+=429; HPLC t_{R}=2.09 min.

### Example 44

### 4-Cyclopropyl-N-[(2r,5s)-5-hydroxyadamantan-2-yl]-2-(1-oxo-1,4-thiazepan-4-yl)pyrimidine-5-carboxamide

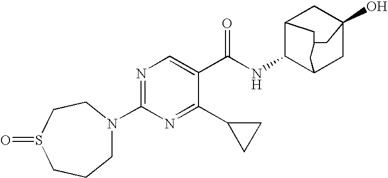

Prepared from Example 43 by the Same Process Used for Example 36

1H NMR (400.132 MHz, CDCl3) δ 1.01-1.07 (2H, m), 1.15-1.21 (2H, m), 1.42 (1H, s), 1.58 (2H, d), 1.67-1.84 (6H, m), 1.94 (2H, d), 2.05-2.15 (1H, m), 2.18 (1H, s), 2.24 (2H, s), 2.43-2.63 (3H, m), 2.85 (1H, t), 3.01-3.13 (1H, m), 3.15 (1H, q), 3.50 (1H, dt), 3.89 (1H, t), 4.18-4.44 (2H, m), 4.22 (1H, d), 6.04 (1H, d), 8.37 (1H, s)

m/z (ESI+) (M+H)+=445; HPLC t_{R}=1.37 min.

### Example 45

### 4-Cyclopropyl-2-(1,1-dioxo-1,4-thiazepan-4-yl)-N-[(2r,5s)-5-hydroxyadamantan-2-yl]pyrimidine-5-carboxamide

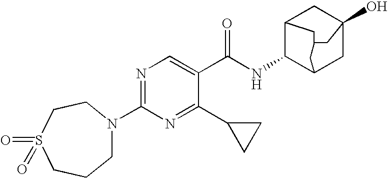

Prepared from Example 43 by the Same Process Used for Example 37

1H NMR (400.132 MHz, CDCl3) δ 1.02-1.09 (2H, m), 1.13-1.19 (2H, m), 1.41 (1H, s), 1.58 (2H, d), 1.68-1.85 (6H, m), 1.95 (2H, d), 2.16-2.28 (5H, m), 2.51 (1H, septet), 2.97 (2H, t), 3.31 (2H, s), 3.94-4.09 (4H, m), 4.22 (1H, d), 6.05 (1H, d), 8.37 (1H, s)

m/z (ESI+) (M+H)+=461; HPLC t_{R}=1.59 min.

### Example 46

### 4-Cyclopropyl-N-[(2r,5s)-5-hydroxyadamantan-2-yl]-2-(3-thia-6-azabicyclo[2.2.1]heptan-6-yl)pyrimidine-5-carboxamide

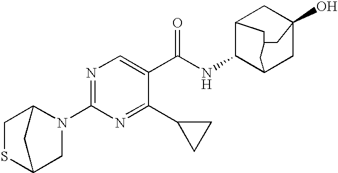

4-cyclopropyl-N-[(2r,5s)-5-hydroxyadamantan-2-yl]-2-methylsulfinylpyrimidine-5-carboxamide (Intermediate 80, 826.3 mg, 2.20 mmol) and 2-thia-5-azabicyclo[2.2.1]heptane (301.2 mg, 2.61 mmol) were dissolved in THF (4 mL) and sealed into a microwave tube. The reaction was heated to 150° C. for 60 minutes in the microwave reactor and cooled to room temperature. The reaction mixture was evaporated to dryness and redissolved in EtOAc (150 mL), and washed sequentially with saturated brine (2×75 mL). The organic layer was dried over MgSO4, filtered and evaporated to afford crude product. The crude product was purified by preparative HPLC (Phenomenex Gemini C18 110A (axia) column, 5μ silica, 30 mm diameter, 100 mm length), using decreasingly polar mixtures of water (containing 0.1% NH3) and MeCN as eluents. Fractions containing the desired compound were evaporated to dryness to afford 4-cyclopropyl-N-[(2r,5s)-5-hydroxyadamantan-2-yl]-2-(3-thia-6-azabicyclo[2.2.1]heptan-6-yl)pyrimidine-5-carboxamide as a white solid.

1H NMR (400.13 MHz, DMSO-d6) δ 0.89-0.95 (2H, m), 0.98-1.01 (2H, m), 1.32 (2H, d), 1.60 (3H, s), 1.63 (1H, s), 1.71 (2H, d), 1.85 (1H, d), 1.93 (1H, d), 1.99 (1H, s), 2.05 (2H, s), 2.23 (1H, d), 2.43 (1H, s), 2.94 (1H, d), 3.04-3.07 (1H, m), 3.27 (2H, s), 3.57 (1H, s), 3.67 (1H, s), 3.78 (1H, d), 3.89-3.93 (1H, m), 4.94 (1H, s), 8.03 (1H, d), 8.19 (1H, s)

m/z (ESI+) (M+H)+=427; HPLC t_{R}=2.03 min.

### Example 47

### 4-Cyclopropyl-N-[(2r,5s)-5-hydroxyadamantan-2-yl]-2-(3-oxo-3 ?4-thia-6-azabicyclo[2.2.1]heptan-6-yl)pyrimidine-5-carboxamide

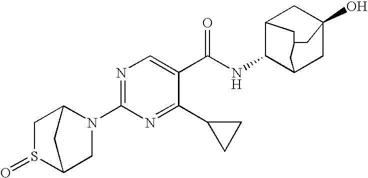

Prepared from Example 46 by the Same Process Used for Example 36

1H NMR (400.13 MHz, CDCl3) δ 0.95-0.98 (2H, m), 1.08-1.16 (2H, m), 1.49 (2H, d), 1.65 (2H, d), 1.70-1.73 (5H, m), 1.85 (2H, d), 2.09 (1H, d), 2.15 (2H, s), 2.28-2.32 (1H, m), 2.38-2.45 (1H, m), 2.66 (1H, d), 3.03 (1H, d), 3.40 (1H, d), 3.60-3.69 (1H, dd) 3.79 (1H, d), 4.09-4.14 (1H, m), 5.05 (1H, bs), 6.12 (1H, d), 8.24 (1H, s)

m/z (ESI+) (M+H)+=443; HPLC t_{R}=1.37 min.

### Example 48

### 4-Cyclopropyl-2-(3,3-dioxo-3λ6-thia-6-azabicyclo[2.2.1]heptan-6-yl)-N-[(2r,5 s)-5-hydroxyadamantan-2-yl]pyrimidine-5-carboxamide

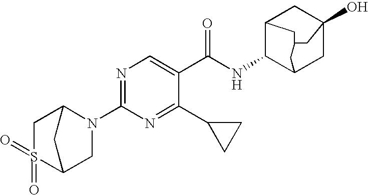

Prepared from Example 46 by the Same Process Used for Example 37

1H NMR (400.13 MHz, CDCl3) δ 0.96-0.99 (2H, m), 1.15-1.19 (2H, m), 1.50 (2H, d), 1.66 (2H, d), 1.71 (3H, s), 1.75 (1H, s), 1.86 (2H, d), 2.10 (1H, s), 2.17 (2H, s), 2.42 (2H, d), 2.62 (1H, d), 3.07-3.11 (1H, m), 3.15-3.20 (1H, m), 3.64 (1H, s), 3.67 (1H, s), 4.11-4.19 (1H, m), 5.01 (1H, bs), 6.07 (1H, d), 8.28 (2H, s)

m/z (ESI+) (M+H)+=459; HPLC t_{R}=1.58 min.

### Example 49

### 2-Amino-4-cyclopropyl-N-[(2r,5s)-5-hydroxyadamantan-2-yl]pyrimidine-5-carboxamide

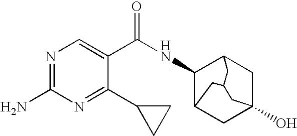

880 ammonia (10 ml, 168.19 mmol) was added to 4-cyclopropyl-N-[(2r,5s)-5-hydroxyadamantan-2-yl]-2-methylsulfonylpyrimidine-5-carboxamide (Intermediate 80, 1.2 g, 3.07 mmol) in dioxane (40 mL) at 20° C. The resulting solution was stirred at 20° C. for 3 days.

The reaction mixture was evaporated to dryness. Purified by preparative HPLC (Phenomenex Gemini C18 110A (axia) column, 5μ silica, 30 mm diameter, 100 mm length), using decreasingly polar mixtures of water (containing 0.5% NH3) and MeCN as eluents. Fractions containing the desired compound were evaporated to dryness to afford the product, which was triturated with ethyl acetate to give 2-amino-4-cyclopropyl-N-[(2r,5s)-5-hydroxyadamantan-2-yl]pyrimidine-5-carboxamide (0.420 g, 41.7%) as a white solid.

1H NMR (400.132 MHz, DMSO) δ 0.86-0.92 (2H, m), 0.97-1.00 (2H, m), 1.29-1.37 (2H, m), 1.60-1.65 (4H, m), 1.68-1.74 (2H, m), 1.91-2.02 (3H, m), 2.03-2.07 (2H, m), 2.38-2.45 (1H, m), 3.89-3.93 (1H, m), 4.43 (1H, s), 6.70 (2H, s), 8.07 (1H, d), 8.11 (1H, s)

m/z (ES+) (M+H)+=329; HPLC t_{R}=1.18 min.

### Example 50

### 4-Cyclopropyl-N-[(2r,5 s)-5-hydroxyadamantan-2-yl]-2-[(3R)-oxolan-3-ylamino]pyrimidine-5-carboxamide

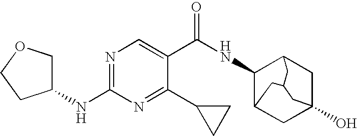

4-cyclopropyl-N-[(2r,5s)-5-hydroxyadamantan-2-yl]-2-methylsulfonylpyrimidine-5-carboxamide (Intermediate 80, 0.3 g, 0.77 mmol), (R)-tetrahydrofuran-3-amine 4-methylbenzenesulfonate (0.298 g, 1.15 mmol) and DIPEA (0.294 mL, 1.69 mmol) were dissolved in THF (5 mL) and sealed into a microwave tube. The reaction was heated to 150° C. for 1 hour in the microwave reactor and cooled to room temperature. The reaction mixture was diluted with DCM (20 ml) and washed with saturated NaHCO₃, then separated through a phase sep tube and the DCM layer evaporated. Purified by preparative HPLC (Phenomenex Gemini C18 110A (axia) column, 5μ silica, 30 mm diameter, 100 mm length), using decreasingly polar mixtures of water (containing 0.5% NH3) and MeCN as eluents. Fractions containing the desired compound were evaporated to dryness to afford the product, 4-cyclopropyl-N-[(2r,5s)-5-hydroxyadamantan-2-yl]-2-[(3R)-oxolan-3-ylamino]pyrimidine-5-carboxamide (0.104 g, 34%). Chiral analysis was carried out using 5 μm Chiralcel OJ-H (250 mm×4.6 mm)-No DH022, eluting with iso-Hexane/EtOH 80/20. The compound appears to have a chiral purity >99%.

1H NMR (400.13 MHz, DMSO-d₆) δ 0.90-0.93 (2H, m), 0.97-1.02 (2H, m), 1.31 (2H, d), 1.59 (3H, s), 1.62 (1H, s), 1.70 (2H, d), 1.82-1.87 (1H, m), 1.91-2.00 (3H, m), 2.03 (2H, s), 2.07-2.12 (1H, m), 2.39-2.44 (1H, m), 3.39-3.48 (1H, m), 3.65-3.71 (1H, m), 3.78-3.85 (2H, m), 3.88-3.92 (1H, m), 4.27 (1H, bs), 4.43 (1H, s), 7.52 (1H, bs), 8.07 (1H, d), 8.15 (1H, s)

m/z (ES+) (M+H)+=399; HPLC t_{R}=1.50 min.

### Example 51

### N-[(2r,5s)-5-hydroxyadamantan-2-yl] 4-cyclopropyl-2-[(3S)-oxolan-3-yl]amino]pyrimidine-5-carboxamide

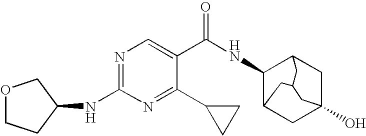

4-cyclopropyl-N-[(2r,5s)-5-hydroxyadamantan-2-yl]-2-methylsulfonylpyrimidine-5-carboxamide (Intermediate 80, 0.3 g, 0.77 mmol), (S)-tetrahydrofuran-3-amine hydrochloride (0.189 g, 1.53 mmol) and DIPEA (0.294 mL, 1.69 mmol) were dissolved in THF (5 mL) and sealed into a microwave tube. The reaction was heated to 150° C. for 1 hour in the microwave reactor and cooled to room temperature. The reaction mixture was diluted with DCM (20 ml) and washed with saturated NaHCO₃, then separated through a phase sep tube and the DCM layer evaporated. Purified by preparative HPLC (Phenomenex Gemini C18 110A (axia) column, 5μ silica, 30 mm diameter, 100 mm length), using decreasingly polar mixtures of water (containing 0.5% NH3) and MeCN as eluents. Fractions containing the desired compound were evaporated to dryness to afford the product, 4-cyclopropyl-N-[(2r,5s)-5-hydroxyadamantan-2-yl]-2-[[(3S)-oxolan-3-yl]amino]pyrimidine-5-carboxamide (0.106 g, 35%). Chiral analysis was carried out using 5 μm Chiralcel OJ-H (250 mm×4.6 mm)-No DG022, eluting with iso-Hexane/EtOH 80/20. The compound appears to have a chiral purity >98%.

1H NMR (400.132 MHz, CDCl3) δ 1.00-1.03 (2H, m), 1.17-1.23 (2H, m), 1.55 (2H, d), 1.69-1.87 (8H, m), 1.94 (2H, d), 2.17 (1H, s), 2.24-2.34 (3H, m), 2.45-2.52 (1H, m), 3.67 (1H, dd), 3.81-3.87 (1H, m), 3.92-3.99 (2H, m), 4.18-4.23 (1H, m), 4.52 (1H, s), 5.32 (1H, d), 6.03 (1H, d), 8.32 (1H, s)

m/z (ES+) (M+H)+=399; HPLC t_{R}=1.50 min.

The following Examples were prepared in a similar manner to Example 46, using Intermediate 80 and an appropriate amine starting material:

| Structure | Ex | Name | ¹H NMR δ | MS m/e MH⁺ |
|---|---|---|---|---|
| 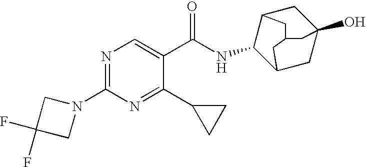 | 52 | 4-cyclopropyl-2- (3,3- difluoroazetidin-1- yl)-N-[(2r,5s)-5- hydroxyadamantan- 2-yl]pyrimidine-5- carboxamide | 1H NMR (400.13 MHz, CDCl3) δ 0.96-0.99 (2H, m), 1.13-1.16 (2H, m), 1.50 (2H, d), 1.63 (1H, s), 1.66 (1H, s), 1.71 (3H, s), 1.74 (1H, s), 1.84 (1H, s), 1.87 (1H, s), 2.09 (1H, s), 2.17 (2H, s), 2.36-2.42 (1H, m), 4.11-4.16 (1H, m), 4.33 (4H, t), 6.08 (1H, d), 8.28 (1H, s) | m/z (ES+) (M + H)+ = 405; HPLC t_{R} = 1.99 min |
| 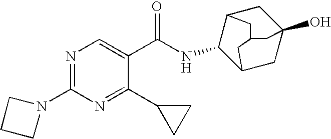 | 53 | 2-(azetidin-1-yl)-4- cyclopropyl-N- [(2r,5s)-5- hydroxyadamantan- 2-yl]pyrimidine-5- carboxamide | 1H NMR (400.13 MHz, CDCl3) δ 0.89-0.93 (2H, m), 1.12-1.15 (2H, m), 1.48 (2H, d), 1.65 (2H, d), 1.69 (3H, s), 1.73 (1H, s), 1.84-1.86 (2H, m), 2.08 (1H, s), 2.15 (2H, s), 2.24-2.31 (2H, m), 2.35-2.42 (1H,m), 4.04 (4H, t), 4.08-4.13 (1H, m), 6.11 (1H, d), 8.24 (1H, s) | m/z (ES+) (M + H)+ = 369; HPLC t_{R} = 1.83 min. |
| 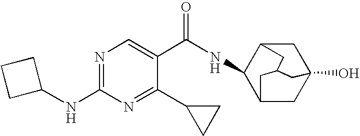 | 54 | (cyclobutylamino)- 4-cyclopropyl-N- [(2r,5s)-5- hydroxyadamantan- 2-yl]pyrimidine-5- carboxamide | 1H NMR (400.13 MHz, DMSO-d₆) d 0.88-0.92 (2H, m), 1.00 (2H, s), 1.31 (2H, d), 1.60-1.72 (8H, m), 1.88-2.03 (7H, m), 2.14-2.22 (2H, m), 2.38-2.45 (1H, m), 3.16 (1H, d), 3.90 (1H, t), 4.40 (1H, s), 7.44 (1H, s), 8.00 (1H, d), 8.12 (1H, s) | m/z (ES+) (M + H)+ = 383; HPLC t_{R} = 2.00 min. |
| 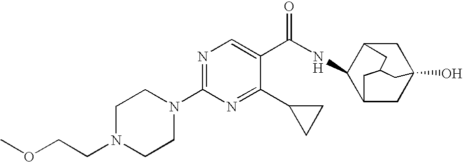 | 55 | 4-cyclopropyl-N- [(2r,5s)-5- hydroxyadamantan- 2-yl]-2-[4-(2- methoxy- ethyl)piperazin-1- yl]pyrimidine-5- carboxamide | 1H NMR (400.13 MHz, DMSO-d₆) d 0.90-0.96 (2H, m), 0.99-1.01 (2H, m), 1.32 (2H, d), 1.62 (4H, d), 1.70 (2H, s), 1.93 (2H, d), 1.99 (1H, s), 2.04 (2H, s), 2.40-2.44 (5H, m), 2.45-2.55 (2H, t), 3.23 (3H, s), 3.41-3.46 (2H, t), 3.68 (4H, t), 3.91 (1H, m), 4.38 (1H, s), 8.03 (1H, d), 8.20 (1H, s) | m/z (ES+) (M + H)+ = 456; HPLC t_{R} = 1.71 min. |
| 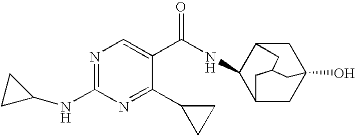 | 56 | 4-cyclopropyl-2- (cyclopropylamino)- N-[(2r,5s)-5- hydroxyadamantan- 2-yl]pyrimidine-5- carboxamide | 1H NMR (400.13 MHz, DMSO-d₆) d 0.40-0.44 (2H, m), 0.60-0.65 (2H, m), 0.89-0.92 (2H, m), 1.01 (2H, d), 1.32 (2H, d), 1.60-1.63 (4H, m), 1.69-1.63 (4H, m), 1.69-1.72 (2H, m), 1.93-1.96 (2H, m), 1.99 (1H, s), 2.04 (2H, s), 2.40-2.46 (1H, m), 2.63-2.70 (1H, m), 3.91 (1H, m), 4.37 (1H, s), 7.35 (1H, s), 8.02 (1H, d), 8.16 (1H, s) | m/z (ES+) (M + H)+ = 369; HPLC t_{R} = 1.72 min. |
| 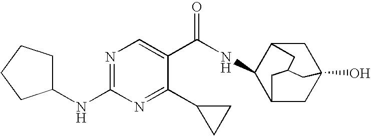 | 57 | 2- (cyclopentylamino)- 4-cyclopropyl-N- [(2r,5s)-5- hydroxyadamantan- 2-yl]pyrimidine-5- carboxamide | 1H NMR (400.13 MHz, DMSO-d₆) δ 0.88-0.91 (2H, m), 1.01 (2H, s), 1.32 (2H, d), 1.40-1.54 (4H, m), 1.60-1.66 (6H, m), 1.70 (2H, d), 1.81-1.87 (2H, m), 1.94 (2H, d), 1.99 (1H, s), 2.04 (2H, s), 2.40-2.46 (1H, m), 3.90 (1H, m), 4.04-4.08 (1H, m), 4.37 (1H, s), 7.17 (1H, s), 7.99 (1H, d), 8.13 (1H, s) | m/z (ES+) (M + H)+ = 397; HPLC t_{R} = 2.12 min. |
| 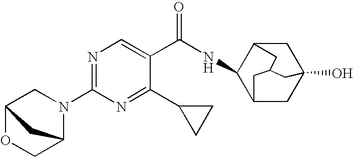 | 58 | 4-cyclopropyl-N- [(2r,5s)-5- hydroxyadamantan- 2-yl]-2-[(1S,4S)-2- oxa-5-azabi- cyclo[2.2.1]hept-5- yl]pyrimidine-5- carboxamide | 1H NMR (400.13 MHz, DMSO-d₆) δ 0.91-0.94 (2H, m), 0.98-1.09 (2H, m), 1.32 (2H, d), 1.62 (4H, d), 1.71 (2H, d), 1.82-1.89 (2H, m), 1.94 (2H, d), 1.99 (1H, s), 2.04 (2H, s), 2.40-2.46 (1H, m), 3.32-3.44 (2H, m), 3.58 (1H, d), 3.76-3.78 (1H, m), 3.91 (1H, m), 4.38 (1H, s), 4.63 (1H,s), 8.04 (1H, d), 8.19 (1H, s) | m/z (ES+) (M + H)+ = 411; HPLC t_{R} = 1.62 min. |
| 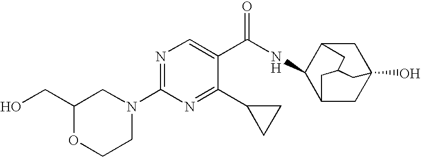 | 59 | 4-cyclopropyl-N- [(2r,5s)-5- hydroxyadamantan- 2-yl]-2-[2-(hydroxy- methyl)morpholin- 4-yl]pyrimidine-5- carboxamide | 1H NMR (400.13 MHz, DMSO-d₆) δ 0.92-0.95 (2H, m), 1.01-1.03 (2H, m), 1.32 (2H, d), 1.62 (4H, d), 1.74 (2H, q), 1.93 (2H, d), 1.99 (1H, s), 2.04 (2H, s), 2.41-2.47 (1H, m), 2.65-2.71 (1H, m), 2.93 (1H, m), 3.32-3.51 (4H, m), 3.87-3.93 (2H, m), 4.37 (2H, d), 4.55 (1H, t), 4.76 (1H, t), 8.06 (1H, d), 8.22 (1H, m) | m/z (ES+) (M + H)+ = 429; HPLC t_{R} = 1.47 min. |
| 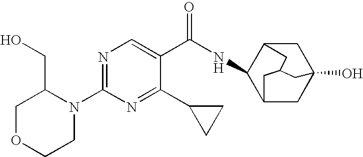 | 60 | 4-cyclopropyl-N- [(2r,5s)-5- hydroxyadamantan- 2-yl]-2-[3- (hydroxy- methyl)morpholin- 4-yl]pyrimidine-5- carboxamide | 1H NMR (400.13 MHz, DMSO-d₆) δ 0.92-0.98 (2H, m), 0.99-1.07 (2H, m), 1.32 (2H, d), 1.61 (3H, d), 1.71 (2H, d), 1.94 (3H, d), 1.99 (1H, s), 2.04 (2H, s), 2.38-2.44 (1H, m), 3.03 (1H, m), 3.31-3.45 (3H, m), 3.66-3.72 (1H, m), 3.85-3.88 (1H, m), 3.92 (1H, d), 4.05 (1H, d), 4.25 (1H, m), 4.38-4.42 (2H, m), 4.81 (1H, t), 8.05 (1H, d), 8.22 (1H, s) | m/z (ES+) (M + H)+ = 429; HPLC t_{R} = 1.52 min |
| 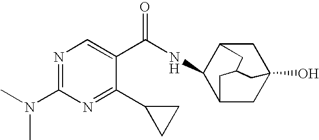 | 61 | 4-cyclopropyl-2- (dimethylamino)-N- [(2r,5s)-5- hydroxyadamantan- 2-yl]pyrimidine-5- carboxamide | 1H NMR (400.13 MHz, DMSO-d₆) δ 0.90-0.94 (2H, m), 1.02-1.05 (2H, m), 1.30-1.34 (2H, m), 1.60-1.63 (4H, m), 1.69-1.72 (2H, m), 1.92-1.96 (2H, m), 1.99 (1H, s), 2.04 (2H, s), 2.47 (1H, m), 3.08 (6H, s), 3.91 (1H, m), 4.37 (1H, s), 7.99 (1H, d), 8.21 (1H, s) | m/z (ES+) (M + H)+ = 357; HPLC t_{R} = 1.85 min. |
| 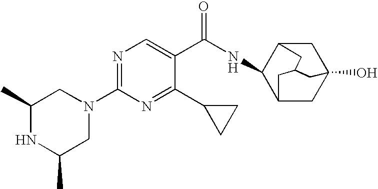 | 62 | 4-cyclopropyl-2- [(3R,5S)-3,5- dimethylpiperazin- 1-yl]-N-[(2r,5s)-5- hydroxyadamantan- 2-yl]pyrimidine-5- carboxamide | 1H NMR (400.13 MHz, DMSO-d₆) δ 0.90-0.96 (2H, m), 0.99 (6H, d), (0.99 (2H, m), 1.32 (2H, d), 1.62 (4H, d), 1.70 (2H, d), 1.93 (2H, d), 1.99 (1H, s), 2.04 (2H, s), 2.22 (1H, s), 2.26-2.32 (2H, m), 2.42-2.46 (1H, m), 2.58-2.66 (2H, m), 3.91 (1H, m), 4.37 (1H, s), 4.46-4.49 (2H, m), 8.01 (1H, d), 8.19 (1H, s) | m/z (ES+) (M + H)+ = 426; HPLC t_{R} = 1.66 min. |
| 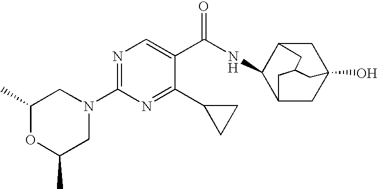 | 63 | 4-cyclopropyl-2- [(2R,6R)-2,6- dimethylmorpholin- 4-yl]-N-[(2r,5s)-5- hydroxyadamantan- 2-yl]pyrimidine-5- carboxamide | 1H NMR (400.13 MHz, DMSO-d₆) δ 0.92-0.95 (2H, m), 0.98-1.13 (2H, m), 1.08 (6H, d), 1.32 (2H, d), 1.62 (4H, d), 1.71 (2H, d), 1.94 (2H, d), 1.99 (1H, s), 2.04 (2H, s), 2.42-2.46 (1H, m), 3.41-3.46 (2H, m), 3.80 (2H, d), 3.92-3.96 (3H, m), 4.37 (1H, s), 8.05 (1H, d), 8.21 (1H, s) | m/z (ES+) (M + H)+ = 427; HPLC t_{R} = 1.94 min. |
| 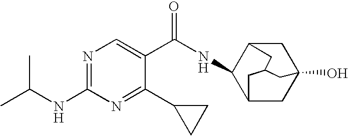 | 64 | 4-cyclopropyl-N- [(2r,5s)-5- hydroxyadamantan- 2-yl]-2-(isopropyl- amino)pyrimidine-5- carboxamide | 1H NMR (400.13 MHz, DMSO-d₆) δ 0.88-0.91 (2H, m), 1.00 (2H, s), 1.11 (6H, d), 1.32 (2H, d), 1.62 (4H, d), 1.70 (2H, d), 1.94 (2H, d), 1.99 (1H, s), 2.04 (2H, s), 2.40-2.47 (1H, m), 3.89-3.98 (2H, m), 4.38 (1H, s), 7.01 (1H, s), 7.98 (1H, d), 8.13 (1H, s) | m/z (ES+) (M + H)+ = 371; HPLC t_{R} = 1.90 min. |
| 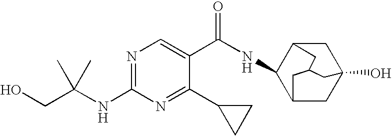 | 65 | 4-cyclopropyl-N- [(2r,5s)-5- hydroxyadamantan- 2-yl]-2-[(2-hydroxy- 1,1-dimethyl- ethyl)amino]pyrimi- dine-5-carboxamide | 1H NMR (400.13 MHz, DMSO-d₆) δ 0.93 (2H, d), 0.96-1.01 (2H, m), 1.27 (6H, s), 1.32 (2H, d), 1.62 (4H, d), 1.70 (2H, d), 1.94 (2H, d), 1.99 (1H, s), 2.05 (2H, d), 2.39-2.45 (1H, m), 3.44 (2H, d), 3.91 (1H, m), 4.37 (1H, s), 4.85 (1H, m), 6.40 (1H, s), 8.02 (1H, d), 8.12 (1H, s) | m/z (ES+) (M + H)+ = 401; HPLC t_{R} = 1.66 min. |
| 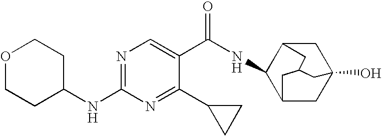 | 66 | 4-cyclopropyl-N- [(2r,5s)-5- hydroxyadamantan- 2-yl]-2-(tetrahydro- 2H-pyran-4- ylamino)pyrimidine- 5-carboxamide | 1H NMR (400.13 MHz, DMSO-d₆) δ 0.89-0.96 (2H, m), 1.00 (2H, s), 1.32 (2H, d), 1.43-1.53 (2H, m), 1.62 (4H, d), 1.70 (2H, d), 1.77 (2H, d), 1.94 (2H, d), 1.99 (1H, s), 2.04 (2H, s), 2.42 (1H, s), 3.32-3.38 (2H, m), 3.84 (3H, d), 3.91 (1H, m), 4.37 (1H, s), 7.18 (1H, s), 8.00 (1H, d), 8.14 (1H, s) | m/z (ES+) (M + H)+ = 413; HPLC t_{R} = 1.60 min. |
| 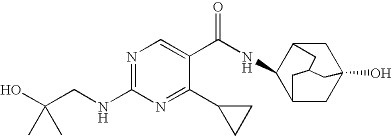 | 67 | 4-cyclopropyl-N- [(2r,5s)-5- hydroxyadamantan- 2-yl]-2-[(2-hydroxy- 2-methylpro- pyl)amino]pyrimi- dine-5-carboxamide | 1H NMR (400.13 MHz, DMSO-d₆) δ 0.90 (2H, m), 1.01 (2H, m), 1.06 (6H, s), 1.32 (2H, d), 1.62 (4H, d), 1.70 (2H, d), 1.94 (2H, d), 1.99 (1H, m), 2.04 (2H,s), 2.43 (1H,m) 3.25 (2H, d), 3.90 (1H, m), 4.37 (1H, s), 4.49 (1H, s), 6.93 (1H, s), 8.02 (1H, d), 8.13 (1H, s) | m/z (ES+) (M + H)+ = 401; HPLC t_{R} = 1.53 min. |
| 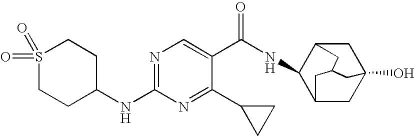 | 68 | 4-cyclopropyl-2- [(1,1- dioxidotetrahydro- 2H-thiopyran-4- yl)amino]-N- [(2r,5s)-5- hydroxyadamantan- 2-yl]pyrimidine-5- carboxamide | 1H NMR (400.13 MHz, DMSO-d₆) δ 0.90-0.94 (2H, m), 1.03 (2H, s), 1.32 (2H, d), 1.62 (4H, d), 1.69-1.76 (2H, m), 1.92-2.04 (7H, m), 2.10-2.15 (2H, m), 2.38-2.43 (1H, m), 3.11 (2H, d), 3.21 (2H, d), 3.91 (1H, m), 4.04 (1H, s), 4.38 (1H, s), 7.39 (1H, s), 8.03 (1H, d), 8.15 (1H, s) | m/z (ES+) (M + H)+ = 461; HPLC t_{R} = 1.48 min. |
| 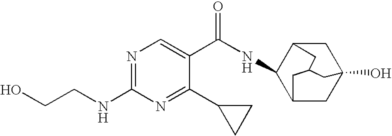 | 69 | 4-cyclopropyl-N- [(2r,5s)-5- hydroxyadamantan- 2-yl]-2-[(2- hydroxy- ethyl)amino]pyrimi- dine-5-carboxamide | 1H NMR (400.13 MHz, DMSO-d₆) δ 0.89-0.96 (2H, m), 1.00 (2H, s), 1.32 (2H, d), 1.62 (4H, d), 1.72 (2H, m), 1.94 (2H, d), 1.99 (1H, s), 2.04 (2H, s), 2.40-2.46 (1H, m), 3.32 (2H, m), 3.46 (2H, q), 3.90 (1H, m), 4.37 (1H, s), 4.59 (1H, t), 7.04 (1H, s), 8.00 (1H, d), 8.13 (1H, s) | m/z (ES+) (M + H)+ = 373; HPLC t_{R} = 1.31 min. |
| 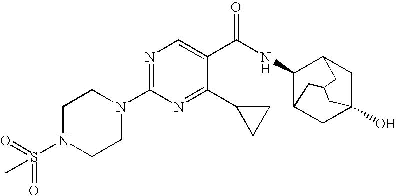 | 70 | 4-cyclopropyl-N- [(2r,5s)-5- hydroxyadamantan- 2-yl]-2-(4- methylsulfonyl- piperazin-1- yl)pyrimidine-5- carboxamide | NMR: 1H NMR (400.132 MHz, CDCl3) δ 1.02-1.08 (2H, m), 1.16-1.21 (2H, m), 1.40 (1H, s), 1.56-1.63 (2H, m), 1.77-1.82 (4H, m), 1.91-1.95 (2H, m), 2.18 (1H, s), 2.24 (2H, s), 2.47-2.54 (1H, m), 2.78 (3H, s), 3.25 (4H, t), 3.95 (4H, t), 4.19-4.24 (1H, m), 6.03 (1H, d), 8.36 (1H, s) | m/z (ES+) (M + H)+ = 476; HPLC t_{R} = 1.73 min. |
| 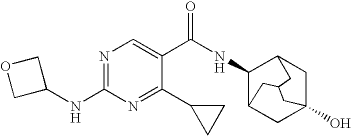 | 71 | 4-cyclopropyl-N- [(2r,5s)-5- hydroxyadamantan- 2-yl]-2-(oxetan-3- ylamino)pyrimidine- 5-carboxamide | 1H NMR (400.13 MHz, DMSO-d₆) d 0.83-0.95 (2H, m), 0.97-1.03 (2H, m), 1.31 (2H, d), 1.61 (4H, d), 1.70 (2H, d), 1.93 (2H, d), 1.98 (1H,s), 2.04 (2H, s), 2.37-2.44 (1H, m), 3.91 (1H, m), 4.37 (1H, s), 4.47 (2H, t), 4.69-4.72 (2H, t), 4.80 (1H, s), 7.93 (1H, s), 8.05 (1H, d), 8.15 (1H, s) | m/z (ES+) (M + H)+ = 385; HPLC t_{R} = 1.41 min. |
| 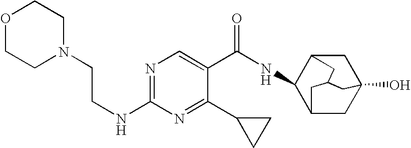 | 72 | 4-cyclopropyl-N- [(2r,5s)-5- hydroxyadamantan- 2-yl]-2- [(2-morpholin-4-yl- ethyl)amino]pyrimi- dine-5-carboxamide | 1H NMR (400 MHz, DMSO) d 0.88-0.81 (2H, m), 0.95 (2H, s), 1.26 (2H, d), 1.56 (4H, d), 1.65 (2H, d), 1.95-1.83 (3H, m), 1.99 (2H, s), 2.39-2.28 (7H, m), 3.35-3.26 (2H, m), 3.56-3.45 (4H, m), 3.90-3.80 (1H, m), 4.32 (1H, s), 7.18-6.81 (1H, m), 7.95 (1H, d), 8.08 (1H, s). | m/z (ES+) (M + H)+ = 442; HPLC t_{R} = 1.46 min. |
| 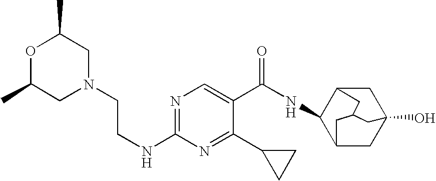 | 73 | 4-cyclopropyl-2- ({2-[(2R,6S)-2,6- dimethylmorpholin- 4-yl]ethyl}amino)- N-[(2r,5s)-5- hydroxyadamantan- 2-yl]pyrimidine-5- carboxamide | 1H NMR (400 MHz, DMSO) d 1.02-0.93 (2H, m), 1.14-1.04 (7H, m), 1.39 (2H, d), 1.74-1.64 (6H, m), 1.77 (2H, d), 2.08-1.97 (3H, m), 2.11 (2H, s), 2.53-2.42 (3H, m), 2.81 (2H, d), 3.33 (1H, s), 3.45-3.37 (2H, m), 3.65-3.55 (2H, m), 3.98 (1H, s), 4.44 (1H, s), 8.07 (1H, d), 8.20 (1H, s). | m/z (ES+) (M + H)+ = 470; HPLC t_{R} = 1.69 min. |
| 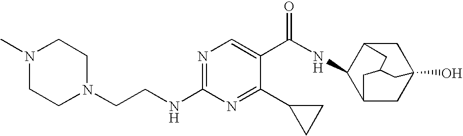 | 74 | 4-cyclopropyl-N- [(2r,5s)-5- hydroxyadamantan- 2-yl]-2-{[2-(4- methylpiperazin-1- yl)ethyl]amino}py- rimidine-5- carboxamide | 1H NMR (400 MHz, DMSO) d 0.90 (2H, d), 1.00 (2H, s), 1.32 (2H, d), 1.61 (4H, d), 1.70 (2H, d), 2.00-1.89 (3H, m), 2.04 (2H, s), 2.13 (3H, s), 2.45-2.22 (11H, m), 3.41-3.11 (2H, m), 3.97-3.84 (1H, m), 4.37 (1H, s), 7.25-6.82 (1H, m), 8.00 (1H, d), 8.13 (1H, s). | m/z (ES+) (M + H)+ = 455; HPLC t_{R} = 1.41 min. |

### Example 75

### 2-(Cyclobutyloxy)-4-cyclopropyl-N-[(2r,5 s)-5-hydroxyadamantan-2-yl]pyrimidine-5-carboxamide

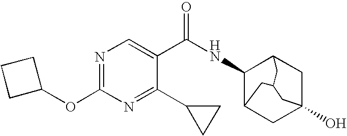

Sodium hydride (30.6 mg, 0.77 mmol) was added to cyclobutanol (0.300 mL, 3.83 mmol) in THF (3 mL) at 20° C. under nitrogen. The resulting solution was stirred at 20° C. for 30 minutes. Then 4-cyclopropyl-N-[(2r,5s)-5-hydroxyadamantan-2-yl]-2-(methylsulfonyl)pyrimidine-5-carboxamide (300 mg, 0.77 mmol) in THF (4 mL) was added dropwise and the solution stirred for a further 2 hrs.

The reaction mixture was diluted with DCM (10 mL), and stirred with water (10 mL) before passing through a phase separation cartridge. The organic layer was evaporated to afford crude product. The crude product was purified by preparative HPLC (Waters XBridge Prep C18 OBD column, 5μ silica, 50 mm diameter, 150 mm length), using decreasingly polar mixtures of water (containing 0.1% NH3) and MeCN as eluents. Fractions containing the desired compound were evaporated to dryness to afford 2-(cyclobutyloxy)-4-cyclopropyl-N-[(2r,5s)-5-hydroxyadamantan-2-yl]pyrimidine-5-carboxamide (146 mg, 49.7%) as a white solid.

1H NMR (400.13 MHz, DMSO-d₆) δ 1.02-1.06 (4H, m), 1.33 (2H, d), 1.61-1.81 (8H, m), 1.91-2.09 (7H, m), 2.33-2.39 (3H, m), 3.95 (1H, m), 4.39 (1H, s), 5.00-5.08 (1H, m), 8.31 (1H, d), 8.35 (1H, s)

m/z (ES+) (M+H)+=384; HPLC t_{R}=2.00 min

The following Examples were prepared in a similar manner to Example 75, using Intermediate 80 and an appropriate starting material:

| Structure | Ex | Name | ¹H NMR δ | MS m/e MH⁺ |
|---|---|---|---|---|
| 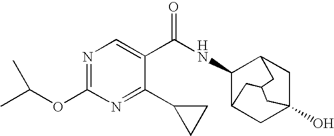 | 76 | 4-cyclopropyl-N- [(2r,5s)-5- hydroxyadamantan-2- yl]-2- isopropoxypyrimidine- 5-carboxamide | 1H NMR (400.13 MHz, DMSO-d₆) δ 1.00-1.08 (4H, m), 1.28 (6H, d), 1.33 (2H, d), 1.62 (4H, d), 1.72 (2H, d), 1.93 (2H, d), 1.99 (1H, s), 2.06 (2H, s), 2.33-2.39 (1H, m), 3.95 (1H, m), 4.39 (1H, s), 5.10-5.16 (1H, m), 8.29 (1H, d), 8.35 (1H, s) | m/z (ES+) (M + H)+ = 372; HPLC t_{R} = 1.87 min. |
| 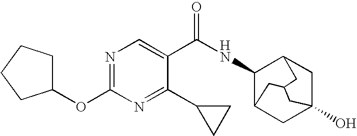 | 77 | 2-(cyclopentyloxy)-4- cyclopropyl-N- [(2r,5s)-5- hydroxyadamantan-2- yl]pyrimidine-5- carboxamide | 1H NMR (400.13 MHz, DMSO-d₆) δ 1.00-1.09 (4H, m), 1.33 (2H, d), 1.57-1.73 (12H, m), 1.89-1.99 (5H, m), 2.06 (2H, s), 2.33-2.39 (1H, m), 3.95 (1H, m), 4.39 (1H, s), 5.25-5.29 (1H, m), 8.29 (1H, d), 8.35 (1H, s) | m/z (ES+) (M + H)+ = 398; HPLC t_{R} = 2.13 min. |
| 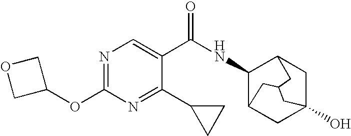 | 78 | 4-cyclopropyl-N- [(2r,5s)-5- hydroxyadamantan-2- yl]-2-(oxetan-3- yloxy)pyrimidine-5- carboxamide | 1H NMR (400.13 MHz, DMSO-d₆) d 1.04-1.08 (3H, m), 1.18-1.23 (1H, m), 1.31-1.38 (2H, m), 1.62 (4H, d), 1.71 (2H, d), 1.92 (2H, d), 1.99 (1H, s), 2.06 (2H, s), 2.31-2.37 (1H, m), 3.95 (1H, m), 4.39 (1H, s), 4.52-4.55 (2H, m), 4.84 (2H, t), 5.46-5.52 (1H, m), 8.34 (1H, d), 8.38 (1H, s) | m/z (ES+) (M + H)+ = 386; HPLC t_{R} = 1.39 min. |

### Example 79

### (4-Cyclopropyl-2-morpholinopyrimidin-5-yl)(3-(pyridin-3-yl)pyrrolidin-1-yl)methanone

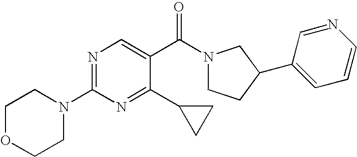

Morpholine (1.985 mL, 22.55 mmol) was added in one portion to (4-cyclopropyl-2-(methylsulfonyl)pyrimidin-5-yl)(3-(pyridin-3-yl)pyrrolidin-1-yl)methanone (Intermediate 82 0.240 g, 0.64 mmol) in THF (2 mL) at 18° C. The resulting solution was stirred at 150° C. for 12 hours. The reaction mixture was diluted with DCM (50 mL), and washed with dil. aq. K2CO3 (20 mL). The organic layer was dried over MgSO4, filtered and evaporated to afford crude product. The crude product was purified by preparative HPLC (Waters XBridge Prep C18 OBD column, 5μ silica, 21 mm diameter, 150 mm length), using decreasingly polar mixtures of water (containing 1% NH3) and MeCN as eluents. Fractions containing the desired compounds were evaporated to dryness to afford (4-cyclopropyl-2-morpholinopyrimidin-5-yl)(3-(pyridin-3-yl)pyrrolidin-1-yl)methanone (0.135 g, 55.2%) and 3-(1-(4-cyclopropyl-2-morpholinopyrimidine-5-carbonyl)pyrrolidin-3-yl)pyridine 1-oxide (0.045 g, 17.66%) as white solids.

1H NMR (400.13 MHz, DMSO-d6) δ 0.92-1.10 (4H, m), 1.99-2.12 (2H, m), 2.24-2.36 (1H, m), 3.36-4.06 (13H, m), 7.31-7.37 (1H, m), 7.68-7.78 (1H, m), 8.22 (1H, d), 8.42-8.56 (2H, m)

m/z (ESI+) (M+H)+=380.22; HPLC t_{R}=1.73 min.

### Intermediate 81

### (4-cyclopropyl-2-(methylthio)pyrimidin-5-yl)(3-(pyridin-3-yl)pyrrolidin-1-yl)methanone

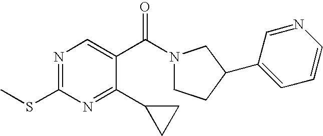

N-Ethyldiisopropylamine (0.741 mL, 4.28 mmol) was added in one portion to 4-cyclopropyl-2-(methylthio)pyrimidine-5-carboxylic acid (Intermediate 24, 1.8 g, 8.56 mmol), 3-(pyrrolidin-3-yl)pyridine (1.269 g, 8.56 mmol) and O-(7-Azabenzotriazol-1-yl)-N,N,N′,N′-tetramethyluronium hexafluorophosphate (3.91 g, 10.27 mmol) in DMF (50 mL) at 18° C. under nitrogen. The resulting mixture was stirred at 18° C. for 18 hours. The reaction mixture was diluted with EtOAc (200 mL), and washed sequentially with water (2×50 mL), and saturated brine (50 mL). The organic layer was dried over MgSO4, filtered and evaporated to afford crude product. The crude product was purified by flash silica chromatography, elution gradient 0 to 100% (MeOH:7M NH3 in MeOH:DCM (1:1:18) in DCM. Pure fractions were evaporated to dryness and dried under high vacuum to afford (4-cyclopropyl-2-(methylthio)pyrimidin-5-yl)(3-(pyridin-3-yl)pyrrolidin-1-yl)methanone (1.920 g, 65.9%) as a tan solid foam.

1H NMR (400.13 MHz, DMSO-d₆) δ 1.03-1.26 (6H, m), 2.02-2.14 (2H, m), 2.25-2.40 (1H, m), 2.44-2.47 (3H, m), 3.34-3.82 (4H, m), 7.31-7.38 (1H, m), 7.68-7.79 (1H, m), 8.42-8.57 (2H, m)

m/z (ESI+) (M+H)+=341; HPLC t_{R}=1.85 min.

### Intermediate 82

### (4-cyclopropyl-2-(methylsulfonyl)pyrimidin-5-yl)(3-(pyridin-3-yl)pyrrolidin-1′-yl)methanone

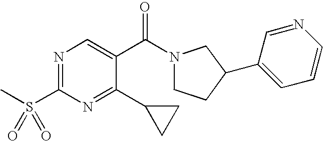

3-Chloroperoxybenzoic acid (70%) (3.16 g, 12.82 mmol) was added in one portion to (4-cyclopropyl-2-(methylthio)pyrimidin-5-yl)(3-(pyridin-3-yl)pyrrolidin-1′-yl)methanone (Intermediate 81, 2.91 g, 8.55 mmol) in DCM (100 mL) at 0° C. The resulting solution was stirred at 20° C. for 24 hours. The reaction mixture was washed sequentially with saturated NaHCO3 (50 mL), 2M NaOH (50 mL), and saturated brine (50 mL). The organic layer was dried over MgSO4, filtered and evaporated to afford crude product. EN01579-03-1, 433 mg). This material was 3 spots by tlc and at least 3 peaks by Lcms. Lcms suggests incorporation of 1, 2 and 3 oxygens which suggests the components are probably the sulphoxide/pyridine, the sulphone/pyridine and the sulphone/pyridine N-oxide. Further extraction of the aqueous gave after drying and evaporation a white solid. Both materials were used without further purification or characterisation.

m/z (ESI+) (M+H)+=373; HPLC t_{R}=1.43 min.

The following Examples were prepared in a similar manner to Example 79, using Intermediate 82 and an appropriate amine starting material:

| Structure | Ex | Name | ¹H NMR δ | MS m/e MH⁺ |
|---|---|---|---|---|
| 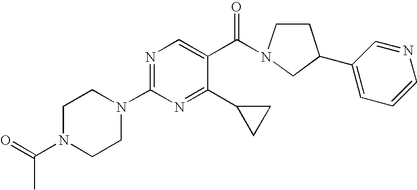 | 80 | 1-(4-(4-cyclopropyl-5-(3- (pyridin-3-yl)pyrrolidine-1- carbonyl)pyrimidin-2- yl)piperazin-1-yl)ethanone | 1H NMR (400.13 MHz, DMSO-d6) δ 0.93-1.10 (4H, m), 2.02-2.13 (5H, m), 2.25-2.36 (1H, m), 3.37-3.58 (7H, m), 3.60-3.73 (5.5H, m), 3.98-4.04 (0.5H, m), 7.31-7.38 (1H, m), 7.68-7.78 (1H, m), 8.23 (1H, d), 8.42-8.46 (1H, m), 8.49-8.56 (1H, m) | m/z (ESI+) (M + H)+ = 421; HPLC t_{R} = 1.6 min. |
| 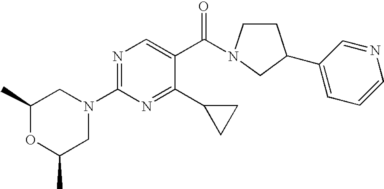 | 81 | (4-cyclopropyl-2-((2S,6R)-2,6- dimethylmorpholino)pyrimidin- 5-yl)(3-(pyridin-3-yl)pyrrolidin- 1-yl)methanone | 1H NMR (400.13 MHz, DMSO-d6) δ 0.92-1.23 (10H, m), 2.03-2.12 (2H, m), 2.25-2.33 (1H, m), 2.43-2.54 (1H, m), 3.31-3.60 (6.5H, m), 3.71-3.76 (1H, m), 3.98-4.02 (0.5H, m), 4.40-4.46 (2H, m), 7.31-7.37 (1H, m), 7.68-7.78 (1H, m), 8.21 (1H, d), 8.42-8.46 (1H, m), 8.49-8.55 (1H, m) | m/z (ESI+) (M + H)+ = 408; HPLC t_{R} = 1.97 min. |

### Example 82

### 4-Cyclobutyl-N-[(2r,5 s)-5-hydroxyadamantan-2-yl]-2-(1-oxo-1,4-thiazinan-4-yl)pyrimidine-5-carboxamide

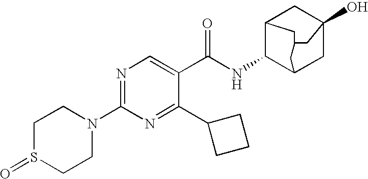

Prepared from Example 41 by the same process used for Example 36.

1H NMR (400.132 MHz, CDCl3) δ 1.44 (1H, s), 1.55-1.58 (2H, m), 1.67-1.73 (2H, m), 1.78-1.83 (4H, m), 1.87-1.95 (3H, m), 2.00-2.10 (1H, m), 2.15-2.29 (5H, m), 2.34-2.44 (2H, m), 2.71-2.90 (4H, m), 3.98 (1H, quintet), 4.14-4.29 (3H, m), 4.63-4.70 (2H, m), 5.87 (1H, d), 8.35 (1H, s)

m/z (ESI+) (M+H)+=445; HPLC t_{R}=1.45 min

### Example 83

### 4-Cyclobutyl-2-(1,1-dioxo-1,4-thiazinan-4-yl)-N-[(2r,5s)-5-hydroxyadamantan-2-yl]pyrimidine-5-carboxamide

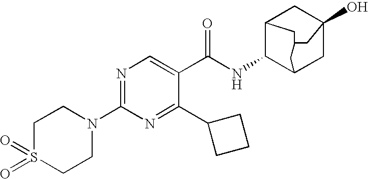

Prepared from Example 41 by the same process used for Example 37.

1H NMR (400.132 MHz, CDCl3) δ 1.38 (1H, s), 1.56-1.61 (2H, m), 1.66-1.74 (2H, m), 1.78-1.83 (4H, m), 1.87-1.96 (3H, m), 2.01-2.10 (1H, m), 2.16-2.29 (5H, m), 2.32-2.41 (2H, m), 3.03-3.09 (4H, m), 3.98 (1H, quintet), 4.16-4.21 (1H, m), 4.42-4.49 (4H, m), 5.83 (1H, d), 8.36 (1H, s)

m/z (ESI+) (M+H)+=461; HPLC t_{R}=1.72 min

### Example 84

### 2-Amino-4-cyclobutyl-N-[(2r,5s)-5-hydroxyadamantan-2-yl]pyrimidine-5-carboxamide

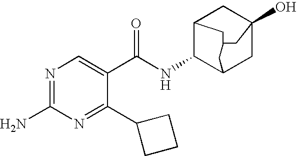

Prepared from Intermediate 72 by the same process used for Example 49

1H NMR (400.13 MHz, DMSO-d₆) δ 1.31 (2H, d), 1.58-1.64 (4H, m), 1.67-1.77 (3H, m), 1.84-1.94 (3H, m), 1.95-1.99 (1H, m), 2.00-2.12 (4H, m), 2.22-2.32 (2H, m), 3.80-3.89 (2H, m), 4.38 (1H, s), 6.77 (2H, s), 7.94 (1H, d), 8.09 (1H, s)

m/z (ESI+) (M+H)+=343; HPLC t_{R}=1.42 min.

### Example 85

### 2-Azetidin-1-yl-4-cyclobutyl-N-[(2r,5s)-5-hydroxyadamantan-2-yl]pyrimidine-5-carboxamide

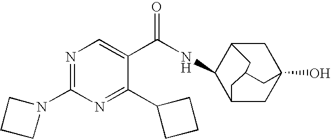

4-cyclobutyl-N-[(2r,5s)-5-hydroxyadamantan-2-yl]-2-(methylsulfonyl)pyrimidine-5-carboxamide (Intermediate 72, 270 mg, 0.67 mmol) and azetidine (125 mg, 1.33 mmol) were dissolved in THF (4 mL) and sealed into a microwave tube. The reaction was heated to 150° C. for 1 hour in the microwave reactor and cooled to room temperature. The reaction mixture was diluted with DCM (10 mL), and stirred with saturated NaHCO3 (10 mL) before passing through a phase separation cartridge. The organic layer was evaporated to afford crude product. The crude product was purified by preparative HPLC (Waters XBridge Prep C18 OBD column, 5μ silica, 50 mm diameter, 150 mm length), using decreasingly polar mixtures of water (containing 0.1% NH3) and MeCN as eluents. Fractions containing the desired compound were evaporated to dryness to afford 2-azetidin-1-yl-4-cyclobutyl-N-[(2r,5s)-5-hydroxyadamantan-2-yl]pyrimidine-5-carboxamide (103 mg, 40.4%) as a white solid.

1H NMR (400.13 MHz, DMSO-d₆) δ 1.31 (2H, d), 1.61 (4H, m), 1.70 (2H, d), 1.76 (1H, m), 1.87-2.01 (6H, m), 2.06-2.13 (2H, m), 2.22-2.35 (4H, m), 3.80-3.89 (2H, m), 4.07 (4H, t), 4.37 (1H, s), 7.96 (1H, d), 8.17 (1H, s)

m/z (ES+) (M+H)+=383; HPLC t_{R}=1.85 min.

### Intermediate 69

### Methyl 4-cyclobutyl-2-(methylthio)pyrimidine-5-carboxylate

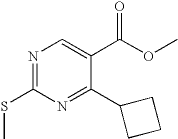

2-Methyl-2-Thiopseudourea Sulfate (1.932 g, 13.88 mmol) was added to (Z)-methyl 2-(cyclobutanecarbonyl)-3-(dimethylamino)acrylate (Intermediate 66, 2.6 g, 12.31 mmol) and sodium acetate (4.24 g, 51.69 mmol) in DMF (10 mL) at 20° C. The resulting solution was stirred at 80° C. for 2 hours. Water was added to the cooled solution. The reaction mixture was diluted with EtOAc (200 mL), and washed sequentially with water (2×100 mL).The organic layer was dried over MgSO4, filtered and evaporated to afford crude product. The crude product was purified by flash silica chromatography, elution gradient 5 to 30% EtOAc in isohexane. Pure fractions were evaporated to dryness to afford methyl 4-cyclobutyl-2-(methylthio)pyrimidine-5-carboxylate (1.300 g, 44.3%) as a white solid.

1H NMR (400.132 MHz, CDCl3) δ 1.86-1.94 (1H, m), 2.00-2.10 (1H, m), 2.26-2.35 (2H, m), 2.41-2.51 (2H, m), 2.65 (3H, s), 3.90 (3H, s), 4.35 (1H, quintet), 8.86 (1H, s)

m/z (ESI+) (M+H)+=239; HPLC t_{R}=2.75 min.

### Intermediate 70

### 4-Cyclobutyl-2-(methylthio)pyrimidine-5-carboxylic acid

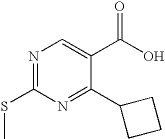

A solution of lithium hydroxide monohydrate (0.458 g, 10.91 mmol) in water (8 mL) was added to a stirred solution of methyl 4-cyclobutyl-2-(methylthio)pyrimidine-5-carboxylate (Intermediate 69, 1.3 g, 5.46 mmol) in THF (16 mL) at 20° C. The resulting mixture was stirred at 20° C. for 24 hours. The THF was evaporated and the aqueous washed with ethyl acetate (100 ml) to remove any impurities. The aqueous was acidified with 1M citric acid and extracted into ethyl acetate (100 ml). The organic layer was washed with brine (50 ml), dried over MgSO4, filtered and evaporated to give 4-cyclobutyl-2-(methylthio)pyrimidine-5-carboxylic acid (1.100 g, 90%) as a white solid.

1H NMR (400.132 MHz, CDCl3) δ 1.87-1.96 (1H, m), 2.02-2.13 (1H, m), 2.31-2.39 (2H, m), 2.44-2.54 (2H, m), 2.67 (3H, s), 4.42 (1H, quintet), 9.00 (1H, s)

m/z (ESI+) (M+H)+=225; HPLC t_{R}=0.82 min.

### Intermediate 71

### 4-cyclobutyl-N-[(2r,5s)-5-hydroxyadamantan-2-yl]-2-methylsulfanylpyrimidine-5-carboxamide

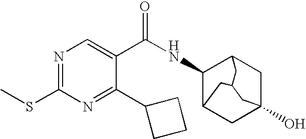

N-Ethyldiisopropylamine (3.39 mL, 19.62 mmol) was added to 4-cyclobutyl-2-(methylthio)pyrimidine-5-carboxylic acid (Intermediate 70, 1.1 g, 4.90 mmol), 4-aminoadamantan-1-ol hydrochloride (1.099 g, 5.40 mmol) and O-(7-Azabenzotriazol-1-yl)-N,N,N′,N′-tetramethyluronium hexafluorophosphate (2.238 g, 5.89 mmol) in DMF (20 mL) at 20° C. under nitrogen. The resulting solution was stirred at 20° C. for 24 hours. The reaction mixture was evaporated to dryness and redissolved in EtOAc (75 mL), and washed sequentially with water (75 mL) and saturated brine (75 mL). The organic layer was dried over MgSO4, filtered and evaporated to afford crude product. The crude product was purified by flash silica chromatography, elution gradient 0 to 6% MeOH in DCM. Pure fractions were evaporated to dryness to afford 4-cyclobutyl-N-[(2r,5s)-5-hydroxyadamantan-2-yl]-2-methylsulfanylpyrimidine-5-carboxamide (1.500 g, 82%) as a white solid.

1H NMR (400.132 MHz, CDCl3) δ 1.55-1.62 (2H, m), 1.66-1.71 (2H, m), 1.78-1.85 (5H, m), 1.91-1.97 (3H, m), 2.00-2.08 (1H, m), 2.15-2.19 (1H, m), 2.23-2.32 (4H, m), 2.43-2.52 (2H, m), 2.62 (3H, s), 3.92-4.00 (1H, m), 4.17-4.22 (1H, m), 5.90 (1H, d), 8.41 (1H, s)

m/z (ESI+) (M+H)+=374; HPLC t_{R}=2.00 min.

### Intermediate 72

### 4-Cyclobutyl-N-[(2r,5s)-5-hydroxyadamantan-2-yl]-2-methylsulfonylpyrimidine-5-carboxamide

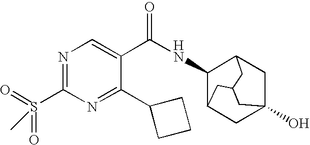

3-Chloroperoxybenzoic acid (70%) (0.937 g, 3.80 mmol) was added in one portion to 4-cyclobutyl-N-[(2r,5s)-5-hydroxyadamantan-2-yl]-2-methylsulfanylpyrimidine-5-carboxamide (Intermediate 71, 0.71 g, 1.90 mmol) in DCM (35 mL) at 0° C. The resulting solution was stirred at 20° C. for 24 hours. The reaction mixture was diluted with DCM (50 mL), and washed sequentially with saturated NaHCO3 (75 mL), 2M NaOH (75 mL), and saturated brine (75 mL). The organic layer was dried over MgSO4, filtered and evaporated to afford crude product. The crude product was purified by flash silica chromatography, elution gradient 0 to 6% MeOH in DCM. Pure fractions were evaporated to dryness to afford 4-cyclobutyl-N-[(2r,5s)-5-hydroxyadamantan-2-yl]-2-methylsulfonylpyrimidine-5-carboxamide (0.560 g, 72.6%) as a white solid.

1H NMR (400.132 MHz, CDCl3) δ 1.44 (1H, s), 1.58-1.65 (2H, m), 1.74-1.87 (6H, m), 1.93-1.98 (3H, m), 2.05-2.15 (1H, m), 2.18-2.30 (3H, m), 2.32-2.39 (2H, m), 2.43-2.55 (2H, m), 3.34 (3H, s), 4.00-4.09 (1H, m), 4.21-4.28 (1H, m), 6.42 (1H, d), 8.71 (1H, s)

m/z (ESI+) (M+H)+=406; HPLC t_{R}=1.59 min.

The following Examples were prepared in a similar manner to Example 46, using Intermediate 72 and an appropriate amine starting material:

| Structure | Ex | Name | ¹H NMR δ | MS m/e MH⁺ |
|---|---|---|---|---|
| 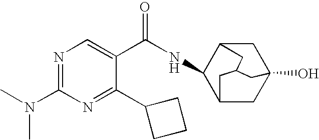 | 86 | 4-cyclobutyl-2-(dimethylamino)-N- [(2r,5s)-5-hydroxyadamantan-2- yl]pyrimidine-5-carboxamide | 1H NMR (400.13 MHz, DMSO-d₆) δ 1.31 (2H, d), 1.61 (4H, d), 1.70 (2H, d), 1.74-1.82 (1H, m), 1.88-2.02 (6H, m), 2.08-2.16 (2H, m), 2.24-2.34 (2H, m), 3.17 (6H, s), 3.84-3.92 (2H, m), 4.37 (1H, s), 7.92 (1H, d), 8.20 (1H, s) | m/z (ES+) (M + H)+ = 371; HPLC t_{R} = 1.97 min. |
| 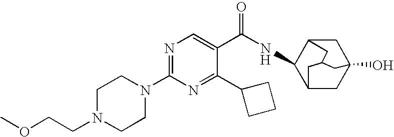 | 87 | 4-cyclobutyl-N-[(2r,5s)-5- hydroxyadamantan-2-yl]-2-[4-(2- methoxyethyl)piperazin-1- yl]pyrimidine-5-carboxamide | 1H NMR (400.13 MHz, DMSO-d₆) δ 1.31 (2H, d), 1.61 (4H, d), 1.70 (2H, d), 1.75-1.79 (1H, m), 1.88-2.01 (6H, m), 2.07-2.15 (2H, m), 2.22-2.31 (2H, m), 2.45-2.52 (6H, m), 3.24 (3H, s), 3.46 (2H, t), 3.79 (4H, t), 3.88 (2H, m), 4.37 (1H, s), 7.95 (1H, d), 8.20 (1H, s) | m/z (ES+) (M + H)+ = 470; HPLC t_{R} = 1.81 min. |
| 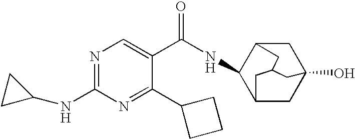 | 88 | 4-cyclobutyl-2- (cyclopropylamino)-N-[(2r,5s)-5- hydroxyadamantan-2- yl]pyrimidine-5-carboxamide | 1H NMR (400.13 MHz, DMSO-d₆) δ 0.46-0.50 (2H, m), 0.64-0.69 (2H, m), 1.31 (2H, d), 1.61 (4H, d), 1.69-1.79 (3H, m), 1.85-2.02 (6H, m), 2.06-2.13 (2H, m), 2.25-2.34 (2H, m), 2.77 (1H, m), 3.81-3.88 (2H, m), 4.37 (1H, s), 7.49 (1H, d), 7.95 (1H, d), 8.16 (1H, s) | m/z (ES+) (M + H)+ = 383; HPLC t_{R} = 1.77 min. |
| 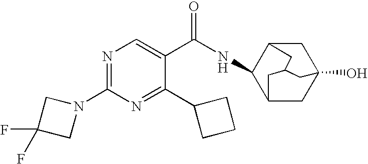 | 89 | 4-cyclobutyl-2-(3,3- difluoroazetidin-1-yl)-N-[(2r,5s)-5- hydroxyadamantan-2- yl]pyrimidine-5-carboxamide | 1H NMR (400.13 MHz, DMSO-d₆) δ 1.32 (2H, d), 1.62 (4H, d), 1.71 (2H, d), 1.78 (1H, m), 1.89-2.03 (6H, m), 2.08-2.16 (2H, m), 2.25-2.35 (2H, m), 3.81-3.91 (2H, m), 4.38 (1H, s), 4.51 (4H, t), 8.09 (1H, d), 8.27 (1H, s) | m/z (ES+) (M + H)+ = 419; HPLC t_{R} = 2.04 min. |
| 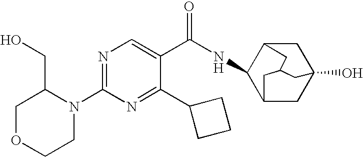 | 90 | 4-cyclobutyl-N-[(2r,5s)-5- hydroxyadamantan-2-yl]-2-[3- (hydroxymethyl)morpholin-4- yl]pyrimidine-5-carboxamide | 1H NMR (400.13 MHz, DMSO-d₆) δ 1.31 (2H, d), 1.61 (4H, d), 1.70 (2H, d), 1.78 (1H, m), 1.90-2.02 (6H, m), 2.08-2.15 (2H, m), 2.19-2.31 (2H, m), 3.08-3.17 (1H, m), 3.38-3.42 (2H, m), 3.44-3.53 (1H, m), 3.71-3.78 (1H, m), 3.82-3.95 (3H, m), 4.09 (1H, d), 4.37 (1H, s), 4.44 (1H, m), 4.55 (1H, m), 4.84 (1H, t), 7.97 (1H, d), 8.22 (1H, s) | m/z (ES+) (M + H)+ = 443; HPLC t_{R} = 1.61 min. |
| 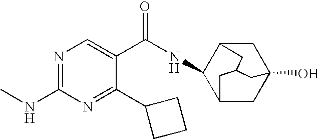 | 91 | 4-cyclobutyl-N-[(2r,5s)-5- hydroxyadamantan-2-yl]-2- (methylamino)pyrimidine-5- carboxamide | 1H NMR (400.13 MHz, DMSO-d₆) δ 1.31 (2H, d), 1.61 (4H, d), 1.69-1.76 (3M, m), 1.87-2.13 (8H, m), 2.29 (2H, m), 2.84 (3H, d), 3.84-3.91 (2H, m), 4.37 (1H, s), 7.22 (1H, d), 7.92 (1H, d), 8.14 (1H, s) | m/z (ES+) (M + H)+ = 357; HPLC t_{R} = 1.58 min. |
| 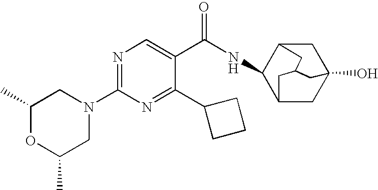 | 92 | 4-cyclobutyl-2-[(2R,6S)-2,6- dimethylmorpholin-4-yl]-N- [(2r,5s)-5-hydroxyadamantan-2- yl]pyrimidine-5-carboxamide | 1H NMR (400.13 MHz, DMSO-d₆) δ 1.16 (6H, d), 1.31 (2H, d), 1.61 (4H, d), 1.70 (2H, d), 1.76-1.81 (1H, m), 1.89-2.01 (6H, m), 2.08-2.16 (2H, m), 2.22-2.32 (2H, m), 2.53-2.59 (2H, m), 3.51-3.58 (2H, m), 3.82-3.90 (2H, m), 4.38 (1H, s), 4.60 (2H, d), 7.97 (1H, d), 8.21 (1H, s) | m/z (ES+) (M + H)+ = 441; HPLC t_{R} = 2.11 min. |
| 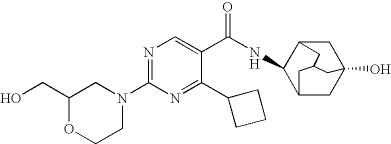 | 93 | 4-cyclobutyl-N-[(2r,5s)-5- hydroxyadamantan-2-yl]-2-[2- (hydroxymethyl)morpholin-4- yl]pyrimidine-5-carboxamide | 1H NMR (400.13 MHz, DMSO-d₆) δ 1.31 (2H, d), 1.61 (4H, d), 1.70 (2H, d), 1.78 (1H, t), 1.89-1.98 (4H, m), 2.02 (2H, s), 2.08-2.16 (2H, m), 2.23-2.33 (2H, m), 2.72-2.78 (1H, m), 2.97-3.04 (1H, m), 3.37-3.45 (2H, m), 3.48-3.55 (2H, m), 3.86 (2H, q), 3.92-3.96 (1H, m), 4.38 (1H, s), 4.53 (1H, d), 4.68 (1H, d), 4.79 (1H, t), 7.97 (1H, d), 8.23 (1H, s) | m/z (ES+) (M + H)+ = 443; HPLC t_{R} = 1.55 min. |
| 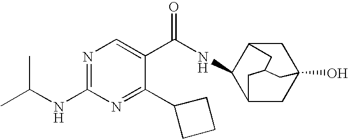 | 94 | 4-cyclobutyl-N-[(2r,5s)-5- hydroxyadamantan-2-yl]-2- (isopropylamino)pyrimidine-5- carboxamide | 1H NMR (400.13 MHz, DMSO-d₆) δ 1.16 (6H, d), 1.31 (2H, d), 1.60-1.63 (4H, m), 1.68-1.77 (3H, m), 1.85-2.01 (6H, m), 2.06-2.13 (2H, m), 2.23-2.33 (2H, m), 3.81-3.89 (2H, m), 4.11 (1H, q), 4.37 (1H, s), 7.15 (1H, d), 7.91 (1H, d), 8.13 (1H, s) | m/z (ES+) (M + H)+ = 385; HPLC t_{R} = 2.11 min. |
| 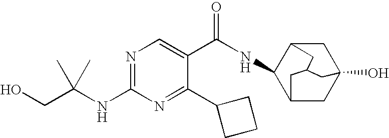 | 95 | 4-cyclobutyl-N-[(2r,5s)-5- hydroxyadamantan-2-yl]-2-[(2- hydroxy-1,1- dimethylethyl)amino]pyrimidine-5- carboxamide | 1H NMR (400.13 MHz, DMSO-d₆) δ 1.31 (2H, d), 1.36 (6H, s), 1.61 (4H, m), 1.70 (2H, d), 1.79 (1H, m), 1.88-2.01 (6H, m), 2.08-2.15 (2H, m), 2.22-2.32 (2H, m), 3.52 (2H, d), 3.80-3.88 (2H, m), 4.37 (1H, s), 4.92 (1H, t), 6.53 (1H, s), 7.95 (1H, d), 8.12 (1H, s) | m/z (ES+) (M + H)+ = 415; HPLC t_{R} = 1.68 min. |
| 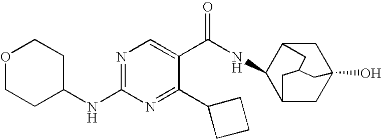 | 96 | 4-cyclobutyl-N-[(2r,5s)-5- hydroxyadamantan-2-yl]-2- (tetrahydro-2H-pyran-4- ylamino)pyrimidine-5-carboxamide | 1H NMR (400.13 MHz, DMSO-d-₆) δ 1.31 (2H, d), 1.48-1.58 (2H, m), 1.61 (4H, m), 1.68-2.01 (11H, m), 2.10 (2H, q), 2.23-2.32 (2H, m), 3.35-3.41 (2H, m), 3.86 (4H, m), 3.98 (1H, m), 4.37 (1H, s), 7.31 (1H, d), 7.92 (1H, d), 8.13 (1H, s) | m/z (ES+) (M + H)+ = 427; HPLC t_{R} = 1.67 min. |
| 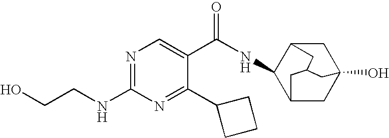 | 97 | 4-cyclobutyl-N-[(2r,5s)-5- hydroxyadamantan-2-yl]-2-[(2- hydroxyethyl)amino]pyrimidine-5- carboxamide | 1H NMR (400.13 MHz, DMSO-d₆) δ 1.31 (2H, d), 1.60-1.63 (4H, m), 1.69-1.77 (3H, m), 1.85-2.01 (6H, m), 2.06-2.13 (2H, m), 2.23-2.32 (2H, m), 3.39 (2H, m), 3.53 (2H, m), 3.81-3.87 (2H, m), 4.37 (1H, s), 4.63 (1H, t), 7.18 (1H, t), 7.93 (1H, d), 8.13 (1H, s) | m/z (ES+) (M + H)+ = 387; HPLC t_{R} = 1.38 min. |
| 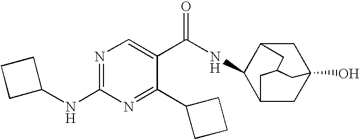 | 98* | N-[(2r,5s)-5-hydroxyadamantan-2- yl] 4-cyclobutyl-2- (cyclobutylamino)pyrimidine-5- carboxamide | 1H NMR (400.13 MHz, DMSO-d₆) δ 1.31 (2H, d), 1.39 (1H, s), 1.60-1.77 (8H, m), 1.87-2.01 (8H, m), 2.09 (2H, m), 2.23-2.33 (4H, m), 3.80-3.89 (2H, m), 4.37-4.42 (2H, m), 4.37-4.42 (2H, m), 7.59 (1H, d), 7.92 (1H, d), 8.12 (1H, s) | m/z (ES+) (M + H)+ = 397; HPLC t_{R} = 2.05 min. |
| 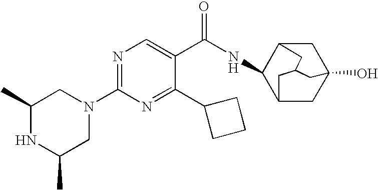 | 99 | 4-cyclobutyl-2-[(3R,5S)-3,5- dimethylpiperazin-1-yl]-N-[(2r,5s)- 5-hydroxyadamantan-2- yl]pyrimidine-5-carboxamide | 1H NMR (400.13 MHz, DMSO-d₆) d 1.02 (6H, d), 1.31 (2H, d), 1.61 (4H, d), 1.70 (2H, d), 1.75-1.81 (1H, m), 1.88-2.01 (6H, m), 2.07-2.15 (2H, m), 2.21-2.31 (3H, m), 2.33-2.39 (2H, m), 2.62-2.70 (2H, m), 3.82-3.90 (2H, m), 4.38 (1H, s), 4.63 (2H, d), 7.93 (1H, d), 8.18 (1H, s) | m/z (ES+) (M + H)+ = 440; HPLC t_{R} = 1.77 min. |
| 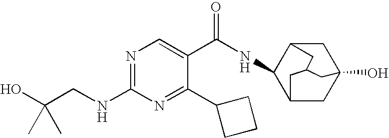 | 100 | 4-cyclobutyl-N-[(2r,5s)-5- hydroxyadamantan-2-yl]-2-[(2- hydroxy-2- methylpropyl)amino]pyrimidine-5- carboxamide | 1H NMR (400.13 MHz, DMSO-d₆) d 1.11 (6H, s), 1.31 (2H, d), 1.61 (4H, d), 1.68-1.77 (3H, m), 1.87-1.98 (4H, m), 2.02 (2H, s), 2.05-2.13 (2H, m), 2.23-2.33 (2H, m), 3.36 (2H, s), 3.83-3.87 (2H, m), 4.37 (1H, s), 4.58 (1H, s), 7.00 (1H, s), 7.95 (1H, d), 8.12 (1H, s) | m/z (ES+) (M + H)+ = 415; HPLC t_{R} = 1.55 min. |
| 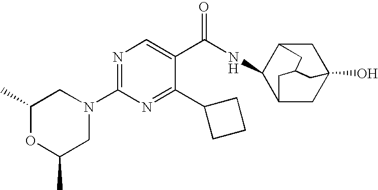 | 101 | 4-cyclobutyl-2-[(2R,6R)-2,6- dimethylmorpholin-4-yl]-N- [(2r,5s)-5-hydroxyadamantan-2- yl]pyrimidine-5-carboxamide | 1H NMR (400.13 MHz, DMSO-d₆) d 1.12 (6H, d), 1.31 (2H, d), 1.61 (4H, d), 1.70 (2H, d), 1.74-1.82 (1H, m), 1.88-1.97 (4H, m), 2.01 (2H, s), 2.08-2.16 (2H, m), 2.20-2.33 (2H, m), 3.54 (2H, m), 3.82-3.92 (4H, m), 3.96-4.03 (2H, m), 4.38 (1H, s), 7.98 (1H, d), 8.21 (1H, s) | m/z (ES+) (M + H)+ = 441; HPLC t_{R} = 2.07 min. |
| 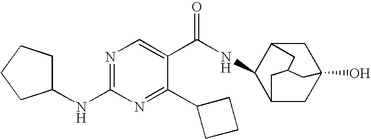 | 102 | 4-cyclobutyl-2-(cyclopentylamino)- N-[(2r,5s)-5-hydroxyadamantan-2- yl]pyrimidine-5-carboxamide | 1H NMR (400.13 MHz, DMSO-d₆) d 1.31 (2H, m), 1.48-1.55 (4H, m), 1.61 (4H, d), 1.68-1.76 (5H, m), 1.85-1.97 (6H, m), 2.01 (2H, s), 2.09 (2H, m), 2.24-2.33 (2H, m), 3.81-3.87 (2H, m), 4.21 (1H, m), 4.37 (1H, s), 7.30 (1H, d), 7.91 (1H, d), 8.12 (1H, s) | m/z (ES+) (M + H)+ = 441; HPLC t_{R} = 2.07 min. |
| 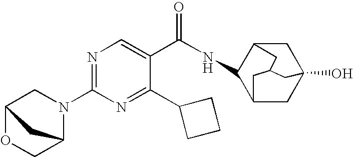 | 103 | 4-cyclobutyl-N-[(2r,5s)-5- hydroxyadamantan-2-yl]-2- [(1S,4S)-2-oxa-5- azabicyclo[2.2.1]hept-5- yl]pyrimidine-5-carboxamide | 1H NMR (400.13 MHz, DMSO-d₆) d 1.31 (2H, d), 1.61 (4H, d), 1.70 (2H, d), 1.76-1.81 (1H, m), 1.86-1.98 (6H, m), 2.02 (2H, s), 2.11 (2H, m), 2.23-2.34 (2H, m), 3.45-3.51 (2H, m), 3.64 (1H, d), 3.81-3.88 (3H, m), 4.38 (1H, s), 4.67 (1H, s), 5.00 (1H, s), 7.96 (1H, d), 8.19 (1H, s) | m/z (ES+) (M + H)+ = 425; HPLC t_{R} = 1.73 min. |
| 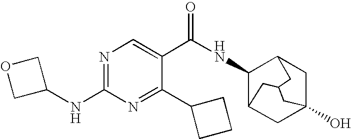 | 104 | 4-cyclobutyl-N-[(2r,5s)-5- hydroxyadamantan-2-yl]-2- (oxetan-3-ylamino)pyrimidine-5- carboxamide | 1H NMR (400.13 MHz, DMSO-d₆) d 1.31 (2H, d), 1.61 (4H, d), 1.68-1.80 (3H, m), 1.89-1.97 (4H, m), 2.01 (2H, m), 2.07-2.14 (2H, m), 2.21-2.31 (2H, m), 3.80-3.88 (2H, m), 4.37 (1H, s), 4.53 (2H, t), 4.77 (2H, t), 4.89-4.95 (1H, m), 7.97 (1H, d), 8.07 (1H, d), 8.15 (1H, s) | m/z (ES+) (M + H)+ = 399; HPLC t_{R} = 1.48 min. |
| 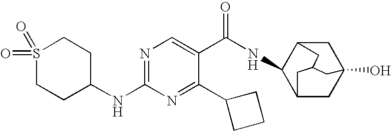 | 105 | 4-cyclobutyl-2-[(1,1- dioxidotetrahydro-2H-thiopyran-4- yl)amino]-N-[(2r,5s)-5- hydroxyadamantan-2- yl]pyrimidine-5-carboxamide | 1H NMR (400.13 MHz, DMSO-d₆) d 1.31 (2H, d), 1.61 (4H, d), 1.68-1.77 (3H, m), 1.89-2.01 (6H, m), 2.11 (6H, m), 2.24-2.34 (3H, m), 3.12-3.23 (3H, m), 3.80-3.87 (2H, m), 4.14-4.17 (1H, m), 4.37 (1H, s), 7.53 (1H, d), 7.95 (1H, d), 8.15 (1H, s) | m/z (ES−) (M − H)− = 473; HPLC t_{R} = 1.53 min. |

### Footnotes

Example 98 may be prepared as follows:

Cyclobutylamine (4.00 mL, 46.85 mmol) was added to 4-cyclobutyl-N-[(2r,5s)-5-hydroxyadamantan-2-yl]-2-methylsulfonylpyrimidine-5-carboxamide (Intermediate 72, 3.8 g, 9.37 mmol) in THF (60 mL). The resulting solution was stirred at 20° C. for 70 hours. The reaction mixture was evaporated to dryness and redissolved in EtOAc (150 mL), and washed sequentially with water (150 mL) and saturated brine (150 mL). The organic layer was dried over MgSO4, filtered and evaporated to afford crude product. The crude gum was triturated with DCM to give a solid which was collected by filtration. The filtrate was purified by flash silica chromatography, elution gradient 0 to 5% MeOH in DCM. Pure fractions were evaporated to dryness to afford 4-cyclobutyl-2-(cyclobutylamino)-N-[(2r,5s)-5-hydroxyadamantan-2-yl]pyrimidine-5-carboxamide as a white foam. The solid from the trituration and the foam were combined and triturated with ethyl acetate to give 4-cyclobutyl-2-(cyclobutylamino)-N-[(2r,5s)-5-hydroxyadamantan-2-yl]pyrimidine-5-carboxamide (2.125 g, 57.2%) as a white solid.

1H NMR (400.132 MHz, CDCl3) δ 1.42 (1H, s), 1.52-1.57 (2H, m), 1.66-1.71 (2H, m), 1.76-1.82 (6H, m), 1.88-2.04 (6H, m), 2.15-2.26 (5H, m), 2.36-2.48 (4H, m), 3.95 (1H, quintet), 4.14-4.21 (1H, m), 4.42-4.59 (1H, m), 5.47 (1H, s), 5.81 (1H, d), 8.28 (1H, s)

m/z (ES+) (M+H)+=397; HPLC t_{R}=2.05 min.

The following Examples were prepared in a similar manner to Example 75, using

Intermediate 72 and an appropriate starting material:

| | | | | MS m/e |
|---|---|---|---|---|
| Structure | Ex | Name | ¹H NMR δ | MH⁺ |
| 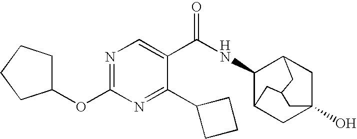 | 106 | 4-cyclobutyl-2- (cyclopentyloxy)-N- [(2r,5s)-5- hydroxyadam antan-2- yl]pyrimidine- 5- carboxamide | 1H NMR (400.13 MHz, DMSO-d₆) d 1.33 (2 H, d), 1.57-1.66 (6 H, m), 1.70-1.84 (7 H, m), 1.88-2.03 (8 H, m), 2.11-2.19 (2 H, m), 2.25-2.35 (2 H, m), 3.84 (1 H, m), 3.92 (1 H, m), 4.39 (1 H, s), 5.38- 5.42 (1 H, m), 8.20 (1 H, d), 8.37 (1 H, s) | m/z (ES+) (M + H)+ = 412; HPLC t_{R} = 2.24 min. |
| 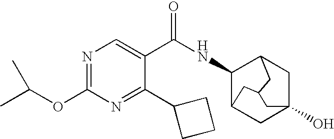 | 107 | 4-cyclobutyl- N-[(2r,5s)-5- hydroxyadam antan-2-yl]-2- isopropoxypyri- midine-5- carboxamide | 1H NMR (400.13 MHz, DMSO-d₆) δ 1.31 (2 H, s), 1.34 (6 H, d), 1.62 (4 H, d), 1.71 (2 H, d), 1.76-1.84 (1 H, m), 1.88-1.98 (4 H, m), 2.02 (2 H, s), 2.11-2.19 (2 H, m), 2.25-2.34 (2 H, m), 3.84 (1 H, m), 3.92 (1 H, m), 4.39 (1 H, s), 5.23-5.30 (1 H, m), 8.19 (1 H, d), 8.37 (1 H, s) | m/z (ES+) (M + H)+ = 386; HPLC t_{R} = 2.02 min. |
| 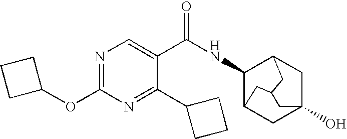 | 108 | 4-cyclobutyl- 2- (cyclobutyloxy)- N-[(2r,5s)- 5- hydroxyadam antan-2- yl]pyrimidine- 5- carboxamide | 1H NMR (400.13 MHz, DMSO-d₆) d 1.32 (2 H, d), 1.61-1.73 (7 H, m), 1.77-1.85 (2 H, m), 1.88-1.99 (4 H, m), 2.01-2.06 (2 H, m), 2.08-2.19 (4 H, m), 2.24-2.34 (2 H, m), 2.40-2.47 (2 H, m), 3.80-3.89 (1 H, m), 3.92 (1 H, m), 4.39 (1 H, s), 5.13-5.20 (1 H, m), 8.21 (1 H, d), 8.37 (1 H, s) | m/z (ES+) (M + H)+ = 398; HPLC t_{R} = 2.11 min. |
| 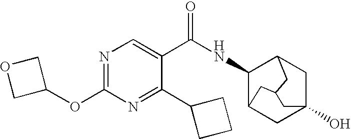 | 109 | 4-cyclobutyl- N-[(2r,5s)-5- hydroxyadam antan-2-yl]-2- (oxetan-3- yloxy)pyrimidine- 5- carboxamide | 1H NMR (400.13 MHz, DMSO-d₆) d 1.33 (2 H, d), 1.62 (4 H, d), 1.71 (2 H, d), 1.75-1.85 (1 H, m), 1.88-2.03 (6 H, m), 2.08-2.19 (2 H, m), 2.22-2.30 (2 H, m), 3.80-3.89 (1 H, m), 3.92 (1 H, m), 4.39 (1 H, s), 4.59-4.62 (2 H, m), 4.91 (2 H, t), 5.58-5.63 (1 H, m), 8.24 (1 H, d), 8.40 (1 H, s) | m/z (ES+) (M + H)+ = 400; HPLC t_{R} = 1.57 min. |

The following Examples were prepared in a similar manner to Example 75, using Intermediate 86 and an appropriate starting material:

| | | | | MS m/e |
|---|---|---|---|---|
| Structure | Ex | Name | ¹H NMR δ | MH⁺ |
| 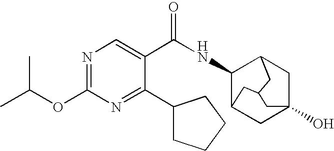 | 110 | 4- cyclopentyl- N-[(2r,5s)-5- hydroxyadam antan-2-yl]- 2-propan-2- yloxypyrimidine- 5- carboxamide | 1H NMR (400.132 MHz, CDCl3) δ 1.40 (7 H, d), 1.57-1.62 (2 H, m), 1.66- 1.72 (4 H, m), 1.78- 2.05 (12 H, m), 2.15- 2.28 (3 H, m), 3.51-3.56 (1 H, m), 4.20-4.24 (1 H, m), 5.26-5.35 (1 H, m), 5.92 (1 H, d), 8.44 (1 H, s) | m/z (ESI+) (M + H)+ = 400; HPLC t_{R} = 2.15 min. |
| 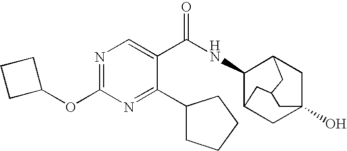 | 111 | 2- cyclobutyloxy-4- cyclopentyl- N-[(2r,5s)-5- hydroxyadam antan-2- yl]pyrimidine- 5- carboxamide | 1H NMR (400.132 MHz, CDCl3) δ 1.41 (1 H, s), 1.59 (2 H, d), 1.63-2.05 (18 H, m), 2.15-2.30 (5 H, m), 2.42-2.51 (2 H, m), 3.51 (1 H, quintet), 4.20 (1 H, d), 5.19 (1 H, quintet), 5.93 (1 H, d), 8.43 (1 H, s) | m/z (ESI+) (M + H)+ = 412; HPLC t_{R} = 2.29 min. |
| 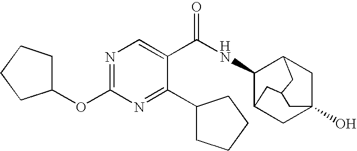 | 112 | 4- cyclopentyl-2- cyclopentyloxy-N- [(2r,Ss)-5- hydroxyadam antan-2- yl]pyrimidine- 5- carboxamide | 1H NMR (400.132 MHz, CDCl3) δ 1.41 (1 H, s), 1.52-2.07 (26 H, m), 2.19 (1 H, s), 2.26 (2 H, s), 3.52 (1 H, t), 4.20 (1 H, d), 5.41 (1 H, septet), 5.93 (1 H, d), 8.44 (1 H, s) | m/z (ESI+) (M + H)+ = 426; HPLC t_{R} = 2.44 min. |
| 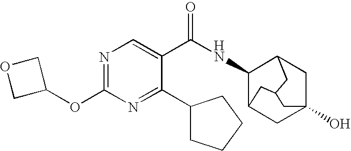 | 113 | 4- cyclopentyl- N-[(2r,5s)-5- hydroxyadamantan- 2-yl]- 2-(oxetan-3- yloxy)pyrimidine-5- carboxamide | 1H NMR (400.132 MHz, CDCl3) δ 1.46 (1 H, s), 1.59 (2 H, d), 1.65-1.74 (4 H, m), 1.77-1.90 (8 H, m), 1.90-2.05 (5 H, m), 2.19 (1 H, s), 2.26 (2 H, s), 3.51 (1 H, quintet), 4.21 (1 H, d), 4.80 (2 H, t), 4.97 (2 H, t), 5.61 (1 H, quintet), 5.96 (1 H, d), 8.44 (1 H, s) | m/z (ESI+) (M + H)+ = 414; HPLC t_{R} = 1.69 min. |

### Intermediate 83

### methyl 4-cyclopentyl-2-(methylthio)pyrimidine-5-carboxylate

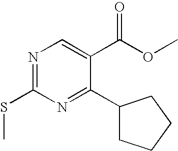

Prepared from Intermediate 53 by the same process used for Intermediate 28

1H NMR (400.132 MHz, CDCl3) δ 1.67-1.72 (2H, m), 1.79-1.92 (4H, m), 1.99-2.05 (2H, m), 2.58 (3H, s), 3.91 (3H, s), 3.99-4.09 (1H, m), 8.85 (1H, s)

m/z (ESI+) (M+H)+=253; HPLC t_{R}=3.04 min.

### Intermediate 84

### 4-cyclopentyl-2-(methylthio)pyrimidine-5-carboxylic acid

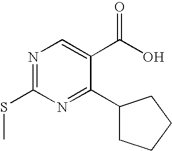

Prepared from Intermediate 83 by the Same Process Used for Intermediate 21

1H NMR (400.132 MHz, CDCl3) δ 1.68-1.75 (2H, m), 1.81-1.96 (4H, m), 2.00-2.10 (2H, m), 2.61 (3H, s), 4.13 (1H, quintet), 9.00 (1H, s), 11.21 (1H, bs)

m/z (ESI+) (M+H)+=239; HPLC t_{R}=1.19 min.

### Intermediate 85

### 4-cyclopentyl-N-[(2r,5s)-5-hydroxyadamantan-2-yl]-2-methylsulfanylpyrimidine-5-carboxamide

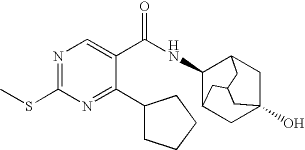

Prepared from Intermediate 84 by the Same Process Used for Example 4

1H NMR (400.132 MHz, CDCl3) δ 1.35-1.42 (1H, m), 1.58-1.62 (2H, m), 1.65-1.72 (4H, m), 1.79-2.01 (12H, m), 2.16-2.21 (1H, m), 2.24-2.27 (2H, m), 2.56 (3H, s), 3.51 (1H, quintet), 4.18-4.23 (1H, m), 5.92 (1H, d), 8.42 (1H, s)

m/z (ESI+) (M+H)+=388; HPLC t_{R}=2.20 min.

### Intermediate 86

### 4-cyclopentyl-N-[(2r,5s)-5-hydroxyadamantan-2-yl]-2-methylsulfonylpyrimidine-5-carboxamide

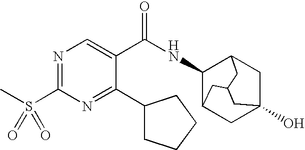

Prepared from Intermediate 85 by the Same Process Used for Example 37

1H NMR (400.132 MHz, CDCl3) δ 1.57-1.63 (2H, m), 1.69-1.99 (15H, m), 2.04-2.09 (2H, m), 2.17-2.23 (1H, m), 2.27-2.33 (2H, m), 3.30 (3H, s), 3.57 (1H, quintet), 4.23-4.27 (1H, m), 6.43 (1H, d), 8.72 (1H, s)

m/z (ESI+) (M+H)+=420; HPLC t_{R}=1.75 min.

### Example 114

### 4-Cyclopentyl-N-[(2s,5r)-5-hydroxyadamantan-2-yl]-2-thiomorpholin-4-ylpyrimidine-5-carboxamide

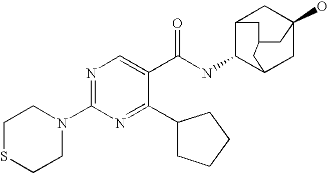

Prepared from Intermediate 86 by the Same Process Used for Example 36

1H NMR (400.13 MHz, DMSO-d6) δ 1.32 (2H, d), 1.56 (1H, d), 1.61 (5H, d), 1.69-1.75 (2H, m), 1.72-1.76 (3H, m), 1.77-1.79 (1H, m), 1.85 (2H, d), 1.89 (1H, d), 1.93 (1H, s), 1.98 (1H, s), 2.02 (2H, s), 2.57-2.60 (4H, m), 3.41-3.49 (1H, m), 3.90 (1H, t), 4.07-4.10 (4H, m), 4.37 (1H, s), 8.07 (1H, d), 8.22 (1H, s)

m/z (ESI+) (M+H)+=443; HPLC t_{R}=2.41 min.

### Example 115

### 4-Cyclopentyl-N-[(2r,5 s)-5-hydroxyadamantan-2-yl]-2-(1-oxo-1,4-thiazinan-4-yl)pyrimidine-5-carboxamide

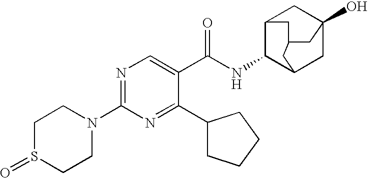

Prepared from Example 114 by the same process used for Example 36

1H NMR (400.132 MHz, CDCl3) δ 1.47 (1H, s), 1.58 (2H, d), 1.64-1.75 (4H, m), 1.75-1.90 (8H, m), 1.91-2.02 (4H, m), 2.18 (1H, s), 2.24 (2H, s), 2.69-2.78 (2H, m), 2.83 (2H, d), 3.56 (1H, quintet), 4.15-4.25 (3H, m), 4.58 (2H, d), 5.92 (1H, d), 8.34 (1H, s)

m/z (ESI+) (M+H)+=459; HPLC t_{R}=1.59 min.

### Example 116

### 4-Cyclopentyl-2-(1,1-dioxo-1,4-thiazinan-4-yl)-N-[(2r,5s)-5-hydroxyadamantan-2-yl]pyrimidine-5-carboxamide

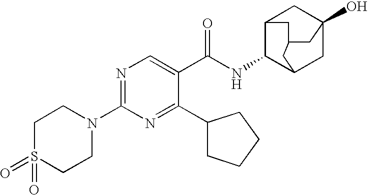

Prepared from Example 114 by the Same Process Used for Example 37

1H NMR (400.132 MHz, CDCl3) δ 1.40 (1H, s), 1.59 (2H, d), 1.64-1.74 (4H, m), 1.76-1.87 (8H, m), 1.90-2.03 (4H, m), 2.19 (1H, s), 2.25 (2H, s), 3.03 (4H, t), 3.55 (1H, quintet), 4.20 (1H, d), 4.37-4.42 (4H, m), 5.89 (1H, d), 8.35 (1H, s)

m/z (ESI+) (M+H)+=475; HPLC t_{R}=1.87 min.

The following Examples were prepared in a similar manner to Example 46, using Intermediate 86 and an appropriate starting material:

| | | | | MS m/e |
|---|---|---|---|---|
| Structure | Ex | Name | ¹H NMR δ | MH+ |
| 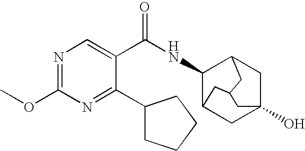 | 117 | 4- cyclopentyl- N-[(2r,5s)-5- hydroxyadam antan-2-yl]- 2- methoxypyrimi- dine-5- carboxamide | 1H NMR (400.132 MHz, CDCl3) δ 1.37 (1 H, s), 1.57-1.62 (2 H, m), 1.66-1.74 (4 H, m), 1.78-2.04 (12 H, m), 2.16-2.29 (3 H, m), 3.51-3.58 (1 H, m), 4.02 (3 H, s), 4.18-4.24 (1 H, m), 5.91 (1 H, d), 8.48 (1 H, s) | m/z (ESI+) (M + H)+ = 372; HPLC t_{R} = 1.78 min |
| 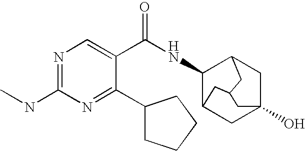 | 118 | 4- cyclopentyl- N-[(2r,5s)-5- hydroxyadam antan-2-yl]- 2- methylamino pyrimidine- 5- carboxamide | 1H NMR (400.132 MHz, CDCl3) δ 1.37 (1 H, s), 1.56-1.60 (2 H, m), 1.63-1.73 (4 H, m), 1.77-1.97 (12 H, m), 2.16-2.27 (3 H, m), 3.02 (3 H, d), 3.50-3.57 (1 H, m), 4.15-4.20 (1 H, m), 5.17 (1 H, d), 5.86 (1 H, d), 8.29 (1 H, s) | m/z (ESI+) (M + H)+ = 371; HPLC t_{R} = 1.74 min |
| 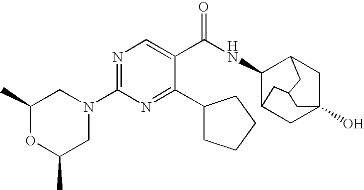 | 119 | 4- cyclopentyl- 2-[(2S,6R)- 2,6- dimethylmor- pholin-4-yl]- N-[(2r,5s)-5- hydroxyadamantan- 2- yl]pyrimidine- 5- carboxamide | 1H NMR (400.132 MHz, CDCl3) δ 1.26 (6 H, d), 1.37 (1 H, s), 1.55-1.60 (2 H, m), 1.64-1.71 (4 H, m), 1.78-1.98 (12 H, m), 2.15-2.26 (3 H, m), 2.56-2.63 (2 H, m), 3.53-3.65 (3 H, m), 4.14-4.19 (1 H, m), 4.62 (2 H, d), 5.87 (1 H, d), 8.30 (1 H, s) | m/z (ESI+) (M + H)+ = 455; HPLC t_{R} = 2.28 min |
| 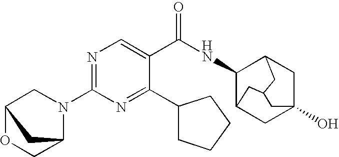 | 120 | 4- cyclopentyl- N-[(2r,5s)-5- hydroxyadam antan-2-yl]- 2-[(1S,4S)-2- oxa-5- azabicyclo[2. 2.1]heptan-5- yl]pyrimidine- 5- carboxamide | 1H NMR (400.132 MHz, MHz, CDCl3) δ 1.37 (1 H, s), 1.55-1.61 (2 H, m), 1.63-1.72 (4 H, m), 1.76-1.99 (14 H, m), 2.14-2.26 (3 H, m), 3.53-3.63 (3 H, m), 3.83-3.91 (2 H, m), 4.15-4.21 (1 H, m), 4.71 (1 H, s), 5.09 (1 H, s), 5.85 (1 H, d), 8.31 (1 H, s) | m/z (ESI+) (M + H)+ = 439; HPLC t_{R} = 1.85 min |
| 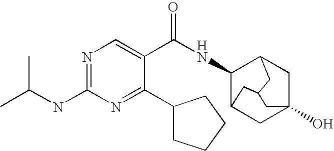 | 121 | 4- cyclopentyl- N-[(2r,5s)-5- hydroxyadam antan-2-yl]- 2-(propan-2- ylamino)pyrimi- dine-5- carboxamide | 1H NMR (400.132 MHz, CDCl3) δ 1.25 (6 H, d), 1.37 (1 H, s), 1.56-1.61 (2 H, m), 1.64-1.73 (4 H, m), 1.77-1.97 (12 H, m), 2.13-2.27 (3 H, m), 3.50-3.57 (1 H, m), 4.10-4.22 (2 H, m), 5.06 (1 H, d), 5.86 (1 H, d), 8.27 (1 H, s) | m/z (ESI+) (M + H)+ = 399; HPLC t_{R} = 2.14 min |
| 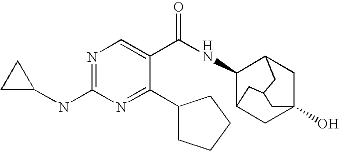 | 122 | 4- cyclopentyl- 2- (cyclopropyl amino)-N- [(2r,5s)-5- hydroxyadam antan-2- yl]pyrimidine- 5- carboxamide | 1H NMR (400.132 MHz, CDCl3) δ 0.54- 0.58 (2 H, m), 0.79- 0.89 (2 H, m), 1.37 (1 H, s), 1.55-1.60 (2 H, m), 1.62-1.72 (4 H, m), 1.77-1.98 (12 H, m), 2.14-2.25 (3 H, m), 2.76-2.82 (1 H, m), 3.52-3.56 (1 H, m), 4.16-4.21 (1 H, m), 5.39 (1 H, s), 5.87 (1 H, d), 8.35 (1 H, s) | m/z (ESI+) (M + H)+ = 397; HPLC t_{R} = 1.93 min |
| 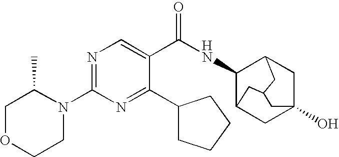 | 123 | 4- cyclopentyl- N-[(2r,5s)-5- hydroxyadam antan-2-[(3S)-3- methylmor- pholin-4- yl]pyrimidine- 5- carboxamide | 1H NMR (400.132 MHz, CDCl3) δ 1.29 (3 H, d), 1.35 (1 H, s), 1.56-1.60 (2 H, m), 1.62-1.73 (4 H, m), 1.76-1.99 (12 H, m), 2.14-2.25 (3 H, m), 3.21-3.31 (1 H, m), 3.51-3.63 (2 H, m), 3.68-3.80 (2 H, m), 3.96-4.00 (1 H, m), 4.15-4.20 (1 H, m), 4.40 (1 H, d), 4.73 (1 H, d), 5.85 (1 H, d), 8.33 (1 H, s) | m/z (ESI+) (M + H)+ = 441; HPLC t_{R} = 2.12 min |
| 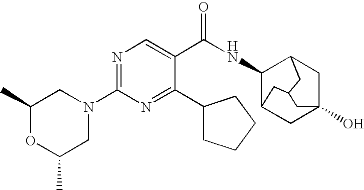 | 124 | 4- cyclopentyl- 2-[(2S,6S)- 2,6- dimethylmor- pholin-4-yl]- N-[(2r,5s)-5- hydroxyadam antan-2- yl]pyrimidine- 5- carboxamide | 1H NMR (400.132 MHz, CDCl3) δ 1.22 (6 H, d), 1.36 (1 H, s), 1.55-1.60 (2 H, m), 1.62-1.72 (4 H, m), 1.78-1.97 (12 H, m), 2.14-2.25 (3 H, m), 3.53-3.66 (3 H, m), 3.94 (2 H, d), 4.03- 4.09 (2 H, m), 4.15- 4.20 (1 H, m), 5.87 (1 H, d), 8.33 (1 H, s) | m/z (ESI+) (M + H)+ = 455; HPLC t_{R} = 2.20 min |
| 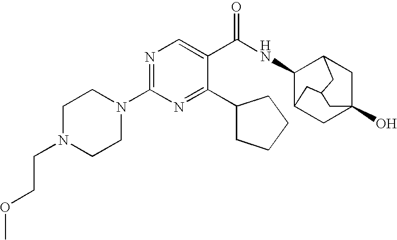 | 125 | 4- cyclopentyl- N-[(2r,5s)-5- hydroxyadam antan-2-yl]- 2-[4-(2- methoxyethyl) piperazin-1- yl]pyrimidine- 5- carboxamide | 1H NMR (400.132 MHz, CDCl3) δ 1.31 (1 H, s), 1.45-1.53 (2 H, m), 1.54-1.67 (4 H, m), 1.68-1.93 (12 H, m), 2.09 (1 H, s), 2.16 (2 H, s), 2.47 (4 H, t), 2.55 (2 H, t), 3.30 (3 H, s), 3.45-3.53 (3 H, m), 3.83 (4 H, t), 4.10 (1 H, d), 5.78 (1 H, d), 8.24 (1 H, s) | m/z (ESI+) (M + H)+ = 484; HPLC t_{R} = 1.13 min. |
| 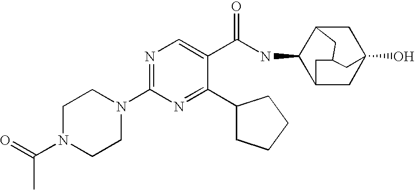 | 126 | 2-(4- acetylpiperazin- 1-yl)-4- cyclopentyl- N-[(2r,5s)-5- hydroxyadam antan-2- yl]pyrimidine- 5- carboxamide | 1H NMR (400.13 MHz, DMSO-d6) δ 1.30-1.33 (2 H, m), 1.53-1.63 (7 H, m), 1.69-1.93 (11 H, m), 2.04 (5 H, s), 3.49- 3.52 (4 H, m), 3.72- 3.74 (2 H, m), 3.80 (2 H, t), 3.90 (1 H, t), 4.38 (1 H, s), 8.06 (1 H, d), 8.23 (1 H, s) | m/z (ESI+) (M + H)+ = 468; HPLC t_{R} = 1.78 min. |
| 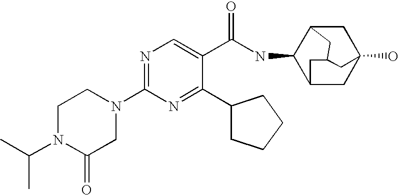 | 127 | 4- cyclopentyl- N-[(2r,5s)-5- hydroxyadam antan-2-yl]- 2-(3-oxo-4- propan-2- ylpiperazin- 1- yl)pyrimidine- 5- carboxamide | 1H NMR (400.13 MHz, DMSO-d6) δ 1.07 (6 H, d), 1.30- 1.33 (2 H, m), 1.59 (6 H, t), 1.69-2.03 (13 H, m), 3.33 (2 H, q), 3.42- 3.50 (1 H, m), 3.89- 3.94 (3 H, m), 4.24 (2 H, s), 4.38 (1 H, s), 4.63- 4.69 (1 H, m), 8.08 (1 H, d), 8.25 (1 H, s) | m/z (ESI+) (M + H)+ = 482; HPLC t_{R} = 2.01 min. |
| 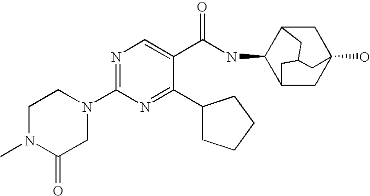 | 128 | 4- cyclopentyl- N-[(2r,5s)-5- hydroxyadam antan-2-yl]- 2-(4-methyl- 3- oxopiperazin- 1- yl)pyrimidine- 5- carboxamide | 1H NMR (400.132 MHz, DMSO-d6) δ 1.30-1.33 (2 H, m), 1.52-1.64 (6 H, m), 1.69-1.93 (10 H, m), 1.98-2.03 (3 H, m), 2.88 (3 H, s), 3.39 (2 H, t), 3.44-3.50 (1 H, m), 3.90 (1 H, t), 3.99-4.06 (2 H, m), 4.26 (2 H, s), 4.38 (1 H, s), 8.09 (1 H, d), 8.25 (1 H, s) | m/z (ESI+) (M + H)+ = 454; HPLC t_{R} = 1.74 min. |
| 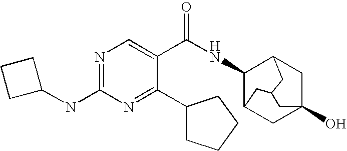 | 129 | 4- cyclopentyl- 2- (cyclobutylamino)- N- [(2r,5s)-5- hydroxyadam antan-2- yl]pyrimidine- 5- carboxamide | 1H NMR (400.132 MHz, CDCl3) δ 1.41 (1 H, s), 1.57 (2 H, d), 1.61-2.00 (18 H, m), 2.17 (1 H, s), 2.23 (2 H, s), 2.37-2.46 (2 H, m), 3.52 (1 H, t), 4.17 (1 H, d), 4.44 (1 H, q), 5.37 (1 H, d), 5.86 (1 H, d), 8.26 (1 H, s) | m/z (ESI+) (M + H)+ = 411; HPLC t_{R} = 2.13 min. |
| 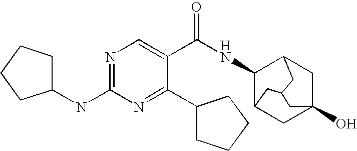 | 130 | 4- cyclopentyl- 2- (cyclopentylamino)- N- [(2r,5s)-5- hydroxyadam antan-2- yl]pyrimidine- 5- carboxamide | 1H NMR (400.132 MHz, CDCl3) δ 1.26 (1 H, s), 1.38-1.53 (3 H, m), 1.57 (2 H, d), 1.61-2.00 (19 H, m), 2.06 (2 H, sextet), 2.17 (1 H, s), 2.23 (2 H, s), 3.53 (1 H, t), 4.17 (1 H, d), 4.27 (1 H, q), 5.22 (1 H, d), 5.87 (1 H, d), 8.28 (1 H, s) | m/z (ESI+) (M + H)+ = 425; HPLC t_{R} = 2.26 min. |
| 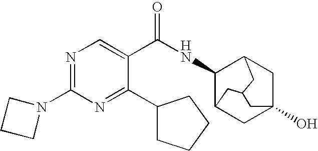 | 131 | 2-(azetidin-1- yl)-4- cyclopentyl- N-[(2r,5s)-5- hydroxyadam antan-2- yl]pyrimidine- 5- carboxamide | 1H NMR (400.132 MHz, CDCl3) δ 1.40 (1 H, s), 1.56 (2 H, d), 1.60-1.73 (4 H, m), 1.74-1.98 (12 H, m), 2.17 (1 H, s), 2.23 (2 H, s), 2.37 (2 H, quintet), 3.53 (1 H, quintet), 4.16 (5 H, m), 5.85 (1 H, d), 8.29 (1 H, s) | m/z (ESI+) (M + H)+ = 397; HPLC t_{R} = 2.06 min. |
| 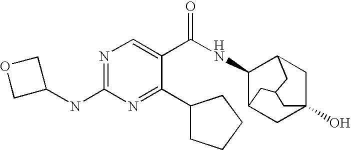 | 132 | 4- cyclopentyl- N-[(2r,5s)-5- hydroxyadam antan-2-yl]- 2-(oxetan-3- ylamino)pyrimi- dine-5- carboxamide | 1H NMR (400.132 MHz, CDCl3) δ 1.25 (1 H, s), 1.57 (2 H, d), 1.62-2.01 (16 H, m), 2.16 (1 H, s), 2.24 (2 H, s), 3.52 (1 H, quintet), 4.17 (1 H, d), 4.59 (2 H, t), 4.97 (2 H, t), 5.10 (1 H, q), 5.64 (1 H, d), 5.86 (1 H, d), 8.27 (1 H, s) | m/z (ESI+) (M + H)+ = 413; HPLC t_{R} = 1.68 min. |
| 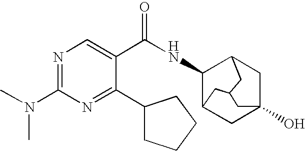 | 133 | 4- cyclopentyl- 2- dimethylamino- N- [(2r,5s)-5- hydroxyadam antan-2- yl]pyrimidine- 5- carboxamide | 1H NMR (400.132 MHz, CDCl3) δ 1.39 (1 H, s), 1.52-1.60 (2 H, m), 1.62-2.02 (16 H, m), 2.16 (1 H, s), 2.23 (2 H, s), 3.20 (6 H, s), 3.59 (1 H, quintet), 4.17 (1 H, d), 5.86 (1 H, d), 8.32 (1 H, s) | m/z (ESI+) (M + H)+ = 385; HPLC t_{R} = 1.75 min |
| 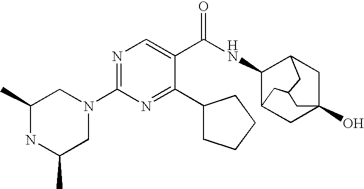 | 134 | 4- cyclopentyl- 2-[(3S,5R)- 3,5- dimethylpiperazin- 1-yl]- N-[(2r,5s)-5- hydroxyadam antan-2- yl]pyrimidine- 5- carboxamide | 1H NMR (400.132 MHz, CDCl3) δ 1.15 (6 H, d), 1.48-2.02 (20 H, m), 2.16 (1 H, s), 2.23 (2 H, s), 2.46 (2 H, d), 2.80-2.90 (2 H, m), 3.57 (1 H, quintet), 4.17 (1 H, d), 4.70 (2 H, d), 5.86 (1 H, d), 8.30 (1 H, s) | m/z (ESI+) (M + H)+ = 454; HPLC t_{R} = 1.95 min. |
| 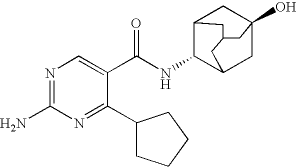 | 135 | 2-amino-4- cyclopentyl- N-[(2r,5s)-5- hydroxyadam antan-2- yl]pyrimidine- 5- carboxamide | 1H NMR (400.13 MHz, DMSO-d₆) δ 1.31 (2 H, d), 1.51- 1.57 (1 H, m), 1.52- 1.55 (1 H, m), 1.59 (3 H, s), 1.63 (1 H, s), 1.68 (1 H, s), 1.70-1.86 (7 H), 1.92 (2 H, d), 1.97 (1 H, s), 2.02 (2 H, s), 3.40 (1 H, q), 3.86- 3.90 (1 H, m), 4.38 (1 H, s), 6.68 (2 H, s), 8.02 (1 H, d), 8.08 (1 H, d) | m/z (ESI+) (M + H)+ = 357; HPLC t_{R} = 1.59 min. |
| 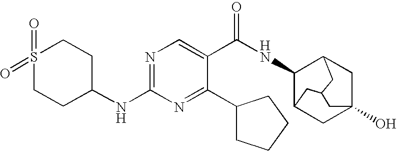 | 136 | 4- cyclopentyl- dioxothian-4- yl)amino]-N- [(2r,5s)-5- hydroxyadam antan-2- yl]pyrimidine- 5- carboxamide | 1H NMR (400.132 MHz, CDCl3) δ 1.38 (1 H, s), 1.57-1.61 (2 H, m), 1.65-1.72 (4 H, m), 1.78-1.83 (8 H, m), 1.90-2.01 (4 H, m), 2.16-2.27 (5 H, m), 2.39-2.47 (2 H, m), 3.09-3.16 (4 H, m), 3.47-3.55 (1 H, m), 4.08-4.22 (2 H, m), 5.17 (1 H, d), 5.87 (1 H, d), 8.28 (1 H, s) | m/z (ES+) (M + H)+ = 489; HPLC t_{R} = 1.71 min. |
| 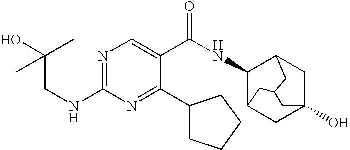 | 137 | 4- cyclopentyl- N-[(2r,5s)-5- hydroxyadam antan-2-yl]- 2-[(2- hydroxy-2- methylpropyl) amino]pyrimi- dine-5- carboxamide | 1H NMR (400.132 MHz, CDCl3) δ 1.26 (6 H, s), 1.39 (1 H, s), 1.58-1.62 (2 H, m), 1.64-1.72 (4 H, m), 1.76-1.87 (8 H, m), 1.90-1.99 (4 H, m), 2.18 (1 H, s), 2.23 (2 H, s), 3.48 (2 H, d), 3.50- 3.58 (2 H, m), 4.14- 4.21 (1 H, m), 5.63 (1 H, t), 5.86 (1 H, d), 8.25 (1 H, s) | m/z (ES+) (M + H)+ = 429; HPLC t_{R} = 1.72 min. |
| 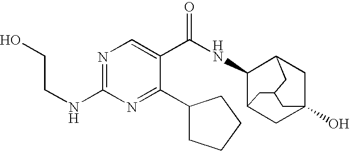 | 138 | 4- cyclopentyl- N-[(2r,5s)-5- hydroxyadam antan-2-yl]- 2-(2- hydroxyethyl amino)pyrimi- dine-5- carboxamide | 1H NMR (400.132 MHz, CDCl3) δ 1.42 (1 H, s), 1.54-1.57 (2 H, m), 1.63-1.74 (4 H, m), 1.77-1.85 (8 H, m), 1.91-2.01 (4 H, m), 2.17 (1 H, s), 2.24 (2 H, s), 3.43 (1 H, s), 3.47-3.55 (1 H, m), 3.59-3.64 (2 H, m), 3.81-3.86 (2 H, m), 4.14-4.20 (1 H, m), 5.64 (1 H, t), 5.91 (1 H, d), 8.25 (1 H, s) | m/z (ES+) (M + H)+ = 401; HPLC t_{R} = 1.53 min. |
| 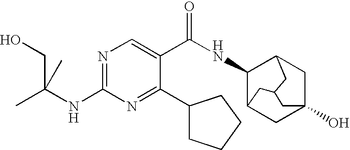 | 139 | 4- cyclopentyl- N-[(2r,5s)-5- hydroxyadam antan-2-yl]- 2-[(1- hydroxy-2- methylpropan- 2- yl)amino]pyrimi- dine-5- carboxamide | 1H NMR (400.132 MHz, CDCl3) δ 1.39 (6 H, s), 1.41 (1 H, s), 1.58-1.60 (2 H, m), 1.63-1.73 (4 H, m), 1.77-1.86 (8 H, m), 1.91-2.01 (4 H, m), 2.18 (1 H, s), 2.24 (2 H, s), 3.50 (1 H, quintet), 3.70 (2 H, s), 4.14- 4.20 (1 H, m), 5.38 (1 H, s), 5.55 (1 H, s), 5.89 (1 H, d), 8.22 (1 H, s) | m/z (ES+) (M + H)+ = 429; HPLC t_{R} = 1.80 min. |
| 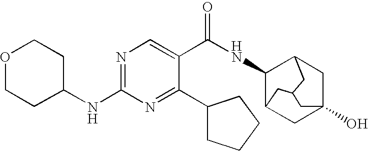 | 140 | 4- cyclopentyl- N-[(2r,5s)-5- hydroxyadam antan-2-yl]- 2-(oxan-4- ylamino)pyrimi- dine-5- carboxamide | 1H NMR (400.132 MHz, CDCl3) δ 1.01 (1 H, s), 1.45 (1 H, s), 1.53-1.57 (3 H, m), 1.65-1.74 (4 H, m), 1.77-1.88 (8 H, m), 1.91-1.97 (4 H, m), 1.99-2.08 (2 H, m), 2.18 (1 H, s), 2.24 (2 H, s), 3.51-3.57 (3 H, m), 3.96-4.10 (3 H, m), 4.15-4.20 (1 H, m), 5.16 (1 H, d), 5.87 (1 H, d), 8.27 (1 H, s) | m/z (ES+) (M + H)+ = 441; HPLC t_{R} = 1.84 min. |
| 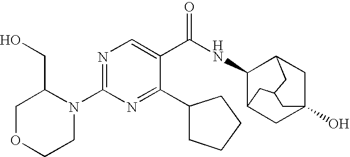 | 141 | 4- cyclopentyl- N-[(2r,5s)-5- hydroxyadam antan-2-yl]- 2-[3- (hydroxymethyl) morpholin- 4- yl]pyrimidine- 5- carboxamide | 1H NMR (400.132 MHz, CDCl3) δ 1.26 (1 H, s), 1.57-1.60 (2 H, m), 1.63-1.73 (4 H, m), 1.77-1.86 (9 H, m), 1.91-2.02 (4 H, m), 2.17 (1 H, s), 2.24 (2 H, s), 3.30- 3.37 (1 H, m), 3.54- 3.61 (2 H, m), 3.64- 3.69 (1 H, m), 3.90- 4.08 (4 H, m), 4.16- 4.20 (1 H, m), 4.54 (1 H, d), 4.71-4.78 (1 H, m), 5.87 (1 H, d), 8.32 (1 H, s) | m/z (ES+) (M + H)+ = 457; HPLC t_{R} = 1.73 min. |
| 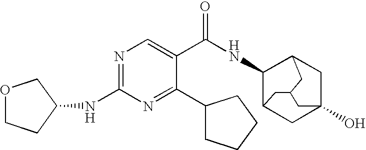 | 142 | 4- cyclopentyl- N-[(2r,5s)-5- hydroxyadam antan-2-yl]- 2-[[(3R)- oxolan-3- yl]amino]pyrimi- dine-5- carboxamide | 1H NMR (400.132 MHz, CDCl3) δ 1.55- 1.61 (4 H, m), 1.64- 1.73 (4 H, m), 1.76- 1.97 (12 H, m), 2.17 (1 H, s), 2.24 (2 H, s), 2.28-2.37 (1 H, m), 3.53 (1 H, quintet), 3.71 (1 H, dd), 3.83-3.89 (1 H, m), 3.94-4.02 (2 H, m), 4.16-4.20 (1 H, m), 4.54-4.62 (1 H, m), 5.36 (1 H, d), 5.87 (1 H, d), 8.28 (1 H, s) | m/z (ES+) (M + H)+ = 427; HPLC T_{R} = 1.76 min. |
| 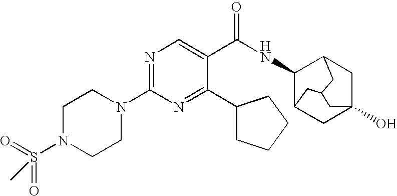 | 143 | 4- cyclopentyl- N-[(2r,5s)-5- hydroxyadam antan-2-yl]- 2-(4- methylsulfonyl- piperazin- 1- yl)pyrimidine- 5- carboxamide | 1H NMR (400.132 MHz, CDCl3) δ 1.38 (1 H, s), 1.56-1.60 (2 H, m), 1.65-1.72 (4 H, m), 1.77-1.86 (8 H, m), 1.91-1.99 (4 H, m), 2.17 (1 H, s), 2.24 (2 H, s), 2.79 (3 H, s), 3.28 (4 H, t), 3.56 (1 H, quintet), 4.01 (4 H, t), 4.15-4.20 (1 H, m), 5.87 (1 H, d), 8.33 (1 H, s) | m/z (ES+) (M + H)+ = 504; HPLC t_{R} = 2.00 min. |
| 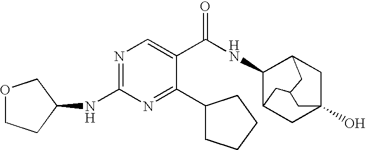 | 144 | 4- cyclopentyl- N-[(2r,5s)-5- hydroxyadam antan-2-yl]- 2-[[(3S)- oxolan-3- yl]amino]pyrimi- dine-5- carboxamide | 1H NMR (400.132 MHz, CDCl3) δ 1.55- 1.60 (4 H, m), 1.63- 1.73 (4 H, m), 1.77- 1.97 (12 H, m), 2.17 (1 H, s), 2.24 (2 H, s), 2.28-2.37 (1 H, m), 3.53 (1 H, quintet), 3.71 (1 H, dd), 3.83-3.89 (1 H, m), 3.94-4.02 (2 H, m), 4.15-4.20 (1 H, m), 4.54-4.63 (1 H, m), 5.37 (1 H, d), 5.87 (1 H, d), 8.28 (1 H, s) | m/z (ES+) (M + H)+ = 427; HPLC t_{R} = 1.79 min. |
| 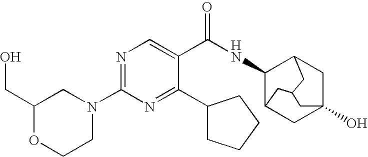 | 145 | 4- cyclopentyl- N-[(2r,Ss)-5- hydroxyadam antan-2-yl]- 2-[2- (hydroxymethyl) morpholin- 4- yl]pyrimidine- 5- carboxamide | 1H NMR (400.132 MHz, CDCl3) δ 1.55- 1.60 (2 H, m), 1.63- 1.73 (5 H, m), 1.77- 1.87 (8 H, m), 1.91- 1.97 (4 H, m), 2.16 (1 H, s), 2.23 (2 H, s), 2.89- 2.97 (2 H, m), 3.06- 3.15 (1 H, m), 3.52- 3.79 (5 H, m), 4.00- 4.06 (1 H, m), 4.17- 4.19 (1 H, m), 4.58 (2 H, d), 5.90 (1 H, d), 8.32 (1 H, s) | m/z (ES+) (M + H)+ = 457; HPLC t_{R} = 1.73 min. |
| 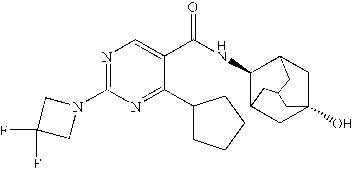 | 146 | 4- cyclopentyl- 2-(3,3- difluoroazetidin- 1-yl)-N- [(2r,5s)-5- hydroxyadam antan-2- yl]pyrimidine- 5- carboxamide | 1H NMR (400.132 MHz, CDCl3) δ 1.53- 1.60 (2 H, m), 1.62- 1.74 (5 H, m), 1.77- 1.88 (8 H, m), 1.91- 1.97 (4 H, m), 2.17 (1 H, s), 2.24 (2 H, s), 3.53 (1 H, quintet), 4.15- 4.20 (1 H, m), 4.46 (4 H, t), 5.92 (1 H, d), 8.33 (1 H, s) | m/z (ES+) (M + H)+ = 433; HPLC t_{R} = 2.21 min. |
| 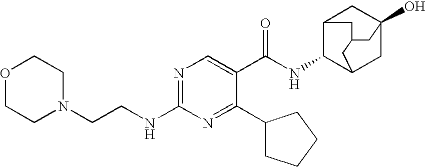 | 147 | 4- cyclopentyl- N-[(2r,5s)-5- hydroxytricyclo [3.3.1.13, 7]dec-2-yl]- 2-[(2- morpholin-4- ylethyl)amino] pyrimidine- 5- carboxamide | 1H NMR (400.13 MHz, DMSO-d₆) δ 1.30-1.37 (2 H, m), 1.54-1.57 (2 H, m), 1.60-1.63 (3 H, m), 1.68-1.73 (3 H, m), 1.82 (2 H, d), 1.92 (2 H, d), 1.98 (1 H, s), 2.02 (2 H, s), 2.39 (4 H, t), 2.45 (2 H, t), 3.16 (4 H, d), 3.40 (2 H, q), 3.56 (4 H, t), 3.88 (1 H, m), 4.06 (1 H, q), 4.38 (1 H, s), 7.09 (1 H, s), 8.01 (1 H, d), 8.12 (1 H, s) | m/z (ES+) (M + H)+ = 470; HPLC t_{R} = 1.68 min. |
| 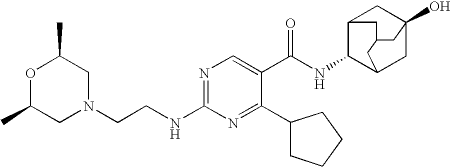 | 148 | 4- cyclopentyl- 2-({2- [(2R,6S)-2,6- dimethylmor- pholin-4- yl]ethyl}amino)- N- [(2r,5s)-5- hydroxytricyclo [3.3.1.13, 7]dec-2- yl]pyrimidine- 5- carboxamide | 1H NMR (400.13 MHz, DMSO-d₆) δ 1.03 (6 H, d), 1.31 (2 H, d), 1.55-1.57 (2 H, m), 1.63 (6 H, m), 1.70- 1.73 (4 H, m), 1.75- 1.84 (4 H, m), 1.92 (2 H, d), 2.00 (3 H, d), 2.42 (2 H, t), 2.75 (2 H, d), 3.37-3.45 (3 H, m), 3.50-3.57 (2 H, m), 3.88 (1 H, t), 4.37 (1 H, s), 7.10 (1 H, s), 8.01 (1 H, d), 8.12 (1 H, s) | m/z (ES+) (M + 2H)+ = 499; HPLC t_{R} = 1.91 min. |

### Example 149

### 4-Cyclopentyl-2-cyclopropyl-N-[(2r,5s)-5-hydroxyadamantan-2-yl]pyrimidine-5-carboxamide

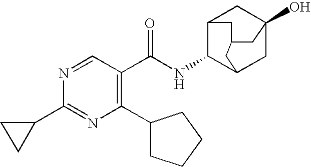

Prepared from Intermediate 88 by the same process used for Example 4

1H NMR (400.132 MHz, CDCl3) δ0.81-0.91 (1H, m), 1.02-1.09 (2H, m), 1.11-1.19 (2H, m), 1.53-1.62 (4H, m), 1.62-1.73 (4H, m), 1.77-2.01 (11H, m), 2.12-2.19 (1H, m), 2.22-2.29 (2H, m), 3.41-3.54 (1H, m), 4.16-4.26 (1H, m), 5.85-5.98 (1H, m), 8.45 (1H, s)

m/z (ESI+) (M+H)+=382.42; HPLC t_{R}=2.17 min.

### Intermediate 87

### methyl 4-cyclopentyl-2-cyclopropylpyrimidine-5-carboxylate

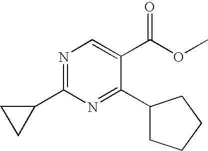

Prepared by the same process used for Intermediate 2 from Methyl 2-(cyclopentanecarbonyl)-3-(dimethylamino)acrylate

1H NMR (400.132 MHz, CDCl3) δ 1.06-1.12 (2H, m), 1.16-1.22 (2H, m), 1.64-1.74 (2H, m), 1.76-1.90 (4H, m), 1.91-2.02 (2H, m), 2.21-2.29 (1H, m), 3.91 (3H, s), 3.92-4.02 (1H, m), 8.89 (1H, s)

m/z (ESI+) (M+H)+=247.34; HPLC t_{R}=2.97 min.

### Intermediate 88

### 4-cyclopentyl-2-cyclopropylpyrimidine-5-carboxylic acid

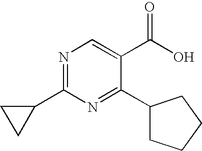

Prepared from Intermediate 87 by the same process used for Intermediate 29

1H NMR (400.132 MHz, CDCl3) δ 1.08-1.17 (2H, m), 1.18-1.29 (2H, m), 1.61-1.76 (2H, m), 1.76-1.93 (4H, m), 1.94-2.06 (2H, m), 2.23-2.34 (1H, m), 4.02-4.14 (1H, m), 9.03 (1H, s)

m/z (ESI+) (M+H)+=233.33; HPLC t_{R}=1.07 min.

### Example 150

### 4-Cyclopentyl-N-[(2r,5s)-5-hydroxyadamantan-2-yl]-2-propan-2-ylpyrimidine-5-carboxamide

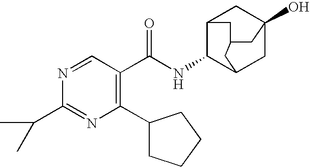

Prepared from Intermediate 90 by the Same Process Used for Example 4

1H NMR (400.132 MHz, CDCl3) δ 1.33 (6H, d), 1.53-1.63 (4H, m), 1.64-1.74 (4H, m), 1.77-2.05 (11H, m), 2.14-2.22 (1H, m), 2.23-2.30 (2H, m), 3.12-3.25 (1H, m), 3.44-3.55 (1H, m), 4.18-4.28 (1H, m), 5.87-6.02 (1H, m), 8.55 (1H, s)

m/z (ESI+) (M+H)+=384.44; HPLC t_{R}=2.24 min.

### Intermediate 89

### methyl 4-cyclopentyl-2-isopropylpyrimidine-5-carboxylate

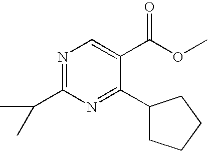

Prepared by the same process used for Intermediate 2 from Methyl 2-(cyclopentanecarbonyl)-3-(dimethylamino)acrylate

1H NMR (400.132 MHz, CDCl3) δ 1.34 (6H, d), 1.64-1.76 (2H, m), 1.79-2.03 (6H, m), 3.14-3.27 (1H, m), 3.92 (3H, s), 3.94-4.02 (1H, m), 8.97 (1H, s)

m/z (ESI+) (M+H)+=249.33; HPLC t_{R}=3.10 min.

### Intermediate 90

### 4-cyclopentyl-2-isopropylpyrimidine-5-carboxylic acid

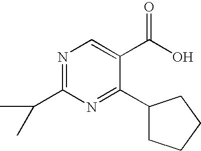

Prepared from Intermediate 89 by the same process used for Intermediate 29

1H NMR (400.132 MHz, CDCl3) δ 1.36 (6H, d), 1.64-1.77 (2H, m), 1.80-2.09 (6H, m), 3.19-3.30 (1H, m), 4.05-4.17 (1H, m), 9.14 (1H, s)

m/z (ESI+) (M+H)+=235.30; HPLC t_{R}=1.05 min.

### Example 151

### 2-(1-Aminocyclopropyl)-4-cyclopentyl-N-[(2r,5s)-5-hydroxyadamantan-2-yl]pyrimidine-5-carboxamide

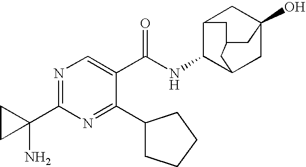

benzyl N-[1-[4-cyclopentyl-5-[(5-hydroxy-2-adamantyl)carbamoyl]pyrimidin-2-yl]cyclopropyl]carbamate (Intermediate 93, 174.4 mg, 0.33 mmol) and palladium, 10% wt on carbon (35.9 mg, 0.03 mmol) in ethanol (25 mL) was stirred under an atmosphere of hydrogen at room temperature and normal pressure over night. The reaction mixture was filtered through celite and the solvent volume reduced. The crude product was purified by preparative HPLC (Phenomenex Luna C18 100A column, 5μ silica, 30 mm diameter, 100 mm length), using decreasingly polar mixtures of water (containing 0.1% NH3) and MeCN as eluents. Fractions containing the desired compound were evaporated to dryness to afford 2-(1-aminocyclopropyl)-4-cyclopentyl-N-[(2r,5 s)-5-hydroxyadamantan-2-yl]pyrimidine-5-carboxamide (60.3 mg, 46.3%) as a white solid.

1H NMR (400.13 MHz, DMSO-d6) δ 1.05 (2H, q), 1.27 (2H, q), 1.33 (2H, d), 1.56-1.62 (4H, m), 1.64 (1H, s), 1.69-1.71 (1H, m), 1.72-1.89 (10H, m), 1.91 (1H, s), 1.98 (1H, s), 2.04 (2H, s), 2.44 (1H, s), 3.34-3.42 (1H, m), 3.93-3.97 (1H, m), 4.40 (1H, s), 8.35 (1H, d), 8.45 (1H, s)

m/z (ESI+) (M+H)+=397; HPLC t_{R}=1.67 min.

### Intermediate 177

### benzyl 1-cyanocyclopropylcarbamate

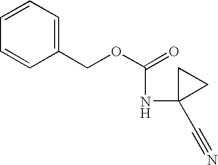

Benzyl chloroformate (4.76 mL, 33.49 mmol) was added dropwise to 1-aminocyclopropanecarbonitrile (2.5 g, 30.45 mmol) and triethylamine (8.48 mL, 60.90 mmol) in DCM (40 mL) at 0° C. over a period of 10 minutes under nitrogen. The resulting solution was stirred at room temperature over night. The reaction mixture was diluted with DCM (100 mL), and washed sequentially with saturated brine (2×75 mL). The organic layer was dried over MgSO4, filtered and evaporated to afford crude The crude product was purified by flash silica chromatography, elution gradient 0 to 40% EtOAc in isohexane. Pure fractions were evaporated to dryness to afford benzyl 1-cyanocyclopropylcarbamate (1.570 g, 23.84%) as a white solid.

1H NMR (400.13 MHz, CDCl₃) δ 1.27-1.31 (2H, m), 1.51-1.56 (2H, m), 5.17 (2H, s), 5.38 (1H, s), 7.32-7.39 (5H, m)

m/z (ESI−) (M−H)-=215; HPLC t_{R}=1.88 min.

### Intermediate 178

### benzyl 1-carbamimidoylcyclopropylcarbamate hydrochloride

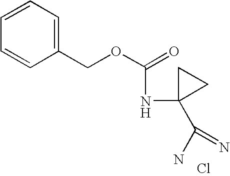

benzyl 1-cyanocyclopropylcarbamate (Intermediate 177, 0.84 g, 3.88 mmol) in dioxane (5 mL) was added to a 4M HCl solution of dioxane (2 mL). The reaction was stirred at room temperature over night. The intermediate was not UV active but the mass peak could be seen in LC/MS. TLC indicated the reaction had gone to completion. The solvent volume was evaporated to dryness. The residue was re dissolved in methanol (3 mL) and 7M NH3 saturated in MeOH (2 mL) was added. The reaction was stirred at room temperature for 2 hours. The solvent volume was evaporated to dryness and used in the next reaction step without further purification or characterisation.

1H NMR (400.13 MHz, DMSO-d₆) δ 0.85-0.88 (2H, m), 1.20-1.23 (2H, m), 5.02 (2H, s), 7.03 (1H, s), 7.16 (1H, s), 7.27-7.37 (5H, m), 7.78 (1H, s)

m/z (ESI+) (M+H)+=234; HPLC t_{R}=1.61 min

### Intermediate 91

### methyl 2-(1-(benzyloxycarbonylamino)cyclopropyl)-4-cyclopentylpyrimidine-5-carboxylate

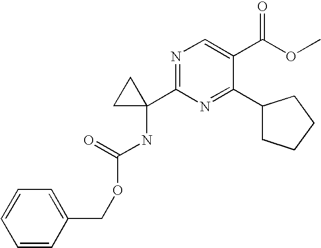

Prepared by the same process used for Intermediate 2 from Methyl 2-(cyclopentanecarbonyl)-3-(dimethylamino)acrylate and benzyl 1-carbamimidoylcyclopropylcarbamate hydrochloride (Intermediate 178)

m/z (ESI+) (M+H)+=396; HPLC t_{R}=2.93 min.

### Intermediate 92

### 2-(1-(benzyloxycarbonylamino)cyclopropyl)-4-cyclopentylpyrimidine-5-carboxylic acid

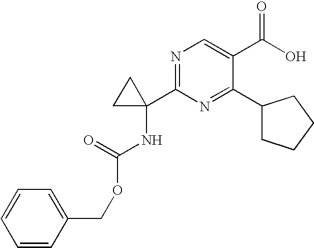

Prepared from Intermediate 91 by the same process used for Intermediate 29

1H NMR (400.13 MHz, CDCl₃) δ 1.19-1.31 (2H, m), 1.39-1.44 (1H, m), 1.53-1.59 (1H, m), 1.68-1.74 (3H, m), 1.72 (3H, s), 1.85-1.91 (2H, m), 3.97-4.05 (1H, m), 5.05 (2H, s), 6.04-6.09 (1H, m), 7.06 (1H, s), 7.16-7.29 (3H, d), 8.93 (1H, s)

m/z (ESI+) (M+H)+=382; HPLC t_{R}=1.87 min.

### Intermediate 93

### benzyl N-[1-[4-cyclopentyl-5-[(5-hydroxy-2-adamantyl)carbamoyl]pyrimidin-2-yl]cyclopropyl]carbamate

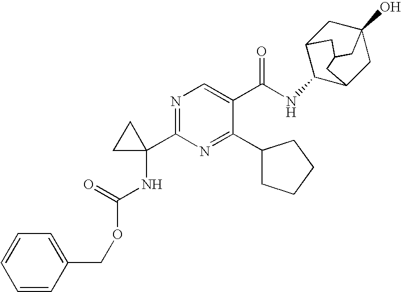

Prepared from Intermediate 92 by the same process used for Example 4

1H NMR (400.13 MHz, CDCl₃) δ 1.31-1.39 (2H, m), 1.46 (1H, m), 1.50 (2H, m), 1.53-1.59 (1H, m), 1.61-1.64 (4H, m), 1.69-1.76 (9H, t), 1.82-1.85 (4H, m), 2.08 (1H, s), 2.14 (2H, s), 4.07-4.12 (1H, m), 5.04 (2H, s), 5.79 (1H, s), 6.04 (1H, d), 7.21-7.33 (5H, m), 8.37 (1H, s)

m/z (ESI+) (M+H)+=531; HPLC t_{R}=2.44 min.

### Example 152

### 2-(Aminomethyl)-4-cyclopentyl-N-[(2r,5s)-5-hydroxyadamantan-2-yl]pyrimidine-5-carboxamide

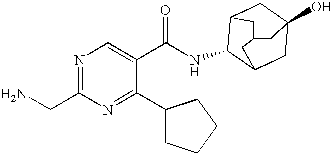

Prepared by the same process used for Example 151 from Intermediate 94

1H NMR (400.13 MHz, CDCl₃) δ 1.58 (2H, d), 1.65-1.76 (3H, m), 1.79 (3H, s), 1.83 (1H, s), 1.84-1.89 (4H, m), 1.90-2.04 (5H, d), 2.17 (1H, s), 2.25 (2H, s), 3.47-3.54 (1H, q), 4.06 (2H, s),4.19-4.24 (1H, m), 6.15 (1H, d), 8.56 (1H, s)

m/z (ESI+) (M+H)+=371; HPLC t_{R}=1.46 min.

### Intermediate 94

### cyano [4-cyclopentyl-5-[(5-hydroxy-2-adamantyl)carbamoyl]pyrimidin-2-yl]

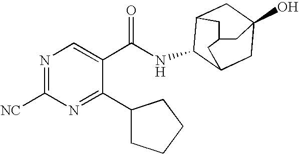

Sodium cyanide (0.148 g, 3.02 mmol) was added in one portion to 4-cyclopentyl-N-[(2r,5s)-5-hydroxyadamantan-2-yl]-2-methylsulfinylpyrimidine-5-carboxamide (Intermediate 86, 1.017 g, 2.52 mmol) in DMA (15 mL) at 0° C. under nitrogen. The resulting solution was stirred at 0° C. for 2 hours. The reaction mixture was quenched with saturated NaHCO3 (50 mL), extracted with DCM (2×100 mL), the organic layer was dried over MgSO4, filtered and evaporated to afford yellow solid. The crude product was purified by flash silica chromatography, elution gradient 0 to 100% EtOAc in isohexane. Pure fractions were evaporated to dryness to afford cyano; [4-cyclopentyl-5-[(5-hydroxy-2-adamantyl)carbamoyl]pyrimidin-2-yl] (0.758 g, 82%) as a yellow oil.

1H NMR (400.13 MHz, CDCl₃) δ 1.57 (2H, d), 1.68-1.77 (5H, m), 1.80 (3H, s), 1.84 (2H, s), 1.87-1.98 (6H, m), 2.18 (1H, s), 2.26 (2H, s), 3.48 (1H, q), 4.19-4.24 (1H, m), 6.59 (1H, d), 8.65 (1H, s)

m/z (ESI−) (M−H)-=365; HPLC t_{R}=2.10 min.

### Example 153

### 4-(3,3-difluorocyclobutyl)-N-[(2s,5r)-5-hydroxyadamantan-2-yl]-2-methylpyrimidine-5-carboxamide

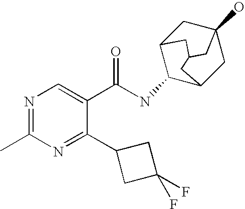

Prepared from Intermediate 102 by the same process used for Example 4

1H NMR (400.13 MHz, DMSO-d₆) δ 1.35 (2H, d), 1.63 (4H, d), 1.72 (2H, d), 1.89 (2H, d), 1.99 (1H, s), 2.06 (2H, s), 2.66 (3H, s), 2.78-3.00 (4H, m), 3.66-3.71 (1H, m), 3.96 (1H, t), 4.41 (1H, s), 8.38 (1H, d), 8.58 (1H, s)

m/z (ESI+) (M+H)+=378; HPLC t_{R}=1.64 min m/z (ESI+) (M+H)+=378; HPLC t_{R}=1.64 min.

### Intermediate 95

### methyl 3-(3,3-difluorocyclobutyl)-3-oxopropanoate

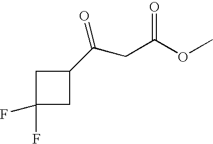

Prepared from 5-(3,3-difluorocyclobutanecarbonyl)-2,2-dimethyl-1,3-dioxane-4,6-dione by the same process used for Intermediate 122

1H NMR (400.13 MHz, CDCl₃) δ 2.69-2.90 (4H, m), 3.21-3.26 (1H, m), 3.49 (2H, s), 3.75 (3H, d)

### Intermediate 96

### (Z)-methyl 2-(3,3-difluorocyclobutanecarbonyl)-3-(dimethylamino)acrylate

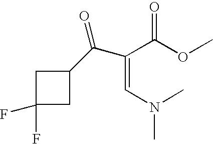

Prepared from methyl 3-(3,3-difluorocyclobutyl)-3-oxopropanoate by the same process used for Intermediate 1

m/z (ESI+) (M+H)+=248; HPLC t_{R}=1.63 min. 5 min Base

### Intermediate 97

### methyl 4-(3,3-difluorocyclobutyl)-2-methylpyrimidine-5-carboxylate

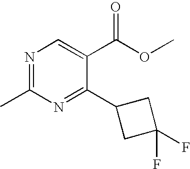

(Z)-methyl 2-(3,3-difluorocyclobutanecarbonyl)-3-(dimethylamino)acrylate (Intermediate 96, 500 mg, 2.02 mmol) was added dropwise to Acetamidine hydrochloride (191 mg, 2.02 mmol), Sodium methoxide (4045 μL, 2.02 mmol) in methanol (20 mL) under nitrogen. The resulting solution was stirred at 60° C. for 4 hours then room temperature for a further 16 hours. The reaction mixture was evaporated to dryness and redissolved in EtOAc (50 mL), and washed sequentially with 2M HCl (25 mL), saturated brine (25 mL). The organic layer was dried over MgSO4, filtered and evaporated to afford crude product. The crude product was purified by flash silica (40 g) chromatography, elution gradient 20 to 50% EtOAc in isohexane. Pure fractions were evaporated to dryness to afford methyl 4-(3,3-difluorocyclobutyl)-2-methylpyrimidine-5-carboxylate (388 mg, 79%) as a colourless oil which solidified on standing.

1H NMR (400.13 MHz, CDCl₃) δ 2.79 (3H, s), 2.85-2.96 (2H, m), 2.99-3.12 (2H, m), 3.94 (3H, s), 4.16-4.21 (1H, m), 9.05 (1H, s)

m/z (ESI+) (M+H)+=243; HPLC t_{R}=2.1 min.

### Intermediate 98

### 4-(3,3-difluorocyclobutyl)-2-methylpyrimidine-5-carboxylic acid

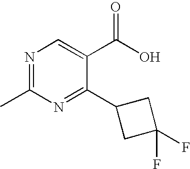

Sodium hydroxide (2.002 mL, 4.00 mmol) was added in one portion to methyl 4-(3,3-difluorocyclobutyl)-2-methylpyrimidine-5-carboxylate (Intermediate 97, 388 mg, 1.60 mmol), in methanol (10 mL) under air. The resulting solution was stirred at 60° C. for 3 hours. The reaction mixture was evaporated to dryness and redissolved in water (10 mL), The solution was acidified with concentrated HCl. The precipitate was collected by filtration, washed with water (10 mL) and dried under vacuum to afford 4-(3,3-difluorocyclobutyl)-2-methylpyrimidine-5-carboxylic acid (330 mg, 90%) as a white solid, which was used without further purification.

1H NMR (400.13 MHz, DMSO-d₆) δ 2.69 (3H, s), 2.82-2.90 (2H, m), 2.94-3.06 (2H, m), 4.16-4.21 (1H, m), 9.00 (1H, s)

m/z (ESI+) (M+H)+=229 HPLC t_{R}=1.63 min.

### Intermediate 99

### methyl 4-(3,3-difluorocyclobutyl)-2-(methylthio)pyrimidine-5-carboxylate

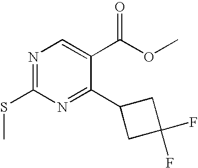

Prepared by the same process used for Intermediate 28 from (Z)-methyl 2-(3,3-difluorocyclobutanecarbonyl)-3-(dimethylamino)acrylate (Intermediate 96)

1H NMR (400.13 MHz, CDCl₃) δ 2.63 (3H, s), 2.88-2.98 (2H, m), 3.00-3.09 (2H, m), 3.92 (3H, s), 4.20-4.25 (1H, m), 8.94 (1H, s)

m/z (ESI+) (M+H)+=275; HPLC t_{R}=2.61 min.

### Intermediate 100

### 4-(3,3-difluorocyclobutyl)-2-methylsulfanylpyrimidine-5-carboxylic acid

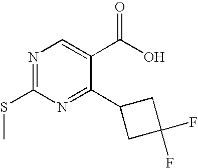

Prepared from Intermediate 99 by the Same Process Used for Intermediate 29

1H NMR (400.13 MHz, DMSO-d₆) δ 2.59 (3H, s), 2.83-2.94 (2H, m), 2.94-3.04 (2H, m), 3.35 (1H, bs), 4.18-4.23 (1H, m), 8.92 (1H, s)

m/z (ESI+) (M+H)+=261; HPLC t_{R}=2.13 min.

### Intermediate 101

### 4-(3,3-difluorocyclobutyl)-N-[(2r,5s)-5-hydroxyadamantan-2-yl]-2-methylsulfanylpyrimidine-5-carboxamide

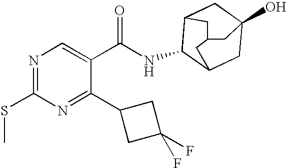

Prepared from Intermediate 100 by the Same Process Used for Example 4

m/z (ESI+) (M+H)+=410; HPLC t_{R}=2.03 min.

### Intermediate 102

### 4-(3,3-difluorocyclobutyl)-N-[(2r,5s)-5-hydroxyadamantan-2-yl]-2-methylsulfinylpyrimidine-5-carboxamide

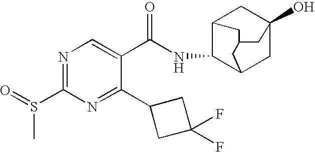

Prepared from Intermediate 101 by the Same Process Used for Intermediate 60

m/z (ESI+) (M+H)+=426; HPLC t_{R}=1.41 min

The following Examples were prepared in a similar manner to Example 46, using Intermediate 102 and an appropriate amine starting material:

| | | | | MS |
|---|---|---|---|---|
| | | | | m/e |
| Structure | Ex | Name | ¹H NMR δ | MH⁺ |
| 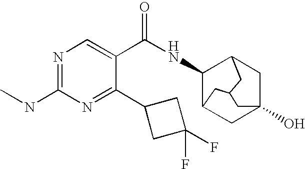 | 154 | 4-(3,3- difluorocyclo- butyl)-N- [(2r,5s)-5- hydroxyadam antan-2-yl]-2- methylamino- pyrimidine-5- carboxamide | 1H NMR (400.132 MHz, CDCl3) δ 1.40 (1 H, s), 1.58 (2 H, d), 1.65-1.74 (2 H, m), 1.75-1.85 (4 H, m), 1.93 (2 H, d), 2.14-2.26 (3 H, m), 2.76-2.90 (2 H, m), 2.90-3.10 (2 H, m), 3.06 (3 H, d), 3.86 (1 H, t), 4.14 (1 H, s), 5.34 (1 H, s), 5.87 (1 H, s), 8.34 (1 H, s) | m/z (ES+) (M + H)+ = 393; HPLC t_{R} = 1.75 min. |
| 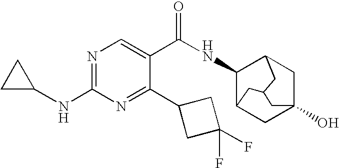 | 155 | 2- (cyclopropyl- amino)-4-(3,3- difluorocyclo- butyl)-N- [(2r,5s)-5- hydroxyadam antan-2- yl]pyrimidine- 5- carboxamide | 1H NMR (400.132 MHz, CDCl3) δ 0.55- 0.61 (2 H, m), 0.79- 0.91 (2 H, m), 1.26 (1 H, s), 1.58 (2 H, d), 1.64- 1.85 (6 H, m), 1.93 (2 H, d), 2.14-2.27 (3 H, m), 2.76-2.90 (3 H, m), 2.91-3.09 (2 H, m), 3.86 (1 H, quintet), 4.15 (1 H, s), 5.56 (1 H, s), 5.88 (1 H, s), 8.39 (1 H, s) | m/z (ES+) (M + H)+ = 419; HPLC t_{R} = 1.91 min. |
| 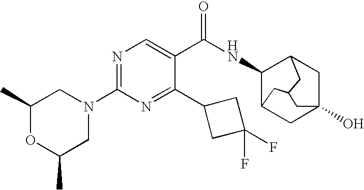 | 156 | 4-(3,3- difluorocyclo- butyl)-2- [(2S,6R)-2,6- dimethylmor- pholin-4-yl]-N- [(2r,5s)-5- hydroxyadam antan-2- yl]pyrimidine- 5- carboxamide | 1H NMR (400.132 MHz, CDCl3) δ 1.27 (6 H, d), 1.42-1.72 (5 H, m), 1.76-1.85 (4 H, m), 1.93 (2 H, d), 2.14-2.25 (3 H, m), 2.65 (2 H, dd), 2.80-3.03 (4 H, m), 3.58-3.68 (2 H, m), 3.89 (1 H, quintet), 4.15 (1 H, d), 4.65 (2 H, d), 5.86 (1 H, d), 8.36 (1 H, s) | m/z (ES+) (M + H)+ = 477; HPLC t_{R} = 2.15 min. |

The following Examples were prepared in a similar manner to Example 75, using Intermediate 102 and an appropriate amine starting material:

| | | | | MS m/e |
|---|---|---|---|---|
| Structure | Ex | Name | ¹H NMR δ | MH+ |
| 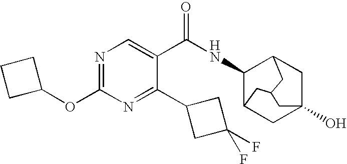 | 157 | 2- cyclobutyloxy- 4-(3,3- difluorocyclo butyl)-N- [(2r,5s)-5- hydroxyada mantan-2- yl]pyrimidine- 5- carboxamide | 1H NMR (400.132 MHz, CDCl3) δ 1.41 (1 H, s), 1.60 (2 H, d), 1.65-1.91 (8 H, m), 1.94 (2 H, d), 2.15- 2.31 (5 H, m), 2.44- 2.56 (2 H, m), 2.80- 3.09 (4 H, m), 3.87 (1 H, quintet), 4.18 (1 H, d), 5.23 (1 H, quintet), 5.95 (1 H, d), 8.50 (1 H, s) | m/z (ES+) (M + H)+ = 434; HPLC t_{R} = 2.16 min. |

### Example 158

### N-[(2r,5s)-5-hydroxyadamantan-2-yl]-2-methyl-4-(oxolan-2-yl)pyrimidine-5-carboxamide

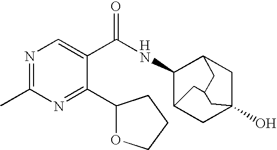

Prepared from Intermediate 110 by the Same Process Used for Example 4

1H NMR (400.132 MHz, CDCl3) δ 1.36 (1H, s), 1.54-1.59 (2H, m), 1.75-1.84 (6H, m), 1.92-1.97 (2H, m), 2.04-2.30 (6H, m), 2.75 (3H, s), 2.79-2.85 (1H, m), 3.88-3.93 (1H, m), 3.97-4.03 (1H, m), 4.21-4.26 (1H, m), 5.12 (1H, t), 7.73 (1H, d), 8.93 (1H, s)

m/z (ESI+) (M+H)+=358; HPLC t_{R}=1.22 min.

### Example 159

### N-[(2r,5s)-5-hydroxyadamantan-2-yl]-4-(oxolan-2-yl)-2-(propan-2-ylamino)pyrimidine-5-carboxamide

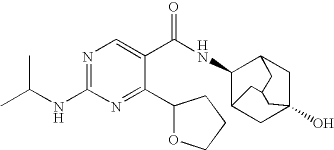

Isopropylamine (0.303 mL, 3.56 mmol) was added to N-[(2r,5s)-5-hydroxyadamantan-2-yl]-2-methylsulfonyl-4-(oxolan-2-yl)pyrimidine-5-carboxamide (Intermediate 110, 300 mg, 0.71 mmol) in THF (5 mL) at 20° C. The resulting solution was stirred at 20° C. for 2 hours.

The reaction mixture was evaporated to dryness. Purified by preparative HPLC (Phenomenex Gemini C18 110A (axia) column, 5μ silica, 30 mm diameter, 100 mm length), using decreasingly polar mixtures of water (containing 0.5% NH3) and MeCN as eluents. Fractions containing the desired compound were evaporated to dryness to afford the product, N-[(2r,5s)-5-hydroxyadamantan-2-yl]-4-(oxolan-2-yl)-2-(propan-2-ylamino)pyrimidine-5-carboxamide (143 mg, 50.2%)

1H NMR (400.132 MHz, CDCl3) δ 1.25 (6H, d), 1.42 (1H, s), 1.48-1.57 (2H, m), 1.75-1.84 (6H, m), 1.90-1.97 (2H, m), 2.02-2.08 (2H, m), 2.14-2.25 (4H, m), 2.76 (1H, bs), 3.86-3.93 (1H, m), 3.98-4.03 (1H, m), 4.13-4.24 (2H, m), 5.08 (1H, t), 5.21 (1H, d), 7.79 (1H, s), 8.69 (1H, bs)

m/z (ES+) (M+H)+=401; HPLC t_{R}=1.78 min.

### Intermediate 104

### (Z)-methyl 3-(dimethylamino)-2-(tetrahydrofuran-2-carbonyl)acrylate

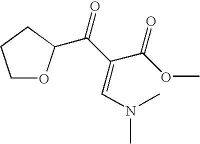

Prepared from methyl 3-oxo-3-(tetrahydrofuran-2-yl)propanoate by the same process used for Intermediate 1;

1H NMR (400.132 MHz, CDCl3) δ 1.87 (2H, quintet), 2.00-2.09 (1H, m), 2.12-2.22 (1H, m), 3.05 (6H, s), 3.73 (3H, s), 3.83-3.89 (1H, m), 3.90-3.96 (1H, m), 4.97 (1H, t), 7.67 (1H, s)

m/z (ESI+) (M+H)+=228; HPLC t_{R}=1.01 min.

### Intermediate 105

### methyl 2-methyl-4-(tetrahydrofuran-2-yl)pyrimidine-5-carboxylate

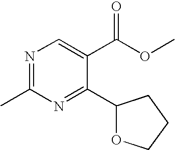

A solution of (Z)-methyl 3-(dimethylamino)-2-(tetrahydrofuran-2-carbonyl)acrylate

(Intermediate 104, 1.4 g, 6.16 mmol) in methanol (5 mL) was added dropwise to a stirred suspension of Acetamidine hydrochloride (0.582 g, 6.16 mmol), and Sodium methoxide (0.5M in MeOH) (12.32 mL, 6.16 mmol) in methanol (25 mL) at 20° C. The resulting solution was stirred at 80° C. for 24 hours. The reaction mixture was evaporated to dryness and redissolved in EtOAc (100 mL), and washed sequentially with water (75 mL) and saturated brine (75 mL). The organic layer was dried over MgSO4, filtered and evaporated to afford crude product. The crude product was purified by flash silica chromatography, elution gradient 40 to 70% EtOAc in isohexane. Pure fractions were evaporated to dryness to afford methyl 2-methyl-4-(tetrahydrofuran-2-yl)pyrimidine-5-carboxylate (0.600 g, 43.8%) as a colourless oil.

1H NMR (400.132 MHz, CDCl3) δ 1.95-2.09 (3H, m), 2.40-2.51 (1H, m), 2.79 (3H, s), 3.94 (3H, s), 3.97-4.03 (1H, m), 4.13-4.20 (1H, m), 5.58-5.62 (1H, m), 8.96 (1H, s)

m/z (ESI+) (M+H)+=223; HPLC t_{R}=1.27 min.

### Intermediate 106

### 2-methyl-4-(tetrahydrofuran-2-yl)pyrimidine-5-carboxylic acid

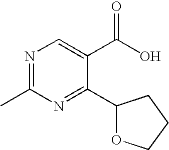

Prepared from Intermediate 105 by the Same Process Used for Intermediate 29

1H NMR (400.132 MHz, CDCl3) δ 2.00-2.15 (3H, m), 2.40-2.54 (1H, m), 2.82 (3H, s), 4.04-4.09 (1H, m), 4.18-4.25 (1H, m), 5.63-5.67 (1H, m), 6.48 (1H, bs), 9.15 (1H, s)

m/z (ESI+) (M+H)+=209; HPLC t_{R}=0.93 min.

### Intermediate 107

### methyl 2-(methylthio)-4-(tetrahydrofuran-2-yl)pyrimidine-5-carboxylate

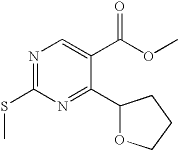

Prepared from Intermediate 104 by the same process used for Intermediate 28;

1H NMR (400.132 MHz, CDCl3) δ 1.94-2.11 (3H, m), 2.38-2.47 (1H, m), 2.60 (3H, s), 3.91 (3H, s), 4.00-4.06 (1H, m), 4.11-4.19 (1H, m), 5.69-5.74 (1H, m), 8.88 (1H, s)

m/z (ESI+) (M+H)+=255; HPLC t_{R}=1.88 min.

### Intermediate 108

### 2-methylsulfanyl-4-(oxolan-2-yl)pyrimidine-5-carboxylic acid

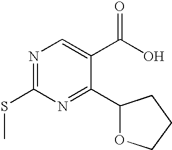

Prepared from Intermediate 107 by the same process used for Intermediate 21;

1H NMR (400.132 MHz, CDCl3) δ 2.00-2.19 (3H, m), 2.39-2.49 (1H, m), 2.62 (3H, s), 4.05-4.10 (1H, m), 4.17-4.23 (1H, m), 5.70-5.74 (1H, m), 6.13 (1H, bs), 9.03 (1H, s)

m/z (ESI+) (M+H)+=241; HPLC t_{R}=0.69 min.

### Intermediate 109

### N-[(2r,5 s)-5-hydroxyadamantan-2-yl]-2-methylsulfanyl-4-(oxolan-2-yl)pyrimidine-5-carboxamide

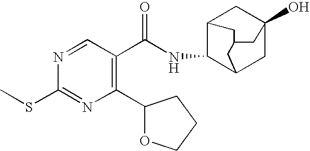

Prepared from Intermediate 108 by the same process used for Example; 4

1H NMR (400.132 MHz, CDCl3) δ 1.50-1.59 (3H, m), 1.75-1.83 (6H, m), 1.90-1.97 (2H, m), 2.03-2.27 (6H, m), 2.59 (3H, s), 2.80-2.91 (1H, m), 3.89-3.93 (1H, m), 3.97-4.02 (1H, m), 4.20-4.26 (1H, m), 5.14 (1H, t), 7.91 (1H, d), 8.86 (1H, s)

m/z (ESI+) (M+H)+=390; HPLC t_{R}=1.73 min.

### Intermediate 110

### N-[(2r,5s)-5-hydroxyadamantan-2-yl]-2-methylsulfonyl-4-(oxolan-2-yl)pyrimidine-5-carboxamide

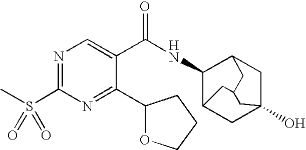

Prepared from Intermediate 109 by the same process used for Example 37;

1H NMR (400.132 MHz, CDCl3) δ 1.50-1.60 (3H, m), 1.74-1.85 (6H, m), 1.90-1.98 (2H, m), 2.08-2.31 (6H, m), 2.79-2.90 (1H, m), 3.36 (3H, s), 3.90-4.04 (2H, m), 4.23-4.30 (1H, m), 5.24 (1H, t), 7.88 (1H, d), 9.17 (1H, s)

m/z (ESI+) (M+H)+=422; HPLC t_{R}=1.22 min.

The following Examples were prepared in a similar manner to Example 159, using Intermediate 110 and an appropriate amine starting material:

| | | | | MS |
|---|---|---|---|---|
| | | | | m/e |
| Structure | Ex | Name | ¹H NMR δ | MH⁺ |
| 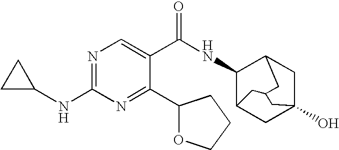 | 160 | 2- (cyclopropyla mino)-N- [(2r,5s)-5- hydroxyadaman- tan-2-yl]-4- (oxolan-2- yl)pyrimidine- 5- carboxamide | 1H NMR (400.132 MHz, CDCl3) δ 0.52- 0.60(2 H, m), 0.77-0.87 (2 H, m), 1.50-1.56(2 H, m), 1.75-1.83(6 H, m), 1.90-1.96(2 H, m), 2.00- 2.24(7 H, m), 2.73- 2.83(2 H, m), 3.86-3.92 (1 H, m), 3.99-4.03(1 H, m), 4.18-4.23(1 H, m), 5.10(1 H, t), 5.80(1 H, s), 7.83(1 H, s), 8.71 (1 H, s) | m/z (ES+) (M + H)+ = 399; HPLC t_{R} = 1.60 min. |
| 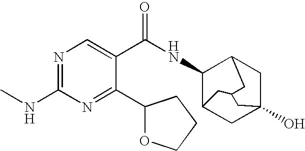 | 161 | N-[(2r,5s)-5- hydroxyadaman- tan-2-yl]-2- methylamino- 4-(oxolan-2- yl)pyrimidine- 5- carboxamide | 1H NMR (400.132 MHz, CDCl3) δ 1.47- 1.57(2 H, m), 1.77-1.83 (6 H, m), 1.89-1.97(2 H, m), 2.01-2.09(2 H, m), 2.13-2.24(5 H, m), 2.78 (1 H, s), 3.03(3 H, d), 3.90(1 H, q), 3.98-4.03 (1 H, m), 4.18-4.22(1 H, m), 5.09(1 H, t), 5.61 (1 H, s), 7.86(1 H, s), 8.68(1 H, s) | m/z (ES+) (M + H)+ = 373; HPLC t_{R} = 1.37 min. |
| 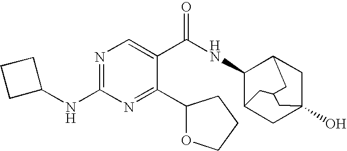 | 162 | 2- (cyclobutylami- no)-N- [(2r,5s)-5- hydroxyadaman- tan-2-yl]-4- (oxolan-2- yl)pyrimidine- 5- carboxamide | 1H NMR (400.132 MHz, CDCl3) δ 1.47- 1.53(3 H, m), 1.73-1.84 (8 H, m), 1.89-1.96(4 H, m), 2.02-2.07(2 H, m), 2.14-2.25(4 H, m), 2.37- 2.46(2 H, m), 2.76(1 H, s), 3.85-3.92(1 H, m), 3.97-4.02(1 H, m), 4.18- 4.24(1 H, m), 4.47(1 H, sextet), 5.07(1 H, t), 5.49 (1 H, d), 7.77(1 H, s), 8.66(1 H, s) | m/z (ES+) (M + H)+ = 413; HPLC t_{R} = 1.87 min. |
| 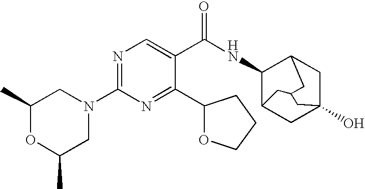 | 163 | 2-[(2S,6R)- 2,6- dimethylmorpho- lin-4-yl]-N- [(2r,5s)-5- hydroxyadaman- tan-2-yl]-4- (oxolan-2- yl)pyrimidine- 5- carboxamide | 1H NMR (400.132 MHz, CDCl3) δ 1.26 (6 H, d), 1.38(1 H, s), 1.48-1.53(2 H, m), 1.75- 1.83(6 H, m), 1.91- 1.97(2 H, m), 2.02-2.11 (2 H, m), 2.14-2.25(4 H, m), 2.60-2.66(2 H, m), 2.74-2.84(1 H, m), 3.58- 3.66(2 H, m), 3.91(1 H, q), 3.99-4.05(1 H, m), 4.20-4.24(1 H, m), 4.63 (2 H, d), 5.08(1 H, t), 7.89(1 H, d), 8.75(1 H, s) | m/z (ES+) (M + H)+ = 457; HPLC t_{R} = 1.96 min. |
| 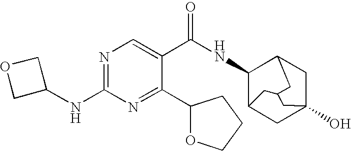 | 164 | N-[(2r,5s)-5- hydroxyadaman- tan-2-yl]-2- (oxetan-3- ylamino)-4- (oxolan-2- yl)pyrimidine- 5- carboxamide | 1H NMR (400.132 MHz, CDCl3) δ 1.48- 1.56(3 H, m), 1.76-1.83 (6 H, m), 1.88-1.96(2 H, m), 2.03-2.11(2 H, m), 2.15-2.23(4 H, m), 2.67- 2.75(1 H, m), 3.87- 3.93(1 H, m), 3.97-4.02 (1 H, m), 4.18-4.24(1 H, m), 4.60(2 H, t), 4.95- 5.00(2 H, m), 5.07(1 H, t), 5.14(1 H, sextet), 5.79 (1 H, d), 7.77(1 H, s), 8.71(1 H, s) | m/z (ES+) (M + H)+ = 415; HPLC t_{R} = 1.31 min. |

The following Examples were prepared in a similar manner to Example 75, using Intermediate 110 and an appropriate amine starting material:

| | | | | MS m/e |
|---|---|---|---|---|
| Structure | Ex | Name | ¹H NMR δ | MH⁺ |
| 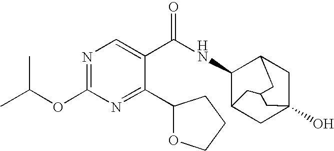 | 165 | N-[(2r,5s)-5- hydroxyadaman- tan-2-yl]-4- (oxolan-2-yl)- 2-propan-2- yloxypyrimidine- 5- carboxamide | 1H NMR (400.132 MHz, CDCl3) δ 1.42(6 H, d), 1.48- 1.59(2 H, m), 1.66(1 H, s), 1.76-1.82(6 H, m), 1.90- 1.97(2 H, m), 2.02-2.28 (6 H, m), 2.75-2.86(1 H, m), 3.86-3.94(1 H, m), 3.97- 4.03(1 H, m), 4.19-4.26 (1 H, m), 5.12(1 H, t), 5.33 (1 H, septet), 7.84(1 H, d), 8.85(1 H, s) | m/z (ES+) (M + H)+ = 402; HPLC t_{R} = 1.78 min. |

### Example 166

### N-[(2r,5s)-5-hydroxyadamantan-2-yl]-2-methylsulfanyl-4-[(2R)-oxolan-2-yl]pyrimidine-5-carboxamide

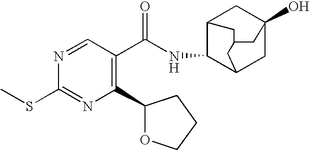

N-Ethyldiisopropylamine (3.57 mL, 20.48 mmol) was added to (R)-2-(methylthio)-4-(tetrahydrofuran-2-yl)pyrimidine-5-carboxylic acid (Intermediate 114, 1.23 g, 5.12 mmol), 4-aminoadamantan-1-ol hydrochloride (1.043 g, 5.12 mmol) and O-(7-Azabenzotriazol-1-yl)-N,N,N′,N′-tetramethyluronium hexafluorophosphate (2.336 g, 6.14 mmol) in DMF (15 mL) at ambient temperature under nitrogen. The resulting solution was stirred at ambient temperature for 16 hours. The reaction mixture was evaporated to dryness and redissolved in EtOAc (50 mL) and washed sequentially with water (10 mL) and saturated brine (10 mL). The organic layer was dried over MgSO4, filtered and evaporated to afford crude product.

The crude product was purified by flash silica chromatography, elution gradient 1 to 6% DCM in MeOH. Pure fractions were evaporated to dryness to afford N-[(2r,5s)-5-hydroxyadamantan-2-yl]-2-methylsulfanyl-4-[(2R)-oxolan-2-yl]pyrimidine-5-carboxamide (1.180 g, 59.2%) as an off-white solid;

1H NMR (400.132 MHz, CDCl3) δ 1.50-1.59 (3H, m), 1.75-1.83 (6H, m), 1.90-1.97 (2H, m), 2.03-2.27 (6H, m), 2.59 (3H, s), 2.80-2.91 (1H, m), 3.89-3.93 (1H, m), 3.97-4.02 (1H, m), 4.20-4.26 (1H, m), 5.14 (1H, t), 7.91 (1H, d), 8.86 (1H, s)

m/z (ES+) (M+H)+=390; HPLC t_{R}=1.69 min.

### Intermediate 112

### (R,Z)-methyl 3-(dimethylamino)-2-(tetrahydrofuran-2-carbonyl)acrylate

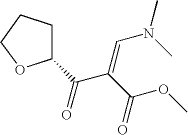

N,N-Dimethylformamide dimethyl acetal (1.668 mL, 12.55 mmol) was added in one portion to (R)-methyl 3-oxo-3-(tetrahydrofuran-2-yl)propanoate (1.8 g, 10.45 mmol) in dioxane (25 mL) at room temperature under nitrogen. The resulting solution was stirred at 100° C. for 2 hours. The reaction mixture was evaporated, to afford crude product. The crude product was purified by flash silica (120 g) chromatography, elution gradient 50 to 100% EtOAc in isohexane. Pure fractions were evaporated to dryness to afford (R,Z)-methyl 3-(dimethylamino)-2-(tetrahydrofuran-2-carbonyl)acrylate (1.800 g, 76%) as a yellow oil.

1H NMR (400.132 MHz, CDCl3) δ 1.83-1.92 (2H, m), 2.00-2.08 (1H, m), 2.12-2.21 (1H, m), 3.04 (6H, s), 3.73 (3H, s), 3.83-3.96 (2H, m), 4.97 (1H, t), 7.67 (1H, s)

m/z (ES+) (M−H)-=226; HPLC t_{R}=1.25 min.

### Intermediate 113

### (R)-methyl 2-(methylthio)-4-(tetrahydrofuran-2-yl)pyrimidine-5-carboxylate

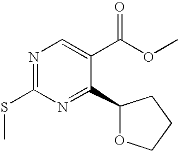

2-Methyl-2-Thiopseudourea Sulfate (1.543 g, 11.09 mmol) was added to (R,Z)-methyl 3-(dimethylamino)-2-(tetrahydrofuran-2-carbonyl)acrylate (Intermediate 112, 1.8 g, 7.92 mmol) and sodium acetate (2.73 g, 33.27 mmol) in DMF (30 mL) at 20° C. The resulting solution was stirred at 80° C. for 3 hours. Water was added to the cooled solution. The reaction mixture was diluted with EtOAc (200 mL), and washed sequentially with water (2×100 mL).The organic layer was dried over MgSO4, filtered and evaporated to afford crude product. The crude product was purified by flash silica chromatography, elution gradient 5 to 30% EtOAc in isohexane. Pure fractions were evaporated to dryness to afford (R)-methyl 2-(methylthio)-4-(tetrahydrofuran-2-yl)pyrimidine-5-carboxylate (1.460 g, 72.5%) as a colourless oil.

1H NMR (400.132 MHz, CDCl3) δ 1.96-2.10 (3H, m), 2.38-2.49 (1H, m), 2.60 (3H, s), 3.91 (3H, s), 4.00-4.05 (1H, m), 4.13-4.19 (1H, m), 5.69-5.74 (1H, m), 8.88 (1H, s)

m/z (ES+) (M+H)+=255; HPLC t_{R}=1.88 min.

### Intermediate 114

### (R)-2-(methylthio)-4-(tetrahydrofuran-2-yl)pyrimidine-5-carboxylic acid

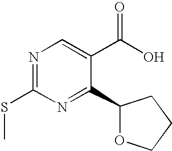

A solution of Lithium hydroxide monohydrate (0.482 g, 11.48 mmol) in water (10 mL) was added to a stirred solution of (R)-methyl 2-(methylthio)-4-(tetrahydrofuran-2-yl)pyrimidine-5-carboxylate (Intermediate 113, 1.46 g, 5.74 mmol) in THF (20 mL) at 20° C. The resulting mixture was stirred at 20° C. for 70 hours. The THF was evaporated and the aqueous washed with ethyl acetate (100 ml) to remove any impurities. The aqueous was acidified with 1M citric acid and extracted into ethyl acetate (100 ml). The organic layer was washed with brine (50 ml), dried over MgSO4, filtered and evaporated to give

(R)-2-(methylthio)-4-(tetrahydrofuran-2-yl)pyrimidine-5-carboxylic acid (1.230 g, 89%) as a white solid;

1H NMR (400.132 MHz, CDCl3) δ 1.99-2.20 (3H, m), 2.39-2.49 (1H, m), 2.62 (3H, s), 4.03-4.11 (1H, m), 4.16-4.22 (1H, m), 5.66-5.70 (1H, m), 9.02 (1H, s)

m/z (ES+) (M+H)+=241; HPLC t_{R}=0.59 min.

### Intermediate 115

### N-[(2r,5s)-5-hydroxyadamantan-2-yl]-2-methylsulfonyl-4-[(2R)-oxolan-2-yl]pyrimidine-5-carboxamide

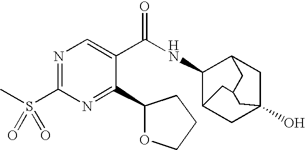

3-Chloroperoxybenzoic acid (70%) (1.392 g, 5.65 mmol) was added in one portion to N-[(2r,5 s)-5-hydroxyadamantan-2-yl]-2-methylsulfanyl-4-[(2R)-oxolan-2-yl]pyrimidine-5-carboxamide (Example 166, 1.1 g, 2.82 mmol) in DCM (45 mL) at 0° C. The resulting solution was stirred at 20° C. for 24 hours. The reaction mixture was diluted with DCM (50 mL), and washed sequentially with saturated NaHCO3 (4×75 mL) and saturated brine (75 mL). The organic layer was dried over MgSO4, filtered and evaporated to afford N-[(2r,5 s)-5-hydroxyadamantan-2-yl]-2-methylsulfonyl-4-[(2R)-oxolan-2-yl]pyrimidine-5-carboxamide (1.180 g, 99%) as a white solid;

1H NMR (400.132 MHz, CDCl3) δ 1.50-1.60 (3H, m), 1.74-1.85 (6H, m), 1.90-1.98 (2H, m), 2.08-2.31 (6H, m), 2.79-2.90 (1H, m), 3.36 (3H, s), 3.90-4.04 (2H, m), 4.23-4.30 (1H, m), 5.24 (1H, t), 7.88 (1H, d), 9.17 (1H, s)

m/z (ES+) (M+H)+=422; HPLC t_{R}=1.31 min.

The following Examples were prepared in a similar manner to Example 159, using Intermediate 115 and an appropriate amine starting material:

| | | | | MS |
|---|---|---|---|---|
| | | | | m/e |
| Structure | Ex | Name | ¹H NMR δ | MH⁺ |
| 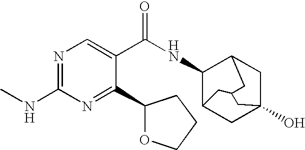 | 167 | N-[(2r,5s)-5- hydroxyada mantan-2- yl]-2- methylamino- 4-[(2R)- oxolan-2- yl]pyrimidine- 5- carboxamide | 1H NMR (400.132 MHz, CDCl3) δ 1.42(1 H, s), 1.52 (2 H, t), 1.76-1.86(6 H, m), 1.90-1.97(2 H, m), 2.02- 2.09(2 H, m), 2.14-2.26 (4 H, m), 2.81(1 H, s), 3.04 (3 H, d), 3.90(1 H, q), 4.01 (1 H, q), 4.18-4.24(1 H, m), 5.09(1 H, t), 5.33(1 H, d), 7.83(1 H, s), 8.72(1 H, s) | m/z (ES+) (M + H)+ = 373; HPLC t_{R} = 1.37 min. |
| 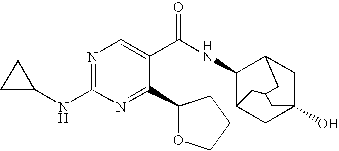 | 170 | 2- (cyclopropyl amino)-N- [(2r,5s)-5- hydroxyada mantan-2- yl]-4-[(2R)- oxolan-2- yl]pyrimidine- 5- carboxamide | 1H NMR (400.132 MHz, CDCl3) δ 0.54-0.58(2 H, m), 0.80-0.86(2 H, m), 1.37(1 H, s), 1.49-1.53 (2 H, m), 1.76-1.85(6 H, m), 1.90-1.97(2 H, m), 2.02-2.09(2 H, m), 2.15- 2.26(4 H, m), 2.72-2.85 (2 H, m), 3.90(1 H, q), 4.01 (1 H, q), 4.18-4.24(1 H, m), 5.10(1 H, t), 5.51(1 H, s), 7.77(1 H, s), 8.73(1 H, s) | m/z (ES+) (M + H)+ = 399; HPLC t_{R} = 1.56 min. |
| 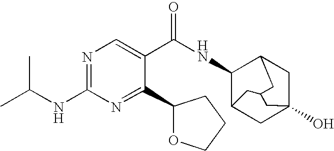 | 171 | N-[(2r,5s)-5- hydroxyada mantan-2- yl]-4-[(2R)- oxolan-2-yl]- 2-(propan-2- ylamino)pyri midine-5- carboxamide | 1H NMR (400.132 MHz, CDCl3) δ 1.25(6 H, $mult$), 1.48-1.58(3 H, m), 1.72-1.86(6 H, m), 1.90-1.97(2 H, m), 2.02- 2.09(2 H, m), 2.13-2.25 (4 H, m), 2.76(1 H, s), 3.89 (1 H, q), 4.01(1 H, q), 4.11- 4.25(2 H, m), 5.08(1 H, t), 5.23-5.27(1 H, m), 7.80 (1 H, s), 8.68(1 H, s) | m/z (ES+) (M + H)+ = 401; HPLC t_{R} = 1.79 min. |
| 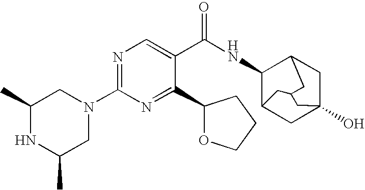 | 172 | 2-[(3S,5R)- 3,5- dimethylpipe razin-1-yl]- N-[(2r,5s)-5- hydroxyada mantan-2- yl]-4-[(2R)- oxolan-2- yl]pyrimidine- 5- carboxamide | 1H NMR (400.132 MHz, CDCl3) δ 1.14(6 H, dd), 1.45-1.57(4 H, m), 1.75- 1.83(6 H, m), 1.90-1.96 (2 H, m), 2.00-2.09(2 H, m), 2.14-2.26(4 H, m), 2.45-2.52(2 H, m), 2.74- 2.90(3 H, m), 3.90(1 H, q), 3.98-4.04(1 H, m), 4.19- 4.24(1 H, m), 4.69-4.74 (2 H, m), 5.08(1 H, t), 7.90 (1 H, d), 8.74(1 H, s) | m/z (ES+) (M + H)+ = 456; HPLC t_{R} = 1.59 min. |
| 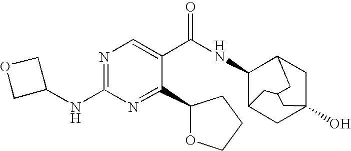 | 173 | N-[(2r,5s)-5- hydroxyada mantan-2- yl]-2- (oxetan-3- ylamino)-4- [(2R)- oxolan-2- yl]pyrimidine- 5- carboxamide | 1H NMR (400.132 MHz, CDCl3) δ 1.41-1.58(3 H, m), 1.75-1.85(6 H, m), 1.90-1.97(2 H, m), 2.03- 2.11(2 H, m), 2.14-2.24 (4 H, m), 2.66-2.78(1 H, m), 3.90(1 H, q), 3.97- 4.03(1 H, m), 4.19-4.24 (1 H, m), 4.60(2 H, t), 4.95- 5.00(2 H, m), 5.07(1 H, t), 5.14(1 H, sextet), 5.83(1 H, d), 7.78(1 H, s), 8.71(1 H, s) | m/z (ES+) (M + H)+ = 415; HPLC t_{R} = 1.31 min. |
| 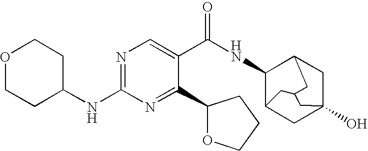 | 174 | N-[(2r,5s)-5- hydroxyada mantan-2- yl]-2-(oxan- 4-ylamino)- 4-[(2R)- oxolan-2- yl]pyrimidine- 5- carboxamide | 1H NMR (400.13 MHz, CDCl₃) δ 1.48-1.62(6 H, m), 1.73-1.84(6 H, m), 1.89-1.97(2 H, m), 1.99- 2.11(4 H, m), 2.14-2.25 (4 H, d), 2.71(1 H, bs), 3.54 (2 H, td), 3.87-3.93(1 H, m), 3.96-4.02(2 H, m), 4.04-4.12(1 H, m), 4.19- 4.23(1 H, m), 5.08(1 H, t), 5.34(1 H, s), 7.76(1 H, bs), 8.69(1 H, s) | m/z (ESI+) (M + H)+ = 443; HPLC t_{R} = 1.56 min. |
| 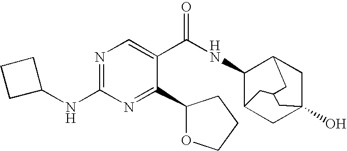 | 175 | 2- (cyclobutayla mino)-N- [(2r,5s)-5- hydroxyada mantan-2- yl]-4-[(2R)- oxolan-2- yl]pyrimidine- 5- carboxamide | 1H NMR (400.13 MHz, DMSO-d₆) δ 1.33(2 H, d), 1.59(3 H, s), 1.61-1.73 (6 H, m), 1.83-1.92(4 H, m), 1.92-2.05(5 H, m), 2.09 (1 H, s), 2.17-2.25(2 H, m), 3.73-3.80(1 H, m), 3.86- 3.95(2 H, m), 4.30-4.38 (1 H, m), 4.43(1 H, s), 5.04 (1 H, bs), 7.69(1 H, d), 8.03 (1 H, d), 8.25(1 H, s) | m/z (ESI+) (M + H)+ = 413; HPLC t_{R} = 1.91 min. |

The following Examples were prepared in a similar manner to Example 75, using Intermediate 115 and an appropriate starting material:

| | | | | MS m/e |
|---|---|---|---|---|
| Structure | Ex | Name | ¹H NMR δ | MH⁺ |
| 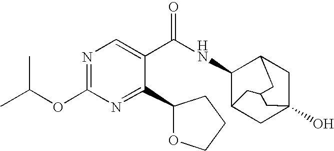 | 176 | N-[(2r,5s)-5- hydroxyadaman- tan-2-yl]- 4-[(2R)- oxolan-2-yl]- 2-propan-2- yloxypyrimidine- 5- carboxamide | 1H NMR (400.132 MHz, CDCl3) δ 1.39(1 H, s), 1.41(6 H, d), 1.50-1.55 (2 H, m), 1.75-1.85(6 H, m), 1.91-2.00(2 H, m), 2.05-2.26(6 H, m), 2.76- 2.86(1 H, m), 3.91(1 H, q), 4.01(1 H, q), 4.20- 4.25(1 H, m), 5.12(1 H, t), 5.33(1 H, septet), 7.84 (1 H, d), 8.86(1 H, s) | m/z (ES+) (M + H)+ = 402; HPLC t_{R} = 1.77 min. |

The following intermediates were used and were prepared as described below.

### Intermediate 117

### (S,Z)-methyl 3-(dimethylamino)-2-(tetrahydrofuran-2-carbonyl)acrylate

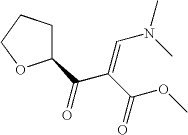

Prepared from (S)-methyl 3-oxo-3-(tetrahydrofuran-2-yl)propanoate by the same process used for Intermediate 1;

1H NMR (400.132 MHz, CDCl3) δ 1.83-1.92 (2H, m), 2.01-2.08 (1H, m), 2.13-2.22 (1H, m), 3.05 (6H, s), 3.74 (3H, s), 3.83-3.96 (2H, m), 4.97 (1H, t), 7.67 (1H, s)

m/z (ES+) (M+H)+=228; HPLC t_{R}=1.27 min.

### Intermediate 118

### (S)-methyl 2-(methylthio)-4-(tetrahydrofuran-2-yl)pyrimidine-5-carboxylate

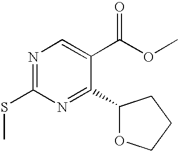

Prepared from Intermediate 117 by the Same Process Used for Intermediate 28

1H NMR (400.132 MHz, CDCl3) δ 1.94-2.10 (3H, m), 2.37-2.46 (1H, m), 2.61 (3H, s), 3.91 (3H, s), 4.00-4.05 (1H, m), 4.12-4.19 (1H, m), 5.68-5.74 (1H, m), 8.88 (1H, s)

m/z (ES+) (M+H)+=255; HPLC t_{R}=1.88 min.

### Intermediate 119

### (S)-2-(methylthio)-4-(tetrahydrofuran-2-yl)pyrimidine-5-carboxylic acid

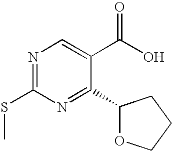

Prepared from Intermediate 118 by the same process used for Intermediate 29;

1H NMR (400.132 MHz, CDCl3) δ 2.00-2.17 (3H, m), 2.39-2.50 (1H, m), 2.62 (3H, s), 4.04-4.13 (1H, m), 4.17-4.24 (1H, m), 5.71-5.77 (1H, m), 7.03 (1H, bs), 9.03 (1H, s)

m/z (ES+) (M+H)+=241; HPLC t_{R}=0.54 min.

### Example 177

### N-[(2r,5s)-5-hydroxyadamantan-2-yl]-2-methylsulfanyl-4-[(2S)-oxolan-2-yl]pyrimidine-5-carboxamide

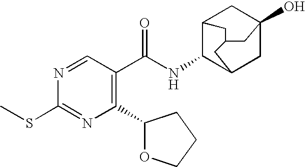

N-Ethyldiisopropylamine (11.89 mL, 68.25 mmol) was added to (S)-2-(methylthio)-4-(tetrahydrofuran-2-yl)pyrimidine-5-carboxylic acid (Intermediate 119, 4.1 g, 17.06 mmol), 4-aminoadamantan-1-ol hydrochloride (3.48 g, 17.06 mmol) and O-(7-Azabenzotriazol-1-yl)-N,N,N′,N′-tetramethyluronium hexafluorophosphate (7.79 g, 20.48 mmol) in DMF (40 mL) at ambient temperature under nitrogen. The resulting solution was stirred at ambient temperature for 16 hours. The reaction mixture was evaporated to dryness and redissolved in EtOAc (50 mL) and washed sequentially with water (100 mL) and saturated brine (100 mL). The organic layer was dried over MgSO4, filtered and evaporated to afford crude product.

The crude product was purified by flash silica chromatography, elution gradient 1 to 6% DCM in MeOH. Pure fractions were evaporated to dryness to afford N-[(2r,5s)-5-hydroxyadamantan-2-yl]-2-methylsulfanyl-4-[(2S)-oxolan-2-yl]pyrimidine-5-carboxamide (3.48 g, 52.4%) as an off-white solid;

1H NMR (400.132 MHz, CDCl3) δ 1.50-1.59 (3H, m), 1.75-1.83 (6H, m), 1.90-1.97 (2H, m), 2.03-2.27 (6H, m), 2.59 (3H, s), 2.80-2.91 (1H, m), 3.89-3.93 (1H, m), 3.97-4.02 (1H, m), 4.20-4.26 (1H, m), 5.14 (1H, t), 7.91 (1H, d), 8.86 (1H, s)

m/z (ES+) (M+H)+=390; HPLC t_{R}=1.69 min.

### Intermediate 121

### N-[(2r,5s)-5-hydroxyadamantan-2-yl]-2-methylsulfonyl-4-[(2S)-oxolan-2-yl]pyrimidine-5-carboxamide

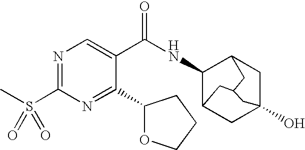

Prepared from Example 177 by the same process used for Example 4;

1H NMR (400.132 MHz, CDCl3) δ 1.50-1.60 (3H, m), 1.74-1.85 (6H, m), 1.90-1.98 (2H, m), 2.08-2.31 (6H, m), 2.79-2.90 (1H, m), 3.36 (3H, s), 3.90-4.04 (2H, m), 4.23-4.30 (1H, m), 5.24 (1H, t), 7.88 (1H, d), 9.17 (1H, s)

m/z (ES+) (M+H)+=422; HPLC t_{R}=1.30 min.

The following Examples were prepared in a similar manner to Example 159, using Intermediate 121 and an appropriate amine starting material:

| | | | | MS |
|---|---|---|---|---|
| | | | | m/e |
| Structure | Ex | Name | ¹H NMR δ | MH⁺ |
| 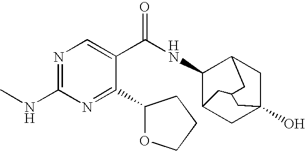 | 178 | N-[(2r,5s)-5- hydroxyada mantan-2- yl]-2- methylamino- 4-[(2S)- oxolan-2- yl]pyrimidine- 5- carboxamide | 1H NMR (400.132 MHz, CDCl3) δ 1.40(1 H, s), 1.52(2 H, t), 1.76-1.85 (6 H, m), 1.90-1.96(2 H, m), 2.01-2.10(2 H, m), 2.14-2.25(4 H, m), 2.78 (1 H, s), 3.04(3 H, d), 3.90(1 H, q), 4.01(1 H, q), 4.19-4.24(1 H, m), 5.09(1 H, t), 5.31(1 H, d), 7.80(1 H, s), 8.71(1 H, s) | m/z (ES+) (M + H)+ = 373; HPLC t_{R} = 1.36 min. |
| 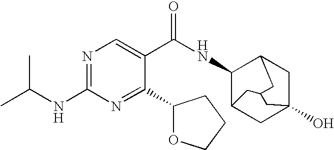 | 179 | N-[(2r,5s)-5- hydroxyada mantan-2- yl]-4-[(2S)- oxolan-2-yl]- 2-(propan-2- ylamino)pyri midine-5- carboxamide | 1H NMR (400.132 MHz, CDCl3) δ 1.25(6 H, dd), 1.40(1 H, s), 1.52(2 H, t), 1.75-1.83(6 H, m), 1.91- 1.96(2 H, m), 2.02- 2.09(2 H, m), 2.13-2.26 (4 H, m), 2.76(1 H, s), 3.89(1 H, q), 4.01(1 H, q), 4.12-4.23(2 H, m), 5.08(1 H, t), 5.20(1 H, d), 7.82(1 H, s), 8.69(1 H, s) | m/z (ES+) (M + H)+ = 401; HPLC t_{R} = 1.78 min. |
| 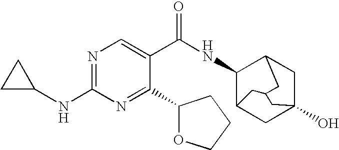 | 180 | 2- (cyclopropyl amino)-N- [(2r,5s)-5- hydroxyada mantan-2- yl]-4-[(2S)- oxolan-2- yl]pyrimidine- 5- carboxamide | 1H NMR (400.132 MHz, CDCl3) δ 0.54-0.58 (2 H, m), 0.81-0.85(2 H, m), 1.42(1 H, s), 1.53 (2 H, t), 1.77-1.86(6 H, m), 1.90-1.97(2 H, m), 2.02-2.09(2 H, m), 2.15- 2.25(4 H, m), 2.71- 2.85(2 H, m), 3.89(1 H, q), 4.01(1 H, q), 4.18- 4.24(1 H, m), 5.10(1 H, t), 5.53(1 H, s), 7.78(1 H, s), 8.74(1 H, s) | m/z (ES+) (M + H)+ = 399; HPLC t_{R} = 1.56 min. |

The following Examples were prepared in a similar manner to Example 75, using Intermediate 121 and an appropriate starting material:

| | | | | MS m/e |
|---|---|---|---|---|
| Structure | Ex | Name | ¹H NMR δ | MH⁺ |
| 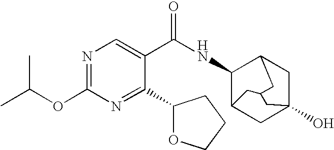 | 181 | N-[(2r,5s)-5- hydroxyada mantan-2- yl]-4-[(2S)- oxolan-2-yl]- 2-propan-2- yloxypyrimi dine-5- carboxamide | 1H NMR (400.132 MHz, CDCl3) δ 1.39(1 H, s), 1.41(6 H, d), 1.49-1.57 (2 H, m), 1.76-1.85(6 H, m), 1.91-1.96(2 H, 2.04-2.26(6 H, m), 2.76- 2.85(1 H, m), 3.91(1 H, q), 4.00(1 H, q), 4.21- 4.25(1 H, m), 5.12(1 H, t), 5.33(1 H, septet), 7.82 (1 H, d), 8.86(1 H, s) | m/z (ES+) (M + H)+ = 402; HPLC t_{R} = 1.75 min. |

### Example 182

### N-[(2r,5 s)-5-Hydroxyadamantan-2-yl] 2-[(2S,6R)-2,6-dimethylmorpholin-4-yl]-4-[(2R)-oxolan-2-yl]pyrimidine-5-carboxamide

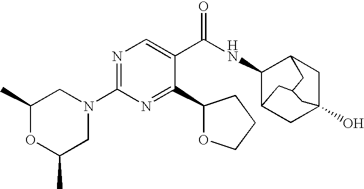

N-[(2r,5s)-5-hydroxyadamantan-2-yl]-2-methylsulfonyl-4-[(2R)-oxolan-2-yl]pyrimidine-5-carboxamide (Intermediate 115, 12.3 g, 29.18 mmol) and (2R,6S)-2,6-dimethylmorpholine (15 mL, 121.12 mmol) were dissolved in THF (150 mL) under N2. The resulting solution was stirred at 20° C. for 24 hours. The reaction mixture was evaporated to dryness and the crude product was purified by flash silica chromatography, elution gradient 1 to 5% MeOH in EtOAc. Pure fractions were evaporated to dryness and triturated with ether to afford N-[(2r,5s)-5-hydroxyadamantan-2-yl] 2-[(2S,6R)-2,6-dimethylmorpholin-4-yl]-4-[(2R)-oxolan-2-yl]pyrimidine-5-carboxamide (7.80 g, 58.5%) as a white solid.

The compound was further purified by chiral chromatography (Merck 100 mm 20 μm Chiralpak AS column, Flow: 150 ml/min) eluting with iso-Hexane/EtOH 70/30. Pure fractions were evaporated to dryness to afford N-[(2r,5s)-5-hydroxyadamantan-2-yl] 2-[(2S,6R)-2,6-dimethylmorpholin-4-yl]-4-[(2R)-oxolan-2-yl]pyrimidine-5-carboxamide 100% enantiomerically pure;

1H NMR (400.132 MHz, CDCl3) δ 1.26 (6H, d), 1.41 (1H, s), 1.48-1.58 (2H, m), 1.75-1.85 (6H, m), 1.89-1.96 (2H, m), 2.01-2.09 (2H, m), 2.14-2.23 (4H, m), 2.62 (2H, t), 2.74-2.82 (1H, m), 3.57-3.66 (2H, m), 3.91 (1H, q), 3.98-4.03 (1H, m), 4.19-4.26 (1H, m), 4.63 (2H, d), 5.08 (1H, t), 7.90 (1H, d), 8.75 (1H, s)

m/z (ES+) (M+H)+=457; HPLC t_{R}=1.96 min.

### Intermediate 173

### (2R,6S)-2,6-dimethylmorpholine-4-carboximidamide

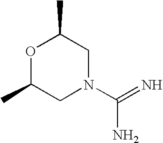

Methyl carbamimidothioate hemisulfate (148 g, 520.95 mmol) was added in one portion to (2S,6R)-2,6-dimethylmorpholine (103 g, 868.26 mmol), in water (5 vol) (500 mL) warmed to 100° C. The resulting slurry was stirred at 100° C. for 1 hour. To the colourless solution was added dropwise Barium chloride dihydrate (127 g, 520.95 mmol) in water (400 ml, 4 vol) and the reaction mixture left heating for another hour and the reaction was cooled to ambient and the white precipitate filtered off through Dicalite and the aqueous filtrates evaporated to dryness then azeotroped with toluene. To the residue was added ethanol (400 ml, 4 vol) and the white solid filtered washed with diethyl ether (200 ml, 2 vol) air dried to give (2R,6S)-2,6-dimethylmorpholine-4-carboximidamide (92 g, 55%) and the mother liquors evaporated and more ethanol (200 ml, 2 vol) charged the white solid filtered off washed with ethanol (200 ml, 2 vol) to give (2R,6S)-2,6-dimethylmorpholine-4-carboximidamide (3.2 g, 2%).

1H NMR (400 MHz, DMSO) δ 1.09 (6H, d), 2.63 (2H, dd), 3.63-3.48 (2H, m), 3.83 (2H, d), 7.68 (4H, s).

### Intermediate 169

### methyl 2-((2S,6R)-2,6-dimethylmorpholino)-4-((R)-tetrahydrofuran-2-yl)pyrimidine-5-carboxylate

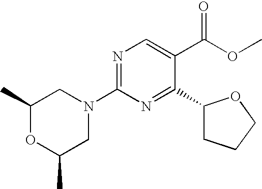

(2R,6S)-2,6-dimethylmorpholine-4-carboximidamide (Intermediate 173, 190 mg, 0.98 mmol) was added in one portion to (R,Z)-methyl 3-(dimethylamino)-2-(tetrahydrofuran-2-carbonyl)acrylate (Intermediate 112, 223 mg, 0.98 mmol), and Sodium acetate (338 mg, 4.12 mmol) in DMF (10 mL) at 20° C. under nitrogen. The resulting suspension was stirred at 80° C. for 4 hours. LC-MS (EN01493-77-C2) shows 7% starting material so additional (2R,6S)-2,6-dimethylmorpholine-4-carboximidamide (20 mg, 0.1 eq) was charged and left stirring for an additional 2 hours LC-MS (EN01493-77-C4) shows 1.6% starting material the reaction was allowed to cool to ambient drowned out with water (100 ml), extracted with ethyl acetate (2×50 ml). The combined organic layers were washed with water (2×50 ml) and the organic layer put down a phase separating cartridge to remove the water. The crude product was purified by flash silica chromatography, elution gradient 30% EtOAc in isohexane. Pure fractions were evaporated to dryness to afford methyl 2-((2S,6R)-2,6-dimethylmorpholino)-4-((R)-tetrahydrofuran-2-yl)pyrimidine-5-carboxylate (213 mg, 67.5%) as a pale yellow oil which solidified on standing;

1H NMR (400 MHz, CDCl) δ 1.19 (6H, d), 2.05-1.78 (3H, m), 2.45-2.26 (1H, m), 2.75-2.48 (2H, m), 3.69-3.46 (2H, m), 3.78 (3H, s), 4.02-3.93 (1H, m), 4.17-4.03 (1H, m), 4.79-4.49 (2H, m), 5.71 (1H, dd), 8.74 (1H, s).

m/z (ES+) (M+H)+=321; HPLC t_{R}=2.27 min.

### Intermediate 170

### 2-((2S,6R)-2,6-dimethylmorpholino)-4-((R)-tetrahydrofuran-2-yl)pyrimidine-5-carboxylic acid

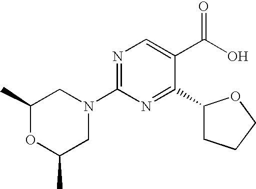

Sodium hydroxide (0.327 mL, 0.65 mmol) was added dropwise to methyl 2-((2S,6R)-2,6-dimethylmorpholino)-4-((R)-tetrahydrofuran-2-yl)pyrimidine-5-carboxylate (Intermediate 169, 105 mg, 0.33 mmol) in methanol (10 mL) under nitrogen. The resulting solution was stirred at 20° C. for 3 hours. LC-MS (EN01493-86-C1) shows 1% product so additional Sodium hydroxide (0.327 mL, 0.65 mmol) was charged after a further 2 hours LC-MS (EN01493-86-C2) shows 2% product so 5N NaOH (0.327 ml, 5 eq) was charged and the reaction stirred overnight. LC-MS (EN01493-86-C3) shows 72% product and 28% SM so the reaction was warmed to 40° C. after 5 hours LC-MS (EN01493-86-C7) shows no starting material. The reaction mixture was evaporated taken up with water (50 ml) and the solution was adjusted to pH3 with 2M HCl. The aqueous layer was extracted with ethyl acetate (2×50 ml) dried and evaporated to provide 2-((2S,6R)-2,6-dimethylmorpholino)-4-((R)-tetrahydrofuran-2-yl)pyrimidine-5-carboxylic acid (99 mg, 99%) as a white solid;

1H NMR (400 MHz, CDCl3) δ 1.20 (6H, d), 2.05-1.82 (3H, m), 2.47-2.25 (1H, m), 2.78-2.48 (2H, m), 3.70-3.47 (2H, m), 4.04-3.96 (1H, m), 4.18-4.04 (1H, m), 4.66 (2H, d), 8.85 (1H, s), 5.78-5.58 (1H, m)

m/z (ES+) (M+H)+=308; HPLC t_{R}=0.89 min.

### Example 183

### 2-[(2S,6R)-2,6-Dimethylmorpholin-4-yl]-N-[(2r,5s)-5-hydroxyadamantan-2-yl]-4-[(2S)-oxolan-2-yl]pyrimidine-5-carboxamide

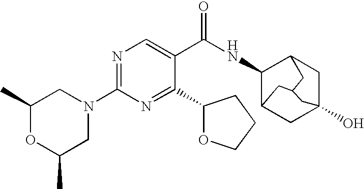

Prepared from Intermediate 172 by the Same Process Used for Example 182

1H NMR (400.13 MHz, CDCl₃) δ 1.26 (6H, d), 1.45-1.60 (3H, m), 1.75-1.84 (6H, m), 1.90-1.98 (2H, m), 2.01-2.11 (2H, m), 2.16-2.20 (2H, m), 2.18-2.23 (2H, m), 2.63 (2H, dd), 2.76-2.81 (1H, m), 3.59-3.66 (2H, m), 3.92 (1H, q), 3.98-4.03 (1H, m), 4.19-4.24 (1H, m), 4.63 (2H, d), 5.08 (1H, t), 7.90 (1H, d), 8.75 (1H, s)

m/z (ESI+) (M+H)+=457; HPLC t_{R}=1.93 min.

### Intermediate 171

### methyl 2-((2S,6R)-2,6-dimethylmorpholino)-4-((S)-tetrahydrofuran-2-yl)pyrimidine-5-carboxylate

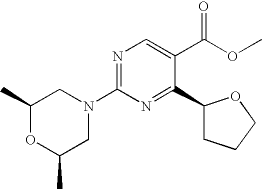

Prepared from Intermediate 117 by the same process used for Intermediate 169;

1H NMR (400.13 MHz, CDCl₃) δ 1.24-1.28 (6H, m), 1.93-2.02 (3H, m), 2.37-2.45 (1H, m), 2.61-2.73 (2H, m), 3.58-3.67 (2H, m), 3.85 (3H, s), 4.04-4.19 (2H, m), 4.69-4.77 (2H, m), 5.75-5.79 (1H, m), 8.80 (1H, s)

m/z (ESI+) (M+H)+=322; HPLC t_{R}=2.14 min.

### Intermediate 172

### 2-((2S,6R)-2,6-dimethylmorpholino)-4-((S)-tetrahydrofuran-2-yl)pyrimidine-5-carboxylic acid

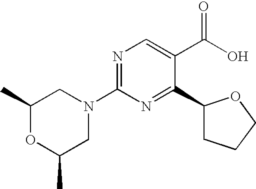

Prepared from Intermediate 171 by the Same Process Used for Intermediate 170

1H NMR (400.13 MHz, DMSO-d₆) δ 1.15 (6H, d), 1.82-1.96 (3H, m), 1.82-1.98 (1H, m), 2.21-2.26 (1H, m), 2.55-2.64 (2H, m), 3.52-3.57 (2H, m), 3.86-3.90 (1H, m), 3.99-4.05 (1H, m), 4.58 (2H, d), 5.66-5.70 (1H, m), 8.72 (1H, s)

m/z (ESI+) (M+H)+=308; HPLC t_{R}=0.93 min.

### Example 184

### 4-(3,3-Difluorocyclopentyl)-N-[(2r,5s)-5-hydroxyadamantan-2-yl]-2-methylpyrimidine-5-carboxamide

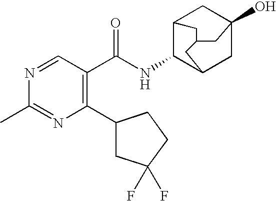

Prepared from Intermediate 125 by the same process used for Example 4;

1H NMR (400.132 MHz, CDCl3) δ 1.55-1.99 (11H, m), 2.02-2.23 (4H, m), 2.24-2.29 (2H, m), 2.30-2.47 (2H, m), 2.56-2.72 (1H, m), 2.73 (3H, s), 3.76-3.89 (1H, m), 4.18-4.26 (1H, m), 5.91-6.03 (1H, m), 8.57 (1H, s)

m/z (ESI+) (M+H)+=392; HPLC t_{R}=1.77 min.

### Intermediate 175

### 5-(3,3-difluorocyclopentanecarbonyl)-2,2-dimethyl-1,3-dioxane-4,6-dione

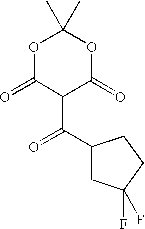

A solution of 3,3-difluorocyclopentanecarbonyl chloride (2.4 g, 14.24 mmol) in dichloromethane (5 mL) was added dropwise to a stirred solution of Isopropylidene malonate (2.257 g, 15.66 mmol) and Pyridine (2.301 mL, 28.47 mmol) in dichloromethane (50 mL) at 0° C., over a period of 10 minutes under nitrogen. The resulting suspension was stirred at 0° C. for 45 minutes then 4 hours at room temperature. The reaction mixture was diluted with DCM and washed sequentially with 1M citric acid, water and saturated brine. The organic layer was dried over MgSO4, filtered and evaporated to afford 5-(3,3-difluorocyclopentanecarbonyl)-2,2-dimethyl-1,3-dioxane-4,6-dione (3.20 g, 81%) as a brown oil which was used in the next stage without further purification.

m/z (ESI−) (M−H)-=275; HPLC t_{R}=2.34 min.

### Intermediate 122

### methyl 3-(3,3-difluorocyclopentyl)-3-oxopropanoate

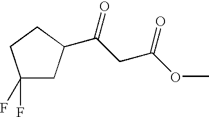

Methanol (50 mL) was added in one portion to a stirred solution of 5-(3,3-difluorocyclopentanecarbonyl)-2,2-dimethyl-1,3-dioxane-4,6-dione (Intermediate 175, 3.2 g, 11.58 mmol) in toluene (100 mL).The reaction was heated to 125° C. and maintained at this for 4 hours. The cooled reaction was evaporated to dryness to afford crude product The crude product was purified by flash silica (120 g) chromatography, elution gradient 0 to 20% EtOAc in isohexane. Pure fractions were evaporated to dryness to afford methyl 3-(3,3-difluorocyclopentyl)-3-oxopropanoate (1.040 g, 43.5%) as a colourless oil;

1H NMR (400.132 MHz, CDCl3) δ 1.89-2.50 (6H, m), 3.20-3.31 (1H, m), 3.51 (2H, s), 3.75 (3H, s)

m/z no obvious mass ion—no major ion peak in + or −ve=; HPLC t_{R}=2.33 min

### Intermediate 123

### (Z)-methyl 2-(3,3-difluorocyclopentanecarbonyl)-3-(dimethylamino)acrylate

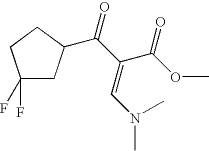

Prepared from methyl methyl 3-(3,3-difluorocyclopentyl)-3-oxopropanoate by the same process used for Intermediate 1;

1H NMR (400.132 MHz, CDCl3) δ 1.85-2.49 (6H, m), 2.60-3.43 (7H, m), 3.75 (3H, s), 7.71 (1H, s)

m/z (ESI+) (M+H)+=262; HPLC t_{R}=1.70 min.

### Intermediate 124

### methyl 4-(3,3-difluorocyclopentyl)-2-methylpyrimidine-5-carboxylate

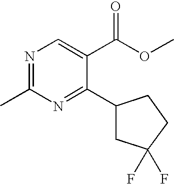

Prepared by the same process used for Intermediate 2 from (Z)-methyl 2-(3,3-difluorocyclopentanecarbonyl)-3-(dimethylamino)acrylate;

1H NMR (400.132 MHz, CDCl3) δ 1.97-2.27 (3H, m), 2.28-2.48 (2H, m), 2.58-2.73 (1H, m), 2.75 (3H, s), 3.94 (3H, s), 4.25-4.36 (1H, m), 9.03 (1H, s)

m/z (ESI+) (M+H)+=257; HPLC t_{R}=2.19 min

### Intermediate 125

### 4-(3,3-difluorocyclopentyl)-2-methylpyrimidine-5-carboxylic acid

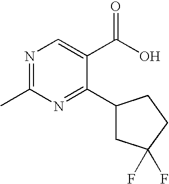

Prepared from Intermediate 124 by the same process used for Intermediate 29;

1H NMR (400.132 MHz, CDCl3) δ 2.00-2.14 (1H, m), 2.15-2.32 (2H, m), 2.33-2.54 (2H, m), 2.60-2.79 (1H, m), 2.81 (3H, s), 4.38-4.49 (1H, m), 7.52-9.12 (1H, m), 9.22 (1H, s)

m/z (ESI+) (M+H)+=243; HPLC t_{R}=1.69 min.

### Example 185

### N-[(2r,5 s)-5-hydroxyadamantan-2-yl]-4-(1-methylcyclopropyl)-2-morpholin-4-ylpyrimidine-5-carboxamide

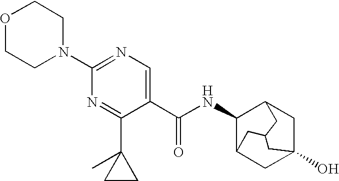

4-(1-methylcyclopropyl)-2-morpholin-4-yl-N-(5-phenylmethoxy-2-adamantyl)pyrimidine-5-carboxamide (Intermediate 128, 0.45 g, 0.90 mmol) and 10% Palladium on carbon (45 mg, 0.04 mmol) in ethanol (10 mL) and THF (10.00 mL) were stirred under an atmosphere of hydrogen at 1 atm and 20° C. for 20 hours. The reaction mixture was filtered through celite and evaporated to give a colourless oil. The crude product was purified by flash silica chromatography, elution gradient 0 to 6% MeOH in DCM. Pure fractions were evaporated to dryness to afford cis N-[(2r,5s)-5-hydroxyadamantan-2-yl]-4-(1-methylcyclopropyl)-2-morpholin-4-ylpyrimidine-5-carboxamide (0.087 g, 23.56%) as a white solid and trans N-[(2r,5s)-5-hydroxyadamantan-2-yl]-4-(1-methylcyclopropyl)-2-morpholin-4-ylpyrimidine-5-carboxamide (0.042 g, 11.37%) as a white solid.

1H NMR (400.132 MHz, CDCl3) δ 0.76-0.79 (2H, m), 1.20-1.26 (2H, m), 1.46 (3H, s), 1.54-1.57 (1H, m), 1.69-1.84 (8H, m), 1.93-1.99 (2H, m), 2.15-2.20 (1H, m), 2.23-2.28 (2H, m), 3.75 (4H, t), 3.85 (4H, t), 4.22-4.27 (1H, m), 6.45 (1H, d), 8.55 (1H, s)

m/z (ESI+) (M+H)+=413; HPLC t_{R}=1.71 min

### Example 186

### N-[(2r,5 s)-5-hydroxyadamantan-2-yl]-4-(1-methylcyclopropyl)-2-morpholin-4-ylpyrimidine-5-carboxamide

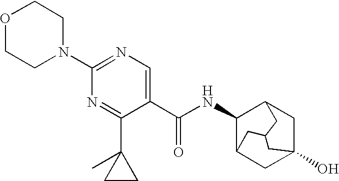

This compound was a byproduct from the synthesis of Example 185;

1H NMR (400.132 MHz, CDCl3) δ 0.75-0.81 (2H, m), 1.21-1.25 (2H, m), 1.46 (3H, s), 1.49-1.51 (1H, m), 1.62-1.84 (10H, m), 2.16-2.19 (1H, m), 2.29-2.33 (2H, m), 3.75 (4H, t), 3.85 (4H, t), 4.14-4.18 (1H, m), 6.48 (1H, d), 8.08 (1H, s)

m/z (ESI+) (M+H)+=413; HPLC t_{R}=1.71 min.

### Intermediate 176

### 4-isocyanato-1-phenylmethoxyadamantane

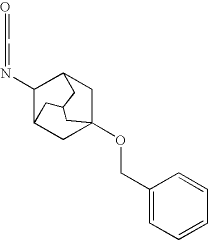

A solution of 20% phosgene in toluene (16.57 ml, 31.5 mmol) was added to 5-phenylmethoxyadamantan-2-amine hydrochloride (4.63 g, 15.76 mmol) and the resulting suspension was stirred at 100° C. for 6 hours with a dry ice condenser to avoid loss of phosgene from the reaction mixture. All of the solid dissolves during the course of the heating. Cooled, filtered and evaporated to give the crude product, 4-isocyanato-1-phenylmethoxyadamantane (4.02 g, 90%) as a red oil. Intermediate 176 was used in the next synthetic step without characterisation.

### Intermediate 126

### 3-(1-methylcyclopropyl)-3-oxo-N-(5-phenylmethoxy-2-adamantyl)propanamide

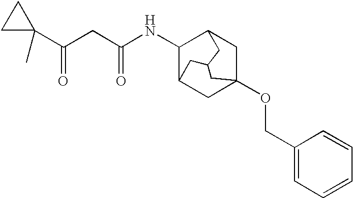

A solution of Lithium bis(trimethylsilyl)amide (15.61 mL, 15.61 mmol) was added to THF (15 mL) and cooled under nitrogen to −78° C. A solution of 1-(1-methylcyclopropyl)ethanone (1.532 g, 15.61 mmol) in THF (5 mL) was added dropwise over a period of 5 minutes under nitrogen. The resulting solution was stirred at −78° C. for 15 minutes. A solution of 4-isocyanato-1-phenylmethoxyadamantane (Intermediate 176, 4.02 g, 14.19 mmol) in THF (10 mL) was added over a period of 5 minutes under nitrogen. The resulting solution was stirred at −78° C. for 1 hour and the allowed to warm to 20° C. over 1 h. The reaction mixture was poured into saturated NH4Cl (250 mL) and extracted with EtOAc (2×150 mL), the organic layer was washed with water (50 mL) and brine (50 mL) dried over MgSO4, filtered and evaporated to afford a yellow oil. The crude product was purified by flash silica chromatography, elution gradient 20 to 60% EtOAc in isohexane. Pure fractions were evaporated to dryness to afford 3-(1-methylcyclopropyl)-3-oxo-N-(5-phenylmethoxy-2-adamantyl)propanamide (2.76 g, 51.0%) as a colourless oil.

1H NMR (400.132 MHz, CDCl3) δ 0.83-0.89 (2H, m), 1.33-1.38 (5H, m), 1.71-2.02 (10H, m), 2.13-2.24 (3H, m), 3.33 (2H, 2×s), 3.93-4.07 (1H, m), 4.51 (2H, 2×s), 7.22-7.39 (5H, m), 7.75-7.86 (1H, m)

m/z (ESI+) (M+H)+=382; HPLC t_{R}=2.59 min.

### Intermediate 127

### (Z)-3-dimethylamino-2-(1-methylcyclopropanecarbonyl)-N-(5-phenylmethoxy-2-adamantyl)prop-2-enamide

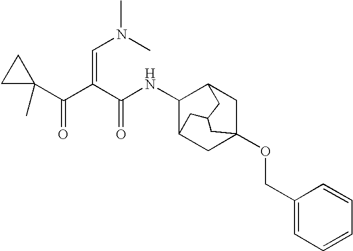

Prepared from Intermediate 126 by the same process used for Intermediate 1;

1H NMR (400.132 MHz, CDCl3) δ 0.62-0.71 (2H, m), 1.01-1.18 (2H, m), 1.36 (3H, s), 1.48-1.53 (1H, m), 1.67-1.79 (3H, m), 1.83-1.90 (4H, m), 1.98-2.06 (2H, m), 2.12-2.18 (2H, m), 2.21-2.26 (1H, m), 3.11 (6H, 2×s), 3.95-4.10 (1H, m), 4.52 (2H, 2×s), 7.21-7.25 (1H, m), 7.29-7.37 (5H, m), 7.90 (1H, d)

m/z (ESI+) (M+H)+=437; HPLC t_{R}=2.23 min.

### Intermediate 128

### 4-(1-methylcyclopropyl)-2-morpholin-4-yl-N-(5-phenylmethoxy-2-adamantyl)pyrimidine-5-carboxamide

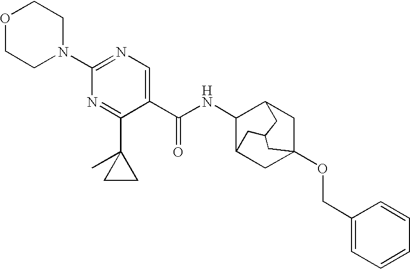

A solution of (Z)-3-dimethylamino-2-(1-methylcyclopropanecarbonyl)-N-(5-phenylmethoxy-2-adamantyl)prop-2-enamide (Intermediate 127, 0.6 g, 1.37 mmol) in methanol (3 mL) was added dropwise to a stirred suspension of Morpholinoformamidine hydrobromide (0.289 g, 1.37 mmol), and Sodium methoxide (0.5M in MeOH) (2.75 mL, 1.37 mmol) in methanol (8 mL) at 20° C. The resulting solution was stirred at 80° C. for 4 hours.

The reaction mixture was evaporated to dryness and redissolved in EtOAc (100 mL), and washed sequentially with water (75 mL) and saturated brine (75 mL). The organic layer was dried over MgSO4, filtered and evaporated to afford crude product. The crude product was purified by flash silica chromatography, elution gradient 40 to 70% EtOAc in isohexane. Pure fractions were evaporated to dryness to afford 4-(1-methylcyclopropyl)-2-morpholin-4-yl-N-(5-phenylmethoxy-2-adamantyl)pyrimidine-5-carboxamide (0.450 g, 65.1%) as a colourless oil.

1H NMR (400.132 MHz, CDCl3) δ 0.76-0.81 (2H, m), 1.21-1.29 (2H, m), 1.46 (3H, 2×s), 1.58-1.64 (1H, m), 1.73-1.97 (7H, m), 2.06-2.11 (1H, m), 2.19-2.23 (1H, m), 2.28-2.37 (2H, m), 3.75 (4H, t), 3.85 (4H, t), 4.17-4.29 (1H, m), 4.51 (2H, 2×s), 6.44-6.56 (1H, m), 7.21-7.26 (1H, m), 7.30-7.35 (5H, m), 8.56 (1H, 2×s)

m/z (ESI+) (M+H)+=503; HPLC t_{R}=2.98 min.

### Example 187

### N-[(2r,5 s)-5-hydroxyadamantan-2-yl]-2-methoxy-4-(1-methylcyclopropyl)pyrimidine-5-carboxamide

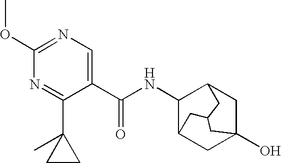

2-methoxy-4-(1-methylcyclopropyl)-N-(5-phenylmethoxy-2-adamantyl)pyrimidine-5-carboxamide (Intermediate 130, 0.17 g, 0.38 mmol) and 10% Palladium on carbon (17 mg, 0.02 mmol) in ethanol (5 mL) and THF (5.00 mL) were stirred under an atmosphere of hydrogen at 1 atm and 20° C. for 20 hours. The reaction mixture was filtered through celite and evaporated and the reaction repeated for a further 24 hrs.

The reaction mixture was filtered through celite and evaporated to give a colourless oil. The crude product was purified by flash silica chromatography, elution gradient 2 to 7% MeOH in DCM. Pure fractions were evaporated to dryness to afford N-[(2r,5s)-5-hydroxyadamantan-2-yl]-2-methoxy-4-(1-methylcyclopropyl)pyrimidine-5-carboxamide (0.080 g, 58.9%) as a white solid;

1H NMR (400.132 MHz, CDCl3) δ 0.83-0.87 (2H, m), 1.25-1.29 (2H, m), 1.43-1.48 (1H, m), 1.49 (3H, s), 1.56-1.59 (1H, m), 1.66-1.87 (8H, m), 1.91-1.98 (1H, m), 2.17-2.36 (3H, m), 4.02 (3H, 2×s), 4.15-4.30 (1H, m), 5.90-6.41 (1H, m), 8.54 (1H, 2×s)

m/z (ESI+) (M+H)+=358; HPLC t_{R}=1.50 min

### Intermediate 129

### 4-(1-methylcyclopropyl)-2-methylsulfanyl-N-(5-phenylmethoxy-2-adamantyl)pyrimidine-5-carboxamide

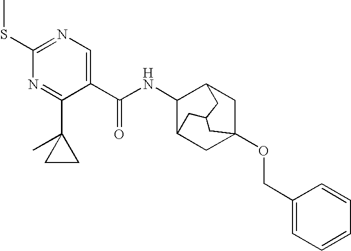

Prepared from Intermediate 127 by the same process used for Intermediate 128;

1H NMR (400.132 MHz, CDCl3) δ 0.83-0.86 (2H, m), 1.26-1.30 (2H, m), 1.49 (3H, 2×s), 1.59-1.66 (1H, m), 1.71-1.97 (8H, m), 2.06-2.11 (1H, m), 2.19-2.24 (1H, m), 2.29-2.38 (2H, m), 2.56 (3H, 2×s), 4.18-4.31 (1H, m), 4.51 (2H, d), 6.30-6.38 (1H, m), 7.22-7.26 (1H, m), 7.30-7.38 (4H, m), 8.59 (1H, 2×s)

m/z (ESI+) (M+H)+=464; HPLC t_{R}=2.83 min.

### Intermediate 130

### 2-methoxy-4-(1-methylcyclopropyl)-N-(5-phenylmethoxy-2-adamantyl)pyrimidine-5-carboxamide

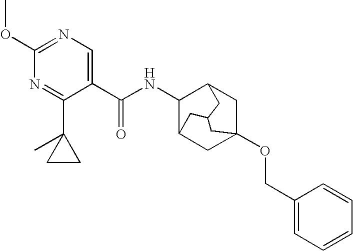

3-Chloroperoxybenzoic acid (70%) (1.276 g, 5.18 mmol) was added in one portion to 4-(1-methylcyclopropyl)-2-methylsulfanyl-N-(5-phenylmethoxy-2-adamantyl)pyrimidine-5-carboxamide (Intermediate 129, 1.2 g, 2.59 mmol) in DCM (50 mL) at 0° C. The resulting solution was stirred at 20° C. for 24 hours. The reaction mixture was diluted with DCM (50 mL), and washed sequentially with saturated NaHCO3 (75 mL), 2M NaOH (75 mL), and saturated brine (75 mL). The organic layer was dried over MgSO4, filtered and evaporated to afford crude product. The crude product was purified by flash silica chromatography, elution gradient 50 to 100% EtOAc in isohexane followed by 20% MeOH in DCM (to flush off the pyrimidone). Pure fractions were evaporated to dryness to afford 2-methoxy-4-(1-methylcyclopropyl)-N-(5-phenylmethoxy-2-adamantyl)pyrimidine-5-carboxamide (0.170 g, 14.68%) as a colourless oil and 2-hydroxy-4-(1-methylcyclopropyl)-N-(5-phenylmethoxy-2-adamantyl)pyrimidine-5-carboxamide (0.330 g, 29.4%) as a white solid;

1H NMR (400.132 MHz, CDCl3) δ 0.81-0.88 (2H, m), 1.23-1.28 (2H, m), 1.49 (3H, 2×s), 1.58-1.61 (1H, m), 1.72-1.97 (8H, m), 2.06-2.11 (1H, m), 2.18-2.25 (1H, m), 2.30-2.38 (2H, m), 4.02 (3H, 2×s), 4.18-4.31 (1H, m), 4.51 (2H, 2×s), 6.37-6.42 (1H, m), 7.22-7.26 (1H, m), 7.30-7.35 (4H, m), 8.61 (1H, 2×s)

m/z (ESI+) M+H+=447; HPLC t_{R}=2.78 min

### Example 188

### N-[(2r,5s)-5-hydroxyadamantan-2-yl]-2-methyl-4-phenylpyrimidine-5-carboxamide

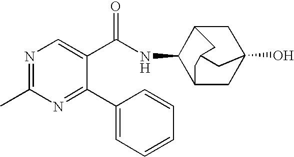

O-(7-Azabenzotriazol-1-yl)-N,N,N′,N′-tetramethyluronium hexafluorophosphate (456 mg, 1.2 mmol) was added in one portion to 2-methyl-4-phenylpyrimidine-5-carboxylic acid (214 mg, 1.00 mmol), 4-aminoadamantan-1-ol hydrochloride (203 mg, 1.00 mmol) and N-Ethyldiisopropylamine (0.522 mL, 3.00 mmol) in DMF (10 mL) at 25° C. under nitrogen. The resulting solution was stirred at 25° C. for 3 hours.

The reaction mixture was concentrated and diluted with EtOAc (100 mL), and washed sequentially with saturated NaHCO3 (100 mL), saturated brine (100 mL), and water (100 mL). The organic layer was dried over MgSO4, filtered and evaporated to afford crude product. The crude product was purified by preparative HPLC (Waters XBridge Prep C18 OBD column, 5μ silica, 30 mm diameter, 100 mm length), using decreasingly polar mixtures of water (containing 0.1% NH3) and MeCN as eluents. Fractions containing the desired compound were evaporated to dryness to afford N-[(2r,5s)-5-hydroxyadamantan-2-yl]-2-methyl-4-phenylpyrimidine-5-carboxamide (189 mg, 52.1%) as a white solid;

1H NMR (400.13 MHz, DMSO-d6) δ 1.16-1.19 (2H, m), 1.47-1.67 (8H, m), 1.85-1.88 (3H, m), 2.69 (3H, s), 3.88 (1H, t), 4.36 (1H, s), 7.39-7.51 (3H, m), 7.69-7.73 (2H, m), 8.29-8.31 (1H, m), 8.64 (1H, s)

m/z (ESI+) (M+H)+=364; HPLC t_{R}=1.42 min.

### Intermediate 131

### ethyl (Z)-2-benzoyl-3-dimethylaminoprop-2-enoate

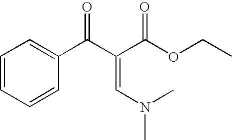

Prepared from ethyl 3-oxo-3-phenylpropanoate by the same process used for Intermediate;

m/z (ESI+) (M+H)+=248; HPLC t_{R}=1.79 min.

### Intermediate 132

### methyl 2-methyl-4-phenylpyrimidine-5-carboxylate

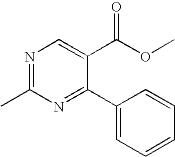

Prepared from Z)-methyl 2-benzoyl-3-(dimethylamino)acrylate by the same process used for Intermediate 2;

1H NMR (400.13 MHz, DMSO-d₆) δ 2.72 (3H, s), 3.71 (3H, s), 7.47-7.55 (3H, m), 7.57-7.60 (2H, m), 9.01 (1H, s)

m/z (ESI+) (M+H)+=229; HPLC t_{R}=1.76 min.

### Intermediate 133

### 2-methyl-4-phenylpyrimidine-5-carboxylic acid

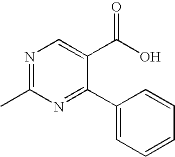

Prepared from Intermediate 132 by the same process used for Intermediate 29;

1H NMR (400.13 MHz, DMSO-d₆) δ 2.71 (3H, s), 7.45-7.53 (3H, m), 7.58-7.63 (2H, m), 8.98 (1H, s), 13.44 (1H, s)

m/z (ESI+) (M+H)+=215; HPLC t_{R}=1.19 min.

### Example 189

### 4-(2-Chlorophenyl)-N-[(2r,5s)-5-hydroxyadamantan-2-yl]-2-methylpyrimidine-5-carboxamide

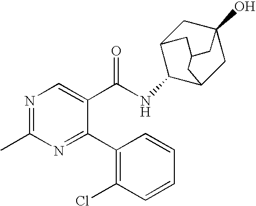

Prepared from Intermediate 136 by the same process used for Example 188;

1H NMR (400.13 MHz, CDCl3) δ 0.95 (2H, d), 1.22 (2H, d), 1.57-1.64 (1H, m), 1.73 (3H, d), 1.80-1.86 (3H, m), 1.83 (2H, d), 2.78 (3H, s), 3.94-3.99 (1H, m), 5.71 (1H, d), 7.38-7.41 (3H, m), 7.44-7.47 (1H, m), 9.10 (1H, s)

m/z (ESI+) (M+H)+=398; HPLC t_{R}=1.53 min.

### Intermediate 134

### (Z)-methyl 2-(2-chlorobenzoyl)-3-(dimethylamino)acrylate

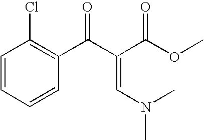

Prepared from methyl 3-(2-chlorophenyl)-3-oxopropanoate/84745/by the same process used for Intermediate 1;

1H NMR (400.13 MHz, CDCl₃) δ 2.91 (3H, bs), 3.25 (3H, bs), 3.38 (3H, s), 7.17-7.22 (2H, m), 7.26-7.29 (1H, m), 7.30-7.33 (1H, m), 7.71 (1H, s)

m/z (ESI+) (M+H)+=268; HPLC t_{R}=1.50 min.

### Intermediate 135

### methyl 4-(2-chlorophenyl)-2-methylpyrimidine-5-carboxylate

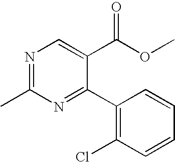

Prepared from (Z)-methyl 2-(2-chlorobenzoyl)-3-(dimethylamino)acrylate by the same process used for Intermediate 2;

1H NMR (400.13 MHz, CDCl₃) δ 2.84 (3H, s), 3.73 (3H, s), 7.37-7.43 (4H, m), 9.19 (1H, s)

m/z (ESI+) (M+H)+=263; HPLC t_{R}=1.90 min.

### Intermediate 136

### 4-(2-chlorophenyl)-2-methylpyrimidine-5-carboxylic acid

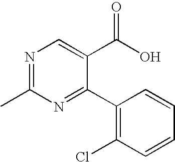

Prepared from Intermediate 135 by the same process used for Intermediate 29;

m/z (ESI+) (M+H)+=249; HPLC t_{R}=1.41 min.

### Example 190

### 4-(Cyclopentylmethyl)-N-[(2r,5 s)-5-hydroxyadamantan-2-yl]-2-methylpyrimidine-5-carboxamide

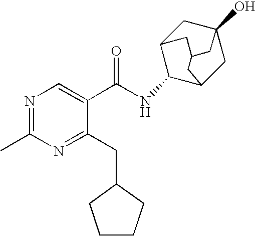

Prepared from Intermediate 140 by the same process used for Example 188;

1H NMR (400.13 MHz, CDCl3) δ 1.18-1.27 (2H, m), 1.47-1.56 (2H, m), 1.57-1.73 (9H, m), 1.81 (2H, s), 1.85 (1H, s), 1.96 (2H, d), 2.19 (1H, s), 2.27 (2H, s), 2.33 (1H, q), 2.74 (3H, s), 2.96 (2H, d), 4.21-4.25 (1H, m), 6.01 (1H, d), 8.57 (1H, s)

m/z (ESI+) (M+H)+=370; HPLC t_{R}=1.68 min.

### Intermediate 138

### (E)-methyl 4-cyclopentyl-2-((dimethylamino)methylene)-3-oxobutanoate

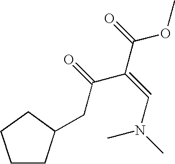

Prepared from methyl 4-cyclopentyl-3-oxobutanoate by the same process used for Intermediate 1;

1H NMR (400.13 MHz, CDCl₃) δ 1.07-1.16 (2H, m), 1.46-1.60 (4H, m), 1.73-1.81 (2H, m), 2.20-2.28 (1H, m), 2.68 (2H, d), 3.01 (6H, bs), 3.73 (3H, s), 7.64 (1H, s)

m/z (ESI+) (M+H)+=240; HPLC t_{R}=1.90 min.

### Intermediate 139

### methyl 4-(cyclopentylmethyl)-2-methylpyrimidine-5-carboxylate

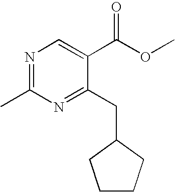

Prepared from Intermediate 138 by the same process used for Intermediate 2;

1H NMR (400.13 MHz, CDCl₃) δ 1.13-1.22 (2H, m), 1.40-1.47 (2H, m), 1.55-1.64 (4H, m), 2.17-2.25 (1H, m), 2.67 (3H, s), 3.09 (2H, d), 3.86 (3H, s), 8.94 (1H, s)

m/z (ESI+) (M+H)+=235; HPLC t_{R}=2.31 min.

### Intermediate 140

### 4-(cyclopentylmethyl)-2-methylpyrimidine-5-carboxylic acid

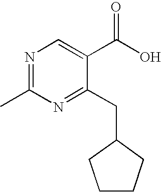

Prepared from Intermediate 139 by the same process used for Intermediate 29;

m/z (ESI+) (M+H)+=221; HPLC t_{R}=0.68 min.

### Example 191

### 4-Butyl-N-[(2r,5s)-5-hydroxyadamantan-2-yl]-2-methylpyrimidine-5-carboxamide

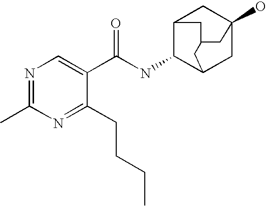

Prepared from Intermediate 144 by the same process used for Example 188;

1H NMR (400.13 MHz, DMSO-d6) δ 0.85 (3H, t), 1.23-1.35 (4H, m), 1.55-1.64 (6H, m), 1.71-1.74 (2H, m), 1.89-1.92 (2H, m), 1.97-2.00 (1H, m), 2.02-2.07 (2H, m), 2.59 (3H, s), 2.75 (2H, t), 3.93-3.98 (1H, m), 4.40 (1H, s), 8.36 (1H, d), 8.48 (1H, s)

m/z (ESI+) (M+H)+=344; HPLC t_{R}=1.45 min.

### Intermediate 142

### (Z)-methyl 2-((dimethylamino)methylene)-3-oxoheptanoate

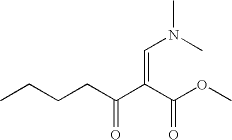

Prepared from methyl 3-oxoheptanoate by the same process used for Intermediate 1 and used without characterisation to prepare Intermediate 143.

### Intermediate 143

### methyl 4-butyl-2-methylpyrimidine-5-carboxylate

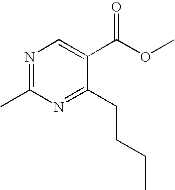

Prepared from (Z)-methyl 2-((dimethylamino)methylene)-3-oxoheptanoate by the same process used for Intermediate 2;

1H NMR (400.13 MHz, DMSO-d6) δ 0.89 (3H, t), 1.30-1.39 (2H, m), 1.57-1.65 (2H, m), 2.64 (3H, s), 3.00 (2H, t), 3.86 (3H, s), 8.96 (1H, s)

m/z (ESI+) (M+H)+=209; HPLC t_{R}=1.94 min.

### Intermediate 144

### 4-butyl-2-methylpyrimidine-5-carboxylic acid

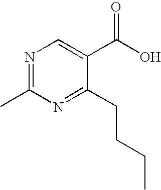

Prepared from Intermediate 143 by the same process used for Intermediate 29;

1H NMR (400.13 MHz, DMSO-d6) δ 0.89 (3H, t), 1.28-1.38 (2H, m), 1.57-1.64 (2H, m), 2.62 (3H, s), 3.01-3.05 (2H, m), 8.94 (1H, s), 13.46 (1H, s)

m/z (ESI+) (M+H)+=195; HPLC t_{R}=1.35 min.

### Example 192

### N-[(2s,5r)-5-Hydroxyadamantan-2-yl]-4-isobutyl-2-methylpyrimidine-5-carboxamide

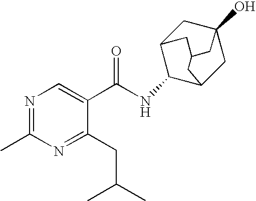

Prepared from Intermediate 148 by the same process used for Example 188;

1H NMR (400.13 MHz, DMSO-d6) δ 0.84 (6H, s), 1.33 (2H, d), 1.63 (4H, d), 1.71-1.74 (2H, m), 1.91 (2H, d), 1.98 (1H, s), 2.04-2.10 (3H, m), 2.60 (3H, s), 2.67 (2H, d), 3.96 (1H, t), 4.40 (1H, s), 8.36 (1H, d), 8.49 (1H, s)

m/z (ESI+) (M+H)+=344; HPLC t_{R}=1.39 min.

### Intermediate 146

### (Z)-methyl 2-((dimethylamino)methylene)-5-methyl-3-oxohexanoate

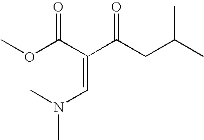

Prepared from methyl 5-methyl-3-oxohexanoate by the same process used for Intermediate 1;

m/z (ESI+) (M+H)+=214; HPLC t_{R}=1.48 min.

### Intermediate 147

### methyl 4-isobutyl-2-methylpyrimidine-5-carboxylate

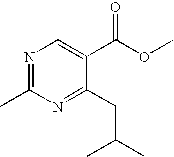

Prepared from (Z)-methyl 2-((dimethylamino)methylene)-5-methyl-3-oxohexanoate by the same process used for Intermediate 2;

1H NMR (400.13 MHz, DMSO-d₆) δ 0.87 (6H, d), 2.02-2.09 (1H, m), 2.63 (3H, s), 2.90 (2H, d), 3.86 (3H, s), 8.95 (1H, s)

m/z (ESI+) (M+H)+=209; HPLC t_{R}=1.82 min.

### Intermediate 148

### 4-isobutyl-2-methylpyrimidine-5-carboxylic acid

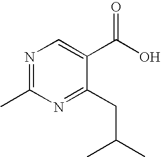

Prepared from Intermediate 147 by the same process used for Intermediate 29;

1H NMR (400.13 MHz, DMSO-d₆) δ 0.86 (6H, d), 2.04-2.11 (1H, m), 2.63 (3H, s), 2.96 (2H, d), 8.94 (1H, s) COOH signal very diffuse and not seen.

m/z (ESI+) (M+H)+=195; HPLC t_{R}=1.24 min.

### Example 193

### 4-(2,2-Dimethylpropyl)-N-[(2r,5s)-5-hydroxyadamantan-2-yl]-2-methylpyrimidine-5-carboxamide

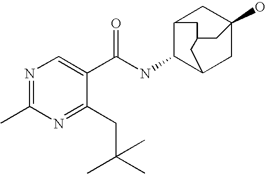

Prepared from Intermediate 152 by the same process used for Example 188;

1H NMR (400.13 MHz, DMSO-d6) δ 0.88 (9H, s), 1.29-1.36 (2H, m), 1.59-1.66 (4H, m), 1.70-1.73 (2H, m), 1.90-2.02 (5H, m), 2.60 (3H, s), 2.81 (2H, s), 3.92-3.97 (1H, m), 4.39 (1H, s), 8.37 (1H, d), 8.52 (1H, s)

m/z (ESI+) (M+H)+=358; HPLC t_{R}=1.62 min

### Intermediate 150

### (Z)-methyl 2-((dimethylamino)methylene)-5,5-dimethyl-3-oxohexanoate

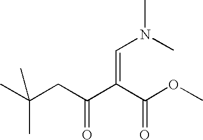

Prepared from methyl 5,5-dimethyl-3-oxohexanoate by the same process used for Intermediate 1 and used without characterisation to prepare Intermediate 151.

### Intermediate 151

### methyl 2-methyl-4-neopentylpyrimidine-5-carboxylate

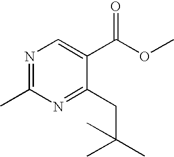

Prepared from (Z)-methyl 2-((dimethylamino)methylene)-5,5-dimethyl-3-oxohexanoate by the same process used for Intermediate 2;

1H NMR (400.13 MHz, DMSO-d6) δ 0.88 (9H, s), 2.64 (3H, s), 3.04 (2H, s), 3.86 (3H, s), 8.94 (1H, s)

m/z (ESI+) (M+H)+=223; HPLC t_{R}=2.08 min.

### Intermediate 152

### 2-methyl-4-neopentylpyrimidine-5-carboxylic acid

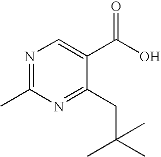

Prepared from Intermediate 151 by the same process used for Intermediate 29;

1H NMR (400.13 MHz, DMSO-d6) δ 0.90 (9H, s), 2.64 (3H, s), 3.10 (2H, s), 8.95 (1H, s), 13.56 (1H, s)

m/z (ESI+) (M+H)+=209; HPLC t_{R}=0.56 min.

### Example 194

### 4-(Cyclopropylmethyl)-N-[(2r,5 s)-5-hydroxyadamantan-2-yl]-2-methylpyrimidine-5-carboxamide

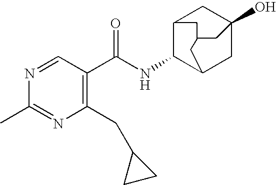

Prepared from Intermediate 156 by the same process used for Example 188;

1H NMR (400.132 MHz, CDCl3) δ 0.27 (2H, q), 0.46-0.51 (2H, m), 1.11-1.19 (1H, m), 1.37 (1H, s), 1.56-1.73 (4H, m), 1.78-1.84 (4H, m), 1.92-1.98 (2H, m), 2.16-2.27 (3H, m), 2.73 (3H, s), 2.84 (2H, d), 4.19-4.25 (1H, m), 5.98 (1H, d), 8.59 (1H, s)

m/z (ESI+) (M+H)+=342; HPLC t_{R}=1.32 min

### Intermediate 154

### (Z)-ethyl 4-cyclopropyl-2-((dimethylamino)methylene)-3-oxobutanoate

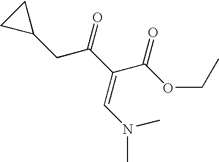

Prepared from ethyl 4-cyclopropyl-3-oxobutanoate by the same process used for Intermediate 1;

1H NMR (400.132 MHz, CDCl3) δ 0.10-0.15 (2H, m), 0.45-0.51 (2H, m), 1.00-1.11 (1H, m), 1.30 (3H, t), 2.60 (2H, d), 2.83-3.20 (6H, m), 4.21 (2H, d),766 (1H, s)

m/z (ESI+) (M+H)+=226; HPLC t_{R}=1.53 min.

### Intermediate 155

### methyl 4-(cyclopropylmethyl)-2-methylpyrimidine-5-carboxylate

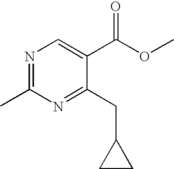

Prepared from (Z)-ethyl 4-cyclopropyl-2-((dimethylamino)methylene)-3-oxobutanoate by the same process used for Intermediate 2;

1H NMR (400.132 MHz, CDCl3) δ 0.19-0.25 (2H, m), 0.36-0.42 (2H, m), 1.06-1.15 (1H, m), 2.69 (3H, s), 2.97 (2H, d), 3.86 (3H, s), 8.97 (1H, s)

m/z (ESI+) (M+H)+=207; HPLC t_{R}=1.70 min.

### Intermediate 156

### 4-(cyclopropylmethyl)-2-methylpyrimidine-5-carboxylic acid

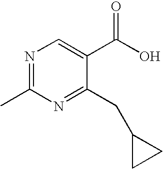

Prepared from Intermediate 155 by the Same Process Used for Intermediate 29

1H NMR (400.132 MHz, CDCl3) δ 0.30-0.35 (2H, m), 0.46-0.51 (2H, m), 1.22-1.28 (1H, m), 2.82 (3H, s), 3.13 (2H, d), 9.21 (1H, s)

m/z (ESI+) (M+H)+=193; HPLC t_{R}=1.13 min.

### Example 195

### 4-Cyclohexyl-N-[(2r,5 s)-5-hydroxyadamantan-2-yl]-2-(methylthio)pyrimidine-5-carboxamide

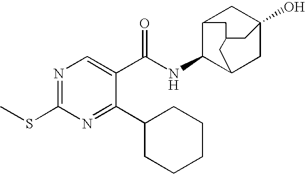

Prepared from Intermediate 158 by the same process used for Example 46;

1H NMR (400.13 MHz, DMSO-d6) δ 1.18-1.28 (3H, m), 1.31-1.37 (2H, m), 1.49-1.78 (13H, m), 1.86-1.93 (2H, m), 1.96-2.00 (1H, m), 2.02-2.07 (2H, m), 2.52 (3H, s), 2.88-2.97 (1H, m), 3.94-3.98 (1H, m), 4.40 (1H, s), 8.36 (1H, d), 8.41 (1H, s)

m/z (ESI+) (M+H)+=402; HPLC t_{R}=2.29 min.

### Intermediate 174

### 4-cyclohexyl-N-[(2r,5 s)-5-hydroxyadamantan-2-yl]-2-(methylsulfonyl)pyrimidine-5-carboxamide

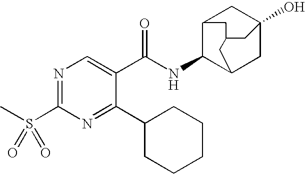

Prepared from Example 195 by the same process used for Example 37;

1H NMR (400.13 MHz, DMSO-d6) δ 1.20-1.31 (3H, m), 1.35-1.39 (2H, m), 1.54-1.88 (15H, m), 1.97-2.02 (1H, m), 2.04-2.10 (2H, m), 2.95-3.01 (1H, m), 3.42 (3H, s), 3.99-4.04 (1H, m), 4.43 (1H, s), 8.61 (1H, d), 8.87 (1H, s)

m/z (ESI+) (M+H)+=434; HPLC t_{R}=1.87 min.

### Example 196

### 4-Cyclohexyl-N-[(2r,5 s)-5-hydroxyadamantan-2-yl]-2-thiomorpholin-4-ylpyrimidine-5-carboxamide

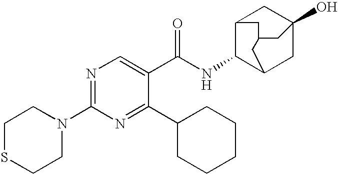

Prepared from Intermediate 174 by the same process used for Example 46;

1H NMR (400.13 MHz, DMSO-d6) δ 1.16-1.34 (5H, m), 1.44-1.53 (2H, m), 1.60-1.76 (11H, m), 1.91-2.03 (5H, m), 2.58-2.60 (4H, m), 2.97-3.02 (1H, m), 3.90 (1H, t), 4.07-4.10 (4H, m), 4.38 (1H, s), 8.08 (1H, d), 8.22 (1H, s)

m/z (ESI+) (M+H)+=457; HPLC t_{R}=2.56 min.

### Example 197

### 4-Cyclohexyl-N-[(2r,5 s)-5-hydroxyadamantan-2-yl]-2-(1-oxidothiomorpholin-4-yl)pyrimidine-5-carboxamide

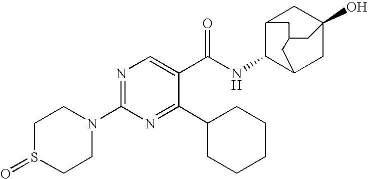

Prepared from Example 196 by the same process used for Example 36;

1H NMR (400.13 MHz, DMSO-d6) δ 1.17-1.34 (5H, m), 1.47-1.55 (2H, m), 1.60-1.77 (11H, m), 1.91-2.06 (5H, m), 2.70-2.77 (2H, m), 2.80-2.87 (2H, m), 2.97-3.05 (1H, m), 3.90-3.98 (3H, m), 4.38 (1H, s), 4.45-4.51 (2H, m), 8.11 (1H, d), 8.26 (1H, s)

m/z (ESI+) (M+H)+=473; HPLC t_{R}=1.69 min.

### Example 198

### 4-Cyclohexyl-2-(1,1-dioxidothiomorpholin-4-yl)-N-[(2r,5s)-5-hydroxyadamantan-2-yl]pyrimidine-5-carboxamide

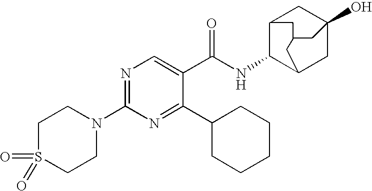

Prepared from Example 196 by the same process used for Example 37;

1H NMR (400.13 MHz, DMSO-d6) δ 1.16-1.34 (5H, m), 1.46-1.54 (2H, m), 1.61-1.77 (11H, m), 1.90-2.04 (5H, m), 2.95-3.05 (1H, m), 3.09-3.17 (4H, m), 3.89-3.94 (1H, m), 4.20-4.27 (4H, m), 4.39 (1H, s), 8.14 (1H, d), 8.28 (1H, s)

m/z (ESI+) (M+H)+=489; HPLC t_{R}=1.98 min.

### Intermediate 157

### methyl 4-cyclohexyl-2-(methylthio)pyrimidine-5-carboxylate

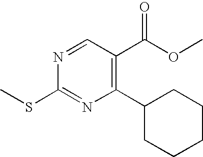

Prepared from Intermediate 61 by the same process used for Intermediate 2;

m/z (ESI+) (M+H)+=267; HPLC t_{R}=3.11 min.

### Intermediate 158

### 4-cyclohexyl-2-(methylthio)pyrimidine-5-carboxylic acid

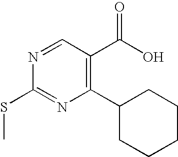

Prepared from Intermediate 157 by the same process used for Intermediate 29;

m/z (ESI+) (M+H)+=253; HPLC t_{R}=2.51 min.

The following Examples were prepared in a similar manner to Example 21, using Intermediate 42 and an appropriate starting material:

| | | | | MS m/e |
|---|---|---|---|---|
| Structure | Ex | Name | ¹H NMR δ | MH⁺ |
| 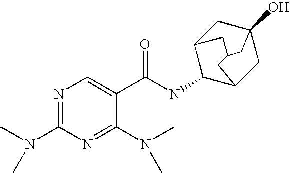 | 199 | 2,4- bis(dimethyla mino)-N- [(2r,5s)-5- hydroxyadaman- tan-2- yl]pyrimidine- 5- carboxamide | 1H NMR (400.132 MHz, CDCl3) δ 1.36(1 H, s), 1.55- 1.60(2 H, m), 1.65-1.80 (6 H, m), 1.90-1.95(2 H, m), 2.12-2.22(3 H, m), 2.99 (6 H, s), 3.17(6 H, s), 4.12- 4.20(1 H, m), 6.40(1 H, d), 8.30(1 H, s) | m/z (ESI+) (M + H)+ = 360; HPLC t_{R} = 1.52 min |
| 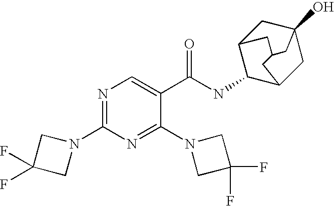 | 200 | 2,4-bis(3,3- difluoroazeti din-1-yl)-N- [(2r,5s)-5- hydroxyadam antan-2- yl]pyrimidine- 5- carboxamide | 1H NMR (400.132 MHz, CDCl3) δ 1.36(1 H, s), 1.55- 1.61(2 H, m), 1.67-1.83 (6 H, m), 1.90-1.96(2 H, m), 2.16-2.22(3 H, m), 4.10- 4.15(1 H, m), 4.42(8 H, t), 5.96(1 H, d), 8.18(1 H, s) | m/z (ESI+) (M + H)+ = 456; HPLC t_{R} = 1.87 min |
| 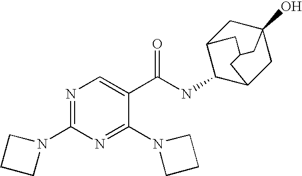 | 201 | 2,4- bis(azetidin- 1-yl)-N- [(2r,5s)-5- hydroxyadam antan-2- yl]pyrimidine- 5- carboxamide | 1H NMR (499.803 MHz, CDCl3) δ 1.38-1.58(3 H, m), 1.67-1.71(2 H, m), 1.75- 1.79(4 H, m), 1.89-1.94 (2 H, m), 2.13-2.20(3 H, m), 2.25-2.36(4 H, m), 4.06- 4.14(9 H, m), 5.95(1 H, d), 8.12(1 H, s) | m/z (ESI+) (M + H)+ = 384; HPLC t_{R} = 1.49 min |

### Example 202

### N-[(2r,5s)-5-hydroxyadamantan-2-yl]-2-methyl-4-propan-2-yloxypyrimidine-5-carboxamide

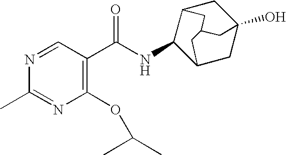

Prepared from Intermediate 161 by the same process used for Example 4;

1H NMR (400.132 MHz, CDCl3) δ 1.41 (1H, s), 1.46 (6H, d), 1.59 (2H, d), 1.75-1.84 (6H, m), 1.94 (2H, d), 2.21 (3H, s), 2.65 (3H, s), 4.26 (1H, d), 5.73 (1H, quintet), 7.96 (1H, d), 9.17 (1H, s)

m/z (ESI+) (M+H)+=346; HPLC t_{R}=1.74 min.

### Intermediate 174

### ethyl 2-methyl-6-oxo-1,6-dihydropyrimidine-5-carboxylate

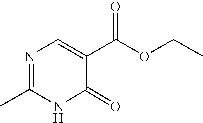

diethyl 2-(ethoxymethylene)malonate (9.35 mL, 46.25 mmol) was added dropwise to acetimidamide hydrochloride (4.37 g, 46.25 mmol), and sodium ethoxide (17.27 mL, 46.25 mmol) in ethanol (50 mL) at room temperature over a period of 5 minutes under nitrogen. The resulting solution was stirred at 60° C. for 6 hours. The reaction mixture was evaporated to dryness and redissolved in EtOAc (50 mL).The precipitate was collected by filtration, washed with EtOH (10 mL) and dried under vacuum to afford ethyl 2-methyl-6-oxo-1,6-dihydropyrimidine-5-carboxylate (4.17 g, 49.5%) as a cream solid, which was used without further purification.

1H NMR (400.13 MHz, DMSO-d₆) δ 1.15-1.23 (3H, t), 2.21 (3H, s), 4.09-4.17 (2H, q), 8.31 (1H, s)

m/z (ESI+) (M+H)+=183; HPLC t_{R}=0.78 min.

### Intermediate 159

### ethyl 4-chloro-2-methylpyrimidine-5-carboxylate

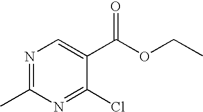

Phosphorus oxychloride (50 mL, 23.33 mmol) was added to ethyl 2-methyl-6-oxo-1,6-dihydropyrimidine-5-carboxylate (Intermediate 174, 4.25 g, 23.33 mmol). The insoluble mixture was refluxed for 30 minutes. The product was soluble in POCl3 where as the starting material was not. The excess POCl3 was removed under vacuum. The mixture was evaporated to dryness and redissolved in EtOAc (100 mL), and washed sequentially with water (75 mL) and saturated brine (75 mL). The organic layer was dried over MgSO4, filtered and evaporated to afford crude product. The crude product was purified by flash silica chromatography, elution gradient 10 to 30% EtOAc in isohexane. Pure fractions were evaporated to dryness to afford ethyl 4-chloro-2-methylpyrimidine-5-carboxylate (2.70 g, 57.7%) as a colourless oil.

1H NMR (400.132 MHz, CDCl3) δ 1.42 (3H, t), 2.78 (3H, s), 4.44 (2H, q), 9.05 (1H, s)

m/z (ESI+) (M+H)+=201; HPLC t_{R}=2.17 min.

### Intermediate 160

### isopropyl 4-isopropoxy-2-methylpyrimidine-5-carboxylate

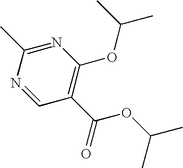

ethyl 4-chloro-2-methylpyrimidine-5-carboxylate (Intermediate 159, 186 mg, 0.93 mmol), Isopropyl alcohol (3549 μl, 46.36 mmol) and Sodium bis(trimethylsilyl)amide (927 μl, 0.93 mmol) were mixed under nitrogen and the reaction was stirred at 20° C. for 2 hours. The reaction mixture was diluted with EtOAc (40 mL), and washed sequentially with water (10 mL), and saturated brine (10 mL). The organic layer was dried over MgSO4, filtered and evaporated to afford crude product, which was used without further purification. Because it was a mixture and had a weak chromaphore it was used straight away in the next step;

m/z (ESI+) (M+H)+=225; HPLC t_{R}=1.98 min 33% (ethyl ester with isopropyl ether plus isopropyl ester with ethyl ether), (M+H)+=239; HPLC t_{R}=2.24 min 67% (isopropyl ester)

### Intermediate 161

### 4-isopropoxy-2-methylpyrimidine-5-carboxylic acid

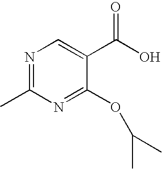

Prepared from Intermediate 160 by the Same Process Used for Intermediate 2

1H NMR (400.132 MHz, CDCl3) δ 1.49 (6H, d), 2.69 (3H, s), 5.73 (1H, quintet), 9.13 (1H, s)

m/z (ESI+) (M−H)-=195; HPLC t_{R}=0.93 min

### Example 203

### 4-Cyclobutyloxy-N-[(2r,5 s)-5-hydroxyadamantan-2-yl]-2-methylpyrimidine-5-carboxamide

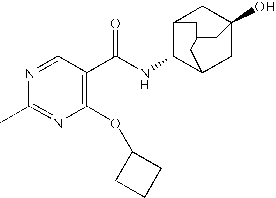

Prepared from Intermediate 163 by the same process used for Example 4;

1H NMR (400.132 MHz, CDCl3) δ 1.42 (1H, s), 1.60 (2H, d), 1.70-1.85 (7H, m), 1.90-1.99 (3H, m), 2.13-2.25 (5H, m), 2.53-2.61 (2H, m), 2.63 (3H, s), 4.27 (1H, d), 5.46 (1H, quintet), 7.95 (1H, d), 9.16 (1H, s)

m/z (ESI+) (M+H)+=358; HPLC t_{R}=1.94 min.

### Intermediate 162

### ethyl 4-cyclobutoxy-2-methylpyrimidine-5-carboxylate

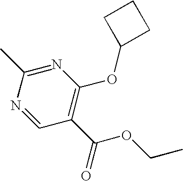

Prepared from Intermediate 159 by the same process used for Intermediate 160;

m/z (ESI+) (M+H)+=237; HPLC t_{R}=2.18 min.

### Intermediate 163

### 4-cyclobutoxy-2-methylpyrimidine-5-carboxylic acid

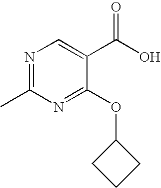

Prepared from Intermediate 162 by the same process used for Intermediate 2;

1H NMR (400.132 MHz, CDCl3) δ 1.70-1.81 (1H, m), 1.88-1.99 (1H, m), 2.21-2.32 (2H, m), 2.51-2.59 (2H, m), 2.68 (3H, s), 5.47 (1H, quintet), 9.09 (1H, s)

m/z (ESI+) (M+H)+=209; HPLC t_{R}=1.18 min.

### Example 204

### 4-Cyclopentyloxy-N-[(2r,5s)-5-hydroxyadamantan-2-yl]-2-methylpyrimidine-5-carboxamide

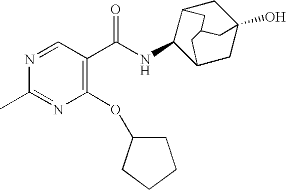

Prepared from Intermediate 165 by the same process used for Example 4;

1H NMR (400.132 MHz, CDCl3) δ 1.42 (1H, s), 1.59 (2H, d), 1.66-1.91 (12H, m), 1.94 (2H, d), 2.06-2.16 (2H, m), 2.17-2.26 (3H, m), 2.64 (3H, s), 4.25 (1H, d), 5.79 (1H, septet), 7.84 (1H, d), 9.15 (1H, s)

m/z (ESI+) (M+H)+=372; HPLC t_{R}=2.04 min.

### Intermediate 164

### cyclopentyl 4-(cyclopentyloxy)-2-methylpyrimidine-5-carboxylate

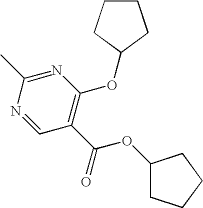

Prepared from Intermediate 159 by the same process used for Intermediate 160

m/z (ESI+) (M+H)+=291; HPLC t_{R}=2.94 min

### Intermediate 165

### 4-(cyclopentyloxy)-2-methylpyrimidine-5-carboxylic acid

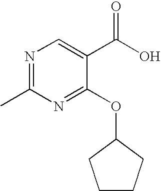

Prepared from Intermediate 164 by the same process used for Intermediate 2;

1H NMR (400.132 MHz, CDCl₃) δ 1.67-1.96 (6H, m), 2.02-2.13 (2H, m), 2.69 (3H, s), 5.81 (1H, septet), 9.10 (1H, s)

m/z (ESI+) (M−H)-=221; HPLC t_{R}=1.33 min

### Example 205

### 2-[(2R,6S)-2,6-Dimethylmorpholin-4-yl]-N-[(2r,5 s)-5-hydroxytricyclo[3.3.1.13.7]dec-2-yl]-4-methoxypyrimidine-5-carboxamide

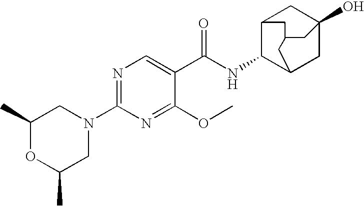

2-chloro-N-[(2r,5s)-5-hydroxytricyclo[3.3.1.13.7]dec-2-yl]-4-methoxypyrimidine-5-carboxamide (Intermediate 166, 0.215 g, 0.64 mmol), and cis-2,6-Dimethylmorpholine (0.157 mL, 1.27 mmol) were suspended in THF (4 mL) and sealed into a microwave tube. The reaction was heated to 50° C. for 30 minutes in the microwave reactor and cooled to RT. The reaction mixture was diluted with EtOAc (20 mL), and washed sequentially with saturated NH4Cl (10 mL) and saturated brine (10 mL). The organic layer was dried over MgSO4, filtered and evaporated to afford crude product. The crude product was purified by preparative HPLC (Waters XBridge Prep C18 OBD column, 5μ silica, 50 mm diameter, 150 mm length), using decreasingly polar mixtures of water (containing 0.5% NH3) and MeCN as eluents. Fractions containing the desired compounds were evaporated to dryness to afford 2-[(2R,6S)-2,6-dimethylmorpholin-4-yl]-N-[(2r,5 s)-5-hydroxytricyclo[3.3.1.13.7]dec-2-yl]-4-methoxypyrimidine-5-carboxamide (0.047 g, 17.73%) as a white solid.

1H NMR (400.13 MHz, DMSO-d₆) δ 1.15 (6H, d), 1.43 (2H, d), 1.63-1.65 (4H, m), 1.69-1.72 (4H, m), 2.00 (2H, s), 2.05 (1H, s), 2.56-2.62 (2H, m), 3.50-3.58 (2H, m), 3.94 (1H, t), 4.02 (3H, s), 4.42 (1H, s), 4.55 (2H, d), 7.63-7.65 (1H, m), 8.61 (1H, s)

m/z (ES+) (M+H)+=417; HPLC t_{R}=1.90 min.

### Intermediate 166

### 2-chloro-N-[(2r,5s)-5-hydroxytricyclo[3.3.1.13.7]dec-2-yl]-4-methoxypyrimidine-5-carboxamide

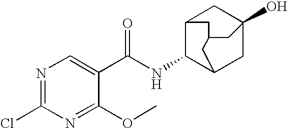

Sodium methoxide (0.050 g, 0.92 mmol) was added in one portion to a solution of 2,4-dichloro-N-[(2s,5r)-5-hydroxyadamantan-2-yl]pyrimidine-5-carboxamide (Intermediate 42, 0.3 g, 0.88 mmol) in THF (30 mL) at 0° C. under nitrogen. The resulting suspension was stirred for 6 hours. The reaction mixture was diluted with EtOAc (75 mL), and washed sequentially with 0.1M HCl (25 mL), water (25 mL), and saturated brine (25 mL). The organic layer was dried over MgSO4, filtered and evaporated to afford crude 2-chloro-N-[(2r,5s)-5-hydroxytricyclo[3.3.1.13.7]dec-2-yl]-4-methoxypyrimidine-5-carboxamide (0.250 g, 84%) product as a yellow solid. Used directly in the next step without further purification.

1H NMR (400.13 MHz, DMSO-d₆) δ 1.38 (2H, d), 1.62-1.65 (5H, m), 1.70-1.76 (2H, m), 1.76 (1H, m), 1.80-1.83 (2H, m), 1.98 (1H, s), 3.91-3.96 (1H, m), 4.02 (3H, s), 4.40 (1H, s), 8.03 (1H, d), 8.64 (1H, d)

m/z (ES+) (M+H)+=338; HPLC t_{R}=1.62 min.

The following Examples were prepared in a similar manner to Example 205, using Intermediate 166 and an appropriate starting material:

| | | | | MS m/e |
|---|---|---|---|---|
| Structure | Ex | Name | ¹H NMR δ | MH⁺ |
| 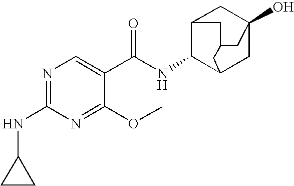 | 206 | 2- (cyclopropyl amino)-N- [(2r,5s)-5- hydroxytricy clo[3.3.1.13, 7]dec-2-yl]- 4- methoxypyri midine-5- carboxamide | 1H NMR (400.13 MHz, DMSO-d₆) δ 0.48-0.56 (2 H, m), 0.69(2 H, d), 1.44 (2 H, d), 1.64(4 H, d), 1.71 (4 H, d), 2.00-2.05(3 H, m), 2.76-2.82(1 H, m), 3.16 (2 H, d), 3.95(2 H, d), 4.43 (1 H, s), 7.64(1 H, d), 7.81- 7.91(1 H, m), 8.55(1 H, s) | m/z (ES+) (M + H)+ = 359; HPLC t_{R} = 1.72 min. |
| 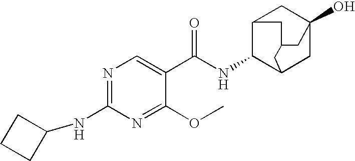 | 207 | 2- (cyclobutyla mino)-N- [(2r,5s)-5- hydroxytricy clo[3.3.1.13, 7]dec-2-yl]- 4- methoxypyri midine-5- carboxamide | 1H NMR (400.13 MHz, DMSO-d₆) δ 1.43(2 H, d), 1.62-1.69(11 H, m), 1.99 (4 H, s), 2.04(2 H, s), 2.24 (2 H, s), 3.96(2 H, d), 4.01 (2 H, s), 4.42(1 H, s), 7.61 (1 H, d), 8.53(1 H, s) | m/z (ES+) (M + H)+ = 373; HPLC t_{R} = 1.92 min. |
| 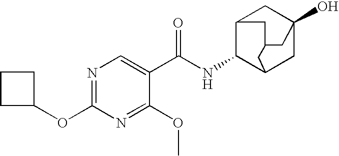 | 208 | 2- (cyclobutyl- oxy)-N- [(2r,5s)-5- hydroxytricy clo[3.3.1.13, 7]dec-2-yl]- 4- methoxypyri midine-5- carboxamide | 1H NMR (400.13 MHz, DMSO-d₆) δ 1.41(2 H, d), 1.62-1.66(5 H, m), 1.67- 1.80(5 H, m), 2.02(3 H, s), 2.05-2.14(2 H, m), 2.38- 2.45(2 H, m), 3.94(1 H, t), 4.02(3 H, s), 4.44(1 H, s), 5.12-5.19(1 H, m), 7.80 (1 H, d), 8.61(1 H, s) | m/z (ES+) (M + H)+ = 374; HPLC t_{R} = 1.95 min. |

### Intermediate 167

### 2-chloro-4-ethoxy-N-[(2r,5 s)-5-hydroxytricyclo[3.3.1.13.7]dec-2-yl]pyrimidine-5-carboxamide

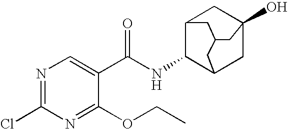

Prepared from Intermediate 42 by the Same Process Used for Intermediate 2

m/z (ES−) M−=350; HPLC t_{R}=1.83 min.

The following Examples were prepared in a similar manner to Example 205, using Intermediate 167 and an appropriate starting material:

| | | | | MS m/e |
|---|---|---|---|---|
| Structure | Ex | Name | ¹H NMR δ | MH⁺ |
| 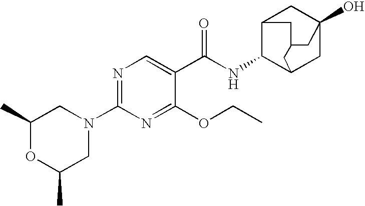 | 209 | 2-[(2S,6R)- 2,6- dimethylmor pholin-4-yl]- 4-ethoxy-N- [(2r,5s)-5- hydroxyada mantan-2- yl]pyrimidine- 5- carboxamide | 1H NMR (400.13 MHz, CDCl₃) δ 1.25(3 H, d), 1.26 (3 H, s), 1.49(3 H, t), 1.56 (2 H, d), 1.66(1 H, s), 1.75- 1.82(7 H, s), 1.92-1.95 (2 H, m), 2.18(3 H, s), 2.64 (2 H, dd), 3.58-3.66(2 H, m), 4.22-4.26(1 H, m), 4.52 (2 H, q), 7.75(1 H, d), 8.95 (1 H, s) | m/z (ESI+) (M + H)+ = 431; HPLC t_{R} = 2.11 min. |
| 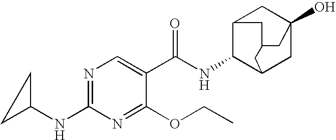 | 210 | 2- (cyclopropyl amino)-4- ethoxy-N- [(2r,5s)-5- hydroxytricy clo[3.3.1.13, 7]dec-2- yl]pyrimidine- 5- carboxamide | 1H NMR (400.13 MHz, DMSO-d₆) δ 0.48-0.52 (2 H, m), 0.66-0.69(2 H, m), 1.40(3 H, t), 1.35-1.47 (2 H, m), 1.64(4 H, d), 1.72 (4 H, d), 2.00(2 H, s), 2.04 (1 H, s), 2.77(1 H, d), 3.98 (1 H, t), 4.43(1 H, s), 4.52 (1 H, s), 7.68(1 H, d), 7.78- 7.90(1 H, m), 8.58(1 H, s) | m/z (ES+) (M + H)+ = 373; HPLC t_{R} = 1.51 min. |
| 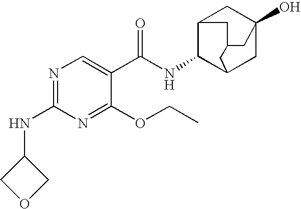 | 211 | 4-ethoxy-N- [(2r,5s)-5- hydroxytricy clo[3.3.1.13, 7]dec-2-yl]- 2-(oxetan-3- ylamino)pyri midine-5- carboxamide | 1H NMR (400.13 MHz, DMSO-d₆) δ 1.39(3 H, t), 1.44(2 H, d), 1.62-1.65 (4 H, m), 1.70-1.73(4 H, m), 1.99(2 H, s), 2.04(1 H, s), 3.16(1 H, d), 3.97(1 H, t), 4.43(1 H, s), 4.45(2 H, q), 4.53(2 H, t), 4.75(2 H, t), 4.90(1 H, s), 7.66(1 H, d), 8.58(1 H, s) | m/z (ES+) (M + H)+ = 389; HPLC t_{R} = 1.45 min. |
| 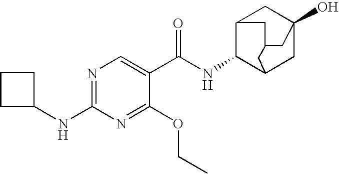 | 212 | 2- (cyclobutyla mino)-4- ethoxy-N- [(2r,5s)-5- hydroxytricy clo[3.3.1.13, 7]dec-2- yl]pyrimidine- 5- carboxamide | 1H NMR (400.13 MHz, DMSO-d₆) δ 1.35-1.45 (5 H, m), 1.55-1.65(5 H, m), 1.70-1.82(5 H, m), 1.99 (4 H, s), 2.04(1 H, s), 2.23 (2 H, s), 3.97(1 H, m), 4.43 (2 H, s), 4.47(2 H, m), 7.65 (1 H, d), 8.04(1 H, d), 8.55 (1 H, s) | m/z (ES+) (M + H)+ = 387; HPLC t_{R} = 2.13 min. |
| 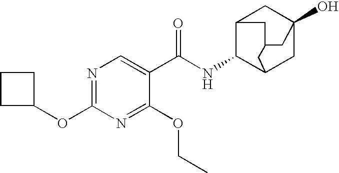 | 213 | 2- (cyclobutyloxy)- 4- ethoxy-N- [(2r,5s)-5- hydroxytricy clo[3.3.1.13, 7]dec-2- yl]pyrimidine- 5- carboxamide | 1H NMR (400.13 MHz, DMSO-d₆) δ 1.37-1.41 (4 H, m), 1.44(1 H, s), 1.64 (5 H, d), 1.70(2 H, s), 1.75- 1.78(3 H, m), 1.82(1 H, d), 2.02(3 H, s), 2.07-2.12 (2 H, m), 2.38-2.44(1 H, m), 3.96(1 H, d), 4.43(1 H, s), 4.49(2 H, t), 5.10-5.17 (1 H, m), 7.80(1 H, d), 8.67 (1 H, s) | m/z (ES+) (M + H)+ = 388; HPLC t_{R} = 2.12 min. |

### Intermediate 168

### 2-chloro-N-[(2r,5s)-5-hydroxytricyclo[3.3.1.13.7]dec-2-yl]-4-(1-methylethoxyl)pyrimidine-5-carboxamide

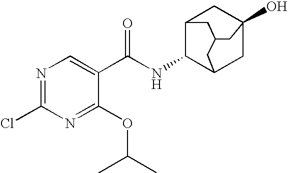

Prepared from Intermediate 42 by the same process used for Intermediate 2;

m/z (ES+) (M+H)+=366; HPLC t_{R}=2.01 min.

The following Examples were prepared in a similar manner to Example 205, using Intermediate 168 and an appropriate starting material:

| | | | | MS m/e |
|---|---|---|---|---|
| Structure | Ex | Name | ¹H NMR δ | MH⁺ |
| 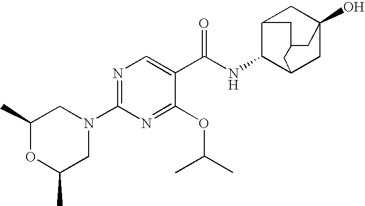 | 214 | 2-[(2R,6S)- 2,6- dimethylmorpho- lin-4-yl]- N-[(2r,5s)-5- hydroxytricyc lo[3.3.1.13,7] dec-2-yl]-4- (1- methylethoxy) pyrimidine- 5- carboxamide | 1H NMR (400.13 MHz, DMSO-d₆) δ 1.14(6 H, d), 1.38 (3 H, s), 1.39(3 H, s), 1.45- 1.48(2 H, m), 1.64(4 H, m), 1.71(4 H, s), 2.00(2 H, s), 2.05 (1 H, s), 2.57-2.63(2 H, m), 3.50-3.58(2 H, m), 3.98(1 H, m), 4.43(1 H, s), 4.50(2 H, s), 5.46-5.52(1 H, m), 7.66(1 H, d), 8.65(1 H, s) | m/z (ES+) (M + H)+ = 445; HPLC t_{R} = 2.17 min. |
| 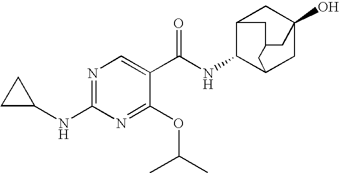 | 215 | 2- (cyclopropyla mino)-N- [(2r,5s)-5- hydroxytricyc lo[3.3.1.13,7] dec-2-yl]-4- (1- methylethoxy) pyrimidine- 5- carboxamide | 1H NMR (400.13 MHz, DMSO-d₆) δ 0.44-0.56(2 H, m), 0.66-0.70(2 H, m), 1.39 (6 H, s), 1.47(2 H, d), 1.64(4 H, d), 1.72(4 H, d), 1.99(2 H, s), 2.05(1 H, s), 2.75(1 H, s), 3.98 (1 H, t), 4.43(1 H, s), 5.53(1 H, s), 7.66-7.68(1 H, m), 7.92 (1 H, s), 8.60(1 H, d) | m/z (ES+) (M + H)+ = 387; HPLC t_{R} = 1.96 min. |
| 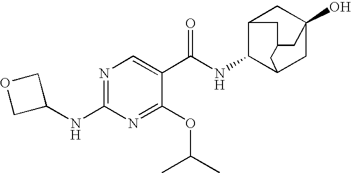 | 216 | N-[(2r,5s)-5- hydroxytricyc lo[3.3.1.13,7] dec-2-yl]-4- (1- methylethoxy)- 2-(oxetan-3- ylamino)pyri midine-5- carboxamide | 1H NMR (400.13 MHz, DMSO-d₆) δ 1.36(3 H, d), 1.38 (3 H, s), 1.45-1.48(2 H, m), 1.63-1.65(4 H, m), 1.69(4 H, d), 1.99(2 H, s), 2.05(1 H, s), 3.97(1 H, t), 4.43(1 H, s), 4.54 (2 H, s), 4.76(2 H, t), 4.88(1 H, s), 5.46(1 H, s), 7.65(1 H, d), 8.45(1 H, s), 8.60(1 H, s) | m/z (ES+) (M + H)+ = 403; HPLC t_{R} = 1.63 min. |
| 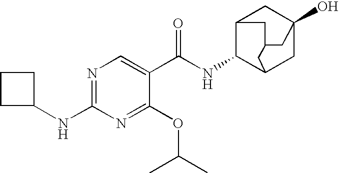 | 217 | 2- (cyclobutyla mino)-N- [(2r,5s)-5- hydroxytricyc lo[3.3.1.13,7] dec-2-yl]-4- (1- methylethoxy) pyrimidine- 5- carboxamide | 1H NMR (400.13 MHz, DMSO-d₆) δ 1.37(4 H, d), 1.39 (2 H, s), 1.45-1.48(2 H, m), 1.63-1.63(3 H, m), 1.65(2 H, s), 1.68(2 H, d), 1.70(3 H, s), 1.99(4 H, s), 2.05(1 H, s), 2.23 (2 H, s), 3.97(1 H, t), 4.27(1 H, d), 4.40-4.43(1 H, m), 5.46- 5.52(1 H, m), 7.65(1 H, d), 8.04(1 H, d), 8.53-8.57(1 H, m) | m/z (ES+) (M + H)+ = 401; HPLC t_{R} = 2.18 min. |
| 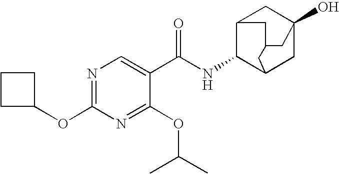 | 218 | 2- (cyclobutyloxy)- N-[(2r,5s)- 5- hydroxytricyc lo[3.3.1.13,7] dec-2-yl]-4- (1- methylethoxy) pyrimidine- 5- carboxamide | 1H NMR (400.13 MHz, DMSO-d₆) δ 1.35-1.40(6 H, m), 1.45(2 H, d), 1.63(3 H, d), 1.66(2 H, s), 1.73(4 H, t), 2.02 (3 H, s), 2.06-2.15(2 H, m), 2.37-2.45(2 H, m), 3.16(1 H, d), 3.97(1 H, t), 4.43(1 H, s), 5.09-5.16(1 H, m), 5.44- 5.50(1 H, m), 7.76(1 H, d), 8.70(1 H, s) | m/z (ES+) (M + H)+ = 402; HPLC t_{R} = 1.77 min. |

### Example 219

### 2-[(2S,6R)-2,6-Dimethylmorpholin-4-yl]-N-[(2r,5s)-5-hydroxyadamantan-2-yl]-4-(methoxymethyl)pyrimidine-5-carboxamide

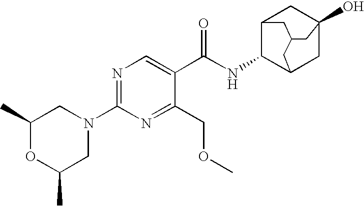

2-((2S,6R)-2,6-Dimethylmorpholino)-4-(methoxymethyl)pyrimidine-5-carboxylic acid (605.9 mg, 2.15 mmol), 2-(3H-[1,2,3]triazolo[4,5-b]pyridin-3-yl)-1,1,3,3-tetramethylisouronium hexafluorophosphate(V) (Intermediate 170, 1.23 g, 3.23 mmol) and N-ethyl-N-isopropylpropan-2-amine (0.737 mL, 4.31 mmol) were dissolved in DMF (50 mL). The resulting solution was stirred at room temperature for 15 minutes. 4-aminoadamantan-1-ol hydrochloride (565.1 mg, 2.77 mmol) was then added and continued to stir at room temperature over night. The reaction mixture was evaporated to dryness and redissolved in EtOAc (150 mL) and washed sequentially with water (2×100 mL) and saturated brine (100 mL). The organic layer was dried over MgSO4, filtered and evaporated to afford crude product. The crude product was purified by flash silica chromatography, elution gradient 0 to 10% MeOH in DCM. Pure fractions were evaporated to dryness to afford 2-[(2S,6R)-2,6-dimethylmorpholin-4-yl]-N-[(2r,5s)-5-hydroxyadamantan-2-yl]-4-(methoxymethyl)pyrimidine-5-carboxamide as a orange solid. The crude product was purified by crystallisation from hot EtOAc to afford 2-[(2S,6R)-2,6-dimethylmorpholin-4-yl]-N-[(2r,5s)-5-hydroxyadamantan-2-yl]-4-(methoxymethyl)pyrimidine-5-carboxamide (723 mg, 78%) as a white solid.

1H NMR (400.13 MHz, CDCl₃) δ 1.27 (6H, d), 1.43-1.53 (2H, m), 1.55 (1H, s), 1.78 (3H, s), 1.80 (2H, s), 1.92-1.95 (2H, m), 2.16 (1H, s), 2.21 (2H, s), 2.63 (2H, dd), 3.48 (3H, s), 3.58-3.66 (2H, m), 4.19-4.23 (1H, m), 4.51 (2H, s), 4.67 (2H, dd), 7.93 (1H, d), 8.84 (1H, s)

m/z (ESI+) (M+H)+=431; HPLC t_{R}=2.88 min.

### Intermediate 169

### Methyl 2-((2S,6R)-2,6-dimethylmorpholino)-4-(methoxymethyl)pyrimidine-5-carboxylate

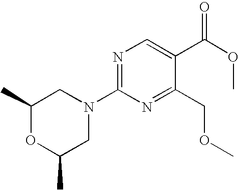

(2R,6S)-2,6-dimethylmorpholine-4-carboximidamide hydrochloride (1.95 g, 10.07 mmol) was added in one portion to (Z)-methyl 2-((dimethylamino)methylene)-4-methoxy-3-oxobutanoate (2.01 g, 9.99 mmol) and sodium acetate (2.04 g, 24.87 mmol) in DMF (15 mL) at 20° C. under nitrogen. The resulting suspension was stirred at 80° C. over night. The reaction mixture was evaporated to dryness and redissolved in EtOAc (100 mL), and washed sequentially with water (2×75 mL) and saturated brine (75 mL). The organic layer was dried over MgSO4, filtered and evaporated to afford crude product.

The crude product was purified by flash silica chromatography, elution gradient 0 to 50% EtOAc in isohexane. Pure fractions were evaporated to dryness to afford methyl 2-((2S,6R)-2,6-dimethylmorpholino)-4-(methoxymethyl)pyrimidine-5-carboxylate (1.598 g, 54%) as a colourless oil which solidified on standing. White Solid.

1H NMR (400.13 MHz, CDCl₃) δ 1.27 (6H, d), 2.67 (2H, dd), 3.52 (3H, s), 3.59-3.67 (2H, m), 3.85 (3H, s), 4.74-4.77 (2H, m), 4.81 (2H, s), 8.82 (1H, s)

m/z (ESI+) (M+H)+=296; HPLC t_{R}=2.73 min.

### Intermediate 170

### 2-((2S,6R)-2,6-Dimethylmorpholino)-4-(methoxymethyl)pyrimidine-5-carboxylic acid

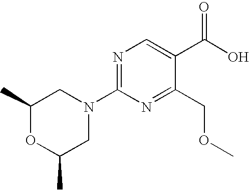

sodium hydroxide (27.1 mL, 54.18 mmol) was added in one portion to methyl 2-((2S,6R)-2,6-dimethylmorpholino)-4-(methoxymethyl)pyrimidine-5-carboxylate (Intermediate 169, 1.60 g, 5.42 mmol) in methanol (70 mL) at 20° C. The resulting suspension was stirred at room temperature over night.

The reaction mixture was evaporated to dryness and redissolved in water (150 mL), which was acidified to pH 4 with 2N HCl. The aqueous layer was washed sequentially with EtOAc (3×100 mL). The organic layer was dried over MgSO4, filtered and evaporated to afford crude 2-((2S,6R)-2,6-dimethylmorpholino)-4-(methoxymethyl)pyrimidine-5-carboxylic acid (0.606 g, 40%) as a white solid, which was used without further purification and characterisation.

1H NMR (400.13 MHz, DMSO-d₆) δ 1.15 (6H, d), 2.62 (2H, dd), 3.35 (3H, s), 3.51-3.59 (2H, m), 4.63 (2H, d), 4.69 (2H, s), 8.73 (1H, s)

m/z (ESI+) (M+H)+=282; HPLC t_{R}=1.12 min.

### Example 220

### 4-Cyclopropyl-2-[(2S,6R)-2,6-dimethylmorpholin-4-yl]-N-[(2r,5s)-5-hydroxyadamantan-2-yl]pyrimidine-5-carboxamide

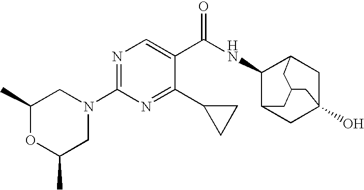

(2R,6S)-2,6-dimethylmorpholine (Intermediate 80, 4.71 g, 40.87 mmol) was added to 4-cyclopropyl-N-[(2r,5s)-5-hydroxyadamantan-2-yl]-2-methylsulfonylpyrimidine-5-carboxamide (3.2 g, 8.17 mmol) in THF (60 mL) at 20° C. under nitrogen. The resulting solution was stirred at 20° C. for 20 hours.

The reaction mixture was evaporated to dryness and redissolved in EtOAc (150 mL), and washed sequentially with water (150 mL) and saturated brine (150 mL). The organic layer was dried over MgSO4, filtered and evaporated to afford crude product. The crude product was purified by flash silica chromatography, elution gradient 1 to 5% MeOH in DCM. Pure fractions were evaporated to dryness to afford the product as a white foam which was triturated with ether to give 4-cyclopropyl-2-[(2S,6R)-2,6-dimethylmorpholin-4-yl]-N-[(2r,5s)-5-hydroxyadamantan-2-yl]pyrimidine-5-carboxamide (2.220 g, 64%) as a white solid.

1H NMR (400.132 MHz, CDCl3) δ 0.99-1.05 (2H, m), 1.18-1.21 (2H, m), 1.24 (6H, d), 1.41 (1H, s), 1.56-1.59 (2H, m), 1.69-1.73 (2H, m), 1.76-1.82 (4H, m), 1.90-1.96 (2H, m), 2.15-2.18 (1H, m), 2.23-2.26 (2H, m), 2.48-2.61 (3H, m), 3.53-3.62 (2H, m), 4.19-4.24 (1H, m), 4.49-4.56 (2H, m), 6.03 (1H, d), 8.37 (1H, s) m/z (ES+) (M+H)+=427; HPLC t_{R}=1.98 min.

### Example 221

### 4-Cyclopropyl-2-(2,6-dimethylmorpholin-4-yl)-N-[(2r,5s)-5-hydroxyadamantan-2-yl]pyrimidine-5-carboxamide

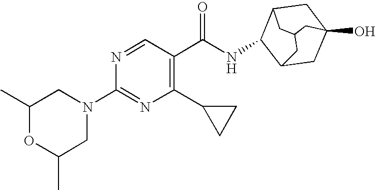

O-(7-Azabenzotriazol-1-yl)-N,N,N′,N′-tetramethyluronium hexafluorophosphate (811 mg, 2.13 mmol) was added to 4-cyclopropyl-2-(2,6-dimethylmorpholino)pyrimidine-5-carboxylic acid (Intermediate 74, 473 mg, 1.71 mmol), 4-aminoadamantan-1-ol hydrochloride (347 mg, 1.71 mmol) and N-Ethyldiisopropylamine (0.654 mL, 3.75 mmol) in DMF (5 mL) at ambient temperature under nitrogen. The resulting solution was stirred at ambient temperature for 16 hours. The reaction mixture was evaporated to dryness and redissolved in EtOAc (50 mL) and washed sequentially with water (10 mL), 1N citric acid (10 mL), saturated NaHCO3 (5 mL) and saturated brine (10 mL). The organic layer was dried over MgSO4, filtered and evaporated to afford crude product. The crude product was purified by preparative HPLC Waters XBridge Prep C18 OBD column, 5μ silica, 50 mm diameter, 150 mm length), using decreasingly polar mixtures of water (containing 0.5% NH3) and MeCN as eluents. Fractions containing the desired compound were evaporated to dryness to afford 4-cyclopropyl-2-(2,6-dimethylmorpholin-4-yl)-N-[(2r,5s)-5-hydroxyadamantan-2-yl]pyrimidine-5-carboxamide (389 mg, 54%) as a white solid.

1H NMR (400.132 MHz, CDCl3) δ 0.92-0.97 (2H, m), 1.11-1.16 (2H, m), 1.18 (6H, s), 1.32 (1H, s), 1.50 (2H, d), 1.59-1.77 (6H, m), 1.87 (2H, d), 2.11 (1H, s), 2.17 (2H, s), 2.40-2.46 (1H, m), 2.49 (2H, d), 3.47-3.56 (2H, m), 4.14 (1H, d), 4.47 (2H, d), 5.96 (1H, d), 8.29 (1H, s)

m/z (ESI+) (M+H)+=427; HPLC t_{R}=1.97 min.

Intermediate 73 may be prepared as follows: methyl 4-cyclopropyl-2-(2,6-dimethylmorpholino)pyrimidine-5-carboxylate

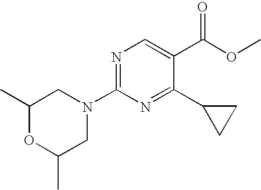

A solution of (Z)-ethyl 2-(cyclopropanecarbonyl)-3-(dimethylamino)acrylate (0.528 g, 2.5 mmol) in methanol (10 mL) was added dropwise to a stirred suspension of 2,6-dimethylmorpholine-4-carboximidamide hydrobromide (0.595 g, 2.50 mmol) and Sodium methoxide 0.5M in methanol (5.00 mL, 2.50 mmol) in methanol (10 mL) at room temperature, over a period of 5 minutes under nitrogen. The resulting suspension was stirred at 70° C. for 4 hours. The reaction mixture was evaporated to dryness and redissolved in EtOAc (50 mL), and washed sequentially with water (10 mL) and saturated brine (10 mL). The organic layer was dried over MgSO4, filtered and evaporated to afford methyl 4-cyclopropyl-2-(2,6-dimethylmorpholino)pyrimidine-5-carboxylate as an oil, which crystallised and was used without purification in the next step.

1H NMR (400.132 MHz, CDCl3) δ 1.00-1.05 (2H, m), 1.14-1.19 (2H, m), 1.24 (6H, d), 2.58 (2H, dd), 3.22 (1H, septet), 3.54-3.63 (2H, m), 3.87 (3H, s), 4.61 (2H, s), 8.75 (1H, s)

m/z (ESI+) (M+H)+=292; HPLC t_{R}=2.72 min methyl ester and (M+H)+=306; HPLC t_{R}=2.98 min ethyl ester

Intermediate 74 may be prepared as follows: 4-cyclopropyl-2-(2,6-dimethylmorpholino)pyrimidine-5-carboxylic acid

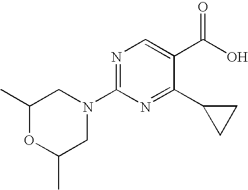

A solution of Lithium hydroxide IM (4.64 mL, 4.64 mmol)was added dropwise to a stirred solution of methyl 4-cyclopropyl-2-(2,6-dimethylmorpholino)pyrimidine-5-carboxylate (Intermediate 73, 676 mg, 2.32 mmol) in tetrahydrofuran (5 mL):methanol (1.7 mL) over a period of 5 minutes. The resulting solution was stirred at 20° C. for 16 hours. The reaction mixture was concentrated and diluted with water (15 mL), and washed sequentially with ethyl acetate (2×10 mL), the aqueous phase was acidified with 2M HCl. The precipitate was collected by filtration, washed with water (10 mL) and dried under vacuum to afford 4-cyclopropyl-2-(2,6-dimethylmorpholino)pyrimidine-5-carboxylic acid (473 mg, 74%) as a white solid, which was used without further purification.

1H NMR (400.132 MHz, CDCl3) δ 1.02-1.08 (2H, m), 1.17-1.22 (2H, m), 1.25 (6H, d), 2.61 (2H, dd), 3.23-3.31 (1H, m), 3.55-3.65 (2H, m), 4.62 (2H, d), 8.87 (1H, s)

m/z (ESI+) (M+H)+=278; HPLC t_{R}=2.13 min.

1. A compound of formula (1): 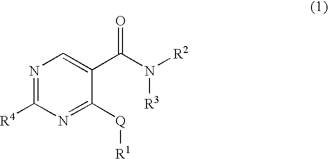
wherein:
Q is O, S, N(R⁸) or a single bond;
R⁸ is selected from hydrogen, C₁₋₄alkyl, C₃₋₅cycloalkyl and C₃₋₅cycloalkylmethyl (each of which is optionally substituted by 1, 2 or 3 fluoro atoms);
R¹ is selected from C₁₋₆alkyl, C₂₋₆alkenyl, C₂₋₆alkynyl, C₃₋₇cycloalkyl, heterocyclyl, heteroaryl, aryl, arylC₁₋₃alkyl, heteroarylC₁₋₃alkyl, C₃₋₇cycloalkylC₁₋₃alkyl, heterocyclylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₙ— (wherein n is 0, 1, 2 or 3), R⁵CON(R^{5′})-, (R^{5′})(R^{5″})N—, (R^{5′})(R^{5″})NC(O)—, R^{5″}C(O)O—, R^{5′}OC(O)—, (R^{5′})(R^{5″})NC(O)N(R^{5′″})-, R⁵SO₂N(R^{5″})-, (R^{5′})(R^{5″})NSO₂— and C₁₋₂alkyl optionally substituted by 1, 2 or 3 substituents independently selected from hydroxy, halo, carboxy and C₁₋₃alkoxy (wherein R⁵ is C₁₋₃alkyl optionally substituted by 1, 2 or 3 substituents independently selected from hydroxyl, halo and cyano; and
R^{5′}, R^{5″} and R^{5′″} are independently selected from hydrogen and C₁₋₃alkyl optionally substituted by 1, 2 or 3 substituents independently selected from hydroxyl, halo, C₁₋₃alkoxy, carboxy and cyano or R^{5′} and R^{5″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano]; or
R¹ and R⁸ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁹ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R² is selected from adamantyl optionally substituted, on available carbon atoms, by 1 or substituents independently selected from R⁶;
R³ is hydrogen;
R⁴ is selected from hydrogen, R¹⁰, —OR¹⁰, —SR¹⁰ and —NR¹¹R¹²;
R¹⁰ is selected from C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, heterocyclylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₚ— (wherein p is 0, 1, 2 or 3), R¹³CON(R^{13′})—, (R^{13′})(R^{13″})N—, (R^{13′})(R^{13″})NC(O)—, R^{13″}C(O)O—, R^{13′}OC(O), (R^{13′})(R^{13″})NC(O)N(R^{13′″}-, R¹³SO₂N(R^{13″})—, (R^{13′})(R^{13″})NSO₂— and C₁₋₂alkyl optionally substituted by 1, 2 or 3 substituents independently selected from hydroxy, halo, carboxy and C₁₋₃alkoxy (wherein R¹³ is C₁₋₃alkyl optionally substituted by 1, 2 or 3 substituents selected from hydroxyl, halo and cyano; and
R^{13′}, R^{13″} and R^{13′″} are independently selected from hydrogen and C₁₋₃alkyl optionally substituted by 1, 2 or 3 substituents independently selected from hydroxyl, halo, C₁₋₃alkoxy, carboxy and cyano or R^{13′} and R^{13″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano];
R¹¹ is selected from hydrogen, C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, heterocyclylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})-, (R^{14′})(R^{14″})NC(O)—, R^{14″}C(O)O—, R^{14′}OC(O)—, (R^{14′})(R^{14″})NC(O)N(R^{14′″})—, R¹⁴SO₂N(R^{14″})-, (R^{14′})(R^{14″})NSO₂— and C₁₋₂alkyl optionally substituted by 1, 2 or 3 substituents independently selected from hydroxy, halo, carboxy and C₁₋₃alkoxy (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo,
C₁₋₃alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano]; and
R¹² is selected from hydrogen, C₁₋₄alkyl, C₃₋₅cycloalkyl and C₃₋₅cycloalkylmethyl (each of which is optionally substituted by 1, 2 or 3 fluoro atoms); or
R¹¹ and R¹² together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted, on available carbon atoms, by 1, 2, or 3 substituents independently selected from R¹⁵ and optionally substituted on an available nitrogen by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R⁶, R⁷, R⁹ and R¹⁵ are independently selected from hydroxyl, halo, oxo, carboxy, cyano, trifluoromethyl, R¹⁶, R¹⁶O—, R¹⁶CO—, R¹⁶C(O)O—, R¹⁶CON(R^{6′}), (R^{16′})(R^{16″})NC(O)—, (R^{16′})(R^{16″})N—, R¹⁶S(O)ₐ—wherein a is 0 to 2, R^{16′}OC(O)—, (R^{16′})(R^{16″})NSO₂—, R¹⁶SO₂N(R^{16″})—, (R^{16′})(R^{16″})NC(O)N(R^{16′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted, on available carbon atoms, by 1, 2 or 3 substituents independently selected from C₁₋₄alkyl, hydroxyl, cyano, trifluoromethyl, trifluoromethoxy, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₄alkylS(O)ᵣ— and C₁₋₄alkylS(O)ᵣC₁₋₄alkyl (wherein r is independently selected 0, 1 and 2) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano];
R¹⁶ is independently selected from, C₁₋₃alkyl optionally substituted by 1, 2 or 3 substituents independently selected from hydroxyl, halo, C₁₋₄alkoxy, carboxy and cyano;
R^{16′}, R^{16″} and R^{16′″} are independently selected from hydrogen and C₁₋₃alkyl optionally substituted by 1, 2, or 3 substituents independently selected from hydroxyl, halo, C₁₋₄alkoxy, carboxy and cyano);
or a pharmaceutically-acceptable salt thereof.

2. A compound of formula (1) as defined in claim 1 or a pharmaceutically-acceptble salt thereof wherein Q is a single bond.

3. A compound of the formula (1) as defined in claim 1 or a pharmaceutically-acceptble salt thereof wherein R¹ is selected from C₃₋₇cycloalkyl and heterocyclyl [each of which is optionally substituted, on available carbon atoms, by 1, 2 or 3 substituents independently selected from C₁₋₃alkyl, halo, cyano, trifluoromethyl, C₁₋₃alkoxy and C₁₋₂alkyl optionally substituted by 1, 2 or 3 substituents independently selected from hydroxy, halo, carboxy and C₁₋₃alkoxy; and optionally substituted, on an available nitrogen, by a substituent independently selected from C₁₋₁₄alkyl and C₂₋₄alkanoyl].

4. A compound of the formula (1) as defined in claim 1 or a pharmaceutically-acceptble salt thereof wherein R⁴ is —NR¹¹R¹² and wherein R¹¹ and R¹² are as defined in claim 1.

5. A compound as defined in claim 1 selected from the following:
4-cyclopropyl-N-[(2s,5r)-5-hydroxyadamantan-2-yl]-2-morpholin-4-ylpyrimidine-5-carboxamide;
4-cyclopropyl-N-[(2r,5s)-5-hydroxyadamantan-2-yl]-2-methylpyrimidine-5-carboxamide;
4-cyclopropyl-N-[(2r,5s)-5-hydroxyadamantan-2-yl]pyrimidine-5-carboxamide;
4-tert-butyl-N-[(2r,5s)-5-hydroxyadamantan-2-yl]-2-morpholin-4-ylpyrimidine-5-carboxamide;
N-[(2r,5s)-5-hydroxyadamantan-2-yl]-4-methyl-2-morpholin-4-ylpyrimidine-5-carboxamide;
4-tert-butyl-N-[(2r,5 s)-5-hydroxyadamantan-2-yl]-2-methylpyrimidine-5-carboxamide;
4-tert-butyl-N-[(2r,5s)-5-hydroxyadamantan-2-yl]pyrimidine-5-carboxamide;
N-[(2r,5s)-5-hydroxyadamantan-2-yl]-2-morpholin-4-yl-4-propylsulfanyl-pyrimidine-5-carboxamide;
N-[(2r,5s)-5-hydroxyadamantan-2-yl]-2-methyl-4-propylsulfanylpyrimidine-5-carboxamide;
N-[(2r,5s)-5-hydroxyadamantan-2-yl]-4-propylsulfanylpyrimidine-5-carboxamide;
4-cyclopropyl-N-[(2r,5s)-5-hydroxyadamantan-2-yl]-2-methylsulfanyl-pyrimidine-5-carboxamide;
N-(2-adamantyl)-4-cyclopropyl-2-methyl-pyrimidine-5-carboxamide;
N-(2-adamantyl)-4-cyclopropyl-2-morpholino-pyrimidine-5-carboxamide;
N-(2-adamantyl)-4-tert-butyl-2-morpholin-4-ylpyrimidine-5-carboxamide;
N-(2-adamantyl)-4-methyl-2-morpholin-4-ylpyrimidine-5-carboxamide;
N-[(2r,5s)-5-hydroxyadamantan-2-yl]-2,4-bis(propylsulfanyl)pyrimidine-5-carboxamide;
2-dimethylamino-N-[(2r,5s)-5-hydroxyadamantan-2-yl]-4-propylsulfanylpyrimidine-5-carboxamide;
4-dimethylamino-N-[(2r,5s)-5-hydroxyadamantan-2-yl]-2-propylsulfanylpyrimidine-5-carboxamide;
{(3S)-1-[5-(cyclohexylcarbamoyl)-4-(propylthio)pyrimidin-2-yl]piperidin-3-yl}acetic acid;
N-[(2r,5s)-5-hydroxyadamantan-2-yl]-2-methylamino-4-propylsulfanylpyrimidine-5-carboxamide;
N-[(2r,5s)-5-hydroxyadamantan-2-yl]-4-methylamino-2-propylsulfanylpyrimidine-5-carboxamide;
2-[(2S,6R)-2,6-dimethylmorpholin-4-yl]-N-[(2r,5s)-5-hydroxyadamantan-2-yl]-4-propylsulfanylpyrimidine-5-carboxamide;
4-[(2S,6R)-2,6-dimethylmorpholin-4-yl]-N-[(2r,5s)-5-hydroxyadamantan-2-yl]-2-propylsulfanylpyrimidine-5-carboxamide;
4-(4-acetylpiperazin-1-yl)-N-[(2r,5s)-5-hydroxyadamantan-2-yl]-2-propylsulfanylpyrimidine-5-carboxamide;
2-(4-acetylpiperazin-1-yl)-N-[(2r,5s)-5-hydroxyadamantan-2-yl]-4-propylsulfanylpyrimidine-5-carboxamide;
2-(4-acetylpiperazin-1-yl)-N-(2-adamantyl)-4-propylsulfanyl-pyrimidine-5-carboxamide;
N-(2-adamantyl)-2-(4-methylsulfonylpiperazin-1-yl)-4-propylsulfanyl-pyrimidine-5-carboxamide;
N-(2-adamantyl)-2-[4-(dimethylcarbamoyl)piperazin-1-yl]-4-propylsulfanyl-pyrimidine-5-carboxamide;
4-cyclopentyl-N-[(2s,5r)-5-hydroxyadamantan-2-yl]-2-morpholin-4-ylpyrimidine-5-carboxamide;
N-[(2s,5r)-5-hydroxyadamantan-2-yl]-2-morpholin-4-yl-4-propoxypyrimidine-5-carboxamide;
4-cyclopropyl-N-[(2r,5s)-5-hydroxyadamantan-2-yl]-2-[(3R)-oxolan-3-ylamino]pyrimidine-5-carboxamide;
N-[(2r,5s)-5-hydroxyadamantan-2-yl] 4-cyclopropyl-2-[(3S)-oxolan-3-yl]amino]pyrimidine-5-carboxamide;
N-[(2s,5r)-5-hydroxyadamantan-2-yl]-2,4-dimorpholin-4-ylpyrimidine-5-carboxamide;
4-cyclopropyl-N-[(2r,5s)-5-hydroxyadamantan-2-yl]-2-methoxypyrimidine-5-carboxamide;
4-cyclopropyl-N-[(2r,5s)-5-hydroxyadamantan-2-yl]-2-methylaminopyrimidine-5-carboxamide;
4-cyclopropyl-N-[(2r,5s)-5-hydroxyadamantan-2-yl]-2-thiomorpholin-4-ylpyrimidine-5-carboxamide
4-cyclopropyl-N-[(2r,5s)-5-hydroxyadamantan-2-yl]-2-(1-oxo-1,4-thiazinan-4-yl)pyrimidine-5-carboxamide;
4-cyclopropyl-2-(1,1-dioxo-1,4-thiazinan-4-yl)-N-[(2r,5s)-5-hydroxyadamantan-2-yl]pyrimidine-5-carboxamide;
4-cyclohexyl-N-[(2r,5s)-5-hydroxyadamantan-2-yl]-2-morpholin-4-ylpyrimidine-5-carboxamide;
4-cyclopentyl-N-[(2r,5 s)-5-hydroxyadamantan-2-yl]-2-methylpyrimidine-5-carboxamide;
4-cyclobutyl-N-[(2r,5 s)-5-hydroxyadamantan-2-yl]-2-morpholin-4-ylpyrimidine-5-carboxamide;
4-cyclobutyl-N-[(2r,5s)-5-hydroxyadamantan-2-yl]-2-thiomorpholin-4-ylpyrimidine-5-carboxamide
4-cyclopropyl-2-(2,6-dimethylmorpholin-4-yl)-N-[(2r,5s)-5-hydroxyadamantan-2-yl]pyrimidine-5-carboxamide;
4-cyclopropyl-2-(3,3-difluoroazetidin-1-yl)-N-[(2r,5s)-5-hydroxyadamantan-2-yl]pyrimidine-5-carboxamide;
2-(azetidin-1-yl)-4-cyclopropyl-N-[(2r,5s)-5-hydroxyadamantan-2-yl]pyrimidine-5-carboxamide;
2-(cyclobutylamino)-4-cyclopropyl-N-[(2r,5s)-5-hydroxyadamantan-2-yl]pyrimidine-5-carboxamide;
4-cyclopropyl-N-[(2r,5s)-5-hydroxyadamantan-2-yl]-2-[4-(2-methoxyethyl)piperazin-1-yl]pyrimidine-5-carboxamide;
4-cyclopropyl-2-(cyclopropylamino)-N-[(2r,5 s)-5-hydroxyadamantan-2-yl]pyrimidine-5-carboxamide;
2-(cyclopentylamino)-4-cyclopropyl-N-[(2r,5s)-5-hydroxyadamantan-2-yl]pyrimidine-5-carboxamide;
4-cyclopropyl-N-[(2r,5s)-5-hydroxyadamantan-2-yl]-2-[(1S,4S)-2-oxa-5-azabicyclo[2.2.1]hept-5-yl]pyrimidine-5-carboxamide;
4-cyclopropyl-N-[(2r,5s)-5-hydroxyadamantan-2-yl]-2-[2-(hydroxymethyl)morpholin-4-yl]pyrimidine-5-carboxamide;
4-cyclopropyl-N-[(2r,5s)-5-hydroxyadamantan-2-yl]-2-[3-(hydroxymethyl)morpholin-4-yl]pyrimidine-5-carboxamide;
4-cyclopropyl-2-(dimethylamino)-N-[(2r,5 s)-5-hydroxyadamantan-2-yl]pyrimidine-5-carboxamide;
4-cyclopropyl-2-[(3R,5S)-3,5-dimethylpiperazin-1-yl]-N-[(2r,5s)-5-hydroxyadamantan-2-yl]pyrimidine-5-carboxamide;
4-cyclopropyl-2-[(2R,6R)-2,6-dimethylmorpholin-4-yl]-N-[(2r,5s)-5-hydroxyadamantan-2-yl]pyrimidine-5-carboxamide;
4-cyclopropyl-N-[(2r,5s)-5-hydroxyadamantan-2-yl]-2-(isopropylamino)pyrimidine-5-carboxamide;
4-cyclopropyl-N-[(2r,5s)-5-hydroxyadamantan-2-yl]-2-[(2-hydroxy-1,1-dimethylethyl)amino]pyrimidine-5-carboxamide;
4-cyclopropyl-N-[(2r,5s)-5-hydroxyadamantan-2-yl]-2-(tetrahydro-2H-pyran-4-ylamino)pyrimidine-5-carboxamide;
4-cyclopropyl-N-[(2r,5s)-5-hydroxyadamantan-2-yl]-2-[(2-hydroxy-2-methylpropyl)amino]pyrimidine-5-carboxamide;
4-cyclopropyl-2-[(1,1-dioxidotetrahydro-2H-thiopyran-4-yl)amino]-N-[(2r,5s)-5-hydroxyadamantan-2-yl]pyrimidine-5-carboxamide;
4-cyclopropyl-N-[(2r,5s)-5-hydroxyadamantan-2-yl]-2-[(2-hydroxyethyl)amino]pyrimidine-5-carboxamide;
4-cyclopropyl-N-[(2r,5s)-5-hydroxyadamantan-2-yl]-2-(4-methylsulfonylpiperazin-1-yl)pyrimidine-5-carboxamide;
4-cyclopropyl-N-[(2r,5s)-5-hydroxyadamantan-2-yl]-2-(oxetan-3-ylamino)pyrimidine-5-carboxamide;
4-cyclopropyl-N-[(2r,5s)-5-hydroxyadamantan-2-yl]-2-[(2-morpholin-4-ylethyl)amino]pyrimidine-5-carboxamide;
4-cyclopropyl-2-({2-[(2R,6S)-2,6-dimethylmorpholin-4-yl]ethyl}amino)-N-[(2r,5s)-5-hydroxyadamantan-2-yl]pyrimidine-5-carboxamide;
4-cyclopropyl-N-[(2r,5s)-5-hydroxyadamantan-2-yl]-2-{[2-(4-methylpiperazin-1-yl)ethyl]amino}pyrimidine-5-carboxamide;
2-(cyclobutyloxy)-4-cyclopropyl-N-[(2r,5s)-5-hydroxyadamantan-2-yl]pyrimidine-5-carboxamide;
4-cyclopropyl-N-[(2r,5s)-5-hydroxyadamantan-2-yl]-2-isopropoxypyrimidine-5-carboxamide;
2-(cyclopentyloxy)-4-cyclopropyl-N-[(2r,5 s)-5-hydroxyadamantan-2-yl]pyrimidine-5-carboxamide;
4-cyclopropyl-N-[(2r,5s)-5-hydroxyadamantan-2-yl]-2-(oxetan-3-yloxyl)pyrimidine-5-carboxamide;
(4-cyclopropyl-2-morpholinopyrimidin-5-yl)(3-(pyridin-3-yl)pyrrolidin-1-yl)methanone 1-(4-(4-cyclopropyl-5-(3-(pyridin-3-yl)pyrrolidine-1-carbonyl)pyrimidin-2-yl)piperazin-1-yl)ethanone;
(4-cyclopropyl-2-((2S,6R)-2,6-dimethylmorpholino)pyrimidin-5-yl)(3-(pyridin-3-yl)pyrrolidin-1-yl)methanone;
4-cyclobutyl-N-[(2r,5 s)-5-hydroxyadamantan-2-yl]-2-(1-oxo-1,4-thiazinan-4-yl)pyrimidine-5-carboxamide;
4-cyclobutyl-2-(1,1-dioxo-1,4-thiazinan-4-yl)-N-[(2r,5 s)-5-hydroxyadamantan-2-yl]pyrimidine-5-carboxamide;
2-amino-4-cyclobutyl-N-[(2r,5 s)-5-hydroxyadamantan-2-yl]pyrimidine-5-carboxamide;
2-azetidin-1-yl-4-cyclobutyl-N-[(2r,5 s)-5-hydroxyadamantan-2-yl]pyrimidine-5-carboxamide;
4-cyclobutyl-2-(dimethylamino)-N-[(2r,5s)-5-hydroxyadamantan-2-yl]pyrimidine-5-carboxamide;
4-cyclobutyl-N-[(2r,5 s)-5-hydroxyadamantan-2-yl]-2-[4-(2-methoxyethyl)piperazin-1-yl]pyrimidine-5-carboxamide;
4-cyclobutyl-2-(cyclopropylamino)-N-[(2r,5 s)-5-hydroxyadamantan-2-yl]pyrimidine-5-carboxamide;
4-cyclobutyl-2-(3,3-difluoroazetidin-1-yl)-N-[(2r,5 s)-5-hydroxyadamantan-2-yl]pyrimidine-5-carboxamide;
4-cyclobutyl-N-[(2r,5s)-5-hydroxyadamantan-2-yl]-2-[3-(hydroxymethyl)morpholin-4-yl]pyrimidine-5-carboxamide;
4-cyclobutyl-N-[(2r,5 s)-5-hydroxyadamantan-2-yl]-2-(methylamino)pyrimidine-5-carboxamide;
4-cyclobutyl-2-[(2R,6S)-2,6-dimethylmorpholin-4-yl]-N-[(2r,5s)-5-hydroxyadamantan-2-yl]pyrimidine-5-carboxamide;
4-cyclobutyl-N-[(2r,5 s)-5-hydroxyadamantan-2-yl]-2-[2-(hydroxymethyl)morpholin-4-yl]pyrimidine-5-carboxamide;
4-cyclobutyl-N-[(2r,5 s)-5-hydroxyadamantan-2-yl]-2-(isopropylamino)pyrimidine-5-carboxamide;
4-cyclobutyl-N-[(2r,5 s)-5-hydroxyadamantan-2-yl]-2-[(2-hydroxy-1,1-dimethylethyl)amino]pyrimidine-5-carboxamide;
4-cyclobutyl-N-[(2r,5s)-5-hydroxyadamantan-2-yl]-2-(tetrahydro-2H-pyran-4-ylamino)pyrimidine-5-carboxamide;
4-cyclobutyl-N-[(2r,5 s)-5-hydroxyadamantan-2-yl]-2-[(2-hydroxyethyl)amino]pyrimidine-5-carboxamide;
N-[(2r,5s)-5-hydroxyadamantan-2-yl] 4-cyclobutyl-2-(cyclobutylamino) pyrimidine-5-carboxamide;
4-cyclobutyl-2-[(3R,5S)-3,5-dimethylpiperazin-1-yl]-N-[(2r,5s)-5-hydroxyadamantan-2-yl]pyrimidine-5-carboxamide;
4-cyclobutyl-N-[(2r,5 s)-5-hydroxyadamantan-2-yl]-2-[(2-hydroxy-2-methylpropyl)amino]pyrimidine-5-carboxamide;
4-cyclobutyl-2-[(2R,6R)-2,6-dimethylmorpholin-4-yl]-N-[(2r,5s)-5-hydroxyadamantan-2-yl]pyrimidine-5-carboxamide;
4-cyclobutyl-2-(cyclopentylamino)-N-[(2r,5 s)-5-hydroxyadamantan-2-yl]pyrimidine-5-carboxamide;
4-cyclobutyl-N-[(2r,5s)-5-hydroxyadamantan-2-yl]-2-[(1S,4S)-2-oxa-5-azabicyclo[2.2.1]hept-5-yl]pyrimidine-5-carboxamide;
4-cyclobutyl-N-[(2r,5 s)-5-hydroxyadamantan-2-yl]-2-(oxetan-3-ylamino)pyrimidine-5-carboxamide;
4-cyclobutyl-2-[(1,1-dioxidotetrahydro-2H-thiopyran-4-yl)amino]-N-[(2r,5s)-5-hydroxyadamantan-2-yl]pyrimidine-5-carboxamide;
4-cyclobutyl-2-(cyclopentyloxy)-N-[(2r,5s)-5-hydroxyadamantan-2-yl]pyrimidine-5-carboxamide;
4-cyclobutyl-N-[(2r,5 s)-5-hydroxyadamantan-2-yl]-2-isopropoxypyrimidine-5-carboxamide;
4-cyclobutyl-2-(cyclobutyloxy)-N-[(2r,5s)-5-hydroxyadamantan-2-yl]pyrimidine-5-carboxamide;
4-cyclobutyl-N-[(2r,5 s)-5-hydroxyadamantan-2-yl]-2-(oxetan-3-yloxyl)pyrimidine-5-carboxamide;
4-cyclopentyl-N-[(2r,5s)-5-hydroxyadamantan-2-yl]-2-propan-2-yloxypyrimidine-5-carboxamide;
2-cyclobutyloxy-4-cyclopentyl-N-[(2r,5s)-5-hydroxyadamantan-2-yl]pyrimidine-5-carboxamide;
4-cyclopentyl-2-cyclopentyloxy-N-[(2r,5s)-5-hydroxyadamantan-2-yl]pyrimidine-5-carboxamide;
4-cyclopentyl-N-[(2r,5s)-5-hydroxyadamantan-2-yl]-2-(oxetan-3-yloxyl)pyrimidine-5-carboxamide;
4-cyclopentyl-N-[(2s,5r)-5-hydroxyadamantan-2-yl]-2-thiomorpholin-4-ylpyrimidine-5-carboxamide;
4-cyclopentyl-N-[(2r,5s)-5-hydroxyadamantan-2-yl]-2-(1-oxo-1,4-thiazinan-4-yl)pyrimidine-5-carboxamide;
4-cyclopentyl-2-(1,1-dioxo-1,4-thiazinan-4-yl)-N-[(2r,5 s)-5-hydroxyadamantan-2-yl]pyrimidine-5-carboxamide;
4-cyclopentyl-N-[(2r,5s)-5-hydroxyadamantan-2-yl]-2-methoxypyrimidine-5-carboxamide;
4-cyclopentyl-N-[(2r,5s)-5-hydroxyadamantan-2-yl]-2-methylaminopyrimidine-5-carboxamide;
4-cyclopentyl-2-[(2S,6R)-2,6-dimethylmorpholin-4-yl]-N-[(2r,5s)-5-hydroxyadamantan-2-yl]pyrimidine-5-carboxamide;
4-cyclopentyl-N-[(2r,5s)-5-hydroxyadamantan-2-yl]-2-[(1S,4S)-2-oxa-5-azabicyclo[2.2.1]heptan-5-yl]pyrimidine-5-carboxamide;
4-cyclopentyl-N-[(2r,5 s)-5-hydroxyadamantan-2-yl]-2-(propan-2-ylamino)pyrimidine-5-carboxamide;
4-cyclopentyl-2-(cyclopropylamino)-N-[(2r,5s)-5-hydroxyadamantan-2-yl]pyrimidine-5-carboxamide;
4-cyclopentyl-N-[(2r,5s)-5-hydroxyadamantan-2-yl]-2-[(3S)-3-methylmorpholin-4-yl]pyrimidine-5-carboxamide;
4-cyclopentyl-2-[(2S,6S)-2,6-dimethylmorpholin-4-yl]-N-[(2r,5s)-5-hydroxyadamantan-2-yl]pyrimidine-5-carboxamide;
4-cyclopentyl-N-[(2r,5s)-5-hydroxyadamantan-2-yl]-2-[4-(2-methoxyethyl)piperazin-1-yl]pyrimidine-5-carboxamide;
2-(4-acetylpiperazin-1-yl)-4-cyclopentyl-N-[(2r,5s)-5-hydroxyadamantan-2-yl]pyrimidine-5-carboxamide;
4-cyclopentyl-N-[(2r,5 s)-5-hydroxyadamantan-2-yl]-2-(3-oxo-4-propan-2-ylpiperazin-1-yl)pyrimidine-5-carboxamide;
4-cyclopentyl-N-[(2r,5s)-5-hydroxyadamantan-2-yl]-2-(4-methyl-3-oxopiperazin-1-yl)pyrimidine-5-carboxamide;
4-cyclopentyl-2-(cyclobutylamino)-N-[(2r,5s)-5-hydroxyadamantan-2-yl]pyrimidine-5-carboxamide;
4-cyclopentyl-2-(cyclopentylamino)-N-[(2r,5s)-5-hydroxyadamantan-2-yl]pyrimidine-5-carboxamide;
2-(azetidin-1-yl)-4-cyclopentyl-N-[(2r,5s)-5-hydroxyadamantan-2-yl]pyrimidine-5-carboxamide;
4-cyclopentyl-N-[(2r,5 s)-5-hydroxyadamantan-2-yl]-2-(oxetan-3-ylamino)pyrimidine-5-carboxamide;
4-cyclopentyl-2-dimethylamino-N-[(2r,5 s)-5-hydroxyadamantan-2-yl]pyrimidine-5-carboxamide;
4-cyclopentyl-2-[(3S,5R)-3,5-dimethylpiperazin-1-yl]-N-[(2r,5s)-5-hydroxyadamantan-2-yl]pyrimidine-5-carboxamide;
2-amino-4-cyclopentyl-N-[(2r,5s)-5-hydroxyadamantan-2-yl]pyrimidine-5-carboxamide;
4-cyclopentyl-2-[(1,1-dioxothian-4-yl)amino]-N-[(2r,5 s)-5-hydroxyadamantan-2-yl]pyrimidine-5-carboxamide;
4-cyclopentyl-N-[(2r,5 s)-5-hydroxyadamantan-2-yl]-2-[(2-hydroxy-2-methylpropyl)amino]pyrimidine-5-carboxamide;
4-cyclopentyl-N-[(2r,5s)-5-hydroxyadamantan-2-yl]-2-(2-hydroxyethylamino)pyrimidine-5-carboxamide;
4-cyclopentyl-N-[(2r,5s)-5-hydroxyadamantan-2-yl]-2-[(1-hydroxy-2-methylpropan-2-yl)amino]pyrimidine-5-carboxamide;
4-cyclopentyl-N-[(2r,5s)-5-hydroxyadamantan-2-yl]-2-(oxan-4-ylamino)pyrimidine-5-carboxamide;
4-cyclopentyl-N-[(2r,5s)-5-hydroxyadamantan-2-yl]-2-[3-(hydroxymethyl)morpholin-4-yl]pyrimidine-5-carboxamide;
4-cyclopentyl-N-[(2r,5s)-5-hydroxyadamantan-2-yl]-2-[[(3R)-oxolan-3-yl]amino]pyrimidine-5-carboxamide;
4-cyclopentyl-N-[(2r,5s)-5-hydroxyadamantan-2-yl]-2-(4-methylsulfonylpiperazin-1-yl)pyrimidine-5-carboxamide;
4-cyclopentyl-N-[(2r,5 s)-5-hydroxyadamantan-2-yl]-2-[[(3S)-oxolan-3-yl]amino]pyrimidine-5-carboxamide;
4-cyclopentyl-N-[(2r,5s)-5-hydroxyadamantan-2-yl]-2-[2-(hydroxymethyl)morpholin-4-yl]pyrimidine-5-carboxamide;
4-cyclopentyl-2-(3,3-difluoroazetidin-1-yl)-N-[(2r,5s)-5-hydroxyadamantan-2-yl]pyrimidine-5-carboxamide;
4-cyclopentyl-N-[(2r,5s)-5-hydroxytricyclo[3.3.1.13.7]dec-2-yl]-2-[(2-morpholin-4-ylethyl)amino]pyrimidine-5-carboxamide;
4-cyclopentyl-2-({2-[(2R,6S)-2,6-dimethylmorpholin-4-yl]ethyl}amino)-N-[(2r,5s)-5-hydroxytricyclo[3.3.1.13.7]dec-2-yl]pyrimidine-5-carboxamide;
4-cyclopentyl-2-cyclopropyl-N-[(2r,5 s)-5-hydroxyadamantan-2-yl]pyrimidine-5-carboxamide;
4-cyclopentyl-N-[(2r,5s)-5-hydroxyadamantan-2-yl]-2-propan-2-ylpyrimidine-5-carboxamide2-(1-aminocyclopropyl)-4-cyclopentyl-N-[(2r,5s)-5-hydroxyadamantan-2-yl]pyrimidine-5-carboxamide;
2-(aminomethyl)-4-cyclopentyl-N-[(2r,5s)-5-hydroxyadamantan-2-yl]pyrimidine-5-carboxamide;
4-(3,3-difluorocyclobutyl)-N-[(2s,5r)-5-hydroxyadamantan-2-yl]-2-methylpyrimidine-5-carboxamide;
4-(3,3-difluorocyclobutyl)-N-[(2r,5 s)-5-hydroxyadamantan-2-yl]-2-methylaminopyrimidine-5-carboxamide;
2-(cyclopropylamino)-4-(3,3-difluorocyclobutyl)-N-[(2r,5s)-5-hydroxyadamantan-2-yl]pyrimidine-5-carboxamide;
4-(3,3-difluorocyclobutyl)-2-[(2S,6R)-2,6-dimethylmorpholin-4-yl]-N-[(2r,5s)-5-hydroxyadamantan-2-yl]pyrimidine-5-carboxamide;
2-cyclobutyloxy-4-(3,3-difluorocyclobutyl)-N-[(2r,5s)-5-hydroxyadamantan-2-yl]pyrimidine-5-carboxamide;
N-[(2r,5s)-5-hydroxyadamantan-2-yl]-2-methyl-4-(oxolan-2-yl)pyrimidine-5-carboxamide;
N-[(2r,5s)-5-hydroxyadamantan-2-yl]-4-(oxolan-2-yl)-2-(propan-2-ylamino)pyrimidine-5-carboxamide;
2-(cyclopropylamino)-N-[(2r,5 s)-5-hydroxyadamantan-2-yl]-4-(oxolan-2-yl)pyrimidine-5-carboxamide;
N-[(2r,5s)-5-hydroxyadamantan-2-yl]-2-methylamino-4-(oxolan-2-yl)pyrimidine-5-carboxamide;
2-(cyclobutylamino)-N-[(2r,5 s)-5-hydroxyadamantan-2-yl]-4-(oxolan-2-yl)pyrimidine-5-carboxamide;
2-[(2S,6R)-2,6-dimethylmorpholin-4-yl]-N-[(2r,5s)-5-hydroxyadamantan-2-yl]-4-(oxolan-2-yl)pyrimidine-5-carboxamide;
N-[(2r,5s)-5-hydroxyadamantan-2-yl]-2-(oxetan-3-ylamino)-4-(oxolan-2-yl)pyrimidine-5-carboxamide;
N-[(2r,5s)-5-hydroxyadamantan-2-yl]-4-(oxolan-2-yl)-2-propan-2-yloxypyrimidine-5-carboxamide;
N-[(2r,5s)-5-hydroxyadamantan-2-yl]-2-methylsulfanyl-4-[(2R)-oxolan-2-yl]pyrimidine-5-carboxamide;
N-[(2r,5s)-5-hydroxyadamantan-2-yl]-2-methylamino-4-[(2R)-oxolan-2-yl]pyrimidine-5-carboxamide;
2-(cyclopropylamino)-N-[(2r,5 s)-5-hydroxyadamantan-2-yl]-4-[(2R)-oxolan-2-yl]pyrimidine-5-carboxamide;
N-[(2r,5s)-5-hydroxyadamantan-2-yl]-4-[(2R)-oxolan-2-yl]-2-(propan-2-ylamino)pyrimidine-5-carboxamide;
2-[(3S,5R)-3,5-dimethylpiperazin-1-yl]-N-[(2r,5s)-5-hydroxyadamantan-2-yl]-4-[(2R)-oxolan-2-yl]pyrimidine-5-carboxamide;
N-[(2r,5s)-5-hydroxyadamantan-2-yl]-2-(oxetan-3-ylamino)-4-[(2R)-oxolan-2-yl]pyrimidine-5-carboxamide;
N-[(2r,5s)-5-hydroxyadamantan-2-yl]-2-(oxan-4-ylamino)-4-[(2R)-oxolan-2-yl]pyrimidine-5-carboxamide;
2-(cyclobutylamino)-N-[(2r,5 s)-5-hydroxyadamantan-2-yl]-4-[(2R)-oxolan-2-yl]pyrimidine-5-carboxamide;
N-[(2r,5s)-5-hydroxyadamantan-2-yl]-4-[(2R)-oxolan-2-yl]-2-propan-2-yloxypyrimidine-5-carboxamide;
N-[(2r,5s)-5-hydroxyadamantan-2-yl]-2-methylsulfanyl-4-[(2S)-oxolan-2-yl]pyrimidine-5-carboxamide;
N-[(2r,5s)-5-hydroxyadamantan-2-yl]-2-methylamino-4-[(2S)-oxolan-2-yl]pyrimidine-5-carboxamide;
N-[(2r,5s)-5-hydroxyadamantan-2-yl]-4-[(2S)-oxolan-2-yl]-2-(propan-2-ylamino)pyrimidine-5-carboxamide;
2-(cyclopropylamino)-N-[(2r,5s)-5-hydroxyadamantan-2-yl]-4-[(2S)-oxolan-2-yl]pyrimidine-5-carboxamide;
N-[(2r,5s)-5-hydroxyadamantan-2-yl]-4-[(2S)-oxolan-2-yl]-2-propan-2-yloxypyrimidine-5-carboxamide;
2-[(2S,6R)-2,6-Dimethylmorpholin-4-yl]-N-[(2r,5s)-5-hydroxyadamantan-2-yl]-4-[(2R)-oxolan-2-yl]pyrimidine-5-carboxamide;
2-[(2S,6R)-2,6-Dimethylmorpholin-4-yl]-N-[(2r,5s)-5-hydroxyadamantan-2-yl]-4-[(2S)-oxolan-2-yl]pyrimidine-5-carboxamide;
4-(3,3-Difluorocyclopentyl)-N-[(2r,5 s)-5-hydroxyadamantan-2-yl]-2-methylpyrimidine-5-carboxamide;
N-[(2r,5s)-5-hydroxyadamantan-2-yl]-4-(1-methylcyclopropyl)-2-morpholin-4-ylpyrimidine-5-carboxamide;
N-[(2r,5s)-5-hydroxyadamantan-2-yl]-4-(1-methylcyclopropyl)-2-morpholin-4-ylpyrimidine-5-carboxamide;
(Z)-3-dimethylamino-2-(1-methylcyclopropanecarbonyl)-N-(5-phenylmethoxy-2-adamantyl)prop-2-enamide;
N-[(2r,5s)-5-hydroxyadamantan-2-yl]-2-methyl-4-phenylpyrimidine-5-carboxamide;
4-(2-Chlorophenyl)-N-[(2r,5s)-5-hydroxyadamantan-2-yl]-2-methylpyrimidine-5-carboxamide;
4-(cyclopentylmethyl)-N-[(2r,5s)-5-hydroxyadamantan-2-yl]-2-methylpyrimidine-5-carboxamide;
4-butyl-N-[(2r,5s)-5-hydroxyadamantan-2-yl]-2-methylpyrimidine-5-carboxamide;
N-[(2s,5r)-5-hydroxyadamantan-2-yl]-4-isobutyl-2-methylpyrimidine-5-carboxamide;
4-(2,2-dimethylpropyl)-N-[(2r,5s)-5-hydroxyadamantan-2-yl]-2-methylpyrimidine-5-carboxamide;
4-(cyclopropylmethyl)-N-[(2r,5s)-5-hydroxyadamantan-2-yl]-2-methylpyrimidine-5-carboxamide
4-cyclohexyl-N-[(2r,5 s)-5-hydroxyadamantan-2-yl]-2-(methylthio)pyrimidine-5-carboxamide;
4-cyclohexyl-N-[(2r,5 s)-5-hydroxyadamantan-2-yl]-2-thiomorpholin-4-ylpyrimidine-5-carboxamide;
4-cyclohexyl-N-[(2r,5s)-5-hydroxyadamantan-2-yl]-2-(1-oxidothiomorpholin-4-yl)pyrimidine-5-carboxamide;
4-cyclohexyl-2-(1,1-dioxidothiomorpholin-4-yl)-N-[(2r,5 s)-5-hydroxyadamantan-2-yl]pyrimidine-5-carboxamide;
2,4-bis(dimethylamino)-N-[(2r,5s)-5-hydroxyadamantan-2-yl]pyrimidine-5-carboxamide;
2,4-bis(3,3-difluoroazetidin-1-yl)-N-[(2r,5s)-5-hydroxyadamantan-2-yl]pyrimidine-5-carboxamide;
2,4-bis(azetidin-1-yl)-N-[(2r,5s)-5-hydroxyadamantan-2-yl]pyrimidine-5-carboxamide;
N-[(2r,5s)-5-hydroxyadamantan-2-yl]-2-methyl-4-propan-2-yloxypyrimidine-5-carboxamide;
4-cyclobutyloxy-N-[(2r,5s)-5-hydroxyadamantan-2-yl]-2-methylpyrimidine-5-carboxamide;
4-cyclopentyloxy-N-[(2r,5 s)-5-hydroxyadamantan-2-yl]-2-methylpyrimidine-5-carboxamide;
2-[(2R,6S)-2,6-Dimethylmorpholin-4-yl]-N-[(2r,5s)-5-hydroxytricyclo[3.3.1.13.7]dec-2-yl]-4-methoxypyrimidine-5-carboxamide;
2-(cyclopropylamino)-N-[(2r,5s)-5-hydroxytricyclo[3.3.1.13.7]dec-2-yl]-4-methoxypyrimidine-5-carboxamide;
2-(cyclobutylamino)-N-[(2r,5s)-5-hydroxytricyclo[3.3.1.13.7]dec-2-yl]-4-methoxypyrimidine-5-carboxamide;
2-(cyclobutyloxy)-N-[(2r,5s)-5-hydroxytricyclo[3.3.1.13.7]dec-2-yl]-4-methoxypyrimidine-5-carboxamide;
2-[(2S,6R)-2,6-dimethylmorpholin-4-yl]-4-ethoxy-N-[(2r,5s)-5-hydroxyadamantan-2-yl]pyrimidine-5-carboxamide;
2-(cyclopropylamino)-4-ethoxy-N-[(2r,5s)-5-hydroxytricyclo[3.3.1.13.7]dec-2-yl]pyrimidine-5-carboxamide;
4-ethoxy-N-[(2r,5s)-5-hydroxytricyclo[3.3.1.13.7]dec-2-yl]-2-(oxetan-3-ylamino)pyrimidine-5-carboxamide;
2-(cyclobutylamino)-4-ethoxy-N-[(2r,5s)-5-hydroxytricyclo[3.3.1.13.7]dec-2-yl]pyrimidine-5-carboxamide;
2-(cyclobutyloxy)-4-ethoxy-N-[(2r,5 s)-5-hydroxytricyclo[3.3.1.13.7]dec-2-yl]pyrimidine-5-carboxamide;
2-[(2R,6S)-2,6-dimethylmorpholin-4-yl]-N-[(2r,5s)-5-hydroxytricyclo[3.3.1.13.7]dec-2-yl]-4-(1-methylethoxyl)pyrimidine-5-carboxamide;
2-(cyclopropylamino)-N-[(2r,5s)-5-hydroxytricyclo[3.3.1.13.7]dec-2-yl]-4-(1-methylethoxyl)pyrimidine-5-carboxamide;
N-[(2r,5s)-5-hydroxytricyclo[3.3.1.13.7]dec-2-yl]-4-(1-methylethoxy)-2-(oxetan-3-ylamino)pyrimidine-5-carboxamide;
2-(cyclobutylamino)-N-[(2r,5 s)-5-hydroxytricyclo[3.3.1.13.7]dec-2-yl]-4-(1-methylethoxyl)pyrimidine-5-carboxamide;
2-(cyclobutyloxy)-N-[(2r,5s)-5-hydroxytricyclo[3.3.1.13.7]dec-2-yl]-4-(1-methylethoxyl)pyrimidine-5-carboxamide;
N-[(2r,5s)-5-hydroxyadamantan-2-yl] 2-[(2S,6R)-2,6-dimethylmorpholin-4-yl]-4-[(2R)-oxolan-2-yl]pyrimidine-5-carboxamide;
4-cyclopropyl-2-[(2S,6R)-2,6-dimethylmorpholin-4-yl]-N-[(2r,5s)-5-hydroxyadamantan-2-yl]pyrimidine-5-carboxamide; and
2-[(2S,6R)-2,6-dimethylmorpholin-4-yl]-N-[(2r,5s)-5-hydroxyadamantan-2-yl]-4-(methoxymethyl)pyrimidine-5-carboxamide;
or a pharmaceutically-acceptable salt thereof.

6. A pharmaceutical composition, which comprises a compound of formula (1), or a pharmaceutically acceptable salt thereof; as claimed in claim 1 in association with a pharmaceutically-acceptable diluent or carrier.

7. A method for producing an 11βHSD1 inhibitory effect in a warm-blooded animal, such as man, in need of such treatment, which comprises administering to said animal an effective amount of a compound of formula (I) wherein:
Q is O, S, N(R⁸) or a single bond;
R⁸ is selected from hydrogen, C₁₋₄alkyl, C₃₋₅cycloalkyl and C₃₋₅cycloalkylmethyl (each of which is optionally substituted by 1, 2 or 3 fluoro atoms);
R¹ is selected from C₁₋₆alkyl, C₂₋₆alkenyl, C₂₋₆alkynyl, C₃₋₇cycloalkyl, heterocyclyl, heteroaryl, aryl, arylC₁₋₃alkyl, heteroarylC₁₋₃alkyl, C₃₋₇cycloalkylC₁₋₃alkyl, heterocyclylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₙ— (wherein n is 0, 1, 2 or 3), R⁵CON(R^{5′})-, (R^{5′})(R^{5″})N—, (R^{5′})(R^{5″})NC(O)—, R^{5′}C(O)O—, R^{5′}OC(O)—, (R^{5′})(R^{5″})NC(O)N(R^{5′″})-, R⁵SO₂N(R^{5″})-, (R^{5′})(R^{5″})NSO₂— and C₁₋₂alkyl optionally substituted by 1, 2 or 3 substituents independently selected from hydroxy, halo, carboxy and C₁₋₃alkoxy (wherein R⁵ is C₁₋₃alkyl optionally substituted by 1, 2 or 3 substituents independently selected from hydroxyl, halo and cyano; and
R^{5′}, R^{5″} and R^{5′″} are independently selected from hydrogen and C₁₋₃alkyl optionally substituted by 1, 2 or 3 substituents independently selected from hydroxyl, halo, C₁₋₃alkoxy, carboxy and cyano or R^{5′} and R^{5″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano]; or
R¹ and R⁸ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁹ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R² is selected from C₃₋₇cycloalkyl(CH₂)ₘ—, and C₆₋₁₂polycycloalkyl(CH₂)ₘ— (wherein m is 0, 1 or 2 and the rings optionally contain 1 or 2 ring atoms independently selected from nitrogen, oxygen and sulphur are optionally substituted, on available carbon atoms, by 1, 2 or 3 substituents independently selected from R⁶ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano);
R³ is selected from hydrogen, C₁₋₄alkyl, C₃₋₅cycloalkyl and C₃₋₅cycloalkylmethyl (each of which is optionally substituted by 1, 2 or 3 fluoro atoms);
R² and R³ together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring wherein the resulting ring system is optionally substituted, on available carbon atoms, by 1, 2, or 3 substituents independently selected from R⁷ and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R⁴ is selected from hydrogen, R¹⁰, —OR¹⁰, —SR¹⁰ and —NR¹¹R¹²;
R¹⁰ is selected from C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, heterocyclylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)ₚ— (wherein p is 0, 1, 2 or 3), R¹³CON(R^{13′})—, (R^{13′})(R^{13″})N—, (R^{13′})(R^{13″})NC(O)—, R^{13″}C(O)O—, R^{13′} OC(O), (R^{13′})(R^{13″})NC(O)N(R^{13′″})-, R¹³SO₂N(R^{13″})—, (R^{13′})(R^{13″})NSO₂— and C₁₋₂alkyl optionally substituted by 1, 2 or 3 substituents independently selected from hydroxy, halo, carboxy and C₁₋₃alkoxy (wherein R¹³ is C₁₋₃alkyl optionally substituted by 1, 2 or 3 substituents selected from hydroxyl, halo and cyano; and
R^{13′}, R^{13″} and R^{13′″} are independently selected from hydrogen and C₁₋₃alkyl optionally substituted by 1, 2 or 3 substituents independently selected from hydroxyl, halo, C₁₋₃alkoxy, carboxy and cyano or R^{13′} and R^{13″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano];
R¹¹ is selected from hydrogen, C₁₋₆alkyl, C₂₋₆alkenyl, C₂₋₆alkynyl, C₃₋₇cycloalkyl, heterocyclyl, arylC₁₋₃alkyl, heteroarylC₁₋₃alkyl, heterocyclylC₁₋₃alkyl, C₃₋₇cycloalkylC₁₋₃alkyl, C₃₋₇cycloalkylC₂₋₃alkenyl and C₃₋₇cycloalkylC₂₋₃alkynyl, [each of which is optionally substituted, on available carbon atoms, by 1, 2 or 3 substituents independently selected from C₁₋₃alkyl, hydroxy, halo, oxo, cyano, trifluoromethyl, C₁₋₃alkoxy, C₁₋₃alkylS(O)_{q}— (wherein q is 0, 1, 2 or 3), R¹⁴CON(R^{14′})-, (R^{14′})(R^{14″})NC(O)—, R^{14″}C(O)O—, R^{14′}OC(O)—, (R^{14′})(R^{14″})NC(O)N(R^{14′″})-, R¹⁴SO₂N(R^{14″})-, (R^{14′})(R^{14″})NSO₂— and C₁₋₂alkyl optionally substituted by 1, 2 or 3 substituents independently selected from hydroxy, halo, carboxy and C₁₋₃alkoxy (wherein R¹⁴ is C₁₋₃alkyl optionally substituted by 1, 2 or 3 substituents independently selected from hydroxyl, halo and cyano; and
R^{14′}, R^{14″} and R^{14′″} are independently selected from hydrogen and C₁₋₃alkyl optionally substituted by 1, 2 or 3 substituents independently selected from hydroxyl, halo, C₁₋₃alkoxy, carboxy and cyano or R^{14′} and R^{14″} together with the nitrogen atom to which they are attached form a 4-7 membered saturated ring) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano]; and
R¹² is selected from hydrogen, C₁₋₄alkyl, C₃₋₅cycloalkyl and C₃₋₅cycloalkylmethyl (each of which is optionally substituted by 1, 2 or 3 fluoro atoms); or
R¹¹ and R¹² together with the nitrogen atom to which they are attached form a saturated mono, bicyclic or bridged ring system optionally containing 1 or 2 additional ring heteroatoms independently selected from nitrogen, oxygen and sulphur and optionally fused to a saturated, partially saturated or unsaturated monocyclic ring (optionally containing 1 or 2 additional ring heteroatoms independently selected from nitrogen, oxygen and sulphur) wherein the resulting ring system is optionally substituted, on available carbon atoms, by 1, 2, or 3 substituents independently selected from R¹⁵ and optionally substituted on an available nitrogen by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano;
R⁶, R⁷, R⁹ and R¹⁵ are independently selected from hydroxyl, halo, oxo, carboxy, cyano, trifluoromethyl, R¹⁶, R¹⁶O—, R¹⁶CO, R¹⁶C(O)O, R¹⁶CON(R^{16′})-, (R^{16′}) (16″)NC(O)—, (R^{16′})(R^{16″})N—, R¹⁶S(O)ₐ— wherein a is 0 to 2, R^{16′}OC(O)—, (R^{16′})(R^{16″})NSO₂—, R¹⁶SO₂N(R^{16″})—, (R^{16′})(R^{16″})NC(O)N(R^{16′″})—, phenyl and heteroaryl [wherein the phenyl and heteroaryl groups are optionally fused to a phenyl, heteroaryl or a saturated or partially-saturated 5- or 6-membered ring optionally containing 1, 2 or 3 heteroatoms independently selected from nitrogen, oxygen and sulphur and the resulting ring system is optionally substituted, on available carbon atoms, by 1, 2 or 3 substituents independently selected from C₁₋₄alkyl, hydroxyl, cyano, trifluoromethyl, trifluoromethoxy, halo, C₁₋₄alkoxy, C₁₋₄alkoxyC₁₋₄alkyl, amino, N—C₁₋₄alkylamino, di-N,N—(C₁₋₄alkyl)amino, N—C₁₋₄alkylcarbamoyl, di-N,N—(C₁₋₄alkyl)carbamoyl, C₁₋₁₄alkylS(O)ᵣ— and C₁₋₄alkylS(O)ᵣC₁₋₄alkyl (wherein r is independently selected 0, 1 and 2) and optionally substituted, on an available nitrogen, by a substituent independently selected from C₁₋₄alkyl, C₂₋₄alkanoyl and C₁₋₄alkanesulphonyl each of which is optionally substituted by 1, 2 or 3 substituent independently selected from hydroxyl, halo, C₁₋₄alkoxy, carboxy and cyano];
R¹⁶ is independently selected from, C₁₋₃alkyl optionally substituted by 1, 2 or 3 substituents independently selected from hydroxyl, halo, C₁₋₄alkoxy, carboxy and cyano;
R^{16′}, R^{16″} and R^{16′″} are independently selected from hydrogen and C₁₋₃alkyl optionally substituted by 1, 2, or 3 substituents independently selected from hydroxyl, halo, C₁₋₄alkoxy, carboxy and cyano);
or a pharmaceutically-acceptable salt thereof.

8. A method according to claim 7 wherein the disease treated by producing an 11βHSD1 inhibitory effect is type 2 diabetes.

9. A method according to claim 7 wherein the disease treated by producing an 11βHSD1 inhibitory effect is obesity.

10. A process for preparing a compound of formula (I) or a pharmaceutically acceptable salt thereof which process (wherein variable groups are, unless otherwise specified, as defined in claim 1) comprises:
i) reacting a compound of formula: 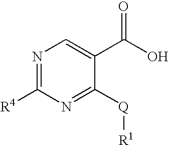
or a reactive derivative thereof with an amine of formula HNR²R³;
ii) reacting together compounds of the formulae: 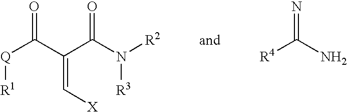
wherein X is dialkylamino or lower alkoxy;
iii) when R⁴ is —SR¹⁰, reacting a compound of the formula: 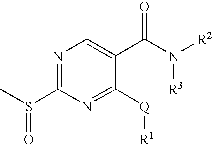
with the appropriate nucleophile to convert —SOMe to —R⁴;
iv) reacting an activated derivative of a compound of the formula: 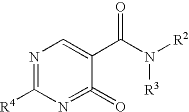
with a nucleophile of the formula Q-R¹;
v) reacting a compound of the formula: 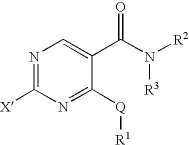
wherein X′ is halo with a nucleophile R⁴;
and thereafter if necessary or desirable:
i) converting a compound of the formula (1) into another compound of the formula (1);
ii) removing any protecting groups;
iii) resolving enantiomers;
iv) forming a pharmaceutically-acceptable salt thereof.
